(19) United States
(12) Patent Application Publication
(10) Pub. No.: US 2011/0009435 A1
(43) Pub. Date: Jan. 13, 2011

(54) HETERO BICYCLIC CARBOXAMIDE DERIVATIVES AND THEIR PHARMACEUTICAL USE AND COMPOSITIONS

(75) Inventors: Mark E. Schnute, Dardenne Prairie, MO (US); Peter G. Ruminski, Ballwin, MO (US); Cathleen E. Hanau, Chesterfield, MO (US); Joseph W. Stobach, Wentzville, MO (US); Jeffery Carroll, Saint Louis, MO (US); Kirby Sample, St. Louis, MO (US)

Correspondence Address: PHARMACIA CORPORATION;c/o Pfizer Inc., Eastern Point Road, MS9114, Groton, CT, 06340, US

(73) Assignee: Pfizer, Inc., New York, NY (US)

(21) Appl. No.: 12/663,157

(22) PCT Filed: May 19, 2008
(86) PCT No.: PCT/IB08/01279

§ 371 Date: Sep. 20, 2010

Related U.S. Application Data
(60) Provisional application No. 60/941,998, filed on Jun. 5, 2007.

(51) Int. Cl. A61K 31/517; C07D 491/04; C07D 239/72; A61K 31/519; A61P 37/00; A61P 17/06; A61P 19/02; A61P 35/00; A61P 11/00; A61P 11/06; A61P 1/06

(52) U.S. Cl. 514/266.24; 544/278; 544/287; 514/258.1

(57) ABSTRACT

The present invention relates to compounds of Formula I

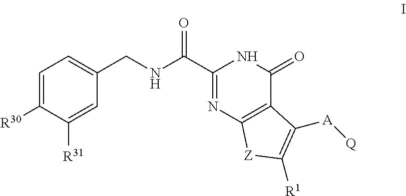

and pharmaceutically acceptable salts thereof, processes for the preparation of, intermediates used in the preparation of, and compositions containing such compounds and the uses of such compounds as anti-inflammatory agents.

### FIELD OF THE INVENTION

This invention relates to hetero bicyclic carboxamide derivatives. Such compounds have been shown to inhibit matrix metalloproteinase enzymes. These compounds are useful for treating diseases resulting from MMP-mediated tissue breakdown such as heart disease, cardiac insufficiency, inflammatory bowel disease, multiple sclerosis, osteoarthritis, rheumatoid arthritis, arthritis other than osteo- or rheumatoid arthritis, heart failure, age-related macular degeneration, chronic obstructive pulmonary disease, asthma, periodontal diseases, psoriasis, atherosclerosis, fibrotic disorders in the kidney, lung, and/or osteoporosis.

### BACKGROUND OF THE INVENTION

Matrix metalloproteinases (sometimes referred to as MMPs) are naturally occurring enzymes found in most mammals. Over-expression and activation of MMPs, or an imbalance between MMPs and inhibitors of MMPs, have been suggested as factors in the pathogenesis of diseases characterized by the breakdown of extracellular matrix or connective tissues.

To date, 24 different members of the MMP family have been identified in vertebrates, 23 of which are found in human, including collagenases (MMP-1, MMP-8, MMP-13), gelatinases (MMP-2 and MMP-9), stromelysins (MMP-3, MMP-10, MMP-11), matrilysins (MMP-7 and MMP-26), membrane-type (MMT-14, MMP-15, MMP-16, MMT-17, MMP-24, MMT-25), as well as metalloelastases (MMP-12, MMP-19, MMP-20, MMP-22, MMP-23) (See Visse and Nagase (2003) Circ. Res. 92:827-839) These enzymes have been implicated with a number of diseases which result from breakdown of connective tissue, including such diseases as rheumatoid arthritis, osteoarthritis, osteoporosis, periodontitis, multiple sclerosis, gingivitis, corneal epidermal and gastric ulceration, atherosclerosis, neointimal proliferation which leads to restenosis and ischemic heart failure, and tumor metastasis. Inhibiting matrix metalloproteinase enzymes, thereby curtailing and/or eliminating the breakdown of connective tissues that results in the disease states, is a recognized method for preventing and treating these and other diseases.

There is a catalytic zinc domain in matrix metalloproteinases that is typically the focal point for inhibitor design. The modification of substrates by introducing zinc-chelating groups has generated potent inhibitors such as peptide hydroxamates and thiol-containing peptides. Peptide hydroxamates and the natural endogenous inhibitors of MMPs (TIMPs) have been used successfully to treat animal models of cancer and inflammation. MMP inhibitors have also been used to prevent and treat congestive heart failure and other cardiovascular diseases; see, e.g., U.S. Pat. No. 5,948,780.

A major limitation on the use of currently known MMP inhibitors is their lack of specificity for any particular enzyme. Recent data has established that specific MMP enzymes are associated with some diseases, with no effect on others. The MMPs are generally categorized based on their substrate specificity, and indeed the collagenase subfamily of MMP-1, MMP-8, and MMP-13 selectively cleave native interstitial collagens, and thus are associated only with diseases linked to such interstitial collagen tissue. This is evidenced by the recent discovery that MMP-13 alone is over-expressed in breast carcinoma, while MMP-1 alone is over expressed in papillary carcinoma (see Chen et al., J. Am. Chem. Soc., 2000; 122:9648-54).

Selective inhibitors of MMP-13 include a compound named WAY 170523, which has been reported by Chen et al., supra, 2000. Other selective inhibitors of MMP-13 are reported in International Patent Application Publication No. WO 05/105760. Other selective inhibitors of MMP-13 are reported in U.S. Patent Application Publication No. US20030229103. Other selective inhibitors of MMP-13 are reported in U.S. Patent Application Publication No. US20040167120. Other selective inhibitors of MMP-13 are reported in U.S. Patent Application Publication No. US20050004111. Other selective inhibitors of MMP-13 are reported in U.S. Patent Application Publication No. US20060173183. Other selective inhibitors of MMP-13 are reported in International Patent Application Publication No. WO 06/128184. Other selective inhibitors of MMP-13 are reported in Co-assigned International Patent Application Publication No. WO 02/64572. Other selective inhibitors of MMP-13 are reported in Co-assigned International Patent Application Publication No. WO 02/64599. U.S. Pat. No. 6,008,243 discloses inhibitors of MMP-13. However, no selective or nonselective inhibitor of MMP-13 has been approved and marketed for the treatment of any disease in any mammal. Accordingly, the need continues to find new low molecular weight compounds that are potent and selective MMP inhibitors, and that have an acceptable therapeutic index of toxicity/potency to make them amenable for use clinically in the prevention and treatment of the associated disease states.

### SUMMARY OF THE INVENTION

The present invention provides compounds of Formula I:

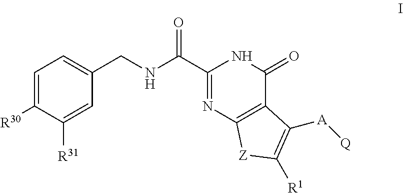

wherein:

Z is —CH═CH— or —S—;

A is —(CH₂)ₘO(CH₂)ₙ, wherein m is 0, 1, 2, or 3 and n is 1, 2, or 3;

Q is

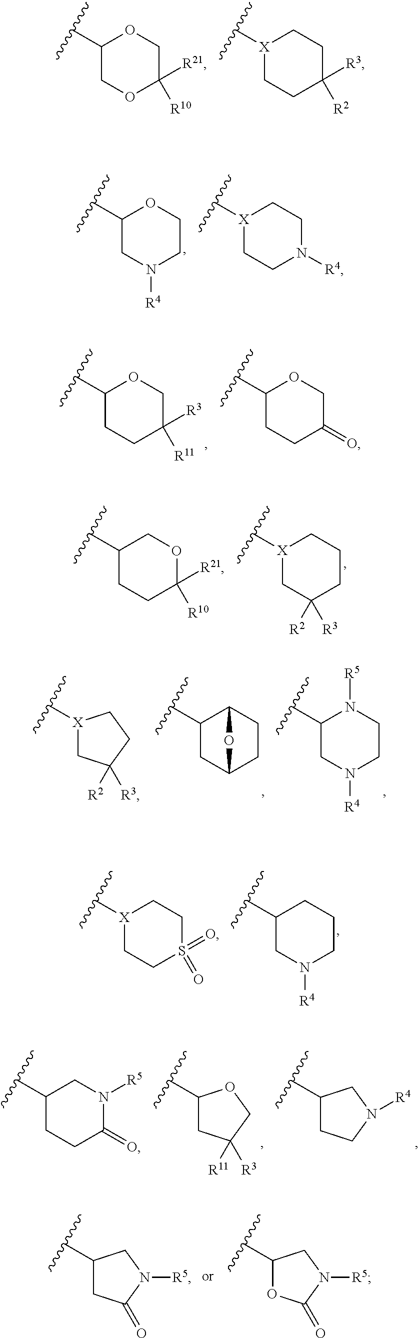

X is N or CH, provided that when X is N, n is 2 or 3;

R¹ is H or F;

R² is H, CN, —OR⁵, R¹², C(═O)R⁷, —NR⁸R³³, —NR⁹C(═O)R⁹, —NR⁸SO₂R³⁴, or —SO₂R¹²;

R³ is H, CN, or —OR²²;

R⁴ is H, —(C₁₋₆ alkylene)R⁶, —C(═O)R⁹, or —SO₂R¹²;

R⁵ is H or —(C₁₋₆ alkyl), wherein said C₁₋₆ alkyl may be substituted by one or more R²⁶ substituents;

R⁶ is H, CN, —OR²³, —SO₂R³⁵, —NR²⁴C(═O)R²³, —NR²⁴SO₂R³⁵, or

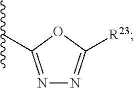

R⁷ is —(C₁₋₆ alkyl), —(C₁₋₆ alkylene)OH, —NHR²⁴, or —OR²⁵;

R⁹ is —(C₁₋₆ alkylene)R²⁸, —NHR²⁴, or —OR²⁵;

R¹⁰ is H, CN, R¹², or —C(═O)R⁷;

R¹¹ is H, CN, —OR⁵, R¹², —C(═O)R⁷, —NR⁸R³³, —NR⁹C(═O)R⁹, —NR⁸SO₂R³⁴, or —SO₂R¹²;

R¹² is —(C₁₋₆ alkyl), wherein said C₁₋₆ alkyl may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of said C₁₋₆ alkyl is not substituted by more than one CN or more than one —OR²³;

R⁸, R²¹, R²², R²⁴, R²⁵, and R³³ are independently H or —(C₁₋₆ alkyl);

R²³ is H, —(C₁₋₆ alkyl), or —(C₁₋₆ alkylene)OH;

R²⁶ is H, OH, halo, NH₂, or SH;

R²⁸ is H or —OR²⁹;

R²⁹ is H or —C(═O)(C₁₋₆ alkyl);

R³⁰ is H or F;

R³¹ is Cl, Br, —OR³², (C₁₋₆ alkyl), —OCH₂CH₂OR²⁵, —(C₃₋₆ cycloalkyl), or CN;

R³² is —(C₁₋₆ alkyl) optionally substituted with one, two, or three F;

R³⁴ is —(C₁₋₆ alkyl); and

R³⁵ is —(C₁₋₆ alkyl) or —(C₁₋₆ hydroxyalkyl);

or a pharmaceutically acceptable salt thereof;

provided that if Q is

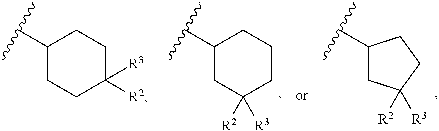

R² and R³ are not both H.

This invention also includes pharmaceutically acceptable salts, solvates and hydrates of compounds of Formula I. This invention also includes all tautomers and stereochemical isomers of these compounds.

This invention also is directed, in part, to a method for treating an MMP-13 mediated disorder in a mammal. Such disorders include rheumatoid arthritis and osteoarthritis. The method comprises administering a compound of Formula I or a pharmaceutically acceptable salt thereof, to the mammal in an amount that is therapeutically-effective to treat the condition.

### DETAILED DESCRIPTION OF THE INVENTION

One embodiment of the invention is a compound of Formula I as shown above.

Another embodiment of the invention is a compound of Formula I as shown above wherein Q is

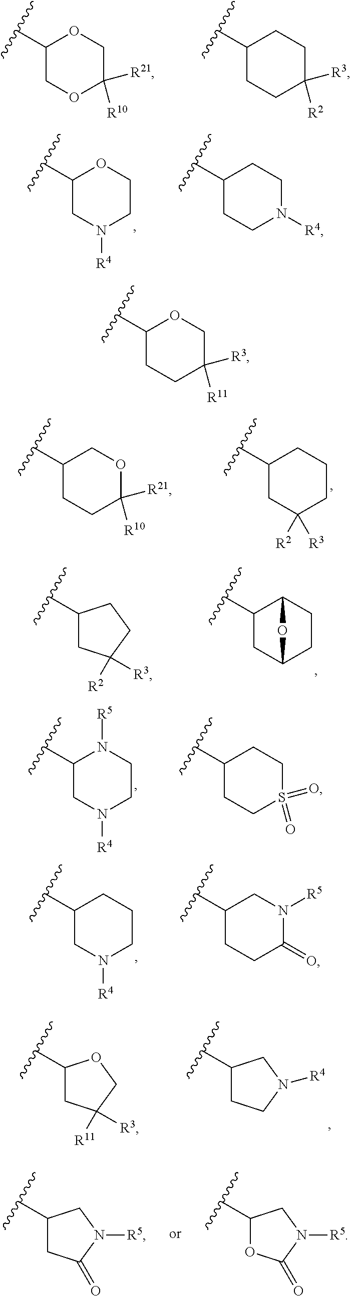

Another embodiment of the invention is a compound of Formula I as shown above wherein:

R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;

R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;

R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;

R⁸, R²¹, R²², R²⁴, R²⁵, and R³³ are independently H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;

R⁹ is —(C₁₋₆ alkylene)R²⁸, —NHR²⁴, or —OR²⁶, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;

R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁶, —NR⁸R³³, —NR²⁴C(═O)R²³, and —NR²⁴SO₂R³⁶, provided that any one carbon atom of R¹² is not substituted by more than one CN or more than one —OR²³;

R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;

R²⁶ is H, OH, F, Cl, Br, NH₂, or SH;

R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;

R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁶, cyclopropyl, cyclobutyl, cyclopentyl, cyclohexyl, or CN;

R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;

R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and

R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

Another embodiment of the invention is a compound of Formula II:

wherein:

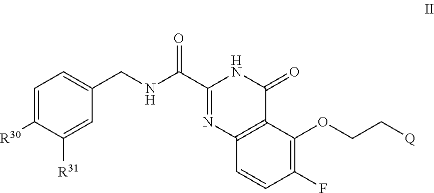

Q is

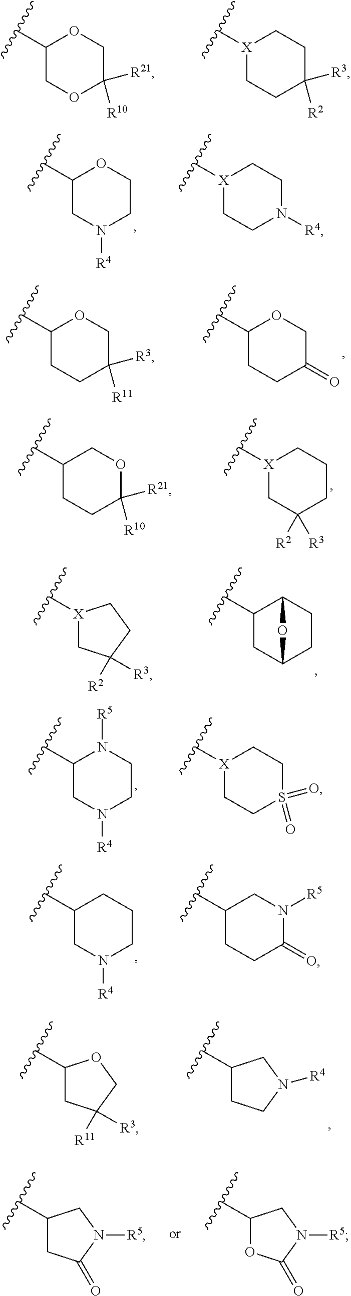

X is N or CH;

R² is H, CN, R¹², C(═O)R⁷, —NR⁸R³³, —NR⁸C(═O)R⁹, —NR⁸SO₂R³⁴, or —SO₂R¹²;

R³ is H, CN, or —OR²²;

R⁴ is H, —(C₁₋₆ alkylene)R⁶, —C(═O)R⁹, or —SO₂R¹²;

R⁵ is H or —(C₁₋₆ alkyl), wherein said C₁₋₆ alkyl may be substituted by one or more R²⁶ substituents;

R⁶ is H, CN, —OR²³, —SO₂R³⁵, —NR²⁴C(═O)R²³, —NR²⁴SO₂R³⁵, or

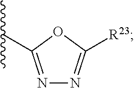

R⁷ is —(C₁₋₆ alkyl), —(C₁₋₆ alkylene)OH, —NHR²⁴, or —OR²⁵;

R⁹ is —(C₁₋₆ alkylene)R²⁸, —NHR²⁴, or —OR²⁵;

R¹⁰ is H, CN, R¹², or —C(═O)R⁷;

R¹¹ is H, CN, R¹², —C(═O)R⁷, —NR⁸R³³, —NR⁸C(═O)R⁹, —NR⁸SO₂R³⁴, or —SO₂R¹²;

R¹² is —(C₁₋₆ alkyl), wherein said C₁₋₆ alkyl may be optionally substituted by one or more substituents selected from CN, —OR²³, —SO₂R³⁶, —NR⁸R³³, —NR²⁴C(═O)R²³, and —NR²⁴SO₂R³⁵, provided that any one carbon atom of said C₁₋₆ alkyl is not substituted by more than one CN or more than one —OR²³;

R⁸, R²¹, R²², R²⁴, R²⁵, and R³³ are independently H or —(C₁₋₆ alkyl);

R²³ is H, —(C₁₋₆ alkyl), or —(C₁₋₆ alkylene)OH;

R²⁶ is H, OH, halo, NH₂, or SH;

R²⁸ is H or —OR²⁹;

R²⁹ is H or —C(═O)(C₁₋₆ alkyl);

R³⁰ is H or F;

R³¹ is Cl, Br, —OR³², (C₁₋₆ alkyl), —OCH₂CH₂OR²⁵, —(C₃₋₆ cycloalkyl), or CN;

R³² is —(C₁₋₆ alkyl) optionally substituted with one, two, or three F;

R³⁴ is —(C₁₋₆ alkyl); and

R³⁵ is —(C₁₋₆ alkyl) or —(C₁₋₆ hydroxyalkyl);

or a pharmaceutically acceptable salt thereof;

provided that if Q is

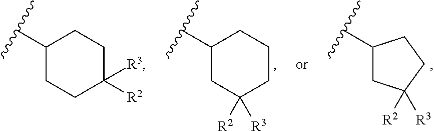

R² and R³ are not both H.

Another embodiment of the invention is a compound of Formula II as shown above

wherein Q is

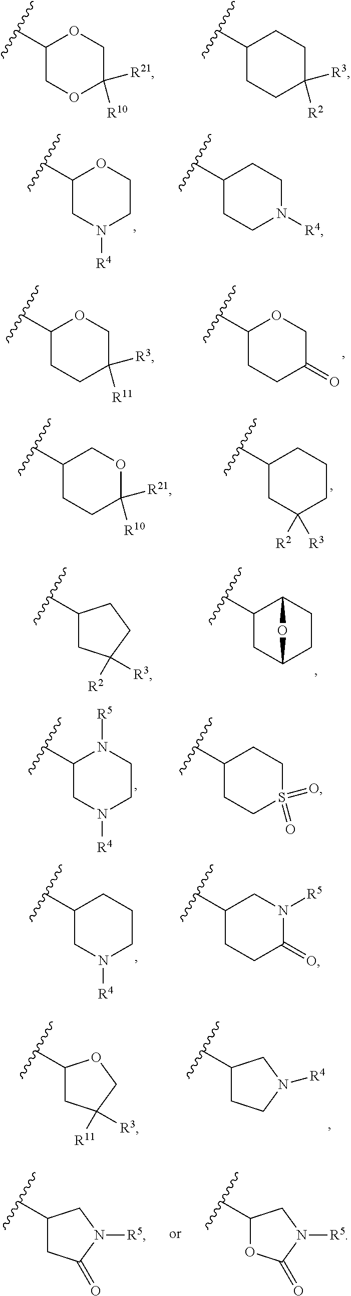

Another embodiment of the invention is a compound of Formula II as shown above

wherein Q is

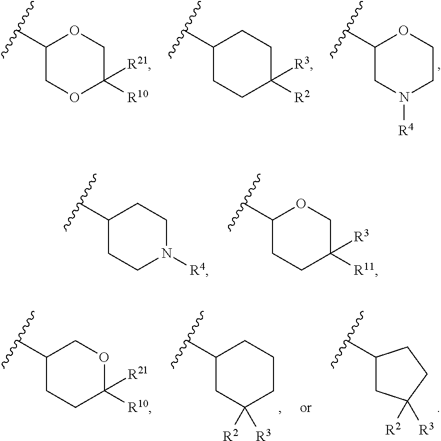

Another embodiment of the invention is a compound of Formula II as shown above

wherein Q is

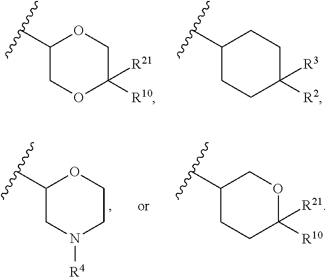

Another embodiment of the invention is a compound of Formula II as shown above

wherein Q is

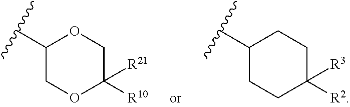

Another embodiment of the invention is a compound of Formula II as shown above wherein R¹⁰ is CN, R¹², or —C(═O)R⁷; and R¹¹ is CN, —OR⁵, R¹², —C(═O)R⁷, —NR⁸C(═O)R⁹, —NR⁸SO₂R³⁴, or —SO₂R¹².

Another embodiment of the invention is a compound of Formula II as shown above wherein Q is

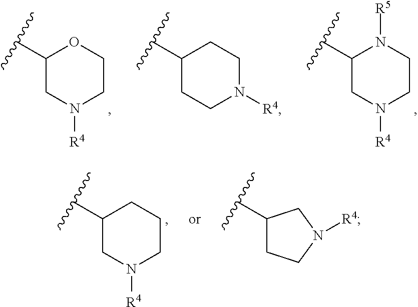

### and R⁴ is —C(═O)R⁹ or —SO₂R¹².

Another embodiment of the invention is a compound of Formula II as shown above wherein Q is

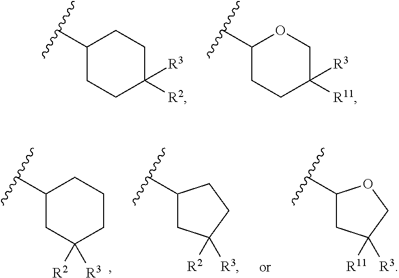

Another embodiment of the invention is a compound of Formula II as shown above wherein Q is

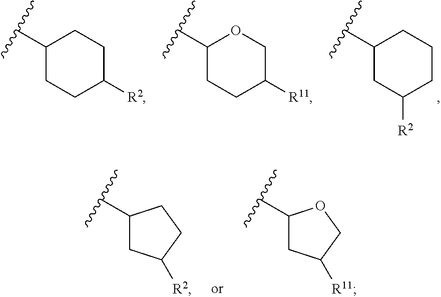

### R² is CN, OH,

R¹², C(═O)R⁷, —NR⁸R³³, —NR⁸C(═O)R⁹, or —NR⁸SO₂R³⁴; and R¹¹ is CN, OH, R¹², —C(═O)R⁷, —NR⁸C(═O)R⁹, or —NR⁸SO₂R³⁴.

Another embodiment of the invention is a compound of Formula II as shown above wherein Q is

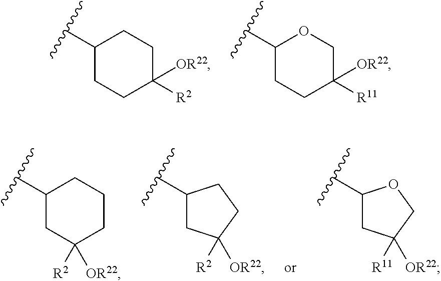

### and R² and R¹¹ are R¹² or —C(═O)R⁷.

Another embodiment of the invention is a compound of Formula II as shown above wherein Q is

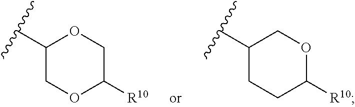

### and R¹⁰ is R¹² or —C(═O)R⁷.

Another embodiment of the invention is a compound of Formula IIA:

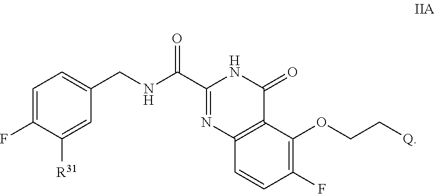

wherein:

Q is

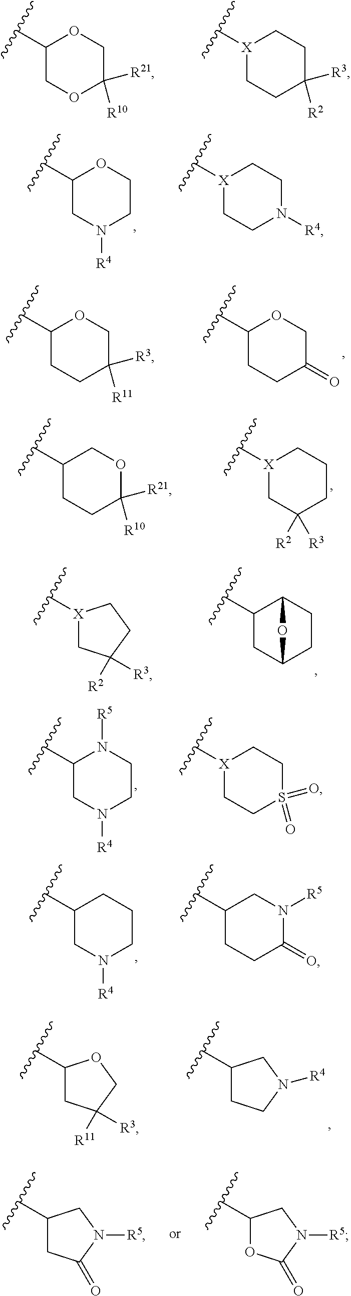

X is N or CH;

R² is H, CN, R¹², —C(═O)R⁷, —NR⁸R³³, —NR⁸C(═O)R⁹, —NR⁸SO₂R³⁴, or —SO₂R¹²;

R³ is H, CN, or —OR²²;

R⁴ is H, —(C₁₋₆ alkylene)R⁶, —C(═O)R⁹, or —SO₂R¹²;

R⁵ is H or —(C₁₋₆ alkyl), wherein said C₁₋₆ alkyl may be substituted by one or more R²⁶ substituents;

R⁶ is H, CN, —OR²³, —SO₂R³⁶, —NR²⁴C(═O)R²³, —NR²⁴SO₂R³⁵, or

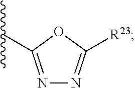

R⁷ is —(C₁₋₆ alkyl), —(C₁₋₆ alkylene)OH, —NHR²⁴, or —OR²⁵;

R⁹ is —(C₁₋₆ alkylene)R²⁸, —NHR²⁴, or —OR²⁵;

R¹⁰ is H, CN, R¹², or —C(═O)R⁷;

R¹¹ is H, CN, —OR⁵, R¹², —C(═O)R⁷, —NR⁸R³³, —NR⁸C(═O)R⁹, —NR⁸SO₂R³⁴, or —SO₂R¹²;

R¹² is —(C₁₋₆ alkyl), wherein said C₁₋₆ alkyl may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of said C₁₋₆ alkyl is not substituted by more than one CN or more than one —OR²³;

R⁸, R²¹, R²², R²⁴, R²⁵, and R³³ are independently H or —(C₁₋₆ alkyl);

R²³ is H, —(C₁₋₆ alkyl), or —(C₁₋₆ alkylene)OH;

R²⁶ is H, OH, halo, NH₂, or SH;

R²⁸ is H or —OR²⁹;

R²⁹ is H or —C(═O)(C₁₋₆ alkyl);

R³¹ is Cl, Br, —OR³², (C₁₋₆ alkyl), —OCH₂CH₂OR²⁵, —(C₃₋₆ cycloalkyl), or CN;

R³² is —(C₁₋₆ alkyl) optionally substituted with one, two, or three F;

R³⁴ is —(C₁₋₆ alkyl); and

R³⁵ is —(C₁₋₆ alkyl) or —(C₁₋₆ hydroxyalkyl);

or a pharmaceutically acceptable salt thereof;

provided that if Q is

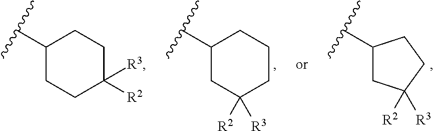

R² and R³ are not both H.

Another embodiment of the invention is a compound of Formula IIB:

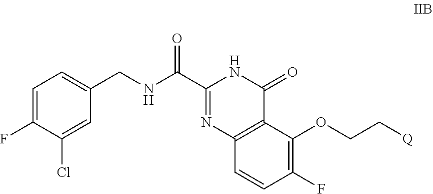

wherein Q, X, R²-R¹², R²¹-R²⁶, R²⁸, R²⁹, and R³²-R³⁵ are as defined above for Formula IIA; or a pharmaceutically acceptable salt thereof;

provided that if Q is

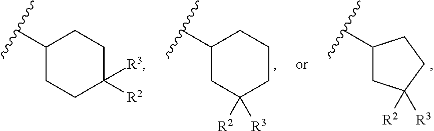

R² and R³ are not both H.

Another embodiment of the invention is a compound of Formula IIC:

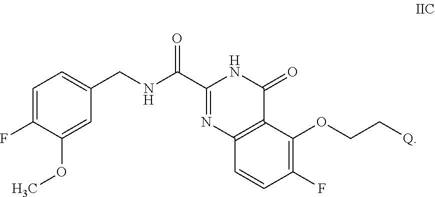

wherein Q, X, R²-R¹², R²¹-R²⁶, R²⁸, R²⁹, and R³²-R³⁵ are as defined above for Formula IIA; or a pharmaceutically acceptable salt thereof;

provided that if Q is

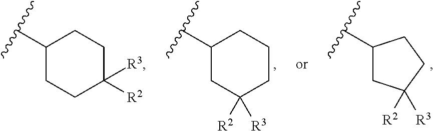

R² and R³ are not both H.

Another embodiment of the invention is a compound of Formula II-1, shown in Table 1.

TABLE 1
II-1

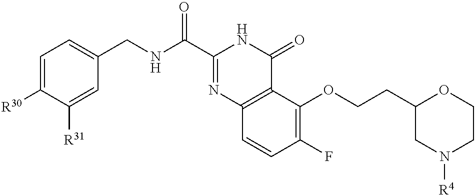

| Compound Name | R⁴ | R³⁰ | R³¹ |
|---|---|---|---|
| N-(4-fluoro-3-methoxybenzyl)-5-[2-(4-acetylmorpholin-2-yl)ethoxy]-6-fluoro-4- | —C(═O)CH₃ | F | —OCH₃ |
| oxo-3,4-dihydroquinazoline-2-carboxamide | | | |
| N-(4-fluoro-3-chlorobenzyl)-5-[2-(4-acetylmorpholin-2-yl)ethoxy]-6-fluoro-4-oxo- | —C(═O)CH₃ | F | Cl |
| 3,4-dihydroquinazoline-2-carboxamide | | | |
| N-(3-methoxybenzyl)-5-[2-(4-acetylmorpholin-2-yl)ethoxy]-6-fluoro-4-oxo-3,4- | —C(═O)CH₃ | H | —OCH₃ |
| dihydroquinazoline-2-carboxamide | | | |
| N-(3-chlorobenzyl)-5-[2-(4-acetylmorpholin-2-yl)ethoxy]-6-fluoro-4-oxo-3,4- | —C(═O)CH₃ | H | Cl |
| dihydroquinazoline-2-carboxamide | | | |
| N-(4-fluoro-3-methoxybenzyl)-6-fluoro-5-[2-(morpholin-2-yl)ethoxy]-4-oxo-3,4- | H | F | —OCH₃ |
| dihydroquinazoline-2-carboxamide | | | |
| N-(4-fluoro-3-chlorobenzyl)-6-fluoro-5-[2-(morpholin-2-yl)ethoxy]-4-oxo-3,4- | H | F | Cl |
| dihydroquinazoline-2-carboxamide | | | |
| N-(3-methoxybenzyl)-6-fluoro-5-[2-(morpholin-2-yl)ethoxy]-4-oxo-3,4- | H | H | —OCH₃ |
| dihydroquinazoline-2-carboxamide | | | |
| N-(3-chlorobenzyl)-6-fluoro-5-[2-(morpholin-2-yl)ethoxy]-4-oxo-3,4- | H | H | Cl |
| dihydroquinazoline-2-carboxamide | | | |
| N-(4-fluoro-3-methoxybenzyl)-6-fluoro-5-{2-[4-(methylsulfonyl)morpholin-2- | —SO₂CH₃ | F | —OCH₃ |
| yl]ethoxy}-4-oxo-3,4-dihydroquinazoline-2-carboxamide | | | |
| N-(4-fluoro-3-chlorobenzyl)-6-fluoro-5-{2-[4-(methylsulfonyl)morpholin-2- | —SO₂CH₃ | F | Cl |
| yl]ethoxy}-4-oxo-3,4-dihydroquinazoline-2-carboxamide | | | |
| N-(3-methoxybenzyl)-6-fluoro-5-{2-[4-(methylsulfonyl)morpholin-2-yl]ethoxy}-4- | —SO₂CH₃ | H | —OCH₃ |
| oxo-3,4-dihydroquinazoline-2-carboxamide | | | |
| N-(3-chlorobenzyl)-6-fluoro-5-{2-[4-(methylsulfonyl)morpholin-2-yl]ethoxy}- | —SO₂CH₃ | H | Cl |
| 4-oxo-3,4-dihydroquinazoline-2-carboxamide | | | |

Another embodiment of the invention is a compound of Formula II-2, shown in Table 2.

TABLE 2
II-2

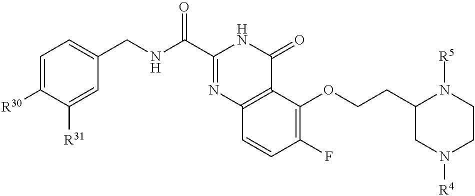

| Compound Name | R⁴ | R⁵ | R³⁰ | R³¹ |
|---|---|---|---|---|
| N-(4-fluoro-3-methoxybenzyl)-5-[2-(4-acetylpiperazin-2-yl)ethoxy]-6-fluoro-4- | —C(═O)CH₃ | H | F | —OCH₃ |
| oxo-3,4-dihydroquinazoline-2-carboxamide | | | | |
| N-(4-fluoro-3-chlorobenzyl)-5-[2-(4-acetylpiperazin-2-yl)ethoxy]-6-fluoro-4- | —C(═O)CH₃ | H | F | Cl |
| oxo-3,4-dihydriquinazoline-2-carboxamide | | | | |
| N-(3-methoxybenzyl)-5-[2-(4-acetylpiperazin-2-yl)ethoxy]-6-fluoro-4-oxo-3,4- | —C(═O)CH₃ | H | H | —OCH₃ |
| dihydroquinazoline-2-carboxamide | | | | |
| N-(3-chlorobenzyl)-5-[2-(4-acetylpiperazin-2-yl)ethoxy]-6-fluoro-4-oxo-3,4- | —C(═O)CH₃ | H | H | Cl |
| dihydroquinazoline-2-carboxamide | | | | |
| N-(4-fluoro-3-methoxybenzyl)-6-fluoro-5-{2-[4-(methylsulfonyl)piperazin-2- | —SO₂CH₃ | H | F | —OCH₃ |
| yl]ethoxy}-4-oxo-3,4-dihydroquinazoline-2-carboxamide | | | | |
| N-(4-fluoro-3-chlorobenzyl)-6-fluoro-5-{2-[4-(methylsulfonyl)piperazin-2- | —SO₂CH₃ | H | F | Cl |
| yl]ethoxy}-4-oxo-3,4-dihydroquinazoline-2-carboxamide | | | | |
| N-(3-methoxybenzyl)-6-fluoro-5-{2-[4-(methylsulfonyl)piperazain-2-yl]ethoxy}- | —SO₂CH₃ | H | H | —OCH₃ |
| 4-oxo-3,4-dihydroquinazoline-2-carboxamide | | | | |
| N-(3-chlorobenzyl)-6-fluoro-5-{2-[4-(methylsulfonyl)piperazin-2-yl]ethoxy}-4- | —SO₂CH₃ | H | H | Cl |
| oxo-3,4-dihydroquinazoline-2-carboxamide | | | | |

Another embodiment of the invention is a compound of Formula II-3, shown in Table 3.

TABLE 3
II-3

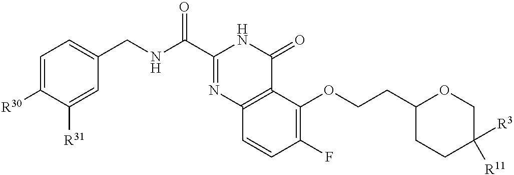

| Compound Name | R³ | R¹¹ | R³⁰ | R³¹ |
|---|---|---|---|---|
| N-(4-fluoro-3-methoxybenzyl)-5-{2-[5-cyano-5-(hydroxymethyl)- | CN | —CH₂OH | F | —OCH₃ |
| tetrahydro-2H-pyran-2-yl]ethoxy}-6-fluoro-4-oxo-3,4- | | | | |
| dihydroquinazoline-2-carboxamide | | | | |
| N-(4-fluoro-3-chlorobenzyl)-5-{2-[5-cyano-5-(hydroxymethyl)- | CN | —CH₂OH | F | Cl |
| tetrahydro-2H-pyran-2-yl]ethoxy}-6-fluoro-4-oxo-3,4- | | | | |
| dihydroquinazoline-2-carboxamide | | | | |
| N-(3-methoxybenzyl)-5-{2-[5-cyano-5-(hydroxymethyl)- | CN | —CH₂OH | H | —OCH₃ |
| tetrahydro-2H-pyran-2-yl]ethoxy}-6-fluoro-4-oxo-3,4- | | | | |
| dihydroquinazoline-2-carboxamide | | | | |
| N-(3-chlorobenzyl)-5-{2-[5-cyano-5-(hydroxymethyl)-tetrahydro- | CN | —CH₂OH | H | Cl |
| 2H-pyran-2-yl]ethoxy}-6-fluoro-4-oxo-3,4-dihydroquinazoline-2- | | | | |
| carboxamide | | | | |
| N-(4-fluoro-3-methoxybenzyl)-6-fluoro-5-{2-[5-(hydroxymethyl)- | H | —CH₂OH | F | —OCH₃ |
| tetrahydro-2H-pyran-2-yl]ethoxy}-4-oxo-3,4-dihydroquinazoline-2- | | | | |
| carboxamide | | | | |
| N-(4-fluoro-3-chlorobenzyl)-6-fluoro-5-{2-[5-(hydroxymethyl)- | H | —CH₂OH | F | Cl |
| tetrahydro-2H-pyran-2-yl]ethoxy}-4-oxo-3,4-dihydroquinazoline-2- | | | | |
| carboxamide | | | | |
| N-(3-methoxybenzyl)-6-fluoro-5-{2-[5-(hydroxymethyl)-tetrahydro- | H | —CH₂OH | H | —OCH₃ |
| 2H-pyran-2-yl]ethoxy}-4-oxo-3,4-dihydroquinazoline-2- | | | | |
| carboxamide | | | | |
| N-(3-chlorobenzyl)-6-fluoro-5-{2-[5-(hydroxymethyl)-tetrahydro- | H | —CH₂OH | H | Cl |
| 2H-pyran-2-yl]ethoxy}-4-oxo-3,4-dihydroquinazoline-2- | | | | |
| carboxamide | | | | |
| N-(4-fluoro-3-methoxybenzyl)-6-fluoro-4-oxo-5-[2-(tetrahydro-2H- | H | H | F | —OCH₃ |
| pyran-2-yl)ethoxy]-3,4-dihydroquinazoline-2-carboxamide | | | | |
| N-(4-fluoro-3-chlorobenzyl)-6-fluoro-4-oxo-5-[2-(tetrahydro-2H- | H | H | F | Cl |
| pyran-2-yl)ethoxy]-3,4-dihydroquinazoline-2-carboxamide | | | | |
| N-(3-methoxybenzyl)-6-fluoro-4-oxo-5-[2-(tetrahydro-2H-pyran-2- | H | H | H | —OCH₃ |
| yl)ethoxy]-3,4-dihydroquinazoline-2-carboxamide | | | | |
| N-(3-chlorobenzyl)-6-fluoro-4-oxo-5-[2-(tetrahydro-2H-pyran-2- | H | H | H | Cl |
| yl)ethoxy]-3,4-dihydroquinazoline-2-carboxamide | | | | |
| N-(4-fluoro-3-methoxybenzyl)-6-fluoro-5-{2-[5-(2- | H | —NHC(═O)CH₂OH | F | —OCH₃ |
| hydroxyacetamido)-tetrahydro-2H-pyran-2-yl]ethoxy}-4-oxo-3,4- | | | | |
| dihydroquinazoline-2-carboxamide | | | | |
| N-(4-fluoro-3-chlorobenzyl)-6-fluoro-5-{2-[5-(2- | H | —NHC(═O)CH₂OH | F | Cl |
| hydroxyacetamido)-tetrahydro-2H-pyran-2-yl]ethoxy}-4-oxo-3,4- | | | | |
| dihydroquinazoline-2-carboxamide | | | | |
| N-(3-methoxybenzyl)-6-fluoro-5-{2-[5-(2-hydroxyacetamido)- | H | —NHC(═O)CH₂OH | H | —OCH₃ |
| tetrahydro-2H-pyran-2-yl]ethoxy}-4-oxo-3,4-dihydroquinazoline-2- | | | | |
| carboxamide | | | | |
| N-(3-chlorobenzyl)-6-fluoro-5-{2-[5-(2-hydroxyacetamido)- | H | —NHC(═O)CH₂OH | H | Cl |
| tetrahydro-2H-pyran-2-yl]ethoxy}-4-oxo-3,4-dihydroquinazoline-2- | | | | |
| carboxamide | | | | |
| N-(4-fluoro-3-methoxybenzyl)-5-[2-(5-acetamido-tetrahydro-2H- | H | —NHC(═O)CH₃ | F | —OCH₃ |
| pyran-2-yl)ethoxy]-6-fluoro-4-oxo-3,4-dihydroquinazoline-2- | | | | |
| carboxamide | | | | |
| N-(4-fluoro-3-chlorobenzyl)-5-[2-(5-acetamido-tetrahydro-2H- | H | —NHC(═O)CH₃ | F | Cl |
| pyran-2-yl)ethoxy]-6-fluoro-4-oxo-3,4-dihydroquinazoline-2- | | | | |
| carboxamide | | | | |
| N-(3-methoxybenzyl)-5-[2-(5-acetamido-tetrahydro-2H-pyran-2- | H | —NHC(═O)CH₃ | H | —OCH₃ |
| yl)ethoxy]-6-fluoro-4-oxo-3,4-dihydroquinazoline-2-carboxamide | | | | |
| N-(3-chlorobenzyl)-5-[2-(5-acetamido-tetrahydro-2H-pyran-2- | H | —NHC(═O)CH₃ | H | Cl |
| yl)ethoxy]-6-fluoro-4-oxo-3,4-dihydroquinazoline-2-carboxamide | | | | |
| N-(4-fluoro-3-methoxybenzyl)-6-fluoro-5-{2-[5- | H | —NHSO₂CH₃ | F | —OCH₃ |
| (methylsulfonamido)-tetrahydro-2H-pyran-2-yl]ethoxy}-4-oxo-3,4- | | | | |
| dihydroquinazoline-2-carboxamide | | | | |
| N-(4-fluoro-3-chlorobenzyl)-6-fluoro-5-{2-[5-(methylsulfonamido)- | H | —NHSO₂CH₃ | F | Cl |
| tetrahydro-2H-pyran-2-yl]ethoxy}-4-oxo-3,4-dihydroquinazoline-2- | | | | |
| carboxamide | | | | |
| N-(3-methoxybenzyl)-6-fluoro-5-{2-[5-(methylsulfonamido)- | H | —NHSO₂CH₃ | H | —OCH₃ |
| tetrahydro-2H-pyran-2-yl]ethoxy}-4-oxo-3,4-dihydroquinazoline-2- | | | | |
| carboxamide | | | | |
| N-(3-chlorobenzyl)-6-fluoro-5-{2-[5-(methylsulfonamido)- | H | —NHSO₂CH₃ | H | Cl |
| tetrahydro-2H-pyran-2-yl]ethoxy}-4-oxo-3,4-dihydroquinazoline-2- | | | | |
| carboxamide | | | | |
| N-(4-fluoro-3-methoxybenzyl)-6-fluoro-5-{2-[5-(hydroxymethyl)-5- | —OCH₃ | —CH₂OH | F | —OCH₃ |
| methoxy-tetrahydro-2H-pyran-2-yl]ethoxy}-4-oxo-3,4- | | | | |
| dihydroquinazoline-2-carboxamide | | | | |
| N-(4-fluoro-3-chlorobenzyl)-6-fluoro-5-{2-[5-(hydroxymethyl)-5- | —OCH₃ | —CH₂OH | F | Cl |
| methoxy-tetrahydro-2H-pyran-2-yl]ethoxy}-4-oxo-3,4- | | | | |
| dihydroquinazoline-2-carboxamide | | | | |
| N-(3-methoxybenzyl)-6-fluoro-5-{2-[5-(hydroxymethyl)-5- | —OCH₃ | —CH₂OH | H | —OCH₃ |
| methoxy-tetrahydro-2H-pyran-2-yl]ethoxy}-4-oxo-3,4- | | | | |
| dihydroquinazoline-2-carboxamide | | | | |
| N-(3-chlorobenzyl)-6-fluoro-5-{2-[5-(hydroxymethyl)-5-methoxy- | —OCH₃ | —CH₂OH | H | Cl |
| tetrahydro-2H-pyran-2-yl]ethoxy}-4-oxo-3,4-dihydroquinazoline-2- | | | | |
| carboxamide | | | | |
| N-(4-fluoro-3-methoxybenzyl)-6-fluoro-5-{2-[5-hydroxy-5- | OH | —CH₂OH | F | —OCH₃ |
| (hydroxymethyl)-tetrahydro-2H-pyran-2-yl]ethoxy}-4-oxo-3,4- | | | | |
| dihydroquinazoline-2-carboxamide | | | | |
| N-(4-fluoro-3-chlorobenzyl)-6-fluoro-5-{2-[5-hydroxy-5- | OH | —CH₂OH | F | Cl |
| (hydroxymethyl)-tetrahydro-2H-pyran-2-yl]ethoxy}-4-oxo-3,4- | | | | |
| dihydroquinazoline-2-carboxamide | | | | |
| N-(3-methoxybenzyl)-6-fluoro-5-{2-[5-hydroxy-5-(hydroxymethyl)- | OH | —CH₂OH | H | —OCH₃ |
| tetrahydro-2H-pyran-2-yl]ethoxy}-4-oxo-3,4-dihydroquinazoline-2- | | | | |
| carboxamide | | | | |
| N-(3-chlorobenzyl)-6-fluoro-5-{2-[5-hydroxy-5-(hydroxymethyl)- | OH | —CH₂OH | H | Cl |
| tetrahydro-2H-pyran-2-yl]ethoxy}-4-oxo-3,4-dihydroquinazoline-2- | | | | |
| carboxamide | | | | |

Another embodiment of the invention is a compound of Formula II-4, shown in Table 4.

TABLE 4
II-4

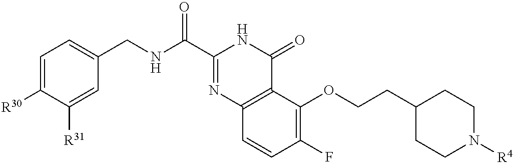

| Compound Name | R⁴ | R³⁰ | R³¹ |
|---|---|---|---|
| 6-fluoro-N-(3-methoxy-4-fluorobenzyl)-4-oxo-5-(2-piperidin-4- | H | F | —OCH₃ |
| ylethoxy)-3,4-dihydroxyquinazoline-2-carboxamide | | | |
| 6-fluoro-N-(3-chloro4-fluorobenzyl)-4-oxo-5-(2-piperidin-4-ylethoxy)- | H | F | Cl |
| 3,4-dihydroxyquinazoline-2-carboxamide | | | |
| 6-fluoro-N-(3-methoxybenzyl)-4-oxo-5-(2-piperidin-4-ylethoxy)-3,4- | H | H | —OCH₃ |
| dihydroquinazoline-2-carboxamide | | | |
| 6-fluoro-N-(3-chlorobenzyl)-4-oxo-5-(2-piperidin-4-ylethoxy)-3,4- | H | H | Cl |
| dihydroquinazoline-2-carboxamide | | | |
| 5-[2-(1-acetylpiperidin-4-yl)ethoxy]-6-fluoro-N-(3-methoxy-4- | —C(═O)CH₃ | F | —OCH₃ |
| fluorobenzyl)-4-oxo-3,4-dihydroquinazoline-2-carboxamide | | | |
| 5-[2-(1-acetylpiperidin-4-yl)ethoxy]-6-fluoro-N-(3-chloro-4- | —C(═O)CH₃ | F | Cl |
| fluorobenzyl)-4-oxo-3,4-dihydroquinazoline-2-carboxamide | | | |
| 5-[2-(1-acetylpiperidin-4-yl)ethoxy]-6-fluoro-N-(3-methoxybenzyl)-4- | —C(═O)CH₃ | H | —OCH₃ |
| oxo-3,4-dihydroquinazoline-2-carboxamide PF-04544352-00 | | | |
| 5-[2-(1-acetylpiperidin-4-yl)ethoxy]-6-fluoro-N-(3-chlorobenzyl)-4- | —C(═O)CH₃ | H | Cl |
| oxo-3,4-dihydroquinazolin-2-carboxamide | | | |
| 2-(4-{2-[(6-fluoro-2-{[(3-methoxy-4-fluorobenzyl)amino]carbonyl}-4- | —C(═O)CH₂OC(═O)CH₃ | F | —OCH₃ |
| oxo-3,4-dihydroquinazolin-5-yl)oxy]ethyl}piperidin-1-yl)-2-oxoethyl | | | |
| acetate | | | |
| 2-(4-{2-[(6-fluoro-2-{[(3-chloro-4-fluorobenzyl)amino]carbonyl}-4-oxo- | —C(═O)CH₂OC(═O)CH₃ | F | Cl |
| 3,4-dihydroquinazolin-5-yl)oxy]ethyl}piperidin-1-yl)-2-oxoethyl | | | |
| acetate | | | |
| 2-(4-{2-[(6-fluoro-2-{[(3-methoxybenzyl)amino]carbonyl}-4-oxo-3,4- | —C(═O)CH₂OC(═O)CH₃ | H | —OCH₃ |
| dihydroquinazolin-5-yl)oxy]ethyl}piperidin-1-yl)-2-oxoethyl acetate | | | |
| 2-(4-{2-[(6-fluoro-2-{[(3-chlorobenzyl)amino]carbonyl}-4-oxo-3,4- | —C(═O)CH₂OC(═O)CH₃ | H | Cl |
| dihydroquinazolin-5-yl)oxy]ethyl}piperidin-1-yl)-2-oxoethyl acetate | | | |
| 6-fluoro-5-{2-[1-(2-hydroxyethyl)piperidin-4-yl]ethoxy}-N-(3-methoxy- | —CH₂CH₂OH | F | —OCH₃ |
| 4-fluorobenzyl)-4-oxo-3,4-dihydroquinazoline-2-carboxamide | | | |
| 6-fluoro-5-{2-[1-(2-hydroxyethyl)piperidin-4-yl]ethoxy}-N-(3-chloro4- | —CH₂CH₂OH | F | Cl |
| fluorobenzyl)-4-oxo-3,4-dihydroquinazoline-2-carboxamide | | | |
| 6-fluoro-5-{2-[1-(2-hydroxyethyl)piperidin-4-yl]ethoxy}-N-(3- | —CH₂CH₂OH | H | —OCH₃ |
| methoxybenzyl)-4-oxo-3,4-dihydroquinazoline-2-carboxamide | | | |
| 6-fluoro-5-{2-[1-(2-hydroxyethyl)piperidin-4-yl]ethoxy}-N-(3- | —CH₂CH₂OH | H | Cl |
| chlorobenzyl)-4-oxo-3,4-dihydroquinazoline-2-carboxamide | | | |
| N-(4-fluoro-3-methoxybenzyl)-6-fluoro-5-(2-{1-[(5-methyl-1,3,4- oxadiazol-2-yl)methyl]piperidin-4-yl}ethoxy)-4-oxo-3,4- dihydroquinazoline-2-carboxamide | 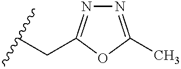 | F | —OCH₃ |
| N-(4-fluoro-3-chlorobenzyl)-6-fluoro-5-(2-{1-[(5-methyl-1,3,4- oxadiazol-2-yl)methyl]piperidin-4-yl}ethoxy)-4-oxo-3,4- dihydroquinazoline-2-carboxamide | 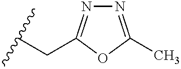 | F | Cl |
| N-(3-methoxybenzyl)-6-fluoro-5-(2-{1-[(5-methyl-1,3,4-oxadiazol-2- yl)methyl]piperidin-4-yl}ethoxy)-4-oxo-3,4-dihydroquinazoline-2- carboxamide | 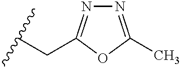 | H | —OCH₃ |
| N-(3-chlorobenzyl)-6-fluoro-5-(2-{1-[(5-methyl-1,3,4-oxadiazol-2- yl)methyl]piperidin-4-yl}ethoxy)-4-oxo-3,4-dihydroquinazoline-2- carboxamide | 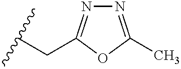 | H | Cl |
| N-(4-fluoro-3-methoxybenzyl)-6-fluoro-5-{2-[1-(2- | —C(═O)CH₂OH | F | —OCH₃ |
| hydroxyacetyl)piperidin-4-yl]ethoxy}-4-oxo-3,4-dihydroquinazoline-2- | | | |
| carboxamide | | | |
| N-(4-fluoro-3-chlorobenzyl)-6-fluoro-5-{2-[1-(2- | —C(═O)CH₂OH | F | Cl |
| hydroxyacetyl)piperidin-4-yl]ethoxy}-4-oxo-3,4-dihydroquinazoline-2- | | | |
| carboxamide | | | |
| N-(3-methoxybenzyl)-6-fluoro-5-{2-[1-(2-hydroxyacetyl)piperidin-4- | —C(═O)CH₂OH | H | —OCH₃ |
| yl]ethoxy}-4-oxo-3,4-dihydroquinazoline-2-carboxamide | | | |
| N-(3-chlorobenzyl)-6-fluoro-5-{2-[1-(2-hydroxyacetyl)piperidin-4- | —C(═O)CH₂OH | H | Cl |
| yl]ethoxy}-4-oxo-3,4-dihydroquinazoline-2-carboxamide | | | |
| N-(4-fluoro-3-methoxybenzyl)-6-fluoro-5-{2-[1- | —SO₂CH₃ | F | —OCH₃ |
| (methylsulfonyl)piperidin-4-yl]ethoxy}-4-oxo-3,4-dihydroquinazoline- | | | |
| 2-carboxamide | | | |
| N-(4-fluoro-3-chlorobenzyl)-6-fluoro-5-{2-[1-(methylsulfonyl)piperidin- | —SO₂CH₃ | F | Cl |
| 4-yl]ethoxy}-4-oxo-3,4-dihydroquinazoline-2-carboxamide | | | |
| N-(3-methoxybenzyl)-6-fluoro-5-{2-[1-(methylsulfonyl)piperidin-4- | —SO₂CH₃ | H | —OCH₃ |
| yl]ethoxy}-4-oxo-3,4-dihydroquinazoline-2-carboxamide | | | |
| N-(3-chlorobenzyl)-6-fluoro-5-{2-[1-(methylsulfonyl)piperidin-4- | —SO₂CH₃ | H | Cl |
| yl]ethoxy}-4-oxo-3,4-dihydroquinazoline-2-carboxamide | | | |

Another embodiment of the invention is a compound of Formula II-5, shown in Table 5.

TABLE 5
II-5

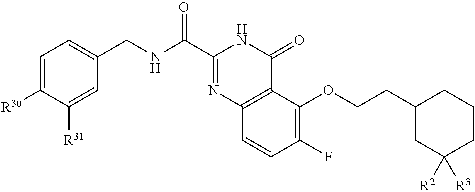

| Compound Name | R² | R³ | R³⁰ | R³¹ |
|---|---|---|---|---|
| N-(4-fluoro-3-methoxybenzyl)-6-fluoro-5-{2-[3-(2- | —NHC(═O)CH₂OH | H | F | —OCH₃ |
| hydroxyacetamido)cyclohexyl]ethoxy}-4-oxo-3,4-dihydroquinazoline- | | | | |
| 2-carboxamide | | | | |
| N-(4-fluoro-3-chlorobenzyl)-6-fluoro-5-{2-[3-(2- | —NHC(═O)CH₂OH | H | F | Cl |
| hydroxyacetamido)cyclohexyl]ethoxy}-4-oxo-3,4-dihydroquinazoline- | | | | |
| 2-carboxamide | | | | |
| N-(3-methoxybenzyl)-6-fluoro-5-{2-[3-(2- | —NHC(═O)CH₂OH | H | H | —OCH₃ |
| hydroxyacetamido)cyclohexyl]ethoxy}-4-oxo-3,4-dihydroquinazoline- | | | | |
| 2-carboxamide | | | | |
| N-(3-chlorobenzyl)-6-fluoro-5-{2-[3-(2- | —NHC(═O)CH₂OH | H | H | Cl |
| hydroxyacetamido)cyclohexyl]ethoxy}-4-oxo-3,4-dihydroquinazoline- | | | | |
| 2-carboxamide | | | | |

Another embodiment of the invention is a compound of Formula II-6, shown in Table 6.

TABLE 6
II-6

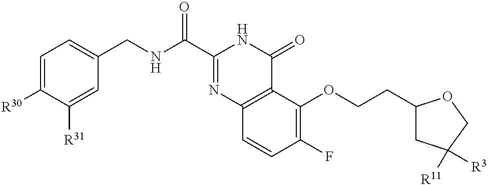

| Compound Name | R³ | R¹¹ | R³⁰ | R³¹ |
|---|---|---|---|---|
| N-(4-fluoro-3-methoxybenzyl)-6-fluoro-5-{2-[4-(2-hydroxyacetamido)- | H | —NHC(═O)CH₂OH | F | —OCH₃ |
| tetrahydrofuran-2-yl]ethoxy}-4-oxo-3,4-dihydroquinazoline-2- | | | | |
| carboxamide | | | | |
| N-(4-fluoro-3-chlorobenzyl)-6-fluoro-5-{2-[4-(2-hydroxyacetamido)- | H | —NHC(═O)CH₂OH | F | Cl |
| tetrahydrofuran-2-yl]ethoxy}-4-oxo-3,4-dihydroquinazoline-2- | | | | |
| carboxamide | | | | |
| N-(3-methoxybenzyl)-6-fluoro-5-{2-[4-(2-hydroxyacetamido)- | H | —NHC(═O)CH₂OH | H | —OCH₃ |
| tetrahydrofuran-2-yl]ethoxy}-4-oxo-3,4-dihydroquinazoline-2- | | | | |
| carboxamide | | | | |
| N-(3-chlorobenzyl)-6-fluoro-5-{2-[4-(2-hydroxyacetamido)- | H | —NHC(═O)CH₂OH | H | Cl |
| tetrahydrofuran-2-yl]ethoxy}-4-oxo-3,4-dihydroquinazoline-2- | | | | |
| carboxamide | | | | |
| N-(4-fluoro-3-methoxybenzyl)-5-[2-(4-acetamido-tetrahydrofuran-2- | H | —NHC(═O)CH₃ | F | —OCH₃ |
| yl)ethoxy]-6-fluoro-4-oxo-3,4-dihydroquinazoline-2-carboxamide | | | | |
| N-(4-fluoro-3-chlorobenzyl)-5-[2-(4-acetamido-tetrahydrofuran-2- | H | —NHC(═O)CH₃ | F | Cl |
| yl)ethoxy]-6-fluoro-4-oxo-3,4-dihydroquinazoline-2-carboxamide | | | | |
| N-(3-methoxybenzyl)-5-[2-(4-acetamido-tetrahydrofuran-2- | H | —NHC(═O)CH₃ | H | —OCH₃ |
| yl)ethoxy]-6-fluoro-4-oxo-3,4-dihydroquinazoline-2-carboxamide | | | | |
| N-(3-chlorobenzyl)-5-[2-(4-acetamido-tetrahydrofuran-2-yl)ethanoxy]-6- | H | —NHC(═O)CH₃ | H | Cl |
| fluoro-4-oxo-3,4-dihydroquinazoline-2-carboxamide | | | | |
| N-(4-fluoro-3-methoxybenzyl)-6-fluoro-5-{2-[4-(methylsulfonamido)- | H | —NHSO₂CH₃ | F | —OCH₃ |
| tetrahydrofuran-2-yl]ethoxy}-4-oxo-3,4-dihydroquinazoline-2- | | | | |
| carboxamide | | | | |
| N-(4-fluoro-3-chlorobenzyl)-6-fluoro-5-{2-[4-(methylsulfonamido)- | H | —NHSO₂CH₃ | F | Cl |
| tetrahydrofuran-2-yl]ethoxy}-4-oxo-3,4-dihydroquinazoline-2- | | | | |
| carboxamide | | | | |
| N-(3-methoxybenzyl)-6-fluoro-5-{2-[4-(methylsulfonamido)- | H | —NHSO₂CH₃ | H | —OCH₃ |
| tetrahydrofuran-2-yl]ethoxy}-4-oxo-3,4-dihydroquinazoline-2- | | | | |
| carboxamide | | | | |
| N-(3-chlorobenzyl)-6-fluoro-5-{2-[4-(methylsulfonamido)- | H | —NHSO₂CH₃ | H | Cl |
| tetrahydrofuran-2-yl]ethoxy}-4-oxo-3,4-dihydroquinazoline-2- | | | | |
| carboxamide | | | | |
| N-(4-fluoro-3-methoxybenzyl)-6-fluoro-5-{2-[4-hydroxy-4- | OH | —CH₂OH | F | —OCH₃ |
| (hydroxymethyl)-tetrahydrofuran-2-yl]ethoxy}-4-oxo-3,4- | | | | |
| dihydroquinazoline-2-carboxamide | | | | |
| N-(4-fluoro-3-chlorobenzyl)-6-fluoro-5-{2-[4-hydroxy-4- | OH | —CH₂OH | F | Cl |
| (hydroxymethyl)-tetrahydrofuran-2-yl]ethoxy}-4-oxo-3,4- | | | | |
| dihydroquinazoline-2-carboxamide | | | | |
| N-(3-methoxybenzyl)-6-fluoro-5-{2-[4-hydroxy-4-(hydroxymethyl)- | OH | —CH₂OH | H | —OCH₃ |
| tetrahydrofuran-2-yl]ethoxy}-4-oxo-3,4-dihydroquinazoline-2- | | | | |
| carboxamide | | | | |
| N-(3-chlorobenzyl)-6-fluoro-5-{2-[4-hydroxy-4-(hydroxymethyl)- | OH | —CH₂OH | H | Cl |
| tetrahydrofuran-2-yl]ethoxy}-4-oxo-3,4-dihydroquinazoline-2- | | | | |
| carboxamide | | | | |

Another embodiment of the invention is a compound of Formula II-7, shown in Table 7.

TABLE 7
II-7

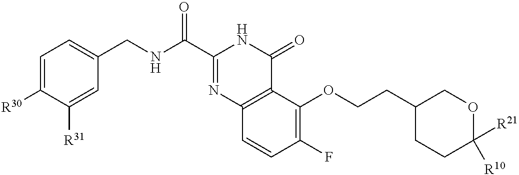

| Compound Name | R¹⁰ | R²¹ | R³⁰ | R³¹ |
|---|---|---|---|---|
| N-(4-fluoro-3-methoxybenzyl)-6-fluoro-5-(2-{6-[(2- | —CH₂NHC(═O)CH₂OH | H | F | —OCH₃ |
| hydroxyacetamido)methyl]-tetrahydro-2H-pyran-3-yl}ethoxy)-4- | | | | |
| oxo-3,4-dihydroquinazoline-2-carboxamide | | | | |
| N-(4-fluoro-3-chlorobenzyl)-6-fluoro-5-(2-{6-[(2- | —CH₂NHC(═O)CH₂OH | H | F | Cl |
| hydroxyacetamido)methyl]-tetrahydro-2H-pyran-3-yl}ethoxy)-4- | | | | |
| oxo-3,4-dihydroquinazoline-2-carboxamide | | | | |
| N-(3-methoxybenzyl)-6-fluoro-5-(2-{6-[(2- | —CH₂NHC(═O)CH₂OH | H | H | —OCH₃ |
| hydroxyacetamido)methyl]-tetrahydro-2H-pyran-3-yl}ethoxy)-4- | | | | |
| oxo-3,4-dihydroquinazoline-2-carboxamide | | | | |
| N-(3-chlorobenzyl)-6-fluoro-5-(2-{6-[(2- | —CH₂NHC(═O)CH₂OH | H | H | Cl |
| hydroxyacetamido)methyl]-tetrahydro-2H-pyran-3-yl}ethoxy)-4- | | | | |
| oxo-3,4-dihydroquinazoline-2-carboxamide | | | | |
| N-(4-fluoro-3-methoxybenzyl)-5-{2-[6-(acetamidomethyl)- | —CH₂NHC(═O)CH₃ | H | F | —OCH₃ |
| tetrahydro-2H-pyran-3-yl]ethoxy}-6-fluoro-4-oxo-3,4- | | | | |
| dihydroquinazoline-2-carboxamide | | | | |
| N-(4-fluoro-3-chlorobenzyl)-5-{2-[6-(acetamidomethyl)- | —CH₂NHC(═O)CH₃ | H | F | Cl |
| tetrahydro-2H-pyran-3-yl}ethoxy)-4-6-fluoro-4-oxo-3,4- | | | | |
| dihydroquinazoline-2-carboxamide | | | | |
| N-(3-methoxybenzyl)-5-{2-[6-(acetamidomethyl)-tetrahydro-2H- | —CH₂NHC(═O)CH₃ | H | H | —OCH₃ |
| pyran-3-yl]ethoxy)-6-fluoro-4-oxo-3,4-dihydroquinazoline-2- | | | | |
| carboxamide | | | | |
| N-(3-chlorobenzyl)-5-{2-[6-(acetamidomethyl)-tetrahydro-2H- | —CH₂NHC(═O)CH₃ | H | H | Cl |
| pyran-3-yl]ethoxy}-6-fluoro-4-oxo-3,4-dihydroquinazoline-2- | | | | |
| carboxamide | | | | |
| N-(4-fluoro-3-methoxybenzyl)-6-fluoro-5-{2-[6- | —CH₂NHSO₂CH₃ | H | F | —OCH₃ |
| (methylsulfonamidomethyl)-tetrahydro-2H-pyran-3-yl]ethoxy}-4- | | | | |
| oxo-3,4-dihydroquinazoline-2-carboxamide | | | | |
| N-(4-fluoro-3-chlorobenzyl)-6-fluoro-5-{2-[6- | —CH₂NHSO₂CH₃ | H | F | Cl |
| methylsulfonamidomethyl)-tetrahydro-2H-pyran-3-yl]ethoxy}-4- | | | | |
| oxo-3,4-dihydroquinazoline-2-carboxamide | | | | |
| N-(3-methoxybenzyl)-6-fluoro-5-{2-[6- | —CH₂NHSO₂CH₃ | H | H | —OCH₃ |
| methylsulfonylamidomethyl)-tetrahydro-2H-pyran-3-yl]ethoxy}-4- | | | | |
| oxo-3,4-dihydroquinazoline-2-carboxamide | | | | |
| N-(3-chlorobenzyl)-6-fluoro-5-{2-[6-(methylsulfonamidomethyl)- | —CH₂NHSO₂CH₃ | H | H | Cl |
| tetrahydro-2H-pyran-3-yl]ethoxy}-4-oxo-3,4-dihydroquinazoline- | | | | |
| 2-carboxamide | | | | |
| N-(4-fluoro-3-methoxybenzyl)-6-fluoro-5-{2-[6-(hydroxymethyl)- | —CH₂OH | H | F | —OCH₃ |
| tetrahydro-2H-pyran-3-yl]ethoxy}-4-oxo-3,4-dihydroquinazoline | | | | |
| 2-carboxamide | | | | |
| N-(4-fluoro-3-chlorobenzyl)-6-fluoro-5-{2-[6-(hydroxymethyl)- | —CH₂OH | H | F | Cl |
| tetrahydro-2H-pyran-3-yl]ethoxy}-4-oxo-3,4-dihydroquinazoline- | | | | |
| 2-carboxamide | | | | |
| N-(3-methoxybenzyl)-6-fluoro-5-{2-[6-(hydroxymethyl)- | —CH₂OH | H | H | —OCH₃ |
| tetrahydro-2H-pyran-3-yl]ethoxy}-4-oxo-3,4-dihydroquinazoline- | | | | |
| 2-carboxamide | | | | |
| N-(3-chlorobenzyl)-6-fluoro-5-{2-[6-(hydroxymethyl)-tetrahydro- | —CH₂OH | H | H | Cl |
| 2H-pyran-3-yl]ethoxy}-4-oxo-3,4-dihydroquinazoline-2- | | | | |
| carboxamide | | | | |

Another embodiment of the invention is a compound of Formula II-8, shown in Table 8.

TABLE 8
II-8

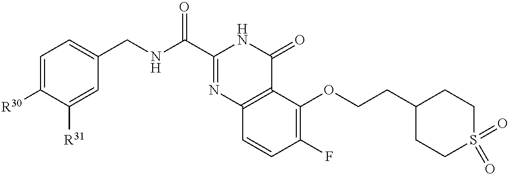

| Compound Name | R³⁰ | R³¹ |
|---|---|---|
| N-(4-fluoro-3-methoxybenzyl)-6-fluoro-4-oxo-5-[2-(tetrahydro-2H-1,1′-dioxothiopyran-4- | F | —OCH₃ |
| yl)ethoxy]-3,4-dihydroquinazoline-2-carboxamide | | |
| N-(4-fluoro-3-methoxybenzyl)-6-fluoro-4-oxo-5-[2-(tetrahydro-2H-1,1′-dioxothiopyran-4- | F | Cl |
| yl)ethoxy]-3,4-dihydroquinazoline-2-carboxamide | | |
| N-(3-methoxybenzyl)-6-fluoro-4-oxo-5-[2-(tetrahydro-2H-1,1′-dioxothiopyran-4-yl)ethoxy]-3,4- | H | —OCH₃ |
| dihydroquinazoline-2-carboxamide | | |
| N-(3-chlorobenzyl)-6-fluoro-4-oxo-5-[2-(tetrahydro-2H-1,1′-dioxothiopyran-4-yl)ethoxy]-3,4- | H | Cl |
| dihydroquinazoline-2-carboxamide | | |

Another embodiment of the invention is a compound of Formula II-9, shown in Table 9.

TABLE 9
II-9

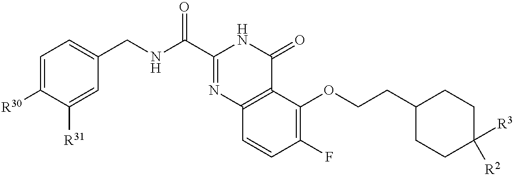

| Compound Name | R² | R³ | R³⁰ | R³¹ |
|---|---|---|---|---|
| 6-fluoro-5-{[4-(hydroxymethyl)cyclohexyl]methoxy}-N-(3- | —CH₂OH | H | F | —OCH₃ |
| methoxy-4-fluorobenzyl)-4-oxo-3,4-dihydroquinazoline-2- | | | | |
| carboxamide | | | | |
| 6-fluoro-5-{[4-(hydroxymethyl)cyclohexyl]methoxy}-N-(3- | —CH₂OH | H | F | Cl |
| chloro-4-fluorobenzyl)-4-oxo-3,4-dihydroquinazoline-2- | | | | |
| carboxamide | | | | |
| 6-fluoro-5-{[4-(hydroxymethyl)cyclohexyl]methoxy}-N-(3- | —CH₂OH | H | H | —OCH₃ |
| methoxybenzyl)-4-oxo-3,4-dihydroquinazoline-2- | | | | |
| carboxamide | | | | |
| 6-fluoro-5-{[4-(hydroxymethyl)cyclohexyl]methoxy}-N-(3- | —CH₂OH | H | H | Cl |
| chlorobenzyl)-4-oxo-3,4-dihydroquinazoline-2-carboxamide | | | | |
| N-(4-fluoro-3-methoxybenzyl)-6-fluoro-5-(2-{4-[(5-methyl- 1,3,4-oxadiazol-2-yl)methyl]cyclohexyl}ethoxy)-4-oxo-3,4- dihydroquinazoline-2-carboxamide | 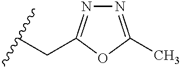 | H | F | —OCH₃ |
| N-(4-fluoro-3-methoxybenzyl)-6-fluoro-5-(2-{4-[(5-methyl- 1,3,4-oxadiazol-2-yl)methyl]cyclohexyl}ethoxy)-4-oxo-3,4- dihydroquinazoline-2-carboxamide | 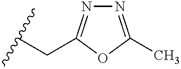 | H | F | Cl |
| N-(3-methoxybenzyl)-6-fluoro-5-(2-{4-[(5-methyl-1,3,4- oxadiazol-2-yl)methyl]cyclohexyl}ethoxy)-4-oxo-3,4- dihydroquinazoline-2-carboxamide | 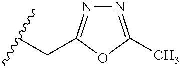 | H | H | —OCH₃ |
| N-(3-chlorobenzyl)-6-fluoro-5-(2-{4-[(5-methyl-1,3,4- oxadiazol-2-yl)methyl]cyclohexyl}ethoxy)-4-oxo-3,4- dihydroquinazoline-2-carboxamide | 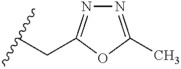 | H | H | Cl |
| N-(4-fluoro-3-methoxybenzyl)-6-fluoro-5-{2-[4- | —CH₂NHSO₂CH₃ | H | F | —OCH₃ |
| (methylsulfonamidomethyl)cyclohexyl]ethoxy}-4-oxo-3,4- | | | | |
| dihydroquinazoline-2-carboxamide | | | | |
| N-(4-fluoro-3-chlorobenzyl)-6-fluoro-5-{2-[4- | —CH₂NHSO₂CH₃ | H | F | Cl |
| (methylsulfonamidomethyl)cyclohexyl]ethoxy}-4-oxo-3,4- | | | | |
| dihydroquinazoline-2-carboxamide | | | | |
| N-(3-methoxybenzyl)-6-fluoro-5-{2-[4- | —CH₂NHSO₂CH₃ | H | H | —OCH₃ |
| (methylsulfonamidomethyl)cyclohexyl]ethoxy}-4-oxo-3,4- | | | | |
| dihydroquinazoline-2-carboxamide | | | | |
| N-(3-methoxybenzyl)-6-fluoro-5-{2-[4- | —CH₂NHSO₂CH₃ | H | H | Cl |
| (methylsulfonamidomethyl)cyclohexyl]ethoxy}-4-oxo-3,4- | | | | |
| dihydroquinazoline-2-carboxamide | | | | |
| N-(4-fluoro-3-methoxybenzyl)-6-fluoro-5-(2-(4-hydroxy-4- | —CH₂OH | OH | F | —OCH₃ |
| (hydroxymethyl)cyclohexyl]ethoxy}-4-oxo-3,4- | | | | |
| dihydroquinazoline-2-carboxamide | | | | |
| N-(4-fluoro-3-chlorobenzyl)-6-fluoro-5-(2-(4-hydroxy-4- | —CH₂OH | OH | F | Cl |
| (hydroxymethyl)cyclohexyl]ethoxy}-4-oxo-3,4- | | | | |
| dihydroquinazoline-2-carboxamide | | | | |
| N-(3-methoxybenzyl)-6-fluoro-5-(2-(4-hydroxy-4- | —CH₂OH | OH | H | —OCH₃ |
| (hydroxymethyl)cyclohexyl]ethoxy}-4-oxo-3,4- | | | | |
| dihydroquinazoline-2-carboxamide | | | | |
| N-(3-chlorobenzyl)-6-fluoro-5-{2-[4-hydroxy-4- | —CH₂OH | OH | H | Cl |
| (hydroxymethyl)cyclohexyl]ethoxy}-4-oxo-3,4- | | | | |
| dihydroquinazoline-2-carboxamide | | | | |
| N-(4-fluoro-3-methoxybenzyl)-5-[2-(4- | CN | H | F | —OCH₃ |
| cyanocyclohexyl)ethoxy]-6-fluoro-4-oxo-3,4- | | | | |
| dihydroquinazoline-2-carboxamide | | | | |
| N-(4-fluoro-3-chlorobenzyl)-5-[2-(4- | CN | H | F | Cl |
| cyanocyclohexyl)ethoxy]-6-fluoro-4-oxo-3,4- | | | | |
| dihydroquinazoline-2-carboxamide | | | | |
| N-(3-methoxybenzyl)-5-[2-(4-cyanocyclohexyl)ethoxy]-6- | CN | H | H | —OCH₃ |
| fluoro-4-oxo-3,4-dihydroquinazoline-2-carboxamide | | | | |
| N-(3-chlorobenzyl)-5-[2-(4-cyanocyclohexyl)ethoxy]-6- | CN | H | H | Cl |
| fluoro-4-oxo-3,4-dihydroquinazoline-2-carboxamide | | | | |
| 2-[4-(2-{2-[(4-fluoro-3-methoxybenzyl)carbamoyl]-6-fluoro- | —NHC(═O)CH₂OC(═O)CH₃ | H | F | —OCH₃ |
| 4-oxo-3,4-dihydroquinazolin-5- | | | | |
| yloxy}ethyl)cyclohexylamino]-2-oxoethyl acetate | | | | |
| 2-[4-(2-{2-[(4-fluoro-3-chlorobenzyl)carbamoyl]-6-fluoro-4- | —NHC(═O)CH₂OC(═O)CH₃ | H | F | Cl |
| oxo-3,4-dihydroquinazolin-5-yloxy}ethyl)cyclohexylamino]- | | | | |
| 2-oxoethyl acetate | | | | |
| 2-[4-(2-{2-[(3-methoxybenzyl)carbamoyl]-6-fluoro-4-oxo- | —NHC(═O)CH₂OC(═O)CH₃ | H | H | —OCH₃ |
| 3,4-dihydroquinazolin-5-yloxy}ethyl)cyclohexylamino]-2- | | | | |
| oxoethyl acetate | | | | |
| 2-[4-(2-{2-[(3-chlorobenzyl)carbamoyl]-6-fluoro-4-oxo-3,4- | —NHC(═O)CH₂OC(═O)CH₃ | H | H | Cl |
| dihydroquinazolin-5-yloxy}ethyl)cyclohexylamino]-2- | | | | |
| oxoethyl acetate | | | | |
| N-(4-fluoro-3-methoxybenzyl)-6-fluoro-5-{2-[4-(2- | —NHC(═O)CH₂OH | H | F | —OCH₃ |
| hydroxyacetamidomethyl)cyclohexyl]ethoxy}-4-oxo-3,4- | | | | |
| dihydroquinazoline-2-carboxamide | | | | |
| N-(4-fluoro-3-chlorobenzyl)-6-fluoro-5-{2-[4-(2- | —NHC(═O)CH₂OH | H | F | Cl |
| hydroxyacetamidomethyl)cyclohexyl]ethoxy}-4-oxo-3,4- | | | | |
| dihydroquinazoline-2-carboxamide | | | | |
| N-(3-methoxybenzyl)-6-fluoro-5-{2-[4-(2- | —NHC(═O)CH₂OH | H | H | —OCH₃ |
| hydroxyacetamidomethyl)cyclohexyl]ethoxy}-4-oxo-3,4- | | | | |
| dihydroquinazoline-2-carboxamide | | | | |
| N-(3-chlorobenzyl)-6-fluoro-5-{2-[4-(2- | —NHC(═O)CH₂OH | H | H | Cl |
| hydroxyacetamidomethyl)cyclohexyl]ethoxy}-4-oxo-3,4- | | | | |
| dihydroquinazoline-2-carboxamide | | | | |
| N-(4-fluoro-3-methoxybenzyl)-5-[2-(4- | —NHC(═O)CH₃ | H | F | —OCH₃ |
| acetamidocyclohexyl)ethoxy]-6-fluoro-4-oxo-3,4- | | | | |
| dihydroquinazoline-2-carboxamide | | | | |
| N-(4-fluoro-3-chlorobenzyl)-5-[2-(4- | —NHC(═O)CH₃ | H | F | Cl |
| acetamidocyclohexyl)ethoxy]-6-fluoro-4-oxo-3,4- | | | | |
| dihydroquinazoline-2-carboxamide | | | | |
| N-(3-methoxybenzyl)-5-[2-(4-acetamidocyclohexyl)ethoxy]- | —NHC(═O)CH₃ | H | H | —OCH₃ |
| 6-fluoro-4-oxo-3,4-dihydroquinazoline-2-carboxamide | | | | |
| N-(3-chlorobenzyl)-5-[2-(4-acetamidocyclohexyl)ethoxy]-6- | —NHC(═O)CH₃ | H | H | Cl |
| fluoro-4-oxo-3,4-dihydroquinazoline-2-carboxamide | | | | |
| N-(4-fluoro-3-methoxybenzyl)-6-fluoro-5-{2-[4- | —NHSO₂CH₃ | H | F | —OCH₃ |
| (methylsulfonamido)cyclohexyl]ethoxy}-4-oxo-3,4- | | | | |
| dihydroquinazoline-2-carboxamide | | | | |
| N-(4-fluoro-3-chlorobenzyl)-6-fluoro-5-{2-[4- | —NHSO₂CH₃ | H | F | Cl |
| (methylsulfonamido)cyclohexyl]ethoxy}-4-oxo-3,4- | | | | |
| dihydroquinazoline-2-carboxamide | | | | |
| N-(3-methoxybenzyl)-6-fluoro-5-{2-[4- | —NHSO₂CH₃ | H | H | —OCH₃ |
| (methylsulfonamido)cyclohexyl]ethoxy}-4-oxo-3,4- | | | | |
| dihydroquinazoline-2-carboxamide | | | | |
| N-(3-chlorobenzyl)-6-fluoro-5-{2-[4- | —NHSO₂CH₃ | H | H | Cl |
| (methylsulfonamido)cyclohexyl]ethoxy}-4-oxo-3,4- | | | | |
| dihydroquinazoline-2-carboxamide | | | | |

Another embodiment of the invention is a compound of Formula II-10, shown in Table 10.

TABLE 10
II-10

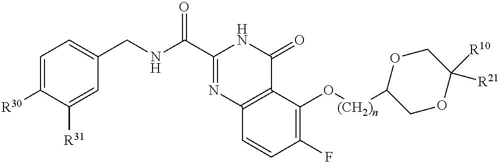

| Compound Name | n | R¹⁰ | R²¹ | R³⁰ | R³¹ |
|---|---|---|---|---|---|
| N-(4-fluoro-3-methoxybenzyl)-6-fluoro-5-{2-[5-(2-hydroxypropan- | 2 | —C(OH)(CH₃)₂ | H | F | —OCH₃ |
| 2-yl)-1,4-dioxan-2-yl]ethoxy}-4-oxo-3,4-dihydroquinazoline-2- | | | | | |
| carboxamide | | | | | |
| N-(4-fluoro-3-chlorobenzyl)-6-fluoro-5-{2-[5-(2-hydroxypropan-2- | 2 | —C(OH)(CH₃)₂ | H | F | Cl |
| yl)-1,4-dioxan-2-yl]ethoxy}-4-oxo-3,4-dihydroquinazoline-2- | | | | | |
| carboxamide | | | | | |
| N-(3-methoxybenzyl)-6-fluoro-5-{2-[5-(2-hydroxypropan-2-yl)- | 2 | —C(OH)(CH₃)₂ | H | H | —OCH₃ |
| 1,4-dioxan-2-yl]ethoxy}-4-oxo-3,4-dihydroquinazoline-2- | | | | | |
| carboxamide | | | | | |
| N-(3-chlorobenzyl)-6-fluoro-5-{2-[5-(2-hydroxypropan-2-yl)-1,4- | 2 | —C(OH)(CH₃)₂ | H | H | Cl |
| dioxan-2-yl]ethoxy}-4-oxo-3,4-dihydroquinazoline-2-carboxamide | | | | | |
| N-(4-fluoro-3-methoxybenzyl)-5-[2-(5-carbamoyl-1,4-dioxan-2- | 2 | —C(═O)NH₂ | H | F | —OCH₃ |
| yl)ethoxy]-6-fluoro-4-oxo-3,4-dihydroquinazoline-2-carboxamide | | | | | |
| N-(4-fluoro-3-chlorobenzyl)-5-[2-(5-carbamoyl-1,4-dioxan-2- | 2 | —C(═O)NH₂ | H | F | Cl |
| yl)ethoxy]-6-fluoro-4-oxo-3,4-dihydroquinazoline-2-carboxamide | | | | | |
| N-(3-methoxybenzyl)-5-[2-(5-carbamoyl-1,4-dioxan-2-yl)ethoxy]- | 2 | —C(═O)NH₂ | H | H | —OCH₃ |
| 6-fluoro-4-oxo-3,4-dihydroquinazoline-2-carboxamide | | | | | |
| N-(3-chlorobenzyl)-5-[2-(5-carbamoyl-1,4-dioxan-2-yl)ethoxy]-6- | 2 | —C(═O)NH₂ | H | H | Cl |
| fluoro-4-oxo-3,4-dihydroquinazoline-2-carboxamide | | | | | |
| 5-(2-{5-[(hydroxymethyl)carbamoyl]-1,4-dioxan-2-yl}ethoxy)-N- | 2 | —C(═O)NHCH₂OH | H | F | —OCH₃ |
| (4-fluoro-3-methoxybenzyl)-6-fluoro-4-oxo-3,4- | | | | | |
| dihydroquinazoline-2-carboxamide | | | | | |
| 5-(2-{5-[(hydroxymethyl)carbamoyl]-1,4-dioxan-2-yl}ethoxy)-N- | 2 | —C(═O)NHCH₂OH | H | F | Cl |
| (4-fluoro-3-chlorobenzyl)-6-fluoro-4-oxo-3,4-dihydroquinazoline- | | | | | |
| 2-carboxamide | | | | | |
| 5-(2-{5-[(hydroxymethyl)carbamoyl]-1,4-dioxan-2-yl}ethoxy)-N- | 2 | —C(═O)NHCH₂OH | H | H | —OCH₃ |
| (3-methoxybenzyl)-6-fluoro-4-oxo-3,4-dihydroquinazoline-2- | | | | | |
| carboxamide | | | | | |
| 5-(2-{5-[(hydroxymethyl)carbamoyl]-1,4-dioxan-2-yl}ethoxy)-N- | 2 | —C(═O)NHCH₂OH | H | H | Cl |
| (3-chlorobenzyl)-6-fluoro-4-oxo-3,4-dihydroquinazoline-2- | | | | | |
| carboxamide | | | | | |
| N-(4-fluoro-3-methoxybenzyl)-6-fluoro-5-{2-[5- | 2 | —C(═O)NHCH₃ | H | F | —OCH₃ |
| (methylcarbamoyl)-1,4-dioxan-2-yl]ethoxy}-4-oxo-3,4- | | | | | |
| dihydroquinazoline-2-carboxamide | | | | | |
| N-(4-fluoro-3-chlorobenzyl)-6-fluoro-5-{2-[5-(methylcarbamoyl)- | 2 | —C(═O)NHCH₃ | H | F | Cl |
| 1,4-dioxan-2-yl]ethoxy}-4-oxo-3,4-dihydroquinazoline-2- | | | | | |
| carboxamide | | | | | |
| N-(3-methoxybenzyl)-6-fluoro-5-{2-[5-(methylcarbamoyl)-1,4- | 2 | —C(═O)NHCH₃ | H | H | —OCH₃ |
| dioxan-2-yl]ethoxy}-4-oxo-3,4-dihydroquinazoline-2-carboxamide | | | | | |
| N-(3-chlorobenzyl)-6-fluoro-5-{2-[5-(methylcarbamoyl)-1,4- | 2 | —C(═O)NHCH₃ | H | H | Cl |
| dioxan-2-yl]ethoxy}-4-oxo-3,4-dihydroquinazoline-2-carboxamide | | | | | |
| 5-(2-{2-[(4-fluoro-3-methoxybenzyl)carbamoyl]-6-fluoro-4-oxo- | 2 | —C(═O)OH | H | F | —OCH₃ |
| 3,4-dihydroquinazolin-5-yloxy}ethyl)-1,4-dioxane-2-carboxylic | | | | | |
| acid | | | | | |
| 5-(2-{2-[(4-fluoro-3-chlorobenzyl)carbamoyl]-6-fluoro-4-oxo-3,4- | 2 | —C(═O)OH | H | F | Cl |
| dihydroquinazolin-5-yloxy}ethyl)-1,4-dioxane-2-carboxylic acid | | | | | |
| 5-(2-{2-[(3-methoxybenzyl)carbamoyl]-6-fluoro-4-oxo-3,4- | 2 | —C(═O)OH | H | H | —OCH₃ |
| dihydroquinazolin-5-yloxy}ethyl)-1,4-dioxane-2-carboxylic acid | | | | | |
| 5-(2-{2-[(3-chlorobenzyl)carbamoyl]-6-fluoro-4-oxo-3,4- | 2 | —C(═O)OH | H | H | Cl |
| dihydroquinazolin-5-yloxy}ethyl)-1,4-dioxane-2-carboxylic acid | | | | | |
| N-(4-fluoro-3-methoxybenzyl)-5-{2-[5-(cyanomethyl)-1,4-dioxan- | 2 | —CH₂CN | H | F | —OCH₃ |
| 2-yl]ethoxy}-6-fluoro-4-oxo-3,4-dihydroquinazoline-2- | | | | | |
| carboxamide | | | | | |
| N-(4-fluoro-3-chlorobenzyl)-5-{2-[5-(cyanomethyl)-1,4-dioxan-2- | 2 | —CH₂CN | H | F | Cl |
| yl]ethoxy}-6-fluoro-4-oxo-3,4-dihydroquinazoline-2-carboxamide | | | | | |
| N-(3-methoxybenzyl)-5-{2-[5-(cyanomethyl)-1,4-dioxan-2- | 2 | —CH₂CN | H | H | —OCH₃ |
| yl]ethoxy}-6-fluoro-4-oxo-3,4-dihydroquinazoline-2-carboxamide | | | | | |
| N-(3-chlorobenzyl)-5-{2-[5-(cyanomethyl)-1,4-dioxan-2- | 2 | —CH₂CN | H | H | Cl |
| yl]ethoxy}-6-fluoro-4-oxo-3,4-dihydroquinazoline-2-carboxamide | | | | | |
| N-(4-fluoro-3-methoxybenzyl)-6-fluoro-5-{2-[5- | 2 | —CH₂NHSO₂CH₃ | H | F | —OCH₃ |
| (methylsulfonamidomethyl)-1,4-dioxan-2-yl]ethoxy}-4-oxo-3,4- | | | | | |
| dihydroquinazoline-2-carboxamide | | | | | |
| N-(4-fluoro-3-chlorobenzyl)-6-fluoro-5-{2-[5- | 2 | —CH₂NHSO₂CH₃ | H | F | Cl |
| (methylsulfonamidomethyl)-1,4-dioxan-2-yl]ethoxy}-4-oxo-3,4- | | | | | |
| dihydroquinazoline-2-carboxamide | | | | | |
| N-(3-methoxybenzyl)-6-fluoro-5-{2-[5- | 2 | —CH₂NHSO₂CH₃ | H | H | —OCH₃ |
| (methylsulfonamidomethyl)-1,4-dioxan-2-yl]ethoxy}-4-oxo-3,4- | | | | | |
| dihydroquinazoline-2-carboxamide | | | | | |
| N-(3-c hlorobenzyl)-6-fluoro-5-{2-[5-(methylsulfonamidomethyl)- | 2 | —CH₂NHSO₂CH₃ | H | H | Cl |
| 1,4-dioxan-2-yl]ethoxy}-4-oxo-3,4-dihydroquinazoline-2- | | | | | |
| carboxamide | | | | | |
| N-(4-fluoro-3-methoxybenzyl)-6-fluoro-5-{2-[5-(hydroxymethyl)- | 2 | —CH₂OH | H | F | —OCH₃ |
| 1,4-dioxan-2-yl]ethoxy}-4-oxo-3,4-dihydroquinazoline-2- | | | | | |
| carboxamide | | | | | |
| N-(4-fluoro-3-chlorobenzyl)-6-fluoro-5-{2-[5-(hydroxymethyl)-1,4- | 2 | —CH₂OH | H | F | Cl |
| dioxan-2-yl]ethoxy}-4-oxo-3,4-dihydroquinazoline-2-carboxamide | | | | | |
| N-(3-methoxybenzyl)-6-fluoro-5-{2-[5-(hydroxymethyl)-1,4- | 2 | —CH₂OH | H | H | —OCH₃ |
| dioxan-2-yl]ethoxy}-4-oxo-3,4-dihydroquinazoline-2-carboxamide | | | | | |
| N-(3-chlorobenzyl)-6-fluoro-5-{2-[5-(hydroxymethyl)-1,4-dioxan- | 2 | —CH₂OH | H | H | Cl |
| 2-yl]ethoxy}-4-oxo-3,4-dihydroquinazoline-2-carboxamide | | | | | |
| N-(4-fluoro-3-methoxybenzyl)-6-fluoro-5-{2-[5- | 2 | —CH₂SO₂CH₃ | H | F | —OCH₃ |
| (methylsulfonylmethyl)-1,4-dioxan-2-yl]ethoxy}-4-oxo-3,4- | | | | | |
| dihydroquinazoline-2-carboxamide | | | | | |
| N-(4-fluoro-3-chlorobenzyl)-6-fluoro-5-{2-[5- | 2 | —CH₂SO₂CH₃ | H | F | Cl |
| (methylsulfonylmethyl)-1,4-dioxan-2-yl]ethoxy}-4-oxo-3,4- | | | | | |
| dihydroquinazoline-2-carboxamide | | | | | |
| N-(3-methoxybenzyl)-6-fluoro-5-{2-[5-(methylsulfonylmethyl)-1,4- | 2 | —CH₂SO₂CH₃ | H | H | —OCH₃ |
| dioxan-2-yl]ethoxy}-4-oxo-3,4-dihydroquinazoline-2-carboxamide | | | | | |
| N-(3-chlorobenzyl)-6-fluoro-5-{2-[5-(methylsulfonylmethyl)-1,4- | 2 | —CH₂SO₂CH₃ | H | H | Cl |
| dioxan-2-yl]ethoxy}-4-oxo-3,4-dihydroquinazoline-2-carboxamide | | | | | |
| N-(4-fluoro-3-methoxybenzyl)-5-[2-(5-cyano-1,4-dioxan-2- | 2 | CN | H | F | —OCH₃ |
| yl)ethoxy]-6-fluoro-4-oxo-3,4-dihydroquinazoline-2-carboxamide | | | | | |
| N-(4-fluoro-3-chlorobenzyl)-5-[2-(5-cyano-1,4-dioxan-2- | 2 | CN | H | F | Cl |
| yl)ethoxy]-6-fluoro-4-oxo-3,4-dihydroquinazoline-2-carboxamide | | | | | |
| N-(3-methoxybenzyl)-5-[2-(5-cyano-1,4-dioxan-2-yl)ethoxy]-6- | 2 | CN | H | H | —OCH₃ |
| fluoro-4-oxo-3,4-dihydroquinazoline-2-carboxamide | | | | | |
| N-(3-chlorobenzyl)-5-[2-(5-cyano-1,4-dioxan-2-yl)ethoxy]-6- | 2 | CN | H | H | Cl |
| fluoro-4-oxo-3,4-dihydroquinazoline-2-carboxamide | | | | | |
| N-(4-fluoro-3-methoxybenzyl)-6-fluoro-5-{2-[5-(2-hydroxypropan- | 1 | —C(OH)(CH₃)₂ | H | F | —OCH₃ |
| 2-yl)-1,4-dioxan-2-yl]methoxy}-4-oxo-3,4-dihydroquinazoline-2- | | | | | |
| carboxamide | | | | | |
| N-(4-fluoro-3-chlorobenzyl)-6-fluoro-5-{2-[5-(2-hydroxypropan-2- | 1 | —C(OH)(CH₃)₂ | H | F | Cl |
| yl)-1,4-dioxan-2-yl]methoxy}-4-oxo-3,4-dihydroquinazoline-2- | | | | | |
| carboxamide | | | | | |
| N-(3-methoxybenzyl)-6-fluoro-5-{2-[5-(2-hydroxypropan-2-yl)- | 1 | —C(OH)(CH₃)₂ | H | H | —OCH₃ |
| 1,4-dioxan-2-yl]methoxy}-4-oxo-3,4-dihydroquinazoline-2- | | | | | |
| carboxamide | | | | | |
| N-(3-chlorobenzyl)-6-fluoro-5-{2-[5-(2-hydroxypropan-2-yl)-1,4- | 1 | —C(OH)(CH₃)₂ | H | H | Cl |
| dioxan-2-yl]methoxy}-4-oxo-3,4-dihydroquinazoline-2- | | | | | |
| carboxamide | | | | | |
| N-(4-fluoro-3-methoxybenzyl)-5-[2-(5-carbamoyl-1,4-dioxan-2- | 1 | —C(═O)NH₂ | H | F | —OCH₃ |
| yl)methoxy]-6-fluoro-4-oxo-3,4-dihydroquinazoline-2- | | | | | |
| carboxamide | | | | | |
| N-(4-fluoro-3-chlorobenzyl)-5-[2-(5-carbamoyl-1,4-dioxan-2- | 1 | —C(═O)NH₂ | H | F | Cl |
| yl)methoxy]-6-fluoro-4-oxo-3,4-dihydroquinazoline-2- | | | | | |
| carboxamide | | | | | |
| N-(3-methoxybenzyl)-5-[2-(5-carbamoyl-1,4-dioxan-2- | 1 | —C(═O)NH₂ | H | H | —OCH₃ |
| yl)methoxy]-6-fluoro-4-oxo-3,4-dihydroquinazoline-2- | | | | | |
| carboxamide | | | | | |
| N-(3-chlorobenzyl)-5-[2-(5-carbamoyl-1,4-dioxan-2-yl)methoxy]- | 1 | —C(═O)NH₂ | H | H | Cl |
| 6-fluoro-4-oxo-3,4-dihydroquinazoline-2-carboxamide | | | | | |
| 5-(2-{5-[(hydroxymethyl)carbamoyl]-1,4-dioxan-2-yl}methoxy)-N- | 1 | —C(═O)NHCH₂OH | H | F | —OCH₃ |
| (4-fluoro-3-methoxybenzyl)-6-fluoro-4-oxo-3,4- | | | | | |
| dihydroquinazoline-2-carboxamide | | | | | |
| 5-(2-{5-[(hydroxymethyl)carbamoyl]-1,4-dioxan-2-yl}methoxy)-N- | 1 | —C(═O)NHCH₂OH | H | F | Cl |
| (4-fluoro-3-chlorobenzyl)-6-fluoro-4-oxo-3,4-dihydroquinazoline- | | | | | |
| 2-carboxamide | | | | | |
| 5-(2-{5-[(hydroxymethyl)carbamoyl]-1,4-dioxan-2-yl}methoxy)-N- | 1 | —C(═O)NHCH₂OH | H | H | —OCH₃ |
| (3-methoxybenzyl)-6-fluoro-4-oxo-3,4-dihydroquinazoline-2- | | | | | |
| carboxamide | | | | | |
| 5-(2-{5-[(hydroxymethyl)carbamoyl]-1,4-dioxan-2-yl}methoxy)-N- | 1 | —C(═O)NHCH₂OH | H | H | Cl |
| (3-chlorobenzyl)-6-fluoro-4-oxo-3,4-dihydroquinazoline-2- | | | | | |
| carboxamide | | | | | |
| N-(4-fluoro-3-methoxybenzyl)-6-fluoro-5-{2-[5- | 1 | —C(═O)NHCH₃ | H | F | —OCH₃ |
| (methylcarbamoyl)-1,4-dioxan-2-yl]methoxy}-4-oxo-3,4- | | | | | |
| dihydroquinazoline-2-carboxamide | | | | | |
| N-(4-fluoro-3-chlorobenzyl)-6-fluoro-5-{2-[5-(methylcarbamoyl)- | 1 | —C(═O)NHCH₃ | H | F | Cl |
| 1,4-dioxan-2-yl]methoxy}-4-oxo-3,4-dihydroquinazoline-2- | | | | | |
| carboxamide | | | | | |
| N-(3-methoxybenzyl)-6-fluoro-5-{2-[5-(methylcarbamoyl)-1,4- | 1 | —C(═O)NHCH₃ | H | H | —OCH₃ |
| dioxan-2-yl]methoxy}-4-oxo-3,4-dihydroquinazoline-2- | | | | | |
| carboxamide | | | | | |
| N-(3-chlorobenzyl)-6-fluoro-5-{2-[5-(methylcarbamoyl)-1,4- | 1 | —C(═O)NHCH₃ | H | H | Cl |
| dioxan-2-yl]methoxy}-4-oxo-3,4-dihydroquinazoline-2- | | | | | |
| carboxamide | | | | | |
| 5-(2-{2-[(4-fluoro-3-methoxybenzyl)carbamoyl]-6-fluoro-4-oxo- | 1 | —C(═O)OH | H | F | —OCH₃ |
| 3,4-dihydroquinazolin-5-yloxy}methyl)-1,4-dioxane-2-carboxylic | | | | | |
| acid | | | | | |
| 5-(2-{2-[(4-fluoro-3-chlorobenzyl)carbamoyl]-6-fluoro-4-oxo-3,4- | 1 | —C(═O)OH | H | F | Cl |
| dihydroquinazolin-5-yloxy}methyl)-1,4-dioxane-2-carboxylic acid | | | | | |
| 5-(2-{2-[(3-methoxybenzyl)carbamoyl]-6-fluoro-4-oxo-3,4- | 1 | —C(═O)OH | H | H | —OCH₃ |
| dihydroquinazolin-5-yloxy}methyl)-1,4-dioxane-2-carboxylic acid | | | | | |
| 5-(2-{2-[(3-chlorobenzyl)carbamoyl]-6-fluoro-4-oxo-3,4- | 1 | —C(═O)OH | H | H | Cl |
| dihydroquinazolin-5-yloxy}methyl)-1,4-dioxane-2-carboxylic acid | | | | | |
| N-(4-fluoro-3-methoxybenzyl)-5-{2-[5-(cyanomethyl)-1,4-dioxan- | 1 | —CH₂CN | H | F | —OCH₃ |
| 2-yl]methoxy}-6-fluoro-4-oxo-3,4-dihydroquinazolione-2- | | | | | |
| carboxamide | | | | | |
| N-(4-fluoro-3-chlorobenzyl)-5-{2-[5-(cyanomethyl)-1,4-dioxan-2- | 1 | —CH₂CN | H | F | Cl |
| yl]methoxy}-6-fluoro-4-oxo-3,4-dihydroquinazolione-2- | | | | | |
| carboxamide | | | | | |
| N-(3-methoxybenzyl)-5-{2-[5-(cyanomethyl)-1,4-dioxan-2- | 1 | —CH₂CN | H | H | —OCH₃ |
| yl]methoxy}-6-fluoro-4-oxo-3,4-dihydroquinazolione-2- | | | | | |
| carboxamide | | | | | |
| N-(3-chlorobenzyl)-5-{2-[5-(cyanomethyl)-1,4-dioxan-2- | 1 | —CH₂CN | H | H | Cl |
| yl]methoxy}-6-fluoro-4-oxo-3,4-dihydroquinazolione-2- | | | | | |
| carboxamide | | | | | |
| N-(4-fluoro-3-methoxybenzyl)-6-fluoro-5-{2-[5- | 1 | —CH₂NHSO₂CH₃ | H | F | —OCH₃ |
| (methylsulfonamidomethyl)-1,4-dioxan-2-yl]methoxy}-4-oxo-3,4- | | | | | |
| dihydroquinazoline-2-carboxamide | | | | | |
| N-(4-fluoro-3-chlorobenzyl)-6-fluoro-5-{2-[5- | 1 | —CH₂NHSO₂CH₃ | H | F | Cl |
| (methylsulfonamidomethyl)-1,4-dioxan-2-yl]methoxy}-4-oxo-3,4- | | | | | |
| dihydroquinazoline-2-carboxamide | | | | | |
| N-(3-methoxybenzyl)-6-fluoro-5-{2-[5- | 1 | —CH₂NHSO₂CH₃ | H | H | —OCH₃ |
| (methylsulfonamidomethyl)-1,4-dioxan-2-yl]methoxy}-4-oxo-3,4- | | | | | |
| dihydroquinazoline-2-carboxamide | | | | | |
| N-(3-chlorobenzyl)-6-fluoro-5-{2-[5-(methylsulfonamidomethyl)- | 1 | —CH₂NHSO₂CH₃ | H | H | Cl |
| 1,4-dioxan-2-yl]methoxy}-4-oxo-3,4-dihydroquinazoline-2- | | | | | |
| carboxamide | | | | | |
| N-(4-fluoro-3-methoxybenzyl)-6-fluoro-5-{2-[5-(hydroxymethyl)- | 1 | —CH₂OH | H | F | —OCH₃ |
| 1,4-dioxan-2-yl]methoxy}-4-oxo-3,4-dihydroquinazoline-2- | | | | | |
| carboxamide | | | | | |
| N-(4-fluoro-3-chlorobenzyl)-6-fluoro-5-{2-[5-(hydroxymethyl)-1,4- | 1 | —CH₂OH | H | F | Cl |
| dioxan-2-yl]methoxy}-4-oxo-3,4-dihydroquinazoline-2- | | | | | |
| carboxamide | | | | | |
| N-(3-methoxybenzyl)-6-fluoro-5-{2-[5-(hydroxymethyl)-1,4- | 1 | —CH₂OH | H | H | —OCH₃ |
| dioxan-2-yl]methoxy}-4-oxo-3,4-dihydroquinazoline-2- | | | | | |
| carboxamide | | | | | |
| N-(3-chlorobenzyl)-6-fluoro-5-{2-[5-(hydroxymethyl)-1,4-dioxan- | 1 | —CH₂OH | H | H | Cl |
| 2-yl]methoxy}-4-oxo-3,4-dihydroquinazoline-2-carboxamide | | | | | |
| N-(4-fluoro-3-methoxybenzyl)-6-fluoro-5-{2-[5- | 1 | —CH₂SO₂CH₃ | H | F | —OCH₃ |
| (methylsulfonylmethyl)-1,4-dioxan-2-yl]methoxy}-4-oxo-3,4- | | | | | |
| dihydroquinazoline-2-carboxamide | | | | | |
| N-(4-fluoro-3-chlorobenzyl)-6-fluoro-5-{2-[5- | 1 | —CH₂SO₂CH₃ | H | F | Cl |
| (methylsulfonylmethyl)-1,4-dioxan-2-yl]methoxy}-4-oxo-3,4- | | | | | |
| dihydroquinazoline-2-carboxamide | | | | | |
| N-(3-methoxybenzyl)-6-fluoro-5-{2-[5-(methylsulfonylmethyl)-1,4- | 1 | —CH₂SO₂CH₃ | H | H | —OCH₃ |
| dioxan-2-yl]methoxy}-4-oxo-3,4-dihydroquinazoline-2- | | | | | |
| carboxamide | | | | | |
| N-(3-chlorobenzyl)-6-fluoro-5-{2-[5-(methylsulfonylmethyl)-1,4- | 1 | —CH₂SO₂CH₃ | H | H | Cl |
| dioxan-2-yl]methoxy}-4-oxo-3,4-dihydroquinazoline-2- | | | | | |
| carboxamide | | | | | |
| N-(4-fluoro-3-methoxybenzyl)-5-[2-(5-cyano-1,4-dioxan-2- | 1 | CN | H | F | —OCH₃ |
| yl)methoxy]-6-fluoro-4-oxo-3,4-dihydroquinazoline-2- | | | | | |
| carboxamide | | | | | |
| N-(4-fluoro-3-chlorobenzyl)-5-[2-(5-cyano-1,4-dioxan-2- | 1 | CN | H | F | Cl |
| yl)methoxy]-6-fluoro-4-oxo-3,4-dihydroquinazoline-2- | | | | | |
| carboxamide | | | | | |
| N-(3-methoxybenzyl)-5-[2-(5-cyano-1,4-dioxan-2-yl)methoxy]-6- | 1 | CN | H | H | —OCH₃ |
| fluoro-4-oxo-3,4-dihydroquinazoline-2-carboxamide | | | | | |
| N-(3-chlorobenzyl)-5-[2-(5-cyano-1,4-dioxan-2-yl)methoxy]-6- | 1 | CN | H | H | Cl |
| fluoro-4-oxo-3,4-dihydroquinazoline-2-carboxamide | | | | | |
| 5-[(5-(aminomethyl)-1,4-dioxan-2-yl)methoxy]-6-fluoro-N-(3- | 1 | —CH₂NH₂ | H | F | —OCH₃ |
| methoxy-4-fluorobenzyl)-4-oxo-3,4-dihydroquinazoline-2- | | | | | |
| carboxamide | | | | | |
| 5-[(5-(aminomethyl)-1,4-dioxan-2-yl)methoxy]-6-fluoro-N-(3- | 1 | —CH₂NH₂ | H | F | Cl |
| chloro-4-fluorobenzyl)-4-oxo-3,4-dihydroquinazoline-2- | | | | | |
| carboxamide | | | | | |
| 5-[(5-(aminomethyl)-1,4-dioxan-2-yl)methoxy]-6-fluoro-N-(3- | 1 | —CH₂NH₂ | H | H | —OCH₃ |
| methoxybenzyl)-4-oxo-3,4-dihydroquinazoline-2-carboxamide | | | | | |
| 5-[(5-(aminomethyl)-1,4-dioxan-2-yl)methoxy]-6-fluoro-N-(3- | 1 | —CH₂NH₂ | H | H | Cl |
| methoxybenzyl)-4-oxo-3,4-dihydroquinazoline-2-carboxamide | | | | | |

Another embodiment of the invention is a compound of Formula II-11, shown in Table 11.

TABLE 11
II-11

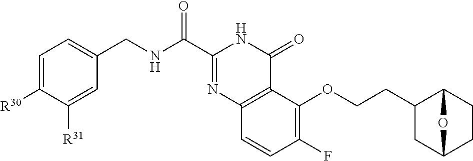

| Compound Name | R³⁰ | R³¹ |
|---|---|---|
| N-(4-fluoro-3-methoxybenzyl)-5-{2-[(1S,4R)-7-oxa-bicyclo[2.2.1]heptan-2-yl]ethoxy}-6-fluoro-4- | F | —OCH₃ |
| oxo-3,4-dihydroquinazoline-2-carboxamide | | |
| N-(4-fluoro-3-chlorobenzyl)-5-{2-[(1S,4R)-7-oxa-bicyclo[2.2.1]heptan-2-yl]ethoxy}-6-fluoro-4- | F | Cl |
| oxo-3,4-dihydroquinazoline-2-carboxamide | | |
| N-(3-methoxybenzyl)-5-{2-[(1S,4R)-7-oxa-bicyclo[2.2.1]heptan-2-yl]ethoxy}-6-fluoro-4-oxo-3,4- | H | —OCH₃ |
| dihydroquinazoline-2-carboxamide | | |
| N-(3-chlorobenzyl)-5-{2-[(1S,4R)-7-oxa-bicyclo[2.2.1]heptan-2-yl]ethoxy}-6-fluoro-4-oxo-3,4- | H | Cl |
| dihydroquinazoline-2-carboxamide | | |

Another embodiment of the invention is a compound of Formula II-12, shown in Table 12.

TABLE 12
II-12

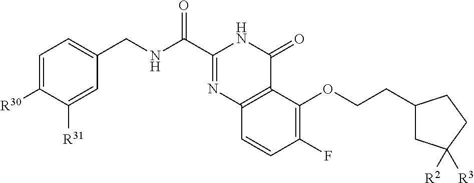

| Compound Name | R² | R³ | R³⁰ | R³¹ |
|---|---|---|---|---|
| N-(4-fluoro-3-methoxybenzyl)-6-fluoro-5-{2-[3-hydroxy-3- | —CH₂OH | OH | F | —OCH₃ |
| (hydroxymethyl)cyclopentyl]ethoxy}-4-oxo-3,4-dihydroquinazoline-2- | | | | |
| carboxamide | | | | |
| N-(4-fluoro-3-chlorobenzyl)-6-fluoro-5-{2-[3-hydroxy-3- | —CH₂OH | OH | F | Cl |
| (hydroxymethyl)cyclopentyl]ethoxy}-4-oxo-3,4-dihydroquinazoline-2- | | | | |
| carboxamide | | | | |
| N-(3-methoxybenzyl)-6-fluoro-5-{2-[3-hydroxy-3- | —CH₂OH | OH | H | —OCH₃ |
| (hydroxymethyl)cyclopentyl]ethoxy}-4-oxo-3,4-dihydroquinazoline-2- | | | | |
| carboxamide | | | | |
| N-(3-chlorobenzyl)-6-fluoro-5-{2-[3-hydroxy-3- | —CH₂OH | OH | H | Cl |
| (hydroxymethyl)cyclopentyl]ethoxy}-4-oxo-3,4-dihydroquinazoline-2- | | | | |
| carboxamide | | | | |
| N-(4-fluoro-3-methoxybenzyl)-5-[2-(3-cyanocyclopentyl)ethoxy]-6- | CN | H | F | —OCH₃ |
| fluoro-4-oxo-3,4-dihydroquinazoline-2-carboxamide | | | | |
| N-(4-fluoro-3-chlorobenzyl)-5-[2-(3-cyanocyclopentyl)ethoxy]-6- | CN | H | F | Cl |
| fluoro-4-oxo-3,4-dihydroquinazoline-2-carboxamide | | | | |
| N-(3-methoxybenzyl)-5-[2-(3-cyanocyclopentyl)ethoxy]-6-fluoro-4- | CN | H | H | —OCH₃ |
| oxo-3,4-dihydroquinazoline-2-carboxamide | | | | |
| N-(3-chlorobenzyl)-5-[2-(3-cyanocyclopentyl)ethoxy]-6-fluoro-4-oxo- | CN | H | H | Cl |
| 3,4-dihydroquinazoline-2-carboxamide | | | | |
| N-(4-fluoro-3-methoxybenzyl)-6-fluoro-5-{2-[3-(2- | —NHC(═O)CH₂OH | H | F | —OCH₃ |
| hydroxyacetamido)cyclopentyl]ethoxy}-4-oxo-3,4- | | | | |
| dihydroquinazoline-2-carboxamide | | | | |
| N-(4-fluoro-3-chlorobenzyl)-6-fluoro-5-{2-[3-(2- | —NHC(═O)CH₂OH | H | F | Cl |
| hydroxyacetamido)cyclopentyl]ethoxy}-4-oxo-3,4- | | | | |
| dihydroquinazoline-2-carboxamide | | | | |
| N-(3-methoxybenzyl)-6-fluoro-5-{2-[3-(2- | —NHC(═O)CH₂OH | H | H | —OCH₃ |
| hydroxyacetamido)cyclopentyl]ethoxy}-4-oxo-3,4- | | | | |
| dihydroquinazoline-2-carboxamide | | | | |
| N-(3-chlorobenzyl)-6-fluoro-5-{2-[3-(2- | —NHC(═O)CH₂OH | H | H | Cl |
| hydroxyacetamido)cyclopentyl]ethoxy}-4-oxo-3,4- | | | | |
| dihydroquinazoline-2-carboxamide | | | | |
| N-(4-fluoro-3-methoxybenzyl)-5-[2-(3-acetamidocyclopentyl)ethoxy]- | —NHC(═O)CH₃ | H | F | —OCH₃ |
| 6-fluoro-4-oxo-3,4-dihydroquinazoline-2-carboxamide | | | | |
| N-(4-fluoro-3-chlorobenzyl)-5-[2-(3-acetamidocyclopentyl)ethoxy]-6- | —NHC(═O)CH₃ | H | F | Cl |
| fluoro-4-oxo-3,4-dihydroquinazoline-2-carboxamide | | | | |
| N-(3-methoxybenzyl)-5-[2-(3-acetamidocyclopentyl)ethoxy]-6-fluoro- | —NHC(═O)CH₃ | H | H | —OCH₃ |
| 4-oxo-3,4-dihydroquinazoline-2-carboxamide | | | | |
| N-(3-chlorobenzyl)-5-[2-(3-acetamidocyclopentyl)ethoxy]-6-fluoro-4- | —NHC(═O)CH₃ | H | H | Cl |
| oxo-3,4-dihydroquinazoline-2-carboxamide | | | | |
| N-(4-fluoro-3-methoxybenzyl)-6-fluoro-5-{2-[3- | —NHSO₂CH₃ | H | F | —OCH₃ |
| (methylsulfonamido)cyclopentyl]ethoxy}-4-oxo-3,4- | | | | |
| dihydroquinazoline-2-carboxamide | | | | |
| N-(4-fluoro-3-chlorobenzyl)-6-fluoro-5-{2-[3- | —NHSO₂CH₃ | H | F | Cl |
| (methylsulfonamido)cyclopentyl]ethoxy}-4-oxo-3,4- | | | | |
| dihydroquinazoline-2-carboxamide | | | | |
| N-(3-methoxybenzyl)-6-fluoro-5-{2-[3- | —NHSO₂CH₃ | H | H | —OCH₃ |
| (methylsulfonamido)cyclopentyl]ethoxy}-4-oxo-3,4- | | | | |
| dihydroquinazoline-2-carboxamide | | | | |
| N-(3-chlorobenzyl)-6-fluoro-5-{2-[3- | —NHSO₂CH₃ | H | H | Cl |
| (methylsulfonamido)cyclopentyl]ethoxy}-4-oxo-3,4- | | | | |
| dihydroquinazoline-2-carboxamide | | | | |

Another embodiment of the invention is a compound selected from the compounds named in Tables 1-12.

Another embodiment of the invention is a compound selected from 6-fluoro-N-(3-methoxybenzyl)-4-oxo-5-(2-piperidin-4-ylethoxy)-3,4-dihydroquinazoline-2-carboxamide; 2-(4-{2-[(6-fluoro-2-{[(3-methoxybenzyl)amino]carbonyl}-4-oxo-3,4-dihydroquinazolin-5-yl)oxy]ethyl}piperidin-1-yl)-2-oxoethyl acetate; 6-fluoro-5-{2-[1-(2-hydroxyethyl)piperidin-4-yl]ethoxy}-N-(3-methoxybenzyl)-4-oxo-3,4-dihydroquinazoline-2-carboxamide; N-(3-methoxybenzyl)-6-fluoro-5-{2-[1-(2-hydroxyacetyl)piperidin-4-yl]ethoxy}-4-oxo-3,4-dihydroquinazoline-2-carboxamide; N-(3-methoxybenzyl)-6-fluoro-5-{2-[1-(methylsulfonyl)piperidin-4-yl]ethoxy}-4-oxo-3,4-dihydroquinazoline-2-carboxamide; 6-fluoro-5-{[4-(hydroxymethyl)cyclohexyl]methoxy}-N-(3-methoxybenzyl)-4-oxo-3,4-dihydroquinazoline-2-carboxamide; N-(3-methoxybenzyl)-6-fluoro-5-{2-[5-(hydroxymethyl)-1,4-dioxan-2-yl]methoxy}-4-oxo-3,4-dihydroquinazoline-2-carboxamide; and 5-[(5-(aminomethyl)-1,4-dioxan-2-yl)methoxy]-6-fluoro-N-(3-methoxybenzyl)-4-oxo-3,4-dihydroquinazoline-2-carboxamide; or a pharmaceutically acceptable salt thereof.

Another embodiment of the invention is a compound selected from 6-fluoro-N-(3-methoxybenzyl)-4-oxo-5-(2-piperidin-4-ylethoxy)-3,4-dihydroquinazoline-2-carboxamide; 2-(4-{2-[(6-fluoro-2-{[(3-methoxybenzyl)amino]carbonyl}-4-oxo-3,4-dihydroquinazolin-5-yl)oxy]ethyl}piperidin-1-yl)-2-oxoethyl acetate; 6-fluoro-5-{2-[1-(2-hydroxyethyl)piperidin-4-yl]ethoxy}-N-(3-methoxybenzyl)-4-oxo-3,4-dihydroquinazoline-2-carboxamide; 6-fluoro-5-[2-(1-glycoloylpiperidin-4-yl)ethoxy]-N-(3-methoxybenzyl)-4-oxo-3,4-dihydroquinazoline-2-carboxamide; 6-fluoro-N-(3-methoxybenzyl)-5-{2-[1-(methylsulfonyl)piperidin-4-yl]ethoxy}-4-oxo-3,4-dihydroquinazoline-2-carboxamide; 6-fluoro-5-{[trans-4-(hydroxymethyl)cyclohexyl]methoxy}-N-(3-methoxybenzyl)-4-oxo-3,4-dihydroquinazoline-2-carboxamide; 6-Fluoro-5-{[(trans-2,5)-5-(hydroxymethyl)-1,4-dioxan-2-yl]methoxy}-N-(3-methoxybenzyl)-4-oxo-3,4-dihydroquinazoline-2-carboxamide; and 5-{[(trans 2,5)-5-(aminomethyl)-1,4-dioxan-2-yl]methoxy}-6-fluoro-N-(3-methoxybenzyl)-4-oxo-3,4-dihydroquinazoline-2-carboxamide; or a pharmaceutically acceptable salt thereof.

Another embodiment of the invention is a compound of Formula III:

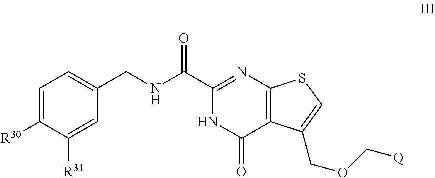

wherein:

Q is

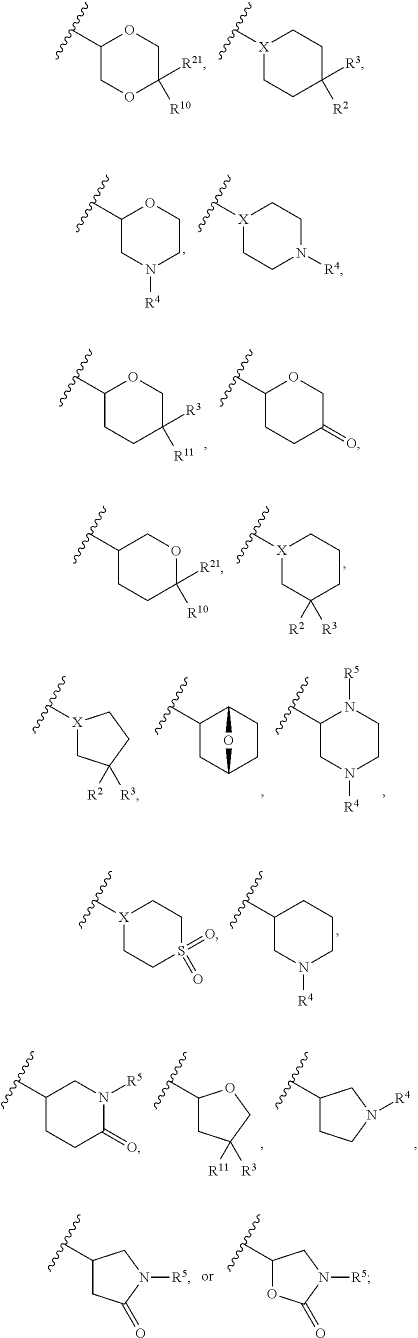

X is N or CH;

R² is H, CN, —OR⁵, R¹², —C(═O)R⁷, —NR⁸R³³, —NR⁸C(═O)R⁹, —NR⁸SO₂R³⁴, or —SO₂R¹²;

R³ is H, CN, or —OR²²;

R⁴ is H, —(C₁₋₆ alkylene)R⁶, —C(═O)R⁹, or —SO₂R¹²;

R⁵ is H or —(C₁₋₆ alkyl), wherein said C₁₋₆ alkyl may be substituted by one or more R²⁶ substituents;

R⁶ is H, CN, —OR²³, —SO₂R³⁵, —NR²⁴C(═O)R²³, —NR²⁴SO₂R³⁵, or

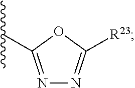

R⁷ is —(C₁₋₆ alkyl), —(C₁₋₆ alkylene)OH, —NHR²⁴, or —OR²⁵;

R⁹ is —(C₁₋₆ alkylene)R²⁸, —NHR²⁴, or —OR²⁵;

R¹⁰ is H, CN, R¹², or —C(═O)R⁷;

R¹¹ is H, CN, —OR⁵, R¹², —C(═O)R⁷, —NR⁸R³³, —NR⁸C(═O)R⁹, —NR⁸SO₂R³⁴, or —SO₂R¹²;

R¹² is —(C₁₋₆ alkyl), wherein said C₁₋₆ alkyl may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of said C₁₋₆ alkyl is not substituted by more than one CN or more than one —OR²³;

R⁸, R²¹, R²², R²⁴, R²⁵, and R³³ are independently H or —(C₁₋₆ alkyl);

R²³ is H, —(C₁₋₆ alkyl), or —(C₁₋₆ alkylene)OH;

R²⁶ is H, OH, halo, NH₂, or SH;

R²⁸ is H or —OR²⁹;

R²⁹ is H or —C(═O)(C₁₋₆ alkyl);

R³⁰ is H or F;

R³¹ is Cl, Br, —OR³², (C₁₋₆ alkyl), —OCH₂CH₂OR²⁵, —(C₃₋₆ cycloalkyl), or CN;

R³² is —(C₁₋₆ alkyl) optionally substituted with one, two, or three F;

R³⁴ is —(C₁₋₆ alkyl); and

R³⁵ is —(C₁₋₆ alkyl) or —(C₁₋₆ hydroxyalkyl);

or a pharmaceutically acceptable salt thereof;

provided that if Q is

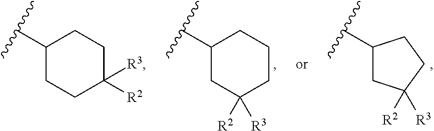

R² and R³ are not both H.

Another embodiment of the invention is a compound of Formula III as shown above wherein Q is

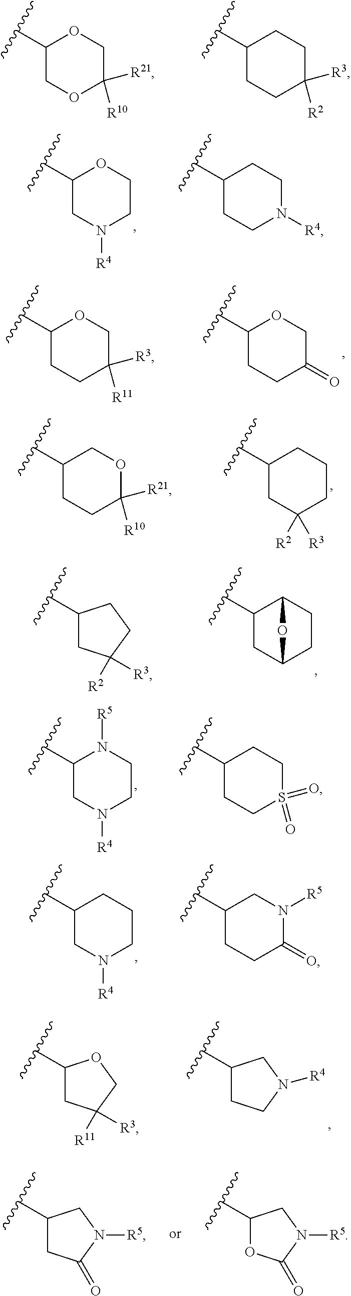

Another embodiment of the invention is a compound of Formula III as shown above wherein Q is

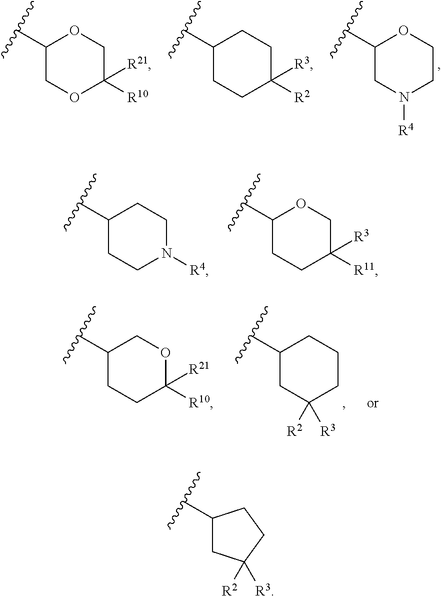

Another embodiment of the invention is a compound of Formula III as shown above wherein Q is

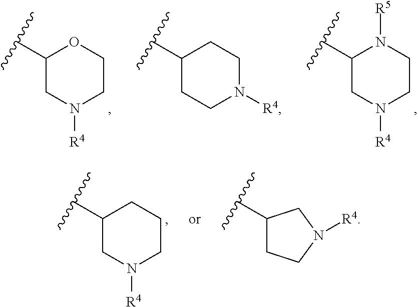

Another embodiment of the invention is a compound of Formula III as shown above wherein Q is

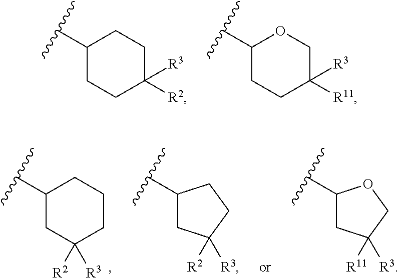

Another embodiment of the invention is a compound of Formula III as shown above wherein Q is

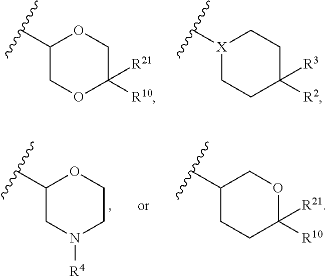

Another embodiment of the invention is a compound of Formula III as shown above wherein Q is

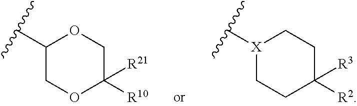

Another embodiment of the invention is a compound of Formula III as shown above wherein Q is

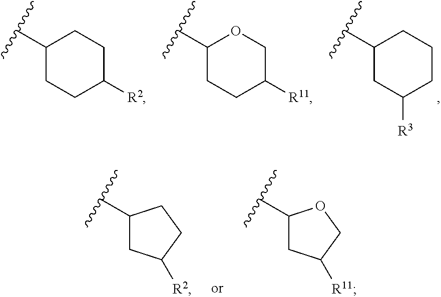

### R² is CN, OH,

R¹², —C(═O)R⁷, —NR⁸C(═O)R⁹, or —NR⁸SO₂R³⁴; and R¹¹ is H, CN, OH, R¹², —C(═O)R⁷, —NR⁸C(═O)R⁹, or —NR⁸SO₂R³⁴.

Another embodiment of the invention is a compound of Formula III as shown above wherein Q is

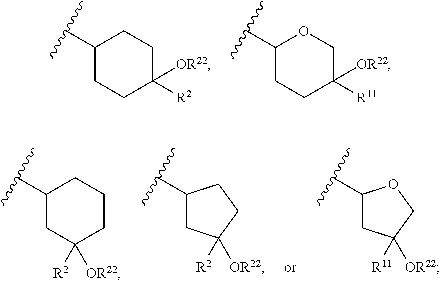

### and R² and R¹¹ are R¹² or —C(═O)R⁷.

Another embodiment of the invention is a compound of Formula III as shown above wherein Q is

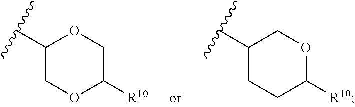

### and R¹⁰ is H, R¹², or —C(═O)R⁷.

Another embodiment of the invention is a compound of Formula IIIA:

wherein:

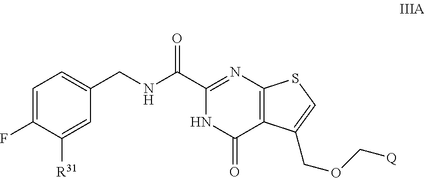

Q is

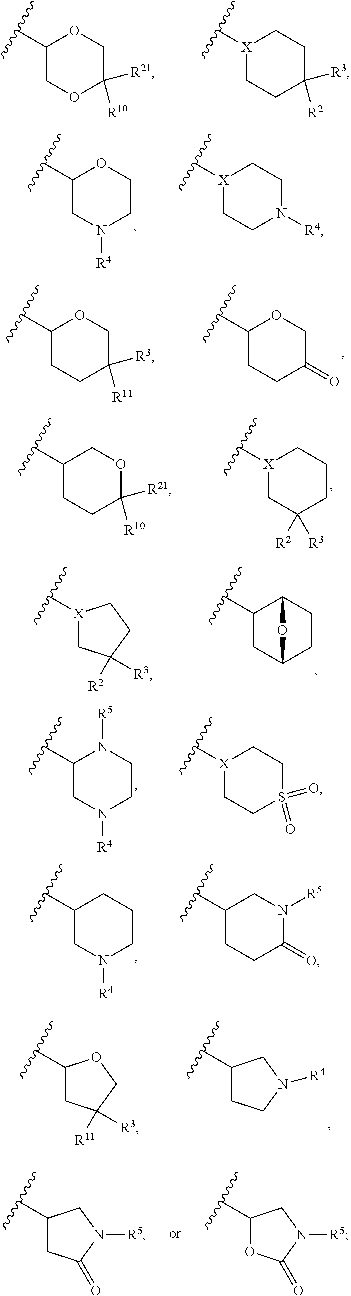

X is N or CH;

R¹ is H or F;

R² is H, CN, R¹², C(═O)R⁷, —NR⁸R³³, —NR⁸C(═O)R⁹, —NR⁸SO₂R³⁴, or —SO₂R¹²;

R³ is H, CN, or —OR²²;

R⁴ is H, —(C₁₋₆ alkylene)R⁶, —C(═O)R⁹, or —SO₂R¹²;

R⁵ is H or —(C₁₋₆ alkyl), wherein said C₁₋₆ alkyl may be substituted by one or more R²⁶ substituents;

R⁶ is H, CN, —OR²³, —SO₂R³⁵, —NR²⁴C(═O)R²³, —NR²⁴SO₂R³⁵, or

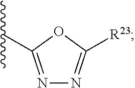

R⁷ is —(C₁₋₆ alkyl), —(C₁₋₆ alkylene)OH, —NHR²⁴, or —OR²⁵;

R⁹ is —(C₁₋₆ alkylene)R²⁸, —NHR²⁴, or —OR²⁵;

R¹⁰ is H, CN, R¹², or —C(═O)R⁷;

R¹¹ is H, CN, R¹², —C(═O)R⁷, —NR⁸R³³, —NR⁸C(═O)R⁹, —NR⁸SO₂R³⁴, or —SO₂R¹²;

R¹² is —(C₁₋₆ alkyl), wherein said C₁₋₆ alkyl may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of said C₁₋₆ alkyl is not substituted by more than one CN or more than one —OR²³;

R⁸, R²¹, R²², R²⁴, R²⁵, and R³³ are independently H or —(C₁₋₆ alkyl);

R²³ is H, —(C₁₋₆ alkyl), or —(C₁₋₆ alkylene)OH;

R²⁶ is H, OH, halo, NH₂, or SH;

R²⁸ is H or —OR²⁹;

R²⁹ is H or —C(═O)(C₁₋₆ alkyl);

R³¹ is Cl, Br, —OR³², (C₁₋₆ alkyl), —OCH₂CH₂OR²⁵, —(C₃₋₆ cycloalkyl), or CN;

R³² is —(C₁₋₆ alkyl) optionally substituted with one, two, or three F;

R³⁴ is —(C₁₋₆ alkyl); and

R³⁵ is —(C₁₋₆ alkyl) or —(C₁₋₆ hydroxyalkyl);

or a pharmaceutically acceptable salt thereof:

provided that if Q is

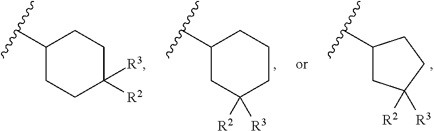

R² and R³ are not both H.

Another embodiment of the invention is a compound of Formula IIIA as shown above wherein R³¹ is Cl or —OCH₃.

Another embodiment of the invention is a compound of Formula III-1, shown in Table 13.

TABLE 13
III-1

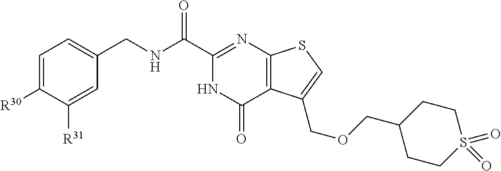

| Compound Name | R³⁰ | R³¹ |
|---|---|---|
| N-(4-fluoro-3-methoxybenzyl)-4-oxo-5-{[(tetrahydro-2H-1,1′-dioxothiopyran-4- | F | —OCH₃ |
| yl)methoxy]methyl}-3,4-dihydrothieno[2,3-d]pyrimidine-2-carboxamide | | |
| N-(4-fluoro-3-chlorobenzyl)-4-oxo-5-{[(tetrahydro-2H-1,1′-dioxothiopyran-4-yl)methoxy]methyl}- | F | Cl |
| 3,4-dihydrothieno[2,3-d]pyrimidine-2-carboxamide | | |
| N-(3-methoxybenzyl)-4-oxo-5-{[(tetrahydro-2H-1,1′-dioxothiopyran-4-yl)methoxy]methyl}-3,4- | H | —OCH₃ |
| dihydrothieno[2,3-d]pyrimidine-2-carboxamide | | |
| N-(3-chlorobenzyl)-4-oxo-5-{[(tetrahydro-2H-1,1′-dioxothiopyran-4-yl)methoxy]methyl}-3,4- | H | Cl |
| dihydrothieno[2,3-d]pyrimidine-2-carboxamide | | |

Another embodiment of the invention is a compound of Formula III-2, shown in Table 14.

TABLE 14
III-2

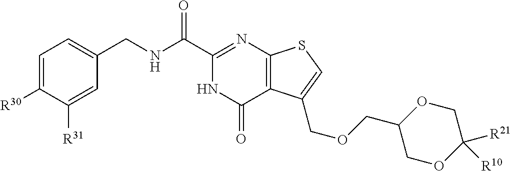

| Compound Name | R¹⁰ | R²¹ | R³⁰ | R³¹ |
|---|---|---|---|---|
| N-(4-fluoro-3-methoxybenzyl)-5-({[5-(2-hydroxypropan-2-yl)-1,4- | —C(OH)(CH₃)₂ | H | F | —OCH₃ |
| dioxan-2-yl]methoxy}methyl)-4-oxo-3,4-dihydrothieno[2,3- | | | | |
| d]pyrimidine-2-carboxamide | | | | |
| N-(4-fluoro-3-chlorobenzyl)-5-({[5-(2-hydroxypropan-2-yl)-1,4-dioxan- | —C(OH)(CH₃)₂ | H | F | Cl |
| 2-yl]methoxy}methyl)-4-oxo-3,4-dihydrothieno[2,3-d]pyrimidine-2- | | | | |
| carboxamide | | | | |
| N-(3-methoxybenzyl)-5-({[5-(2-hydroxypropan-2-yl)-1,4-dioxan-2- | —C(OH)(CH₃)₂ | H | H | —OCH₃ |
| yl]methoxy}methyl)-4-oxo-3,4-dihydrothieno[2,3-d]pyrimidine-2- | | | | |
| carboxamide | | | | |
| N-(3-chlorobenzyl)-5-({[5-(2-hydroxypropan-2-yl)-1,4-dioxan-2- | —C(OH)(CH₃)₂ | H | H | Cl |
| yl]methoxy}methyl)-4-oxo-3,4-dihydrothieno[2,3-d]pyrimidine-2- | | | | |
| carboxamide | | | | |
| 5-[({2-[(4-fluoro-3-methoxybenzyl)carbamoyl]-4-oxo-3,4- | ═C(═O)OH | H | F | —OCH₃ |
| dihydrothieno[2,3-d]pyrimidin-5-yl}methoxy)methyl]-1,4-dioxane-2- | | | | |
| carboxylic acid | | | | |
| 5-[({2-[(4-fluoro-3-chlorobenzyl)carbamoyl]-4-oxo-3,4- | ═C(═O)OH | H | F | Cl |
| dihydrothieno[2,3-d]pyrimidin-5-yl}methoxy)methyl]-1,4-dioxane-2- | | | | |
| carboxylic acid | | | | |
| 5-[({2-[(3-methoxybenzyl)carbamoyl]-4-oxo-3,4-dihydrothieno[2,3- | ═C(═O)OH | H | H | —OCH₃ |
| d]pyrimidin-5-yl}methoxy)methyl]-1,4-dioxane-2-carboxylic acid | | | | |
| 5-[({2-[(3-chlorobenzyl)carbamoyl]-4-oxo-3,4-dihydrothieno[2,3- | ═C(═O)OH | H | H | Cl |
| d]pyrimidin-5-yl}methoxy)methyl]-1,4-dioxane-2-carboxylic acid | | | | |
| N-(4-fluoro-3-methoxybenzyl)-5-{[(5-carbamoyl-1,4-dioxan-2- | —C(═O)NH₂ | H | F | —OCH₃ |
| yl)methoxy]methyl}-4-oxo-3,4-dihydrothieno[2,3-d]pyrimidine-2- | | | | |
| carboxamide | | | | |
| N-(4-fluoro-3-chlorobenzyl)-5-{[(5-carbamoyl-1,4-dioxan-2- | —C(═O)NH₂ | H | F | Cl |
| yl)methoxy]methyl}-4-oxo-3,4-dihydrothieno[2,3-d]pyrimidine-2- | | | | |
| carboxamide | | | | |
| N-(3-methoxybenzyl)-5-{[(5-carbamoyl-1,4-dioxan-2- | —C(═O)NH₂ | H | H | —OCH₃ |
| yl)methoxy]methyl}-4-oxo-3,4-dihydrothieno[2,3-d]pyrimidine-2- | | | | |
| carboxamide | | | | |
| N-(3-chlorobenzyl)-5-{[(5-carbamoyl-1,4-dioxan-2- | —C(═O)NH₂ | H | H | Cl |
| yl)methoxy]methyl}-4-oxo-3,4-dihydrothieno[2,3-d]pyrimidine-2- | | | | |
| carboxamide | | | | |
| 5-[({5-[(hydroxymethyl)carbamoyl)-1,4-dioxan-2-yl)methoxy)methyl)- | —C(═O)NHCH₂OH | H | F | —OCH₃ |
| N-(3-methoxybenzyl)-4-oxo-3,4-dihydrothieno[2,3-d]pyrimidine-2- | | | | |
| carboxamide | | | | |
| 5-[({5-[(hydroxymethyl)carbamoyl)-1,4-dioxan-2-yl)methoxy)methyl)- | —C(═O)NHCH₂OH | H | F | Cl |
| N-(3-chlorobenzyl)-4-oxo-3,4-dihydrothieno[2,3-d]pyrimidine-2- | | | | |
| carboxamide | | | | |
| 5-[({5-[(hydroxymethyl)carbamoyl)-1,4-dioxan-2-yl)methoxy)methyl)- | —C(═O)NHCH₂OH | H | H | —OCH₃ |
| N-(3-methoxybenzyl)-4-oxo-3,4-dihydrothieno[2,3-d]pyrimidine-2- | | | | |
| carboxamide | | | | |
| 5-[({5-[(hydroxymethyl)carbamoyl)-1,4-dioxan-2-yl)methoxy)methyl)- | —C(═O)NHCH₂OH | H | H | Cl |
| N-(3-chlorobenzyl)-4-oxo-3,4-dihydrothieno[2,3-d]pyrimidine-2- | | | | |
| carboxamide | | | | |
| N-(3-methoxybenzyl)-5-({[5-(methylcarbamoyl)-1,4-dioxan-2- | —C(═O)NHCH₃ | H | F | —OCH₃ |
| yl]methoxy}methyl)-4-oxo-3,4-dihydrothieno[2,3-d]pyrimidine-2- | | | | |
| carboxamide | | | | |
| N-(3-chlorobenzyl)-5-({[5-(methylcarbamoyl)-1,4-dioxan-2- | —C(═O)NHCH₃ | H | F | Cl |
| yl]methoxy}methyl)-4-oxo-3,4-dihydrothieno[2,3-d]pyrimidine-2- | | | | |
| carboxamide | | | | |
| N-(3-methoxybenzyl)-5-({[5-(methylcarbamoyl)-1,4-dioxan-2- | —C(═O)NHCH₃ | H | H | —OCH₃ |
| yl]methoxy}methyl)-4-oxo-3,4-dihydrothieno[2,3-d]pyrimidine-2- | | | | |
| carboxamide | | | | |
| N-(3-chlorobenzyl)-5-({[5-(methylcarbamoyl)-1,4-dioxan-2- | —C(═O)NHCH₃ | H | H | Cl |
| yl]methoxy}methyl)-4-oxo-3,4-dihydrothieno[2,3-d]pyrimidine-2- | | | | |
| carboxamide | | | | |
| N-(4-fluoro-3-methoxybenzyl)-5-({[5-(cyanomethyl)-1,4-dioxan-2- | —CH₂CN | H | F | —OCH₃ |
| yl]methoxy}methyl)-4-oxo-3,4-dihydrothieno[2,3-d]pyrimidine-2- | | | | |
| carboxamide | | | | |
| N-(4-fluoro-3-chlorobenzyl)-5-({[5-(cyanomethyl)-1,4-dioxan-2- | —CH₂CN | H | F | Cl |
| yl]methoxy}methyl)-4-oxo-3,4-dihydrothieno[2,3-d]pyrimidine-2- | | | | |
| carboxamide | | | | |
| N-(3-methoxybenzyl)-5-({[5-(cyanomethyl)-1,4-dioxan-2- | —CH₂CN | H | H | —OCH₃ |
| yl]methoxy}methyl)-4-oxo-3,4-dihydrothieno[2,3-d]pyrimidine-2- | | | | |
| carboxamide | | | | |
| N-(3-chlorobenzyl)-5-({[5-(cyanomethyl)-1,4-dioxan-2- | —CH₂CN | H | H | Cl |
| yl]methoxy}methyl)-4-oxo-3,4-dihydrothieno[2,3-d]pyrimidine-2- | | | | |
| carboxamide | | | | |
| N-(4-fluoro-3-methoxybenzyl)-5-({[5-(methylsulfonamidomethyl)-1,4- | —CH₂NHSO₂CH₃ | H | F | —OCH₃ |
| dioxan-2-yl]methoxy}methyl)-4-oxo-3,4-dihydrothieno[2,3- | | | | |
| d]pyrimidine-2-carboxamide | | | | |
| N-(4-fluoro-3-chlorobenzyl)-5-({[5-(methylsulfonamidomethyl)-1,4- | —CH₂NHSO₂CH₃ | H | F | Cl |
| dioxan-2-yl]methoxy}methyl)-4-oxo-3,4-dihydrothieno[2,3- | | | | |
| d]pyrimidine-2-carboxamide | | | | |
| N-(3-methoxybenzyl)-5-({[5-(methylsulfonamidomethyl)-1,4-dioxan- | —CH₂NHSO₂CH₃ | H | H | —OCH₃ |
| 2-yl]methoxy}methyl)-4-oxo-3,4-dihydrothieno[2,3-d]pyrimidine-2- | | | | |
| carboxamide | | | | |
| N-(3-chlorobenzyl)-5-({[5-(methylsulfonamidomethyl)-1,4-dioxan-2- | —CH₂NHSO₂CH₃ | H | H | Cl |
| yl]methoxy}methyl)-4-oxo-3,4-dihydrothieno[2,3-d]pyrimidine-2- | | | | |
| carboxamide | | | | |
| N-(4-fluoro-3-methoxybenzyl)-5-({[5-(hydroxymethyl)-1,4-dioxan-2- | —CH₂OH | H | F | —OCH₃ |
| yl]methoxy}methyl)-4-oxo-3,4-dihydrothieno[2,3-d]pyrimidine-2- | | | | |
| carboxamide | | | | |
| N-(4-fluoro-3-chlorobenzyl)-5-({[5-(hydroxymethyl)-1,4-dioxan-2- | —CH₂OH | H | F | Cl |
| yl]methoxy}methyl)-4-oxo-3,4-dihydrothieno[2,3-d]pyrimidine-2- | | | | |
| carboxamide | | | | |
| N-(3-methoxybenzyl)-5-({[5-(hydroxymethyl)-1,4-dioxan-2- | —CH₂OH | H | H | —OCH₃ |
| yl]methoxy}methyl)-4-oxo-3,4-dihydrothieno[2,3-d]pyrimidine-2- | | | | |
| carboxamide | | | | |
| N-(3-chlorobenzyl)-5-({[5-(hydroxymethyl)-1,4-dioxan-2- | —CH₂OH | H | H | Cl |
| yl]methoxy}methyl)-4-oxo-3,4-dihydrothieno[2,3-d]pyrimidine-2- | | | | |
| carboxamide | | | | |
| N-(4-fluoro-3-methoxybenzyl)-5-({[5-(methylsulfonylmethyl)-1,4- | —CH₂SO₂CH₃ | H | F | —OCH₃ |
| dioxan-2-yl]methoxy}methyl)-4-oxo-3,4-dihydrothieno[2,3- | | | | |
| d]pyrimidine-2-carboxamide | | | | |
| N-(4-fluoro-3-chlorobenzyl)-5-({[5-(methylsulfonylmethyl)-1,4-dioxan- | —CH₂SO₂CH₃ | H | F | Cl |
| 2-yl]methoxy}methyl)-4-oxo-3,4-dihydrothieno[2,3-d]pyrimidine-2- | | | | |
| carboxamide | | | | |
| N-(3-methoxybenzyl)-5-({[5-(methylsulfonylmethyl)-1,4-dioxan-2- | —CH₂SO₂CH₃ | H | H | —OCH₃ |
| yl]methoxy}methyl)-4-oxo-3,4-dihydrothieno[2,3-d]pyrimidine-2- | | | | |
| carboxamide | | | | |
| N-(3-chlorobenzyl)-5-({[5-(methylsulfonylmethyl)-1,4-dioxan-2- | —CH₂SO₂CH₃ | H | H | Cl |
| yl]methoxy}methyl)-4-oxo-3,4-dihydrothieno[2,3-d]pyrimidine-2- | | | | |
| carboxamide | | | | |
| N-(4-fluoro-3-methoxybenzyl)-5-{[(5-cyano-1,4-dioxan-2- | CN | H | F | —OCH₃ |
| yl)methoxy]methyl}-4-oxo-3,4-dihydrothieno[2,3-d]pyrimidine-2- | | | | |
| carboxamide | | | | |
| N-(4-fluoro-3-chlorobenzyl)-5-{[(5-cyano-1,4-dioxan-2- | CN | H | F | Cl |
| yl)methoxy]methyl}-4-oxo-3,4-dihydrothieno[2,3-d]pyrimidine-2- | | | | |
| carboxamide | | | | |
| N-(3-methoxybenzyl)-5-{[(5-cyano-1,4-dioxan-2-yl)methoxy]methyl}- | CN | H | H | —OCH₃ |
| 4-oxo-3,4-dihydrothieno[2,3-d]pyrimidine-2-carboxamide | | | | |
| N-(3-chlorobenzyl)-5-{[(5-cyano-1,4-dioxan-2-yl)methoxy]methyl}-4- | CN | H | H | Cl |
| oxo-3,4-dihydrothieno[2,3-d]pyrimidine-2-carboxamide | | | | |

Another embodiment of the invention is a compound of Formula III-3, shown in Table 15.

TABLE 15
III-3

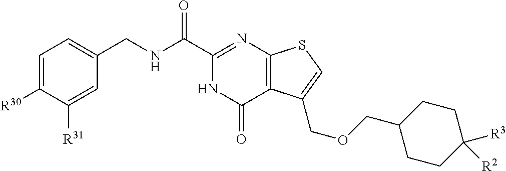

| Compound Name | R² | R³ | R³⁰ | R³¹ |
|---|---|---|---|---|
| 5-({[4-aminomethylcyclohexyl]methoxy}methyl)-N-(3- | —CH₂NH₂ | H | F | —OCH₃ |
| methoxy-4-fluorobenzyl)-4-oxo-3,4-dihydrothieno[2,3- | | | | |
| d]pyrimidine-2-carboxamide | | | | |
| 5-({[4-aminomethylcyclohexyl]methoxy}methyl)-N-(3-chloro- | —CH₂NH₂ | H | F | Cl |
| 4-fluorobenzyl)-4-oxo-3,4-dihydrothieno[2,3-d]pyrimidine-2- | | | | |
| carboxamide | | | | |
| 5-({[4-aminomethylcyclohexyl]methoxy}methyl)-N-(3- | —CH₂NH₂ | H | H | —OCH₃ |
| methoxybenzyl)-4-oxo-3,4-dihydrothieno[2,3-d]pyrimidine-2- | | | | |
| carboxamide | | | | |
| 5-({[4-aminomethylcyclohexyl]methoxy}methyl)-N-(3- | —CH₂NH₂ | H | H | Cl |
| chlorobenzyl)-4-oxo-3,4-dihydrothieno[2,3-d]pyrimidine-2- | | | | |
| carboxamide | | | | |
| N-(4-fluoro-3-methoxybenzyl)-5-({[4- | —C(═O)NHCH₃ | H | F | —OCH₃ |
| (methylcarbamoyl)cyclohexyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[4- | —C(═O)NHCH₃ | H | F | Cl |
| (methylcarbamoyl)cyclohexyl]methoxy}methyl)-4-oxo-3,4- | | | | |
| dihydrothieno[2,3-d]pyrimidine-2-carboxamide | | | | |
| N-(3-methoxybenzyl)-5-({[4- | —C(═O)NHCH₃ | H | H | —OCH₃ |
| (methylcarbamoyl)cyclohexyl]methoxy}methyl)-4-oxo-3,4- | | | | |
| dihydrothieno[2,3-d]pyrimidine-2-carboxamide | | | | |
| N-(3-chlorobenzyl)-5-({[4- | —C(═O)NHCH₃ | H | H | Cl |
| (methylcarbamoyl)cyclohexyl]methoxy}methyl)-4-oxo-3,4- | | | | |
| dihydrothieno[2,3-d]pyrimidine-2-carboxamide | | | | |
| 5-[({4- | —C(═O)NHCH₂OH | H | F | —OCH₃ |
| [(hydroxymethyl)carbamoyl]cyclohexyl}methoxy)methyl]-N- | | | | |
| (4-fluoro-3-methoxybenzyl)-4-oxo-3,4-dihydrothieno[2,3- | | | | |
| d]pyrimidine-2-carboxamide | | | | |
| 5-[({4- | —C(═O)NHCH₂OH | H | F | Cl |
| [(hydroxymethyl)carbamoyl]cyclohexyl}methoxy)methyl]-N- | | | | |
| (4-fluoro-3-chlorobenzyl)-4-oxo-3,4-dihydrothieno[2,3- | | | | |
| d]pyrimidine-2-carboxamide | | | | |
| 5-[({4- | —C(═O)NHCH₂OH | H | H | —OCH₃ |
| [(hydroxymethyl)carbamoyl]cyclohexyl}methoxy)methyl]-N- | | | | |
| (3-methoxybenzyl)-4-oxo-3,4-dihydrothieno[2,3-d]pyrimidine- | | | | |
| 2-carboxamide | | | | |
| 5-[({4- | —C(═O)NHCH₂OH | H | H | Cl |
| [(hydroxymethyl)carbamoyl]cyclohexyl}methoxy)methyl]-N- | | | | |
| (3-chlorobenzyl)-4-oxo-3,4-dihydrothieno[2,3-d]pyrimidine-2- | | | | |
| carboxamide | | | | |
| 2-{4-[({2-[(4-fluoro-3-methoxybenzyl)carbamoyl]-4-oxo-3,4- | —NHC(═O)CH₂OC(═O)CH₃ | H | F | —OCH₃ |
| dihydrothieno[2,3-d]pyrimidin-5- | | | | |
| yl}methoxy)methyl]cyclohexylamino}-2-oxoethyl acetate | | | | |
| 2-{4-[({2-[(4-fluoro-3-chlorobenzyl)carbamoyl]-4-oxo-3,4- | —NHC(═O)CH₂OC(═O)CH₃ | H | F | Cl |
| dihydrothieno[2,3-d]pyrimidin-5- | | | | |
| yl}methoxy)methyl]cyclohexylamino}-2-oxoethyl acetate | | | | |
| 2-{4-[({2-[(3-methoxybenzyl)carbamoyl]-4-oxo-3,4- | —NHC(═O)CH₂OC(═O)CH₃ | H | H | —OCH₃ |
| dihydrothieno[2,3-d]pyrimidin-5- | | | | |
| yl}methoxy)methyl]cyclohexylamino}-2-oxoethyl acetate | | | | |
| 2-{4-[({2-[(3-chlorobenzyl)carbamoyl]-4-oxo-3,4- | —NHC(═O)CH₂OC(═O)CH₃ | H | H | Cl |
| dihydrothieno[2,3-d]pyrimidin-5- | | | | |
| yl}methoxy)methyl]cyclohexylamino}-2-oxoethyl acetate | | | | |
| 2-[4-({2-[(4-fluoro-3-methoxybenzyl)carbamoyl]-4-oxo-3,4- | —C(═O)OH | H | F | —OCH₃ |
| dihydrothieno[2,3-d]pyrimidin-5- | | | | |
| yl}methoxy)methyl]cyclohexanecarboxylic acid | | | | |
| 2-[4-({2-[(4-fluoro-3-chlorobenzyl)carbamoyl]-4-oxo-3,4- | —C(═O)OH | H | F | Cl |
| dihydrothieno[2,3-d]pyrimidin-5- | | | | |
| yl}methoxy)methyl]cyclohexanecarboxylic acid | | | | |
| 2-[4-({2-[(3-methoxybenzyl)carbamoyl]-4-oxo-3,4- | —C(═O)OH | H | H | —OCH₃ |
| dihydrothieno[2,3-d]pyrimidin-5- | | | | |
| yl}methoxy)methyl]cyclohexanecarboxylic acid | | | | |
| 2-[4-({2-[(3-chlorobenzyl)carbamoyl]-4-oxo-3,4- | —C(═O)OH | H | H | Cl |
| dihydrothieno[2,3-d]pyrimidin-5- | | | | |
| yl}methoxy)methyl]cyclohexanecarboxylic acid | | | | |
| N-(4-fluoro-3-methoxybenzyl)-5-({[4-(2- | —NHC(═O)CH₂OH | H | F | —OCH₃ |
| hydroxyacetamido)cyclohexyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[4-(2- | —NHC(═O)CH₂OH | H | F | Cl |
| hydroxyacetamido)cyclohexyl]methoxy}methyl)-4-oxo-3,4- | | | | |
| dihydrothieno[2,3-d]pyrimidine-2-carboxamide | | | | |
| N-(3-methoxybenzyl)-5-({[4-(2- | —NHC(═O)CH₂OH | H | H | —OCH₃ |
| hydroxyacetamido)cyclohexyl]methoxy}methyl)-4-oxo-3,4- | | | | |
| dihydrothieno[2,3-d]pyrimidine-2-carboxamide | | | | |
| N-(3-chlorobenzyl)-5-({[4-(2- | —NHC(═O)CH₂OH | H | H | Cl |
| hydroxyacetamido)cyclohexyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[4-(2-hydroxypropan-2- | —C(OH)(CH₃)₂ | H | F | —OCH₃ |
| yl)cyclohexyl]methoxy}methyl)-4-oxo-3,4-dihydrothieno[2,3- | | | | |
| d]pyrimidine-2-carboxamide | | | | |
| N-(4-fluoro-3-chlorobenzyl)-5-({[4-(2-hydroxypropan-2- | —C(OH)(CH₃)₂ | H | F | Cl |
| yl)cyclohexyl]methoxy}methyl)-4-oxo-3,4-dihydrothieno[2,3- | | | | |
| d]pyrimidine-2-carboxamide | | | | |
| N-(3-methoxybenzyl)-5-({[4-(2-hydroxypropan-2- | —C(OH)(CH₃)₂ | H | H | —OCH₃ |
| yl)cyclohexyl]methoxy}methyl)-4-oxo-3,4-dihydrothieno[2,3- | | | | |
| d]pyrimidine-2-carboxamide | | | | |
| N-(3-chlorobenzyl)-5-({[4-(2-hydroxypropan-2- | —C(OH)(CH₃)₂ | H | H | Cl |
| yl)cyclohexyl]methoxy}methyl)-4-oxo-3,4-dihydrothieno[2,3- | | | | |
| d]pyrimidine-2-carboxamide | | | | |
| N-(4-fluoro-3-methoxybenzyl)-5-({[4- | —CH₂OH | H | F | —OCH₃ |
| (hydroxymethyl)cyclohexyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[4- | —CH₂OH | H | F | Cl |
| (hydroxymethyl)cyclohexyl]methoxy}methyl)-4-oxo-3,4- | | | | |
| dihydrothieno[2,3-d]pyrimidine-2-carboxamide | | | | |
| N-(3-methoxybenzyl)-5-({[4- | —CH₂OH | H | H | —OCH₃ |
| (hydroxymethyl)cyclohexyl]methoxy}methyl)-4-oxo-3,4- | | | | |
| dihydrothieno[2,3-d]pyrimidine-2-carboxamide | | | | |
| N-(3-chlorobenzyl)-5-({[4- | —CH₂OH | H | H | Cl |
| (hydroxymethyl)cyclohexyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[4- | —NHSO₂CH₃ | H | F | —OCH₃ |
| (methylsulfonamido)cyclohexyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[4- | —NHSO₂CH₃ | H | F | Cl |
| (methylsulfonamido)cyclohexyl]methoxy}methyl)-4-oxo-3,4- | | | | |
| dihydrothieno[2,3-d]pyrimidine-2-carboxamide | | | | |
| N-(3-methoxybenzyl)-5-({[4- | —NHSO₂CH₃ | H | H | —OCH₃ |
| (methylsulfonamido)cyclohexyl]methoxy}methyl)-4-oxo-3,4- | | | | |
| dihydrothieno[2,3-d]pyrimidine-2-carboxamide | | | | |
| N-(3-chlorobenzyl)-5-({[4- | —NHSO₂CH₃ | H | H | Cl |
| (methylsulfonamido)cyclohexyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[4- | —CH₂NHSO₂CH₃ | H | F | —OCH₃ |
| (methylsulfonamidomethyl)cyclohexyl]methoxy}methyl)-4- | | | | |
| oxo-3,4-dihydrothieno[2,3-d]pyrimidine-2-carboxamide | | | | |
| N-(4-fluoro-3-chlorobenzyl)-5-({[4- | —CH₂NHSO₂CH₃ | H | F | Cl |
| (methylsulfonamidomethyl)cyclohexyl]methoxy}methyl)-4- | | | | |
| oxo-3,4-dihydrothieno[2,3-d]pyrimidine-2-carboxamide | | | | |
| N-(3-methoxybenzyl)-5-({[4- | —CH₂NHSO₂CH₃ | H | H | —OCH₃ |
| (methylsulfonamidomethyl)cyclohexyl]methoxy}methyl)-4- | | | | |
| oxo-3,4-dihydrothieno[2,3-d]pyrimidine-2-carboxamide | | | | |
| N-(3-chlorobenzyl)-5-({[4- | —CH₂NHSO₂CH₃ | H | H | Cl |
| (methylsulfonamidomethyl)cyclohexyl]methoxy}methyl)-4- | | | | |
| oxo-3,4-dihydrothieno[2,3-d]pyrimidine-2-carboxamide | | | | |
| N-(4-fluoro-3-methoxybenzyl)-5-({[4- | —SO₂CH₃ | H | F | —OCH₃ |
| (methylsulfonyl)cyclohexyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[4- | —SO₂CH₃ | H | F | Cl |
| (methylsulfonyl)cyclohexyl]methoxy}methyl)-4-oxo-3,4- | | | | |
| dihydrothieno[2,3-d]pyrimidine-2-carboxamide | | | | |
| N-(3-methoxybenzyl)-5-({[4- | —SO₂CH₃ | H | H | —OCH₃ |
| (methylsulfonyl)cyclohexyl]methoxy}methyl)-4-oxo-3,4- | | | | |
| dihydrothieno[2,3-d]pyrimidine-2-carboxamide | | | | |
| N-(3-chlorobenzyl)-5-({[4- | —SO₂CH₃ | H | H | Cl |
| (methylsulfonyl)cyclohexyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[4- | —CH₂SO₂CH₃ | H | F | —OCH₃ |
| (methylsulfonylmethyl)cyclohexyl]methoxy}methyl)-4-oxo- | | | | |
| 3,4-dihydrothieno[2,3-d]pyrimidine-2-carboxamide | | | | |
| N-(4-fluoro-3-chlorobenzyl)-5-({[4- | —CH₂SO₂CH₃ | H | F | Cl |
| (methylsulfonylmethyl)cyclohexyl]methoxy}methyl)-4-oxo- | | | | |
| 3,4-dihydrothieno[2,3-d]pyrimidine-2-carboxamide | | | | |
| N-(3-methoxybenzyl)-5-({[4- | —CH₂SO₂CH₃ | H | H | —OCH₃ |
| (methylsulfonylmethyl)cyclohexyl]methoxy}methyl)-4-oxo- | | | | |
| 3,4-dihydrothieno[2,3-d]pyrimidine-2-carboxamide | | | | |
| N-(3-chlorobenzyl)-5-({[4- | —CH₂SO₂CH₃ | H | H | Cl |
| (methylsulfonylmethyl)cyclohexyl]methoxy}methyl)-4-oxo- | | | | |
| 3,4-dihydrothieno[2,3-d]pyrimidine-2-carboxamide | | | | |
| N-(4-fluoro-3-methoxybenzyl)-5-({[4- | —NHC(═O)CH₃ | H | F | —OCH₃ |
| acetamidocyclohexyl)methoxy]methyl}-4-oxo-3,4- | | | | |
| dihydrothieno[2,3-d]pyrimidine-2-carboxamide | | | | |
| N-(4-fluoro-3-chlorobenzyl)-5-{[(4- | —NHC(═O)CH₃ | H | F | Cl |
| acetamidocyclohexyl)methoxy]methyl}-4-oxo-3,4- | | | | |
| dihydrothieno[2,3-d]pyrimidine-2-carboxamide | | | | |
| N-(3-methoxybenzyl)-5-{[(4- | —NHC(═O)CH₃ | H | H | —OCH₃ |
| acetamidocyclohexyl)methoxy]methyl}-4-oxo-3,4- | | | | |
| dihydrothieno[2,3-d]pyrimidine-2-carboxamide | | | | |
| N-(3-chlorobenzyl)-5-{[(4- | —NHC(═O)CH₃ | H | H | Cl |
| acetamidocyclohexyl)methoxy]methyl}-4-oxo-3,4- | | | | |
| dihydrothieno[2,3-d]pyrimidine-2-carboxamide | | | | |
| N-(4-fluoro-3-methoxybenzyl)-5-{[(4- | —C(═O)NH₂ | H | F | —OCH₃ |
| carbamoylcyclohexyl)methoxy]methyl}-4-oxo-3,4- | | | | |
| dihydrothieno[2,3-d]pyrimidine-2-carboxamide | | | | |
| N-(4-fluoro-3-chlorobenzyl)-5-{[(4- | —C(═O)NH₂ | H | F | Cl |
| carbamoylcyclohexyl)methoxy]methyl}-4-oxo-3,4- | | | | |
| dihydrothieno[2,3-d]pyrimidine-2-carboxamide | | | | |
| N-(3-methoxybenzyl)-5-{[(4- | —C(═O)NH₂ | H | H | —OCH₃ |
| carbamoylcyclohexyl)methoxy]methyl}-4-oxo-3,4- | | | | |
| dihydrothieno[2,3-d]pyrimidine-2-carboxamide | | | | |
| N-(3-chlorobenzyl)-5-{[(4- | —C(═O)NH₂ | H | H | Cl |
| carbamoylcyclohexyl)methoxy]methyl}-4-oxo-3,4- | | | | |
| dihydrothieno[2,3-d]pyrimidine-2-carboxamide | | | | |
| N-(4-fluoro-3-methoxybenzyl)-5-{[(4- | CN | H | F | —OCH₃ |
| cyanocyclohexyl)methoxy]methyl}-4-oxo-3,4- | | | | |
| dihydrothieno[2,3-d]pyrimidine-2-carboxamide | | | | |
| N-(4-fluoro-3-chlorobenzyl)-5-{[(4- | CN | H | F | Cl |
| cyanocyclohexyl)methoxy]methyl}-4-oxo-3,4- | | | | |
| dihydrothieno[2,3-d]pyrimidine-2-carboxamide | | | | |
| N-(3-methoxybenzyl)-5-{[(4- | CN | H | H | —OCH₃ |
| cyanocyclohexyl)methoxy]methyl}-4-oxo-3,4- | | | | |
| dihydrothieno[2,3-d]pyrimidine-2-carboxamide | | | | |
| N-(3-chlorobenzyl)-5-{[(4-cyanocyclohexyl)methoxy]methyl}- | CN | H | H | Cl |
| 4-oxo-3,4-dihydrothieno[2,3-d]pyrimidine-2-carboxamide | | | | |
| N-(4-fluoro-3-methoxybenzyl)-5-{[(4- | OH | H | F | —OCH₃ |
| hydroxycyclohexyl)methoxy]methyl}-4-oxo-3,4- | | | | |
| dihydrothieno[2,3-d]pyrimidine-2-carboxamide | | | | |
| N-(4-fluoro-3-chlorobenzyl)-5-{[(4- | OH | H | F | Cl |
| hydroxycyclohexyl)methoxy]methyl}-4-oxo-3,4- | | | | |
| dihydrothieno[2,3-d]pyrimidine-2-carboxamide | | | | |
| N-(3-methoxybenzyl)-5-{[(4- | OH | H | H | —OCH₃ |
| hydroxycyclohexyl)methoxy]methyl}-4-oxo-3,4- | | | | |
| dihydrothieno[2,3-d]pyrimidine-2-carboxamide | | | | |
| N-(3-chlorobenzyl)-5-{[(4- | OH | H | H | Cl |
| hydroxycyclohexyl)methoxy]methyl}-4-oxo-3,4- | | | | |
| dihydrothieno[2,3-d]pyrimidine-2-carboxamide | | | | |
| N-(4-fluoro-3-methoxybenzyl)-5-{[(4- | NH₂ | H | F | —OCH₃ |
| aminocyclohexyl)methoxy]methyl}-4-oxo-3,4- | | | | |
| dihydrothieno[2,3-d]pyrimidine-2-carboxamide | | | | |
| N-(4-fluoro-3-chlorobenzyl)-5-{[(4- | NH₂ | H | F | Cl |
| aminocyclohexyl)methoxy]methyl}-4-oxo-3,4- | | | | |
| dihydrothieno[2,3-d]pyrimidine-2-carboxamide | | | | |
| N-(3-methoxybenzyl)-5-{[(4- | NH₂ | H | H | —OCH₃ |
| aminocyclohexyl)methoxy]methyl}-4-oxo-3,4- | | | | |
| dihydrothieno[2,3-d]pyrimidine-2-carboxamide | | | | |
| N-(3-chlorobenzyl)-5-{[(4-aminocyclohexyl)methoxy]methyl}- | NH₂ | H | H | Cl |
| 4-oxo-3,4-dihydrothieno[2,3-d]pyrimidine-2-carboxamide | | | | |

Another embodiment of the invention is a compound of Formula III-4, shown in Table 16.

TABLE 16
III-4

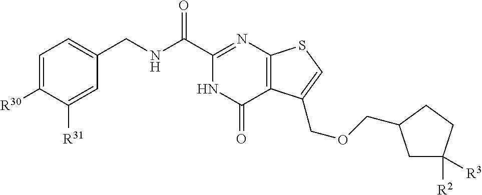

| Compound Name | R² | R³ | R³⁰ | R³¹ |
|---|---|---|---|---|
| N-(4-fluoro-3-methoxybenzyl)-5-({[3- | —C(═O)NHCH₃ | H | F | —OCH₃ |
| (methylcarbamoyl)cyclopentyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[3- | —C(═O)NHCH₃ | H | F | Cl |
| (methylcarbamoyl)cyclopentyl]methoxy}methyl)-4-oxo-3,4- | | | | |
| dihydrothieno[2,3-d]pyrimidine-2-carboxamide | | | | |
| N-(3-methoxybenzyl)-5-({[3- | —C(═O)NHCH₃ | H | H | —OCH₃ |
| (methylcarbamoyl)cyclopentyl]methoxy}methyl)-4-oxo-3,4- | | | | |
| dihydrothieno[2,3-d]pyrimidine-2-carboxamide | | | | |
| N-(3-chlorobenzyl)-5-({[3- | —C(═O)NHCH₃ | H | H | Cl |
| (methylcarbamoyl)cyclopentyl]methoxy}methyl)-4-oxo-3,4- | | | | |
| dihydrothieno[2,3-d]pyrimidine-2-carboxamide | | | | |
| 5-[({3-[(hydroxymethyl)carbamoyl]cyclopentyl}methoxy)methyl]-N-(4- | —C(═O)NHCH₂OH | H | F | —OCH₃ |
| fluoro-3-methoxybenzyl)-4-oxo-3,4-dihydrothieno[2,3-d]pyrimidine-2- | | | | |
| carboxamide | | | | |
| 5-[({3-[(hydroxymethyl)carbamoyl]cyclopentyl}methoxy)methyl]-N-(4- | —C(═O)NHCH₂OH | H | F | Cl |
| fluoro-3-chlorobenzyl)-4-oxo-3,4-dihydrothieno[2,3-d]pyrimidine-2- | | | | |
| carboxamide | | | | |
| 5-[({3-[(hydroxymethyl)carbamoyl]cyclopentyl}methoxy)methyl]-N-(3- | —C(═O)NHCH₂OH | H | H | —OCH₃ |
| methoxybenzyl)-4-oxo-3,4-dihydrothieno[2,3-d]pyrimidine-2- | | | | |
| carboxamide | | | | |
| 5-[({3-[(hydroxymethyl)carbamoyl]cyclopentyl}methoxy)methyl]-N-(3- | —C(═O)NHCH₂OH | H | H | Cl |
| chlorobenzyl)-4-oxo-3,4-dihydrothieno[2,3- d]pyrimidine-2- | | | | |
| carboxamide | | | | |
| 3-[({2-[(4-fluoro-3-methoxybenzyl)carbamoyl]-4-oxo-3,4- | —C(═O)OH | H | F | —OCH₃ |
| dihydrothieno[2,3-d]pyrimidin-5- | | | | |
| yl}methoxy)methyl]cyclopentanecarboxylic acid | | | | |
| 3-[({2-[(4-fluoro-3-chlorobenzyl)carbamoyl]-4-oxo-3,4- | —C(═O)OH | H | F | Cl |
| dihydrothieno[2,3-d]pyrimidin-5- | | | | |
| yl}methoxy)methyl]cyclopentanecarboxylic acid | | | | |
| 3-[({2-[(3-methoxybenzyl)carbamoyl]-4-oxo-3,4-dihydrothieno[2,3- | —C(═O)OH | H | H | —OCH₃ |
| d]pyrimidin-5-yl}methoxy)methyl]cyclopentanecarboxylic acid | | | | |
| 3-[({2-[(3-chlorobenzyl)carbamoyl]-4-oxo-3,4-dihydrothieno[2,3- | —C(═O)OH | H | H | Cl |
| d]pyrimidin-5-yl}methoxy)methyl]cyclopentanecarboxylic acid | | | | |
| N-(4-fluoro-3-methoxybenzyl)-5-({[3-(2- | —NHC(═O)CH₂OH | H | F | —OCH₃ |
| hydroxyacetamido)cyclopentyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[3-(2- | —NHC(═O)CH₂OH | H | F | Cl |
| hydroxyacetamido)cyclopentyl]methoxy}methyl)-4-oxo-3,4- | | | | |
| dihydrothieno[2,3-d]pyrimidine-2-carboxamide | | | | |
| N-(3-methoxybenzyl)-5-({[3-(2- | —NHC(═O)CH₂OH | H | H | —OCH₃ |
| hydroxyacetamido)cyclopentyl]methoxy}methyl)-4-oxo-3,4- | | | | |
| dihydrothieno[2,3-d]pyrimidine-2-carboxamide | | | | |
| N-(3-chlorobenzyl)-5-({[3-(2- | —NHC(═O)CH₂OH | H | H | Cl |
| hydroxyacetamido)cyclopentyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[3-(2-hydroxypropan-2- | —C(OH)(CH₃)₂ | H | F | —OCH₃ |
| yl)cyclopentyl]methoxy}methyl)-4-oxo-3,4-dihydrothieno[2,3- | | | | |
| d]pyrimidine-2-carboxamide | | | | |
| N-(4-fluoro-3-chlorobenzyl)-5-({[3-(2-hydroxypropan-2- | —C(OH)(CH₃)₂ | H | F | Cl |
| yl)cyclopentyl]methoxy}methyl)-4-oxo-3,4-dihydrothieno[2,3- | | | | |
| d]pyrimidine-2-carboxamide | | | | |
| N-(3-methoxybenzyl)-5-({[3-(2-hydroxypropan-2- | —C(OH)(CH₃)₂ | H | H | —OCH₃ |
| yl)cyclopentyl]methoxy}methyl)-4-oxo-3,4-dihydrothieno[2,3- | | | | |
| d]pyrimidine-2-carboxamide | | | | |
| N-(3-chlorobenzyl)-5-({[3-(2-hydroxypropan-2- | —C(OH)(CH₃)₂ | H | H | Cl |
| yl)cyclopentyl]methoxy}methyl)-4-oxo-3,4-dihydrothieno[2,3- | | | | |
| d]pyrimidine-2-carboxamide | | | | |
| N-(4-fluoro-3-methoxybenzyl)-5-({[3- | —CH₂OH | H | F | —OCH₃ |
| (hydroxymethyl)cyclopentyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[3- | —CH₂OH | H | F | Cl |
| (hydroxymethyl)cyclopentyl]methoxy}methyl)-4-oxo-3,4- | | | | |
| dihydrothieno[2,3-d]pyrimidine-2-carboxamide | | | | |
| N-(3-methoxybenzyl)-5-({[3- | —CH₂OH | H | H | —OCH₃ |
| (hydroxymethyl)cyclopentyl]methoxy}methyl)-4-oxo-3,4- | | | | |
| dihydrothieno[2,3-d]pyrimidine-2-carboxamide | | | | |
| N-(3-chlorobenzyl)-5-({[3- | —CH₂OH | H | H | Cl |
| (hydroxymethyl)cyclopentyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[3- | —NHSO₂CH₃ | H | F | —OCH₃ |
| (methylsulfonamido)cyclopentyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[3- | —NHSO₂CH₃ | H | F | Cl |
| (methylsulfonamido)cyclopentyl]methoxy}methyl)-4-oxo-3,4- | | | | |
| dihydrothieno[2,3-d]pyrimidine-2-carboxamide | | | | |
| N-(3-methoxybenzyl)-5-({[3- | —NHSO₂CH₃ | H | H | —OCH₃ |
| (methylsulfonamido)cyclopentyl]methoxy}methyl)-4-oxo-3,4- | | | | |
| dihydrothieno[2,3-d]pyrimidine-2-carboxamide | | | | |
| N-(3-chlorobenzyl)-5-({[3- | —NHSO₂CH₃ | H | H | Cl |
| (methylsulfonamido)cyclopentyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[3- | —CH₂NHSO₂CH₃ | H | F | —OCH₃ |
| (methylsulfonamidomethyl)cyclopentyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[3- | —CH₂NHSO₂CH₃ | H | F | Cl |
| (methylsulfonamidomethyl)cyclopentyl]methoxy}methyl)-4-oxo-3,4- | | | | |
| dihydrothieno[2,3-d]pyrimidine-2-carboxamide | | | | |
| N-(3-methoxybenzyl)-5-({[3- | —CH₂NHSO₂CH₃ | H | H | —OCH₃ |
| (methylsulfonamidomethyl)cyclopentyl]methoxy}methyl)-4-oxo-3,4- | | | | |
| dihydrothieno[2,3-d]pyrimidine-2-carboxamide | | | | |
| N-(3-chlorobenzyl)-5-({[3- | —CH₂NHSO₂CH₃ | H | H | Cl |
| (methylsulfonamidomethyl)cyclopentyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[3- | —SO₂CH₃ | H | F | —OCH₃ |
| (methylsulfonyl)cyclopentyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[3- | —SO₂CH₃ | H | F | Cl |
| (methylsulfonyl)cyclopentyl]methoxy}methyl)-4-oxo-3,4- | | | | |
| dihydrothieno[2,3-d]pyrimidine-2-carboxamide | | | | |
| N-(3-methoxybenzyl)-5-({[3- | —SO₂CH₃ | H | H | —OCH₃ |
| (methylsulfonyl)cyclopentyl]methoxy}methyl)-4-oxo-3,4- | | | | |
| dihydrothieno[2,3-d]pyrimidine-2-carboxamide | | | | |
| N-(3-chlorobenzyl)-5-({[3- | —SO₂CH₃ | H | H | Cl |
| (methylsulfonyl)cyclopentyl]methoxy}methyl)-4-oxo-3,4- | | | | |
| dihydrothieno[2,3-d]pyrimidine-2-carboxamide | | | | |
| N-(4-fluoro-3-methoxybenzyl)-5-({[3- | —CH₂SO₂CH₃ | H | F | —OCH₃ |
| (methylsulfonylmethyl)cyclopentyl]methoxy}methyl)-4-oxo-3,4- | | | | |
| dihydrothieno[2,3-d]pyrimidine-2-carboxamide | | | | |
| N-(4-fluoro-3-chlorobenzyl)-5-({[3- | —CH₂SO₂CH₃ | H | F | Cl |
| (methylsulfonylmethyl)cyclopentyl]methoxy}methyl)-4-oxo-3,4- | | | | |
| dihydrothieno[2,3-d]pyrimidine-2-carboxamide | | | | |
| N-(3-methoxybenzyl)-5-({[3- | —CH₂SO₂CH₃ | H | H | —OCH₃ |
| (methylsulfonylmethyl)cyclopentyl]methoxy}methyl)-4-oxo-3,4- | | | | |
| dihydrothieno[2,3-d]pyrimidine-2-carboxamide | | | | |
| N-(3-chlorobenzyl)-5-({[3- | —CH₂SO₂CH₃ | H | H | Cl |
| (methylsulfonylmethyl)cyclopentyl]methoxy}methyl)-4-oxo-3,4- | | | | |
| dihydrothieno[2,3-d]pyrimidine-2-carboxamide | | | | |
| N-(4-fluoro-3-methoxybenzyl)-5-{[(3- | —NHC(═O)CH₃ | H | F | —OCH₃ |
| acetamidocyclopentyl)methoxy]methyl}-4-oxo-3,4-dihydrothieno[2,3- | | | | |
| d]pyrimidine-2-carboxamide | | | | |
| N-(4-fluoro-3-chlorobenzyl)-5-{[(3- | —NHC(═O)CH₃ | H | F | Cl |
| acetamidocyclopentyl)methoxy]methyl}-4-oxo-3,4-dihydrothieno[2,3- | | | | |
| d]pyrimidine-2-carboxamide | | | | |
| N-(3-methoxybenzyl)-5-{[(3-acetamidocyclopentyl)methoxy]methyl}-4- | —NHC(═O)CH₃ | H | H | —OCH₃ |
| oxo-3,4-dihydrothieno[2,3-d]pyrimidine-2-carboxamide | | | | |
| N-(3-chlorobenzyl)-5-{[(3-acetamidocyclopentyl)methoxy]methyl}-4- | —NHC(═O)CH₃ | H | H | Cl |
| oxo-3,4-dihydrothieno[2,3-d]pyrimidine-2-carboxamide | | | | |
| N-(4-fluoro-3-methoxybenzyl)-5-{[(3- | —C(═O)NH₂ | H | F | —OCH₃ |
| carbamoylcyclopentyl)methoxy]methyl}-4-oxo-3,4-dihydrothieno[2,3- | | | | |
| d]pyrimidine-2-carboxamide | | | | |
| N-(4-fluoro-3-chlorobenzyl)-5-{[(3- | —C(═O)NH₂ | H | F | Cl |
| carbamoylcyclopentyl)methoxy]methyl}-4-oxo-3,4-dihydrothieno[2,3- | | | | |
| d]pyrimidine-2-carboxamide | | | | |
| N-(3-methoxybenzyl)-5-{[(3-carbamoylcyclopentyl)methoxy]methyl}-4- | —C(═O)NH₂ | H | H | —OCH₃ |
| oxo-3,4-dihydrothieno[2,3-d]pyrimidine-2-carboxamide | | | | |
| N-(3-chlorobenzyl)-5-{[(3-carbamoylcyclopentyl)methoxy]methyl}-4- | —C(═O)NH₂ | H | H | Cl |
| oxo-3,4-dihydrothieno[2,3-d]pyrimidine-2-carboxamide | | | | |
| N-(4-fluoro-3-methoxybenzyl)-5-{[(3- | CN | H | F | —OCH₃ |
| cyanocyclopentyl)methoxy]methyl}-4-oxo-3,4-dihydrothieno[2,3- | | | | |
| d]pyrimidine-2-carboxamide | | | | |
| N-(4-fluoro-3-chlorobenzyl)-5-{[(3-cyanocyclopentyl)methoxy]methyl}- | CN | H | F | Cl |
| 4-oxo-3,4-dihydrothieno[2,3-d]pyrimidine-2-carboxamide | | | | |
| N-(3-methoxybenzyl)-5-{[(3-cyanocyclopentyl)methoxy]methyl}-4-oxo- | CN | H | H | —OCH₃ |
| 3,4-dihydrothieno[2,3-d]pyrimidine-2-carboxamide | | | | |
| N-(3-chlorobenzyl)-5-{[(3-cyanocyclopentyl)methoxy]methyl}-4-oxo- | CN | H | H | Cl |
| 3,4-dihydrothieno[2,3-d]pyrimidine-2-carboxamide | | | | |
| N-(4-fluoro-3-methoxybenzyl)-5-{[(3- | OH | H | F | —OCH₃ |
| hydroxycyclopentyl)methoxy]methyl}-4-oxo-3,4-dihydrothieno[2,3- | | | | |
| d]pyrimidine-2-carboxamide | | | | |
| N-(4-fluoro-3-chlorobenzyl)-5-{[(3- | OH | H | F | Cl |
| hydroxycyclopentyl)methoxy]methyl}-4-oxo-3,4-dihydrothieno[2,3- | | | | |
| d]pyrimidine-2-carboxamide | | | | |
| N-(3-methoxybenzyl)-5-{[(3-hydroxycyclopentyl)methoxy]methyl}-4- | OH | H | H | —OCH₃ |
| oxo-3,4-dihydrothieno[2,3-d]pyrimidine-2-carboxamide | | | | |
| N-(3-chlorobenzyl)-5-{[(3-hydroxycyclopentyl)methoxy]methyl}-4-oxo- | OH | H | H | Cl |
| 3,4-dihydrothieno[2,3-d]pyrimidine-2-carboxamide | | | | |

Another embodiment of the invention is a compound of Formula III-5, shown in Table 17.

TABLE 17
III-5

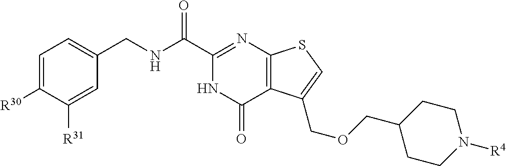

| Compound Name | R⁴ | R³⁰ | R³¹ |
|---|---|---|---|
| N-(4-fluoro-3-methoxybenzyl)-5-({[1-(2-hydroxyacetyl)piperidin-4- | —C(═O)CH₂OH | F | —OCH₃ |
| yl]methoxy}methyl)-4-oxo-3,4-dihydrothieno[2,3-d]pyrimidine-2- | | | |
| carboxamide | | | |
| N-(4-fluoro-3-chlorobenzyl)-5-({[1-(2-hydroxyacetyl)piperidin-4- | —C(═O)CH₂OH | F | Cl |
| yl]methoxy}methyl)-4-oxo-3,4-dihydrothieno[2,3-d]pyrimidine-2- | | | |
| carboxamide | | | |
| N-(3-methoxybenzyl)-5-({[1-(2-hydroxyacetyl)piperidin-4- | —C(═O)CH₂OH | H | —OCH₃ |
| yl]methoxy}methyl)-4-oxo-3,4-dihydrothieno[2,3-d]pyrimidine-2- | | | |
| carboxamide | | | |
| N-(3-chlorobenzyl)-5-({[1-(2-hydroxyacetyl)piperidin-4- | —C(═O)CH₂OH | H | Cl |
| yl]methoxy}methyl)-4-oxo-3,4-dihydrothieno[2,3-d]pyrimidine-2- | | | |
| carboxamide | | | |
| N-(4-fluoro-3-methoxybenzyl)-5-{[(1-acetylpiperidin-4- | —C(═O)CH₃ | F | —OCH₃ |
| yl)methoxy]methyl}-4-oxo-3,4-dihydrothieno[2,3-d]pyrimidine-2- | | | |
| carboxamide | | | |
| N-(4-fluoro-3-chlorobenzyl)-5-{[(1-acetylpiperidin-4- | —C(═O)CH₃ | F | Cl |
| yl)methoxy]methyl}-4-oxo-3,4-dihydrothieno[2,3-d]pyrimidine-2- | | | |
| carboxamide | | | |
| N-(3-methoxybenzyl)-5-{[(1-acetylpiperidin-4-yl)methoxy]methyl}-4- | —C(═O)CH₃ | H | —OCH₃ |
| oxo-3,4-dihydrothieno[2,3-d]pyrimidine-2-carboxamide | | | |
| N-(3-chlorobenzyl)-5-{[(1-acetylpiperidin-4-yl)methoxy]methyl}-4-oxo- | —C(═O)CH₃ | H | Cl |
| 3,4-dihydrothieno[2,3-d]pyrimidine-2-carboxamide | | | |
| N-(4-fluoro-3-methoxybenzyl)-5-[({1-[(5-methyl-1,3,4-oxadiazol-2- yl)methyl]piperidin-4-yl}methoxy)methyl]-4-oxo-3,4-dihydrothieno[2,3- d]pyrimidine-2-carboxamide | 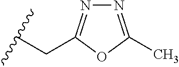 | F | —OCH₃ |
| N-(4-fluoro-3-chlorobenzyl)-5-[({1-[(5-methyl-1,3,4-oxadiazol-2- yl)methyl]piperidin-4-yl}methoxy)methyl]-4-oxo-3,4-dihydrothieno[2,3- d]pyrimidine-2-carboxamide | 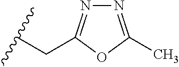 | F | Cl |
| N-(3-methoxybenzyl)-5-[({1-[(5-methyl-1,3,4-oxadiazol-2- yl)methyl]piperidin-4-yl}methoxy)methyl]-4-oxo-3,4-dihydrothieno[2,3- d]pyrimidine-2-carboxamide | 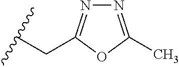 | H | —OCH₃ |
| N-(3-chlorobenzyl)-5-[({1-[(5-methyl-1,3,4-oxadiazol-2- yl)methyl]piperidin-4-yl}methoxy)methyl]-4-oxo-3,4-dihydrothieno[2,3- d]pyrimidine-2-carboxamide | 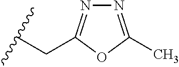 | H | Cl |
| N-(3-methoxy-4-fluorobenzyl)-4-oxo-5-[(2-piperidin-4-ylethoxy)methyl]- | H | F | —OCH₃ |
| 3,4-dihydrothieno[2,3-d]pyrimidine-2-carboxamide hydrochloride | | | |
| N-(3-chloro-4-fluorobenzyl)-4-oxo-5-[(2-piperidin-4-ylethoxy)methyl]- | H | F | Cl |
| 3,4-dihydrothieno[2,3-d]pyrimidine-2-carboxamide hydrochloride | | | |
| N-(3-methoxybenzyl)-4-oxo-5-[(2-piperidin-4-ylethoxy)methyl]-3,4- | H | H | —OCH₃ |
| dihydrothieno[2,3-d]pyrimidine-2-carboxamide hydrochloride | | | |
| N-(3-chlorobenzyl)-4-oxo-5-[(2-piperidin-4-ylethoxy)methyl]-3,4- | H | H | Cl |
| dihydrothieno[2,3-d]pyrimidine-2-carboxamide hydrochloride | | | |
| N-(4-fluoro-3-methoxybenzyl)-5-[({1-[2-(methylsulfonyl)ethyl]piperidin- | —CH₂CH₂SO₂CH₃ | F | —OCH₃ |
| 4-yl}methoxy)methyl]-4-oxo-3,4-dihydrothieno[2,3-d]pyrimidine-2- | | | |
| carboxamide | | | |
| N-(4-fluoro-3-chlorobenzyl)-5-[({1-[2-(methylsulfonyl)ethyl]piperidin-4- | —CH₂CH₂SO₂CH₃ | F | Cl |
| yl}methoxy)methyl]-4-oxo-3,4-dihydrothieno[2,3-d]pyrimidine-2- | | | |
| carboxamide | | | |
| N-(3-methoxybenzyl)-5-[({1-[2-(methylsulfonyl)ethyl]piperidin-4- | —CH₂CH₂SO₂CH₃ | H | —OCH₃ |
| yl}methoxy)methyl]-4-oxo-3,4-dihydrothieno[2,3-d]pyrimidine-2- | | | |
| carboxamide | | | |
| N-(3-chlorobenzyl)-5-[({1-[2-(methylsulfonyl)ethyl]piperidin-4- | —CH₂CH₂SO₂CH₃ | H | Cl |
| yl}methoxy)methyl]-4-oxo-3,4-dihydrothieno[2,3-d]pyrimidine-2- | | | |
| carboxamide | | | |
| N-(4-fluoro-3-methoxybenzyl)-5-({[1-(2-hydroxyacetyl)piperidin-4- | —C(═O)CH₂OH | F | —OCH₃ |
| yl]methoxy}methyl)-4-oxo-3,4-dihydrothieno[2,3-d]pyrimidine-2- | | | |
| carboxamide | | | |
| N-(4-fluoro-3-chlorobenzyl)-5-({[1-(2-hydroxyacetyl)piperidin-4- | —C(═O)CH₂OH | F | Cl |
| yl]methoxy}methyl)-4-oxo-3,4-dihydrothieno[2,3-d]pyrimidine-2- | | | |
| carboxamide | | | |
| N-(3-methoxybenzyl)-5-({[1-(2-hydroxyacetyl)piperidin-4- | —C(═O)CH₂OH | H | —OCH₃ |
| yl]methoxy}methyl)-4-oxo-3,4-dihydrothieno[2,3-d]pyrimidine-2- | | | |
| carboxamide | | | |
| N-(3-chlorobenzyl)-5-({[1-(2-hydroxyacetyl)piperidin-4- | —C(═O)CH₂OH | H | Cl |
| yl]methoxy}methyl)-4-oxo-3,4-dihydrothieno[2,3-d]pyrimidine-2- | | | |
| carboxamide | | | |
| N-(4-fluoro-3-methoxybenzyl)-5-({[1-(2-hydroxyethyl)piperidin-4- | —CH₂CH₂OH | F | —OCH₃ |
| yl]methoxy}methyl)-4-oxo-3,4-dihydrothieno[2,3-d]pyrimidine-2- | | | |
| carboxamide | | | |
| N-(4-fluoro-3-chlorobenzyl)-5-({[1-(2-hydroxyethyl)piperidin-4- | —CH₂CH₂OH | F | Cl |
| yl]methoxy}methyl)-4-oxo-3,4-dihydrothieno[2,3-d]pyrimidine-2- | | | |
| carboxamide | | | |
| N-(3-methoxybenzyl)-5-({[1-(2-hydroxyethyl)piperidin-4- | —CH₂CH₂OH | H | —OCH₃ |
| yl]methoxy}methyl)-4-oxo-3,4-dihydrothieno[2,3-d]pyrimidine-2- | | | |
| carboxamide | | | |
| N-(3-chlorobenzyl)-5-({[1-(2-hydroxyethyl)piperidin-4- | —CH₂CH₂OH | H | Cl |
| yl]methoxy}methyl)-4-oxo-3,4-dihydrothieno[2,3-d]pyrimidine-2- | | | |
| carboxamide | | | |
| N-(4-fluoro-3-methoxybenzyl)-5-({[1-(methylsulfonyl)piperidin-4- | —SO₂CH₃ | F | —OCH₃ |
| yl]methoxy}methyl)-4-oxo-3,4-dihydrothieno[2,3-d]pyrimidine-2- | | | |
| carboxamide | | | |
| N-(4-fluoro-3-chlorobenzyl)-5-({[1-(methylsulfonyl)piperidin-4- | —SO₂CH₃ | F | Cl |
| yl]methoxy}methyl)-4-oxo-3,4-dihydrothieno[2,3-d]pyrimidine-2- | | | |
| carboxamide | | | |
| N-(3-methoxybenzyl)-5-({[1-(methylsulfonyl)piperidin-4- | —SO₂CH₃ | H | —OCH₃ |
| yl]methoxy}methyl)-4-oxo-3,4-dihydrothieno[2,3-d]pyrimidine-2- | | | |
| carboxamide | | | |
| N-(3-chlorobenzyl)-5-({[1-(methylsulfonyl)piperidin-4- | —SO₂CH₃ | H | Cl |
| yl]methoxy}methyl)-4-oxo-3,4-dihydrothieno[2,3-d]pyrimidine-2- | | | |
| carboxamide | | | |
| N-(4-fluoro-3-methoxybenzyl)-5-({[1-(methylsulfonylmethyl)piperidin-4- | —CH₂SO₂CH₃ | F | —OCH₃ |
| yl]methoxy}methyl)-4-oxo-3,4-dihydrothieno[2,3-d]pyrimidine-2- | | | |
| carboxamide | | | |
| N-(4-fluoro-3-chlorobenzyl)-5-({[1-(methylsulfonylmethyl)piperidin-4- | —CH₂SO₂CH₃ | F | Cl |
| yl]methoxy}methyl)-4-oxo-3,4-dihydrothieno[2,3-d]pyrimidine-2- | | | |
| carboxamide | | | |
| N-(3-methoxybenzyl)-5-({[1-(methylsulfonylmethyl)piperidin-4- | —CH₂SO₂CH₃ | H | —OCH₃ |
| yl]methoxy}methyl)-4-oxo-3,4-dihydrothieno[2,3-d]pyrimidine-2- | | | |
| carboxamide | | | |
| N-(3-chlorobenzyl)-5-({[1-(methylsulfonylmethyl)piperidin-4- | —CH₂SO₂CH₃ | H | Cl |
| yl]methoxy}methyl)-4-oxo-3,4-dihydrothieno[2,3-d]pyrimidine-2- | | | |
| carboxamide | | | |
| N-(4-fluoro-3-methoxybenzyl)-5-{[(1-acetylpiperidin-4- | —C(═O)CH₃ | F | —OCH₃ |
| yl)methoxy]methyl}-4-oxo-3,4-dihydrothieno[2,3-d]pyrimidine-2- | | | |
| carboxamide | | | |
| N-(4-fluoro-3-chlorobenzyl)-5-{[(1-acetylpiperidin-4- | —C(═O)CH₃ | F | Cl |
| yl)methoxy]methyl}-4-oxo-3,4-dihydrothieno[2,3-d]pyrimidine-2- | | | |
| carboxamide | | | |
| N-(3-methoxybenzyl)-5-{[(1-acetylpiperidin-4-yl)methoxy]methyl}-4- | —C(═O)CH₃ | H | —OCH₃ |
| oxo-3,4-dihydrothieno[2,3-d]pyrimidine-2-carboxamide | | | |
| N-(3-chlorobenzyl)-5-{[(1-acetylpiperidin-4-yl)methoxy]methyl}-4-oxo- | —C(═O)CH₃ | H | Cl |
| 3,4-dihydrothieno[2,3-d]pyrimidine-2-carboxamide | | | |

Another embodiment of the invention is a compound of Formula III-6, shown in Table 18.

TABLE 18
III-6

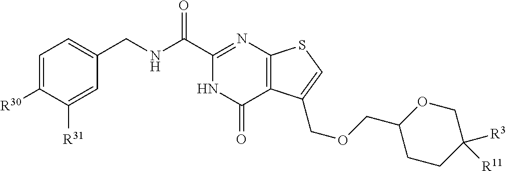

| Compound Name | R³ | R¹¹ | R³⁰ | R³¹ |
|---|---|---|---|---|
| N-(4-fluoro-3-methoxybenzyl)-4-oxo-5-{[(tetrahydro-2H-pyran-2- | H | H | F | —OCH₃ |
| yl)methoxy]methyl}-3,4-dihydrothieno[2,3-d]pyrimidine-2-carboxamide | | | | |
| N-(4-fluoro-3-chlorobenzyl)-4-oxo-5-{[(tetrahydro-2H-pyran-2- | H | H | F | Cl |
| yl)methoxy]methyl}-3,4-dihydrothieno[2,3-d]pyrimidine-2-carboxamide | | | | |
| N-(3-methoxybenzyl)-4-oxo-5-{[(tetrahydro-2H-pyran-2-yl)methoxy]methyl}-3,4- | H | H | H | —OCH₃ |
| dihydrothieno[2,3-d]pyrimidine-2-carboxamide | | | | |
| N-(3-chlorobenzyl)-4-oxo-5-{[(tetrahydro-2H-pyran-2-yl)methoxy]methyl}-3,4- | H | H | H | Cl |
| dihydrothieno[2,3-d]pyrimidine-2-carboxamide | | | | |
| N-(4-fluoro-3-methoxybenzyl)-5-({[5-(hydroxymethyl)-tetrahydro-2H-pyran-2- | H | —CH₂OH | F | —OCH₃ |
| yl]methoxy}methyl)-4-oxo-3,4-dihydrothieno[2,3-d]pyrimidine-2-carboxamide | | | | |
| N-(4-fluoro-3-chlorobenzyl)-5-({[5-(hydroxymethyl)-tetrahydro-2H-pyran-2- | H | —CH₂OH | F | Cl |
| yl]methoxy}methyl)-4-oxo-3,4-dihydrothieno[2,3-d]pyrimidine-2-carboxamide | | | | |
| N-(3-methoxybenzyl)-5-({[5-(hydroxymethyl)-tetrahydro-2H-pyran-2- | H | —CH₂OH | H | —OCH₃ |
| yl]methoxy}methyl)-4-oxo-3,4-dihydrothieno[2,3-d]pyrimidine-2-carboxamide | | | | |
| N-(3-chlorobenzyl)-5-({[5-(hydroxymethyl)-tetrahydro-2H-pyran-2- | H | —CH₂OH | H | Cl |
| yl]methoxy}methyl)-4-oxo-3,4-dihydrothieno[2,3-d]pyrimidine-2-carboxamide | | | | |

Another embodiment of the invention is a compound of Formula III-7, shown in Table 19.

TABLE 19
III-7

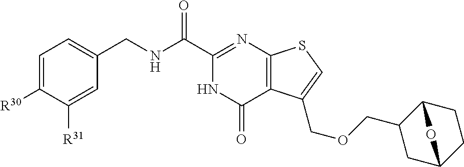

| Compound Name | R³⁰ | R³¹ |
|---|---|---|
| N-(4-fluoro-3-methoxybenzyl)-5-[(7-oxa-bicyclo[2.2.1]heptan-2-ylmethoxy)methyl]-4-oxo- | F | —OCH₃ |
| 3,4-dihydrothieno[2,3-d]pyrimidine-2-carboxamide | | |
| N-(4-fluoro-3-chlorobenzyl)-5-[(7-oxa-bicyclo[2.2.1]heptan-2-ylmethoxy)methyl]-4-oxo-3,4- | F | Cl |
| dihydrothieno[2,3-d]pyrimidine-2-carboxamide | | |
| N-(3-methoxybenzyl)-5-[(7-oxa-bicyclo[2.2.1]heptan-2-ylmethoxy)methyl]-4-oxo-3,4- | H | —OCH₃ |
| dihydrothieno[2,3-d]pyrimidine-2-carboxamide | | |
| N-(3-chlorobenzyl)-5-[(7-oxa-bicyclo[2.2.1]heptan-2-ylmethoxy)methyl]-4-oxo-3,4- | H | Cl |
| dihydrothieno[2,3-d]pyrimidine-2-carboxamide | | |

Another embodiment of the invention is a compound of Formula III-8, shown in Table 20.

TABLE 20
III-8

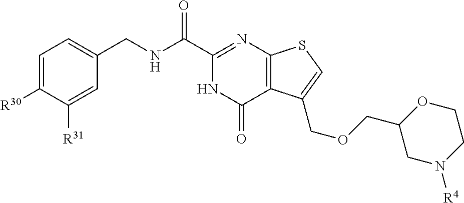

| Compound Name | R⁴ | R³⁰ | R³¹ |
|---|---|---|---|
| N-(4-fluoro-3-methoxybenzyl)-5-({[4-(methylsulfonyl)morpholin-2- | —SO₂CH₃ | F | —OCH₃ |
| yl]methoxy}methyl)-4-oxo-3,4-dihydrothieno[2,3-d]pyrimidine-2-carboxamide | | | |
| N-(4-fluoro-3-chlorobenzyl)-5-({[4-(methylsulfonyl)morpholin-2- | —SO₂CH₃ | F | Cl |
| yl]methoxy}methyl)-4-oxo-3,4-dihydrothieno[2,3-d]pyrimidine-2-carboxamide | | | |
| N-(3-methoxybenzyl)-5-({[4-(methylsulfonyl)morpholin-2-yl]methoxy}methyl)-4- | —SO₂CH₃ | H | —OCH₃ |
| oxo-3,4-dihydrothieno[2,3-d]pyrimidine-2-carboxamide | | | |
| N-(3-chlorobenzyl)-5-({[4-(methylsulfonyl)morpholin-2-yl]methoxy}methyl)-4-oxo- | —SO₂CH₃ | H | Cl |
| 3,4-dihydrothieno[2,3-d]pyrimidine-2-carboxamide | | | |
| N-(4-fluoro-3-methoxybenzyl)-5-{[(4-acetylmorpholin-2-yl)methoxy]methyl}-4-oxo- | —C(═O)CH₃ | F | —OCH₃ |
| 3,4-dihydrothieno[2,3-d]pyrimidine-2-carboxamide | | | |
| N-(4-fluoro-3-chlorobenzyl)-5-{[(4-acetylmorpholin-2-yl)methoxy]methyl}-4-oxo- | —C(═O)CH₃ | F | Cl |
| 3,4-dihydrothieno[2,3-d]pyrimidine-2-carboxamide | | | |
| N-(3-methoxybenzyl)-5-{[(4-acetylmorpholin-2-yl)methoxy]methyl}-4-oxo-3,4- | —C(═O)CH₃ | H | —OCH₃ |
| dihydrothieno[2,3-d]pyrimidine-2-carboxamide | | | |
| N-(3-chlorobenzyl)-5-{[(4-acetylmorpholin-2-yl)methoxy]methyl}-4-oxo-3,4- | —C(═O)CH₃ | H | Cl |
| dihydrothieno[2,3-d]pyrimidine-2-carboxamide | | | |

Another embodiment of the invention is a compound of Formula III-9, shown in Table 21.

TABLE 21
III-9

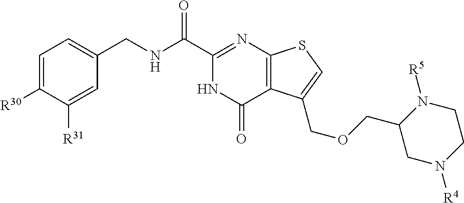

| Compound Name | R⁴ | R⁵ | R³⁰ | R³¹ |
|---|---|---|---|---|
| N-(4-fluoro-3-methoxybenzyl)-5-({[4-(methylsulfonyl)piperazin-2- | —SO₂CH₃ | H | F | —OCH₃ |
| yl]methoxy}methyl)-4-oxo-3,4-dihydrothieno[2,3-d]pyrimidine-2-carboxamide | | | | |
| N-(4-fluoro-3-chlorobenzyl)-5-({[4-(methylsulfonyl)piperazin-2- | —SO₂CH₃ | H | F | Cl |
| yl]methoxy}methyl)-4-oxo-3,4-dihydrothieno[2,3-d]pyrimidine-2-carboxamide | | | | |
| N-(3-methoxybenzyl)-5-({[4-(methylsulfonyl)piperazin-2-yl]methoxy}methyl)- | —SO₂CH₃ | H | H | —OCH₃ |
| 4-oxo-3,4-dihydrothieno[2,3-d]pyrimidine-2-carboxamide | | | | |
| N-(3-chlorobenzyl)-5-({[4-(methylsulfonyl)piperazin-2-yl]methoxy}methyl)-4- | —SO₂CH₃ | H | H | Cl |
| oxo-3,4-dihydrothieno[2,3-d]pyrimidine-2-carboxamide | | | | |
| N-(4-fluoro-3-methoxybenzyl)-5-({[4-acetylpiperazin-2-yl)methoxy]methyl}-4- | —C(═O)CH₃ | H | F | —OCH₃ |
| oxo-3,4-dihydrothieno[2,3-d]pyrimidine-2-carboxamide | | | | |
| N-(4-fluoro-3-chlorobenzyl)-5-({[4-acetylpiperazin-2-yl)methoxy]methyl}-4- | —C(═O)CH₃ | H | F | Cl |
| oxo-3,4-dihydrothieno[2,3-d]pyrimidine-2-carboxamide | | | | |
| N-(3-methoxybenzyl)-5-({[4-acetylpiperazin-2-yl)methoxy]methyl}-4-oxo-3,4- | —C(═O)CH₃ | H | H | —OCH₃ |
| dihydrothieno[2,3-d]pyrimidine-2-carboxamide | | | | |
| N-(3-chlorobenzyl)-5-({[4-acetylpiperazin-2-yl)methoxy]methyl}-4-oxo-3,4- | —C(═O)CH₃ | H | H | Cl |
| dihydrothieno[2,3-d]pyrimidine-2-carboxamide | | | | |

Another embodiment of the invention is a compound of Formula III-10, shown in Table 22.

TABLE 22
III-10

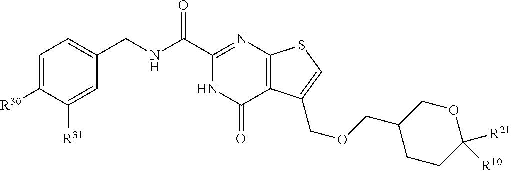

| Compound Name | R¹⁰ | R²¹ | R³⁰ | R³¹ |
|---|---|---|---|---|
| N-(4-fluoro-3-methoxybenzyl)-5-({[6-(hydroxymethyl)-tetrahydro-2H-pyran-3- | —CH₂OH | H | F | —OCH₃ |
| yl]methoxy}methyl)-4-oxo-3,4-dihydrothieno[2,3-d]pyrimidine-2-carboxamide | | | | |
| N-(4-fluoro-3-chlorobenzyl)-5-({[6-(hydroxymethyl)-tetrahydro-2H-pyran-3- | —CH₂OH | H | F | Cl |
| yl]methoxy}methyl)-4-oxo-3,4-dihydrothieno[2,3-d]pyrimidine-2-carboxamide | | | | |
| N-(3-methoxybenzyl)-5-({[6-(hydroxymethyl)-tetrahydro-2H-pyran-3- | —CH₂OH | H | H | —OCH₃ |
| yl]methoxy}methyl)-4-oxo-3,4-dihydrothieno[2,3-d]pyrimidine-2-carboxamide | | | | |
| N-(3-chlorobenzyl)-5-({[6-(hydroxymethyl)-tetrahydro-2H-pyran-3- | —CH₂OH | H | H | Cl |
| yl]methoxy}methyl)-4-oxo-3,4-dihydrothieno[2,3-d]pyrimidine-2-carboxamide | | | | |

Another embodiment of the invention is a compound selected from the compounds named in Tables 13-22.

Another embodiment of the invention is a compound selected from 5-[({2-[(4-fluoro-3-methoxybenzyl)carbamoyl]-4-oxo-3,4-dihydrothieno[2,3-d]pyrimidin-5-yl}methoxy)methyl]-1,4-dioxane-2-carboxylic acid; 5-[({2-[(3-methoxybenzyl)carbamoyl]-4-oxo-3,4-dihydrothieno[2,3-d]pyrimidin-5-yl}methoxy)methyl]-1,4-dioxane-2-carboxylic acid; N-(4-fluoro-3-methoxybenzyl)-5-{[(5-carbamoyl-1,4-dioxan-2-yl)methoxy]methyl}-4-oxo-3,4-dihydrothieno[2,3-d]pyrimidine-2-carboxamide; N-(4-fluoro-3-methoxybenzyl)-5-({[5-(hydroxymethyl)-1,4-dioxan-2-yl]methoxy}methyl)-4-oxo-3,4-dihydrothieno[2,3-d]pyrimidine-2-carboxamide; N-(3-methoxybenzyl)-5-({[5-(hydroxymethyl)-1,4-dioxan-2-yl]methoxy}methyl)-4-oxo-3,4-dihydrothieno[2,3-d]pyrimidine-2-carboxamide; 5-({[4-aminomethylcyclohexyl]methoxy}methyl)-N-(3-methoxybenzyl)-4-oxo-3,4-dihydrothieno[2,3-d]pyrimidine-2-carboxamide; 2-{4-[({2-[(4-fluoro-3-methoxybenzyl)carbamoyl]-4-oxo-3,4-dihydrothieno[2,3-d]pyrimidin-5-yl}methoxy)methyl]cyclohexylamino}-2-oxoethyl acetate; 2-{4-[({2-[(3-methoxybenzyl)carbamoyl]-4-oxo-3,4-dihydrothieno[2,3-d]pyrimidin-5-yl}methoxy)methyl]cyclohexylamino}-2-oxoethyl acetate; 2-[4-({2-[(4-fluoro-3-methoxybenzyl)carbamoyl]-4-oxo-3,4-dihydrothieno[2,3-d]pyrimidin-5-yl}methoxy)methyl]cyclohexanecarboxylic acid; 2-[4-({2-[(3-methoxybenzyl)carbamoyl]-4-oxo-3,4-dihydrothieno[2,3-d]pyrimidin-5-yl}methoxy)methyl]cyclohexanecarboxylic acid; N-(4-fluoro-3-methoxybenzyl)-5-({[4-(2-hydroxyacetamido)cyclohexyl]methoxy}methyl)-4-oxo-3,4-dihydrothieno[2,3-d]pyrimidine-2-carboxamide; N-(3-methoxybenzyl)-5-({[4-(2-hydroxy acetamido)cyclohexyl]methoxy}methyl)-4-oxo-3,4-dihydrothieno[2,3-d]pyrimidine-2-carboxamide; N-(4-fluoro-3-methoxybenzyl)-5-({[4-(hydroxymethyl)cyclohexyl]methoxy}methyl)-4-oxo-3,4-dihydrothieno[2,3-d]pyrimidine-2-carboxamide; N-(3-methoxybenzyl)-5-({[4-(hydroxymethyl)cyclohexyl]methoxy}methyl)-4-oxo-3,4-dihydrothieno[2,3-d]pyrimidine-2-carboxamide; N-(4-fluoro-3-methoxybenzyl)-5-({[4-(methylsulfonamido)cyclohexyl]methoxy}methyl)-4-oxo-3,4-dihydrothieno[2,3-d]pyrimidine-2-carboxamide; N-(3-methoxybenzyl)-5-({[4-(methylsulfonamido)cyclohexyl]methoxy}methyl)-4-oxo-3,4-dihydrothieno[2,3-d]pyrimidine-2-carboxamide; N-(3-methoxybenzyl)-5-({[4-(methylsulfonamidomethyl)cyclohexyl]methoxy}methyl)-4-oxo-3,4-dihydrothieno[2,3-d]pyrimidine-2-carboxamide; N-(4-fluoro-3-methoxybenzyl)-5-({[4-acetamidocyclohexyl)methoxy]methyl}-4-oxo-3,4-dihydrothieno[2,3-d]pyrimidine-2-carboxamide; N-(3-methoxybenzyl)-5-{[(4-acetamidocyclohexyl)methoxy]methyl}-4-oxo-3,4-dihydrothieno[2,3-d]pyrimidine-2-carboxamide; N-(4-fluoro-3-methoxybenzyl)-5-{[(4-carbamoylcyclohexyl)methoxy]methyl}-4-oxo-3,4-dihydrothieno[2,3-d]pyrimidine-2-carboxamide; N-(4-fluoro-3-methoxybenzyl)-5-{[(4-cyanocyclohexyl)methoxy]methyl}-4-oxo-3,4-dihydrothieno[2,3-d]pyrimidine-2-carboxamide; N-(3-methoxybenzyl)-5-{[(4-cyanocyclohexyl)methoxy]methyl}-4-oxo-3,4-dihydrothieno[2,3-d]pyrimidine-2-carboxamide; N-(3-methoxybenzyl)-5-{[(4-aminocyclohexyl)methoxy]methyl}-4-oxo-3,4-dihydrothieno[2,3-d]pyrimidine-2-carboxamide; N-(3-methoxybenzyl)-5-{[(1-acetylpiperidin-4-yl)methoxy]methyl}-4-oxo-3,4-dihydrothieno[2,3-d]pyrimidine-2-carboxamide; N-(3-methoxybenzyl)-4-oxo-5-[(2-piperidin-4-ylethoxy)methyl]-3,4-dihydrothieno[2,3-d]pyrimidine-2-carboxamide hydrochloride; and N-(3-methoxybenzyl)-5-({[1-(methylsulfonyl)piperidin-4-yl]methoxy}methyl)-4-oxo-3,4-dihydrothieno[2,3-d]pyrimidine-2-carboxamide; or a pharmaceutically acceptable salt thereof.

Another embodiment of the invention is a compound selected from Trans-5-(((2-((4-Fluoro-3-methoxybenzyl)carbamoyl)-4-oxo-3,4-dihydrothieno[2,3-d]pyrimidin-5-yl)methoxy)methyl)-1,4-dioxane-2-carboxylic acid; Trans-5-(((2-((3-Methoxybenzyl)carbamoyl)-4-oxo-3,4-dihydrothieno[2,3-d]pyrimidin-5-yl)methoxy)methyl)-1,4-dioxane-2-carboxylic acid; N-(4-Fluoro-3-methoxybenzyl)-5-(((trans-5-carbamoyl-1,4-dioxan-2-yl)methoxy)methyl)-4-oxo-3,4-dihydrothieno[2,3-d]pyrimidine-2-carboxamide; N-(4-Fluoro-3-methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide; N-(3-Methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide; 5-({[trans-4-aminomethylcyclohexyl]methoxy}methyl)-N-(3-methoxybenzyl)-4-oxo-3,4-dihydrothieno[2,3-d]pyrimidine-2-carboxamide; 2-((trans-1,4)-4-(((2-((4-fluoro-3-methoxybenzyl)carbamoyl)-4-oxo-3,4-dihydrothieno[2,3-d]pyrimidin-5-yl)methoxy)methyl)cyclohexylamino)-2-oxoethyl acetate; 2-((trans-1,4)-4-(((2-((3-methoxybenzyl)carbamoyl)-4-oxo-3,4-dihydrothieno[2,3-d]pyrimidin-5-yl)methoxy)methyl)cyclohexylamino)-2-oxoethyl acetate; Trans-4-(((2-((4-Fluoro-3-methoxybenzyl)carbamoyl)-4-oxo-3,4-dihydrothieno[2,3-d]pyrimidin-5-yl)methoxy)methyl)cyclohexanecarboxylic acid; Trans-4-(((2-((3-Methoxybenzyl)carbamoyl)-4-oxo-3,4-dihydrothieno[2,3-d]pyrimidin-5-yl)methoxy)methyl)cyclohexanecarboxylic acid; N-(4-Fluoro-3-methoxybenzyl)-5-((((trans-1,4)-4-(2-hydroxyacetamido)cyclohexyl)methoxy)methyl)-4-oxo-3,4-dihydrothieno[2,3-d]pyrimidine-2-carboxamide; N-(3-Methoxybenzyl)-5-((((trans-1,4)-4-(2-hydroxyacetamido)cyclohexyl)methoxy)methyl)-4-oxo-3,4-dihydrothieno[2,3-d]pyrimidine-2-carboxamide; N-(4-Fluoro-3-methoxybenzyl)-5-(((trans-4-(hydroxymethyl)cyclohexyl)methoxy)methyl)-4-oxo-3,4-dihydrothieno[2,3-d]pyrimidine-2-carboxamide; N-(3-methoxybenzyl)-5-(((trans-4-(hydroxymethyl)cyclohexyl)methoxy)methyl)-4-oxo-3,4-dihydrothieno[2,3-d]pyrimidine-2-carboxamide; N-(4-Fluoro-3-methoxybenzyl)-5-[({trans-4-[(methylsulfonyl)amino]cyclohexyl}methoxy)methyl]-4-oxo-3,4-dihydrothieno[2,3-d]pyrimidine-2-carboxamide; N-(3-Methoxybenzyl)-5-[({trans-4-[(methylsulfonyl)amino]cyclohexyl}methoxy)methyl]-4-oxo-3,4-dihydrothieno[2,3-d]pyrimidine-2-carboxamide; N-(3-methoxybenzyl)-5-[({trans-4-[(methylsulfonyl)aminomethyl]cyclohexyl}methoxy)methyl]-4-oxo-3,4-dihydrothieno[2,3-d]pyrimidine-2-carboxamide; 5-({[trans-4-(Acetylamino)cyclohexyl]methoxy}methyl)-N-(4-fluoro-3-methoxybenzyl)-4-oxo-3,4-dihydrothieno[2,3-d]pyrimidine-2-carboxamide; 5-({[trans-4-(Acetylamino)cyclohexyl]methoxy}methyl)-N-(3-methoxybenzyl)-4-oxo-3,4-dihydrothieno[2,3-d]pyrimidine-2-carboxamide; N-(4-Fluoro-3-methoxybenzyl)-5-(((trans-4-carbamoylcyclohexyl)methoxy)methyl)-4-oxo-3,4-dihydrothieno[2,3-d]pyrimidine-2-carboxamide; N-(4-Fluoro-3-methoxybenzyl)-5-(((trans-4-cyanocyclohexyl)methoxy)methyl)-4-oxo-3,4-dihydrothieno[2,3-d]pyrimidine-2-carboxamide; N-(3-Methoxybenzyl)-5-(((trans-4-cyanocyclohexyl)methoxy)methyl)-4-oxo-3,4-dihydrothieno[2,3-d]pyrimidine-2-carboxamide; 5-({[trans-4-Aminocyclohexyl]methoxy}methyl)-N-(3-methoxybenzyl)-4-oxo-3,4-dihydrothieno[2,3-d]pyrimidine-2-carboxamide; 5-{[2-(1-Acetylpiperidin-4-yl)ethoxy]methyl}-N-(3-methoxybenzyl)-4-oxo-3,4-dihydrothieno[2,3-d]pyrimidine-2-carboxamide; N-(3-methoxybenzyl)-4-oxo-5-[(2-piperidin-4-ylethoxy)methyl]-3,4-dihydrothieno[2,3-d]pyrimidine-2-carboxamide hydrochloride; and N-(3-methoxybenzyl)-5-({2-[1-(methylsulfonyl)piperidin-4-yl]ethoxy}methyl)-4-oxo-3,4-dihydrothieno[2,3-d]pyrimidine-2-carboxamide; or a pharmaceutically acceptable salt thereof.

Another embodiment of the invention is a pharmaceutical composition comprising a compound as described hereinabove, or a pharmaceutically acceptable salt thereof, admixed with a pharmaceutically acceptable carrier, excipient, or diluent.

Another embodiment of the invention is a method for inhibiting an MMP-13 enzyme in an animal, comprising administering to the animal an MMP-13 inhibiting amount of a compound described hereinabove, or a pharmaceutically acceptable salt thereof.

Another embodiment of the invention is a method for treating a disease mediated by an MMP-13 enzyme, comprising administering to a patient suffering from such a disease a nontoxic effective amount of a compound described hereinabove, or a pharmaceutically acceptable salt thereof.

Another embodiment of the invention is a method for treating arthritis, comprising administering to a patient suffering from an arthritis disease a nontoxic antiarthritic effective amount of a compound described hereinabove, or a pharmaceutically acceptable salt thereof.

Another embodiment of the invention is a method for treating osteoarthritis, comprising administering to a patient suffering from osteoarthritis a nontoxic effective amount of a compound described hereinabove, or a pharmaceutically acceptable salt thereof.

Another embodiment of the invention is a method for treating rheumatoid arthritis, comprising administering to a patient suffering from rheumatoid arthritis a nontoxic effective amount of a compound described hereinabove, or a pharmaceutically acceptable salt thereof.

Another embodiment of the invention is a method for treating psoriatic arthritis, comprising administering to a patient suffering from psoriatic arthritis a nontoxic effective amount of a compound described hereinabove, or a pharmaceutically acceptable salt thereof.

Another embodiment of the invention is a method for treating a cancer, comprising administering to a patient suffering from a cancer a nontoxic anti-cancer effective amount of a compound described hereinabove, or a pharmaceutically acceptable salt thereof.

Another embodiment of the invention is a method for treating inflammation, comprising administering to a patient suffering from inflammation a nontoxic effective amount of a compound described hereinabove, or a pharmaceutically acceptable salt thereof.

Another embodiment of the invention is a method for treating chronic obstructive pulmonary disease, comprising administering to a patient suffering from chronic obstructive pulmonary disease a nontoxic effective amount of a compound described hereinabove, or a pharmaceutically acceptable salt thereof.

Another embodiment of the invention is a method for treating psoriasis, comprising administering to a patient suffering from psoriasis a nontoxic effective amount of a compound described hereinabove, or a pharmaceutically acceptable salt thereof.

Another embodiment of the invention is a method for treating asthma, comprising administering to a patient suffering from asthma a nontoxic effective amount of a compound described hereinabove, or a pharmaceutically acceptable salt thereof.

Another embodiment of the invention is a method for treating inflammatory bowel disease, comprising administering to a patient suffering from inflammatory bowel disease a nontoxic effective amount of a compound described hereinabove, or a pharmaceutically acceptable salt thereof.

For the purposes of this invention, the term “arthritis”, which is synonymous with the phrase “arthritic condition”, includes osteoarthritis, rheumatoid arthritis, degenerative joint disease, spondyloarthropathies, gouty arthritis, systemic lupus erythematosus, juvenile arthritis, and psoriatic arthritis. An allosteric inhibitor of MMP-13 having an anti-arthritic effect is a compound as defined above that inhibits the progress, prevents further progress, or reverses progression, in part or in whole, of any one or more symptoms of any one of the arthritic diseases and disorders listed above.

As used herein, the term “alkyl” refers to a straight or branched chain monovalent hydrocarbon radical. For example, a C₁₋₆ alkyl radical is a straight or branched chain monovalent hydrocarbon radical having 1 to 6 carbon atoms. Examples of C₁₋₆ alkyl radicals include methyl, ethyl, 1-propyl, 2-propyl, 1-butyl, 2-butyl, 2,2-dimethylethyl, 1-pentyl, 2-pentyl, 2,2-dimethylpropyl, and 1-hexyl.

As used herein, the term “alkylene” refers to a straight or branched chain divalent hydrocarbon radical. For example, a C₁₋₆ alkylene radical is a straight or branched chain divalent hydrocarbon radical having 1 to 6 carbon atoms. Examples of C₁₋₆ alkylene radicals include methylene, ethylene, 1-propylene, 2-propylene, 1-butylene, 2-butylene, 2,2-dimethylethylene, 1-pentylene, 2-pentylene, 2,2-dimethylpropylene, and 1-hexylene.

As used herein, the term “cycloalkyl” refers to a cyclic monovalent hydrocarbon radical. For example, a C₃₋₆ cycloalkyl radical is a cyclic monovalent hydrocarbon radical having 1 to 6 carbon atoms. Examples of C₃₋₆ cycloalkyl radicals include cyclopropyl, cyclobutyl, cyclopentyl, and cyclohexyl.

The term “IC₅₀” means the concentration of a compound, usually expressed as μM or nM, required to inhibit an enzyme's catalytic activity by 50%.

As used herein, the phrase “cartilage damage” means a disorder of hyaline cartilage and subchondral bone characterized by hypertrophy of tissues in and around the involved joints, which may or may not be accompanied by deterioration of hyaline cartilage surface.

The phrase “treating”, which is related to the terms “treat” and “treated”, means administration of an invention combination as defined above that inhibits the progress, prevents further progress, or reverses progression, in part or in whole, of any one or more symptoms of any one of the diseases and disorders listed above.

The phrase “invention compound” means a compound of Formula I, or a pharmaceutically acceptable salt thereof, as fully defined above.

The term “NSAID” is an acronym for the phrase “nonsteroidal anti-inflammatory drug”, which means any compound which inhibits cyclooxygenase-1 (“COX-1”) and cyclooxygenase-2. Most NSAIDs fall within one of the following five structural classes: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxicam. Other useful NSAIDs include aspirin, acetominophen, indomethacin, and phenylbutazone. Selective inhibitors of cyclooxygenase-2 as described above may be considered to be NSAIDs also.

A selective inhibitor of COX-2 is a compound that inhibits COX-2 selectively versus COX-1 such that a ratio of IC₅₀ for a compound with COX-1 divided by a ratio of IC₅₀ for the compound with COX-2 is greater than, or equal to, 5, where the ratios are determined in one or more assays. All that is required to determine whether a compound is a selective COX-2 inhibitor is to assay a compound in one of a number of well know assays in the art.

The term “drugs”, which is synonymous with the phrases “active components”, “active compounds”, and “active ingredients”, includes celecoxib, or a pharmaceutically acceptable salt thereof, valdecoxib, or a pharmaceutically acceptable salt thereof, and an allosteric inhibitor of MMP-13, and may further include one or two of the other therapeutic agents described above.

An allosteric inhibitor of MMP-13 is any compound of Formula I that binds allosterically into the S1′ site of the MMP-13 enzyme, including the S1′ channel, and/or the S1″ site, without ligating, coordinating, or binding the catalytic zinc of the MMP-13.

Certain of the invention compounds possess one or more chiral centers, and each center may exist in the R or S configuration. The scope of the present invention encompasses any diastereomeric, enantiomeric, or epimeric form of invention compound, as well as mixtures thereof. Compounds of Formula I may be prepared as single enantiomer or as a mixture of individual enantiomers which includes racemic mixtures. Methods to obtain preferentially a single enantiomer from a mixture of individual enantiomers or a racemic mixture are well known to those ordinarily skilled in the art of organic chemistry. Such methods include but are not limited to preferential crystallization of diastereomeric salts (e.g. tartrate or camphor sulfonate), covalent derivatization by a chiral, non-racemic reagent followed by separation of the resulting diastereomers by common methods (e.g. crystallization, chromatographic separation, or distillation) and chemical reversion to scalemic compound, Simulated Moving Bed technology, or high/medium-pressure liquid chromatography employing a chiral stationary phase (Eliel, E. L. Stereochemistry of Organic Compounds, 1994; Subramanian, G. Chiral Separation Techniques: A Practical Approach, 2001). These techniques may be performed on the final compounds of Formula I or on any intermediates to compounds of Formula I which bear a stereogenic center. Also, to facilitate separation by any of the methods described above, the compounds of Formula I or any intermediates to the compounds of Formula I which bear a stereogenic center may be transiently reacted with an achiral reagent, separated, and then reverted to scalemic compound by standard synthetic techniques.

Additionally, certain invention compounds may exist as geometric isomers such as the entgegen (E) and zusammen (Z) isomers of 1,2-disubstituted alkenyl groups or cis and trans isomers of disubstituted cyclic groups. Any cis, trans, syn, anti, entgegen (E), or zusammen (Z) isomer of a compound of Formula I, as well as mixtures thereof, is encompassed within the scope of the present invention.

Certain invention compounds can exist as two or more tautomeric forms. Tautomeric forms of the invention compounds may interchange, for example, via enolization/de-enolization, 1,2-hydride, 1,3-hydride, or 1,4-hydride shifts, and the like. Any tautomeric form of a compound of Formula I, as well as mixtures thereof, is encompassed within the scope of the present invention.

Some compounds of the present invention have cycloalkyl groups, which may be substituted at more than one carbon atom, in which case all geometric forms thereof, both cis and trans, and mixtures thereof, are within the scope of the present invention.

Encompassed within the scope of the present invention are isotopically-labelled compounds of Formula I, which are identical to those recited above, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as 2H, 3H, 13C, 14C, 15N, 18O, 17O, 31P, 32P, 35S, 18F and 36Cl, respectively. Compounds of the present invention and pharmaceutically acceptable salts of said compounds which contain the aforementioned isotopes and/or other isotopes of other atoms are within the scope of this invention. Certain isotopically labelled compounds of the present invention, for example those into which radioactive isotopes such as 3H and 14C are incorporated, are useful in drug and/or substrate tissue distribution assays. Tritiated, i.e., 3H and carbon-14, i.e., 14C, isotopes are particularly preferred for their ease of preparation and detectability. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by carrying out the procedures incorporated by reference above or disclosed in the Schemes and/or in the Examples and Preparations below, by substituting a readily available isotopically labelled reagent for a non-isotopically labelled reagent.

Some of the invention compounds are capable of further forming nontoxic pharmaceutically acceptable salts, including, but not limited to, acid addition and/or base salts. The acid addition salts are formed from basic invention compounds, whereas the base addition salts are formed from acidic invention compounds. All of these forms are within the scope of the compounds useful in the invention.

Pharmaceutically acceptable acid addition salts of the basic invention compounds include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977; 66:1).

An acid addition salt of a basic invention compound is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the invention compounds and their respective acid addition salt forms are equivalent for purposes of the present invention.

A nontoxic pharmaceutically acceptable base addition salt of an acidic invention compound may be prepared by contacting the free acid form of the compound with a metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic invention compound may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the invention compounds differ from their respective salt forms somewhat in certain physical properties such as solubility, crystal structure, hygroscopicity, and the like, but otherwise the salts are equivalent to their respective free acid for purposes of the present invention.

Certain invention compounds can exist in unsolvated forms as well as solvated forms, including hydrated forms. In general, the solvated forms, including hydrated forms, are equivalent to unsolvated forms and are encompassed within the scope of the present invention.

The invention also provides pharmaceutical compositions comprising a compound of Formula I, or a pharmaceutically acceptable salt thereof, as defined above, together with a pharmaceutically acceptable carrier, diluent, or excipient.

The present invention also relates to the formulation of a compound of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

A therapeutically effective amount, or, simply, effective amount, of a compound of Formula I will generally be from about 1 to about 300 mg/kg of subject body weight of the compound of Formula I, or a pharmaceutically acceptable salt thereof. Typical doses will be from about 10 to about 5000 mg/day for an adult subject of normal weight for each component of the combination. In a clinical setting, regulatory agencies such as, for example, the Food and Drug Administration (“FDA”) in the U.S. may require a particular therapeutically effective amount.

In determining what constitutes a nontoxic effective amount or a therapeutically effective amount of a compound of Formula I for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including the Food and Drug Administration guidelines, or guidelines from an equivalent agency, published clinical studies, the subject's (e.g., mammal's) age, sex, weight and general condition, as well as the type and extent of the disease, disorder or condition being treated, and the use of other medications, if any, by the subject. As such, the administered dose may fall within the ranges or concentrations recited above, or may vary outside them, ie, either below or above those ranges, depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the compound of Formula I that are less than optimum for a particular subject. Thereafter, the dosage can be increased by small increments until the optimum effect under the circumstance is reached. For convenience, the total daily dosage may be divided and administered in portions during the day, if desired.

Pharmaceutical compositions, described briefly here and more fully below, of an invention combination may be produced by formulating the invention combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Alternatively, the compounds of Formula I may be formulated separately.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat any of the above-listed diseases and disorders.

The percentage of the active ingredients of a compound of Formula I, or a pharmaceutically acceptable salt thereof, in the foregoing compositions can be varied within wide limits, but for practical purposes it is preferably present in a total concentration of at least 10% in a solid composition and at least 2% in a primary liquid composition. The most satisfactory compositions are those in which a much higher proportion of the active ingredients are present, for example, up to about 95%.

Preferred routes of administration of a compound of Formula I are oral or parenteral. However, another route of administration may be preferred depending upon the condition being treated. For example, topical administration or administration by injection may be preferred for treating conditions localized to the skin or a joint. Administration by transdermal patch may be preferred where, for example, it is desirable to effect sustained dosing.

It should be appreciated that the different routes of administration may require different dosages. For example, a useful intravenous (“IV”) dose is between 5 and 50 mg, and a useful oral dosage is between 20 and 800 mg, of a compound of Formula I, or a pharmaceutically acceptable salt thereof. The dosage is within the dosing range used in treatment of the above-listed diseases, or as would be determined by the needs of the patient as described by the physician.

Compounds of Formula I may be administered in any form. Preferably, administration is in unit dosage form. A unit dosage form of the compound of Formula Ito be used in this invention may also comprise other compounds useful in the therapy of diseases described above. A further description of pharmaceutical formulations useful for administering the compounds of Formula I and invention combinations is provided below.

The invention also provides combinations, comprising a compound of Formula I, or a pharmaceutically acceptable salt thereof, together with another pharmaceutically active component as described herein.

The active components of the invention combinations, may be formulated together or separately and may be administered together or separately. The particular formulation and administration regimens used may be tailored to the particular patient and condition being treated by a practitioner of ordinary skill in the medical or pharmaceutical arts.

For the treatment of rheumatoid arthritis, the compounds of the invention may be combined with agents such as TNF-α inhibitors such as anti-TNF monoclonal antibodies and TNF receptor immunoglobulin molecules (such as Enbrel®), low dose methotrexate, lefunimide, hydroxychloroquine, d-penicillamine, auranofin or parenteral or oral gold.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as etoricoxib and rofecoxib, analgesics and intraarticular therapies such as corticosteroids and hyaluronic acids such as hyalgan and synvisc.

The active ingredient of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, ILra, H1-receptor antagonists; kinin-B1- and B2-receptor antagonists; prostaglandin inhibitors such as PGD-, PGF- PGI2- and PGE-receptor antagonists; thromboxane A2 (TXA2-) inhibitors; 5- and 12-lipoxygenase inhibitors; leukotriene LTC4 LTD4/LTE4- and LTB4-inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The compounds of the present invention may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The compounds of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α2-adrenergic agonists such as clonidine, α-adrenergic receptor antagonists such as prazosin and HMG-CoA-reductase inhibitors (anti-hypercholesterolemics) such as lovastatin or atorvastatin.

The compounds of the present invention may also be administered in combination with one or more antibiotic, antifungal, antiprotozoal, antiviral or similar therapeutic agents.

The compounds of the present invention may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metrifonate.

The compounds of the present invention may also be used in combination with osteoporosis agents such as roloxifene, lasofoxifene, droloxifene or fosomax and immunosuppressant agents such as FK-506 and rapamycin.

Compounds of Formula I may be used in combination with a COX-2 selective inhibitor, more preferably celecoxib, valdecoxib, parecoxib, lumiracoxib, or rofecoxib, or with compounds such as etanercept, infliximab, leflunomide, or methotrexate, and the like.

Compounds of Formula I may be used in combination with biological therapeutics useful for treating arthritic conditions, including CP-870, etanercept (a tumor necrosis factor alpha (“TNF-alpha”) receptor immunoglobulin molecule; trade names ENBREL® and ENBREL ENTANERCEPT® by Immunex Corporation, Seattle, Wash.), infliximab (an anti-TNF-alpha chimeric IgG 1K monoclonal antibody; tradename REMICADE® by Centocor, Inc., Malvern, Pa.), methotrexate (tradename RHEUMATREX® by American Cyanamid Company, Wayne, N.J.), and adalimumab (a human monoclonal anti-TNF-alpha antibody; tradename HUMIRA® by Abbott Laboratories, Abbott Park, Ill.).

The invention also provides methods of inhibiting an MMP-13 enzyme in an animal, comprising administering to the animal a compound of Formula I, or a pharmaceutically acceptable salt thereof.

The invention also provides methods of treating a disease mediated by an MMP-13 enzyme in a patient, comprising administering to the patient a compound of Formula I, or a pharmaceutically acceptable salt thereof.

The invention also provides methods of treating diseases such as heart disease, multiple sclerosis, osteo and rheumatoid arthritis, arthritis other than osteo- or rheumatoid arthritis, cardiac insufficiency, inflammatory bowel disease, heart failure, age-related macular degeneration, chronic obstructive pulmonary disease, asthma, periodontal diseases, psoriasis, atherosclerosis, and osteoporosis in a patient, comprising administering to the patient a compound of Formula I, or a pharmaceutically acceptable salt thereof.

Other mammalian diseases and disorders which are treatable by administration of an invention combination alone, or contained in a pharmaceutical composition as defined below, include: fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

One of ordinary skill in the art will appreciate that the compounds of the invention are useful in treating a diverse array of diseases. One of ordinary skill in the art will also appreciate that when using the compounds of the invention in the treatment of a specific disease that the compounds of the invention may be combined with various existing therapeutic agents used for that disease.

This invention also relates to a method of or a pharmaceutical composition for treating inflammatory processes and diseases comprising administering a compound of this invention to a mammal, including a human, cat, livestock or dog, wherein said inflammatory processes and diseases are defined as above and said inhibitory compound is used in combination with one or more other therapeutically active agents under the following conditions:
A) where a joint has become seriously inflamed as well as infected at the same time by bacteria, fungi, protozoa and/or virus, said inhibitory compound is administered in combination with one or more antibiotic, antifungal, antiprotozoal and/or antiviral therapeutic agents;
B) where a multi-fold treatment of pain and inflammation is desired, said inhibitory compound is administered in combination with inhibitors of other mediators of inflammation, comprising one or more members independently selected from the group consisting essentially of:
   1. NSAIDs;
   2. H₁-receptor antagonists;
   3. kinin-B₁- and B₂-receptor antagonists;
   4. prostaglandin inhibitors selected from the group consisting of PGD-, PGF- PGI₂- and PGE-receptor antagonists;
   5. thromboxane A₂ (TXA₂-) inhibitors;
   6 5-, 12- and 15-lipoxygenase inhibitors;
   7. leukotriene LTC₄-, LTD₄/LTE₄- and LTB₄-inhibitors;
   8. PAF-receptor antagonists;
   9. gold in the form of an aurothio group together with one or more hydrophilic groups;
   10. immunosuppressive agents selected from the group consisting of cyclosporine, azathioprine and methotrexate;
   11. anti-inflammatory glucocorticoids;
   12. penicillamine;
   13. hydroxychloroquine;
   14. anti-gout agents including colchicine; xanthine oxidase inhibitors including allopurinol; and uricosuric agents selected from probenecid, sulfinpyrazone and benzbromarone;
C) where older mammals are being treated for disease conditions, syndromes and symptoms found in geriatric mammals, said inhibitory compound is administered in combination with one or more members independently selected from the group consisting essentially of:
   1. cognitive therapeutics to counteract memory loss and impairment;
   2. anti-hypertensives and other cardiovascular drugs intended to offset the consequences of atherosclerosis, hypertension, myocardial ischemia, angina, congestive heart failure and myocardial infarction, selected from the group consisting of:
      a. diuretics;
      b. vasodilators;
      c. β-adrenergic receptor antagonists;
      d. angiotensin-II converting enzyme inhibitors (ACE-inhibitors), alone or optionally together with neutral endopeptidase inhibitors;
      e. angiotensin II receptor antagonists;
      f. renin inhibitors;
      g. calcium channel blockers;
      h. sympatholytic agents;
      i. α₂-adrenergic agonists;
      j. α-adrenergic receptor antagonists; and
      k. HMG-CoA-reductase inhibitors (anti-hypercholesterolemics);
   3. vinca alkaloids selected from:
      a. vinblastine; and
      b. vincristine;
   4. growth hormone secretagogues;
   5. strong analgesics;
   6. local and systemic anesthetics; and
   7. H₂-receptor antagonists, proton pump inhibitors and other gastroprotective agents.

The invention method is useful in human and veterinary medicines for treating mammals suffering from one or more of the above-listed diseases and disorders.

An allosteric inhibitor of MMP-13 may be readily identified by one of ordinary skill in the pharmaceutical or medical arts by assaying an alkyne test compound for inhibition of MMP-13 as described, for example, in Biological Methods 1 or 2 of International Patent Application Pub. No. WO 04/014366, the content of which is herein incorporated by reference. Allosteric inhibition of MMP-13 may be identified by one of ordinary skill in the pharmaceutical or medical arts by assaying the test invention compound for inhibition of MMP-13 in the presence of an inhibitor to the catalytic zinc of MMP-13 as described, for example, in Biological Methods 3 or 4 of International Patent Application Pub. No. WO 04/014366, the content of which is herein incorporated by reference.

Further, a compound having an anti-inflammatory, an analgesic, anti-arthritic, or a cartilage damage inhibiting effect, or any combination of these effects, may be readily identified by one of ordinary skill in the pharmaceutical or medical arts by assaying the compound in any number of well known assays for measuring determining the compound's effects on cartilage and or other joint tissue damage, arthritis, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of a compound or control vehicle may be administered with a cartilage-damaging agent to cartilage such as IL-1, and the cartilage damage inhibiting effects in both tests studied by gross examination or histopathologic examination of the cartilage, or by measurement of biological markers of cartilage damage such as, for example, proteoglycan content or hydroxyproline content, or by biomarkers of type II collagen degradation such as CTX-II or TIINE (Sunyer et al., Osteo. Cartilage 12 (2004) (Suppl. B), p. P84). Further, in vivo studies to assay cartilage damage and/or joint degeneration may be performed as follows: an amount of a compound or control vehicle may be administered with a cartilage damaging agent to an animal or may be administered in the absence of cartilage damaging agents, to animals that have surgery-induced or spontaneous OA lesions in the knee. Examples of surgery-induced animal models include the rat medial meniscus tear model (Bendele, J. Musculoskelet. Neuronal. Interact. 1 (2001) (4), 363-76) or the dog anterior cruciate ligament transaction model (Bendele, supra). The effects of the compound being assayed in the animals for effects on cartilage integrity and/or joint structure may be evaluated by gross examination or histopathologic examination of the affected joint(s), and response to compounds further characterized by measurement of biological markers of cartilage damage such as, for example, proteoglycan content or hydroxyproline content or biomarkers of type II collagen degradation such as CTX-II or TIINE in biological fluids such as urine, plasma, serum or synovial fluids. Effect of the compounds may also be assessed by observation of the effects in an acute model on functional limitations of the affected joint.

Several methods of identifying a compound with cartilage damage inhibiting properties are described below. The amount to be administered in an assay is dependent upon the particular assay employed, but in any event is not higher than the well known maximum amount of a compound that the particular assay can effectively accommodate.

Similarly, compounds having pain-alleviating properties may be identified using any one of a number of in vivo animal models of pain.

Still similarly, compounds having anti-inflammatory properties may be identified using any one of a number of in vivo animal models of inflammation. For example, for an example of inflammation models, see U.S. Pat. No. 6,329,429, which is incorporated herein by reference.

Still similarly, compounds having anti-arthritic properties may be identified using any one of a number of in vivo animal models of arthritis. For example, for an example of arthritis models, see also U.S. Pat. No. 6,329,429.

Other embodiments of the present invention are compounds described herein, or a pharmaceutically acceptable salt thereof, that are ≧10, ≧20, ≧50, ≧100, or ≧1000 times more potent versus MMP-13 than versus at least two of any other MMP enzyme or TACE.

Still other aspects of the present invention are compounds of Formula I, or a pharmaceutically acceptable salt thereof, that are selective inhibitors of MMP-13 versus 2, 3, 4, 5, 6, or 7 other MMP enzymes, or versus TACE and 1, 2, 3, 4, 5, 6, or 7 other MMP enzymes.

It should be appreciated that selectivity of a compound of Formula I, or a pharmaceutically acceptable salt thereof, is a multidimensional characteristic that includes the number of other MMP enzymes and TACE over which selectivity for MMP-13 inhibition is present and the degree of selectivity of inhibition of MMP-13 over another particular MMP or TACE, as measured by, for example, the IC₅₀ in μM of the compound for the inhibition of the other MMP enzyme or TACE divided by the IC₅₀ in μM of the compound for the inhibition of MMP-13.

As discussed above, one aspect of the present invention is novel compounds that are selective inhibitors of the enzyme MMP-13. A selective inhibitor of MMP-13, as used in the present invention, is a compound that is ≧5× more potent in vitro versus MMP-13 than versus at least one other matrix metalloproteinase enzyme such as, for example, MMP-1, MMP-2, MMP-3, MMP-7, MMP-8, MMP-9, or MMP-14, or versus TACE. A preferred aspect of the present invention is novel compounds that are selective inhibitors of MMP-13 versus MMP-1.

The invention provides a compound of Formula I, or a pharmaceutically acceptable salt thereof, which has an IC₅₀ with any MMP enzyme that is less than or equal to 50 μM. Preferred are compounds of Formula I, or a pharmaceutically acceptable salt thereof, which have an IC₅₀ with a human full-length MMP-13 (“hMMP-13FL”) or a human MMP-13 catalytic domain (“hMMP-13CD”) that is less than or equal to 50 μM. More preferred are compounds of Formula I, or a pharmaceutically acceptable salt thereof, which have an IC₅₀ with a human full-length MMP-13 (“hMMP-13FL”) or a human MMP-13 catalytic domain (“hMMP-13CD”) that is less than or equal to 10 μM. Examples of biological methods useful for determining IC₅₀s for compounds with an MMP are described herein.

The advantages of using an invention compound in a method of the instant invention include the nontoxic nature of the compounds at and substantially above therapeutically effective doses, their ease of preparation, the fact that the compounds are well-tolerated, and the ease of topical, IV, or oral administration of the drugs.

Another important advantage is that the present invention compounds more effectively target a particular disease that is responsive to inhibition of MMP-13 with fewer undesirable side effects than similar compounds that inhibit MMP-13 that are not invention compounds. This is so because the instant invention compounds of Formula I, or a pharmaceutically acceptable salt thereof, do not directly, or indirectly via a bridging water molecule, ligate, coordinate to, or bind to the catalytic zinc cation of MMP-13, but instead bind at a different location from where natural substrate binds to MMP-13. The binding requirements of an allosteric MMP-13 binding site are unique to MMP-13, and account for the specificity of the invention compounds for inhibiting MMP-13 over any other MMP enzyme. See J. Chem. Biol., 2005(12), 181-189. Indeed, prior art inhibitors of MMP-13 bind to the catalytic zinc cations of other MMP enzymes as well as to the catalytic zinc cation of MMP-13, and are consequently significantly less selective inhibitors of MMP-13 enzyme.

The invention compounds which are invention compounds, and pharmaceutically acceptable salts thereof, are thus therapeutically superior to other inhibitors of MMP-13, or even TACE, because of fewer undesirable side effects from inhibition of the other MMP enzymes or TACE. For example, virtually all prior art MMP inhibitors tested clinically to date have exhibited an undesirable side effect known as muscoloskeletal syndrome (“MSS”). MSS is associated with administering an inhibitor of multiple MMP enzymes or an inhibitor of a particular MMP enzyme such as MMP-1. MSS will be significantly reduced in type and severity by administering the invention compound instead of any prior art MMP-13 inhibitor, or a pharmaceutically acceptable salt thereof. The invention compounds are superior to similar compounds that interact with the catalytic zinc cation of the MMP-13 enzyme as discussed above, even if similar compounds show some selectivity for the MMP-13.

### General Synthetic Procedures

Intermediates for the synthesis of a compound of Formula I, or a pharmaceutically acceptable salt thereof, may be prepared by one of ordinary skill in the art of organic chemistry by adapting various synthetic procedures incorporated by reference above or that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods, 1989, by Wiley-Interscience; the text Advanced Organic Chemistry, 4th edition, by Jerry March, Wiley-Interscience, New York, 1992; or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the invention compounds may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A. G., Switzerland, or Lancaster Synthesis Ltd, United Kingdom.

Syntheses of some invention compounds may utilize starting materials, intermediates, or reaction products that contain a reactive functional group. During chemical reactions, a reactive functional group may be protected from reacting by a protecting group that renders the reactive functional group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of a compound of Formula I, or a pharmaceutically acceptable salt thereof, and then later remove them. Procedures for introducing and removing protecting groups are known and referenced such as, for example, in Protective Groups in Organic Synthesis, 2nd ed., Greene T.W. and Wuts P.G., John Wiley & Sons, New York: N.Y., 1991, which is hereby incorporated by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

The following schemes are representative of the methods that can be used to prepare compounds of Formula I. Starting materials may be obtained by procedures described in the schemes, by procedures well known to one of ordinary skill in organic chemistry, and/or may be obtained commercially.

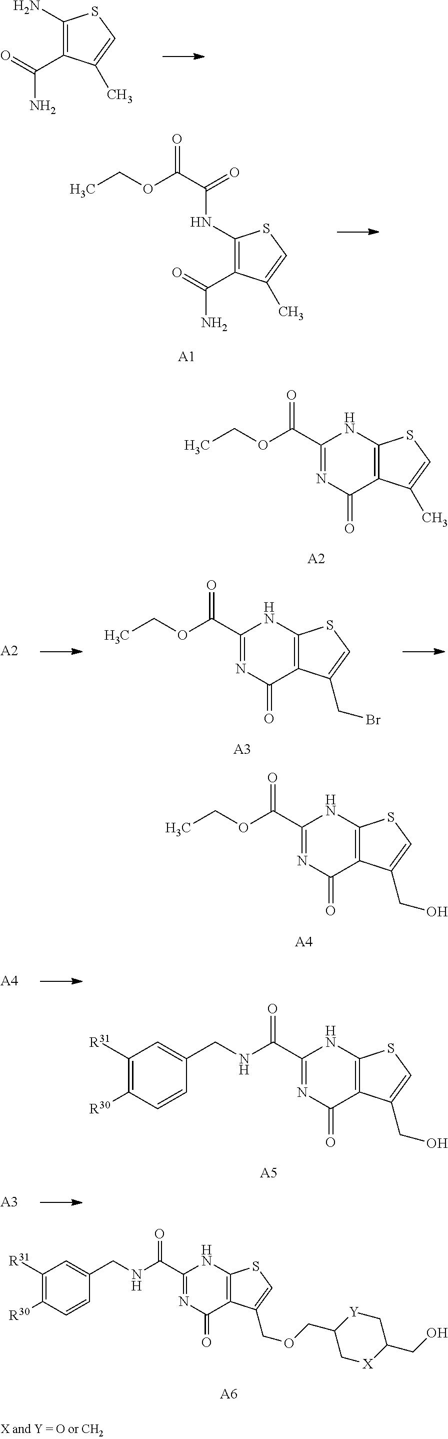

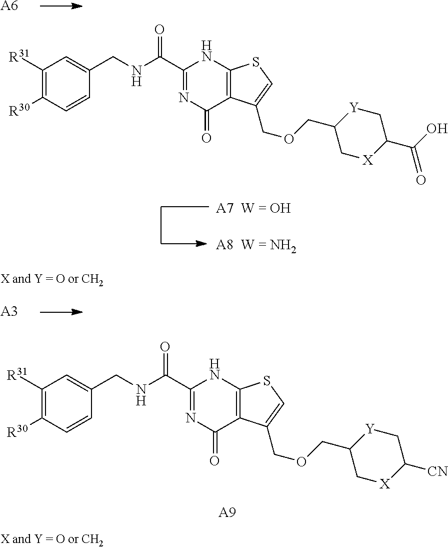

As shown in Scheme A, 2-amino-4-methylthiophene-3-carboxamide is acylated with ethyl chlorooxoacetate to give ethyl 2-(3-carbamoyl-4-methylthiophen-2-ylamino)-2-oxoacetate A1 which is subsequently cyclized to provide ester A2. Ester A2 is reacted with N-bromosuccinimide and 2,2′-azobis(isobutyronitrile) to afford bromide A3. Bromide A3 is converted to the alcohol A4 by treatment with aqueous potassium carbonate. The ester A4 is converted to the amides A5 by reaction with substituted benzylamines. The alcohols A5 can be alkylated with various electrophiles in the presence of base to provide compounds of the invention. Alternatively, the bromide of formula A3 can be reacted with various alcohols (where the alcohol may be exemplified by but is not limited to 1,4-dioxane-2,5-diylmethanol or 1,4-cyclohexanedimethanol) in the presence of base followed by reaction with substituted benzylamines to provide amides A6 of the invention. Alcohols A6 can be oxidized to the acids A7 by treatment with pyridinium dichromate. Treatment of acids A7 with oxalyl chloride followed by reaction with ammonia affords the primary amides of formula A8. Conversion of the amides A8 by treatment the phosphorous oxychloride provides the nitriles A9 of the present invention.

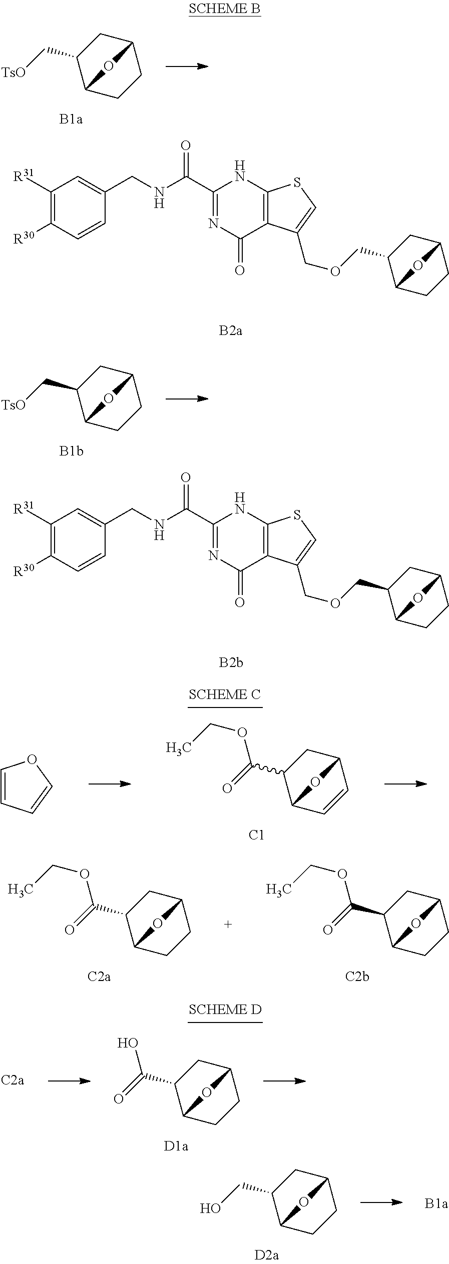

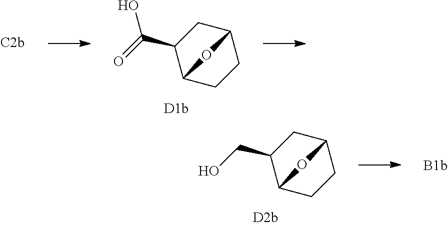

Compounds of formulas B2a and B2b (Scheme B) are prepared by reaction of the corresponding tosylates B1a or B1b with alcohols of the formula A5 in the presence of a base. Tosylates B1a or B1b are prepared as described in Schemes C and D. Furan is reacted with ethyl acrylate in the presence of zinc iodide to afford the cycloadduct C1. Alkene C1 is treated with hydrogen and palladium on carbon in ethanol to provide a mixture of isomers C2a and C2b which are separated by silica gel chromatography. According to Scheme D, each isomer may be saponified with aqueous sodium hydroxide to provide the acids which may be reacted with 1,1′-carbonyldiimidazole and sodium borohydride to afford the alcohols D2a and D2b. The desired tosylates B1a or B1b are provided by reacting the resulting alcohols with toluene sulfonyl chloride in the presence of an amine base.

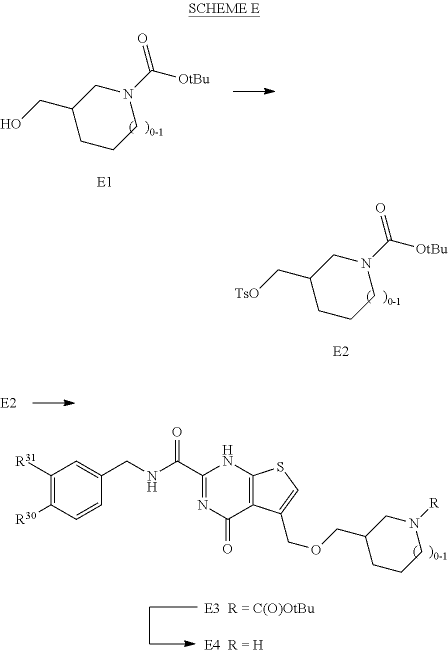

According to Scheme E, N-Boc 3-(hydroxymethyl)pyrrolidine (prepared according to Scheme F) or N-Boc 3-(hydroxymethyl)piperidine (commercially available from CHN Technologies, Woburn, Mass.) is reacted with toluenesulfonyl chloride in the presence of an amine base to provide tosylates of the formula E2. Alcohols of the formula A5 are then reacted with the corresponding tosylates E2 in the presence of a base to provide the N-Boc-protected amines E3. The protecting group is removed by reacting compounds of the formula E3 with trifluoroacetic acid, and the resulting amines E4 may be reacted with alkyl halides, alkyl acid chlorides, and alkyl sulfonyl chlorides in the presence of an amine base to provide compounds of the invention.

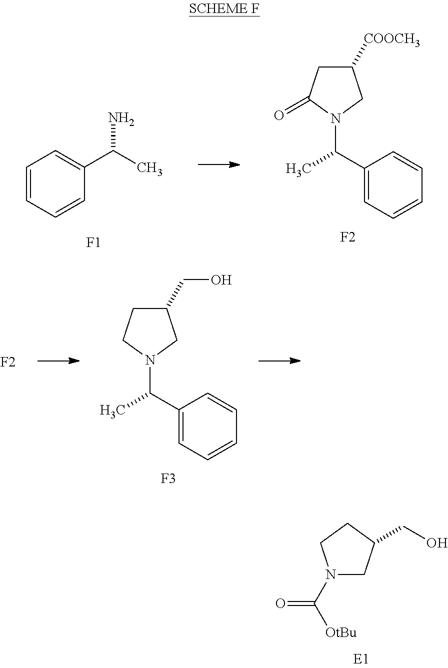

Specific compounds of the formula E1 described above may be prepared as described in Scheme F. (S)—N-Boc 3-(hydroxymethyl)pyrrolidine is prepared from (R)-1-phenylethanamine by cyclization with dimethyl 2-oxosuccinate in refluxing toluene to provide F2. Lactam F2 is then reduced with lithium aluminium hydride to provide pyrrolidine F3. Compound F3 is reacted with hydrogen gas in the presence of Pd(OH)₂ on carbon, and the resulting amine is treated with di-tert-butyl dicarbonate to provide E1. In a similar manner, (R)—N-Boc 3-(hydroxymethyl)pyrrolidine is prepared from (S)-1-phenylethanamine.

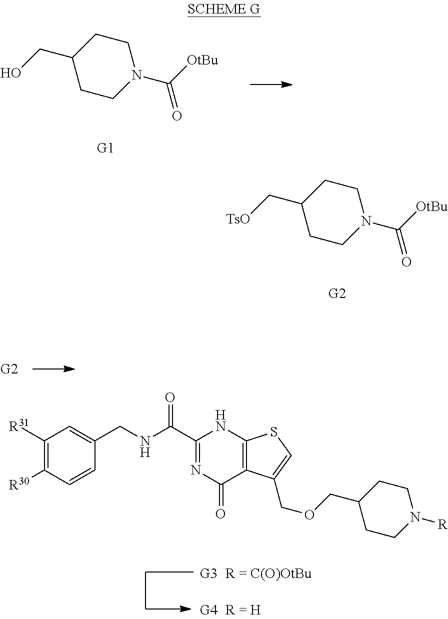

According to Scheme G, N-Boc 4-(hydroxymethyl)piperidine is reacted with toluenesulfonyl chloride in the presence of an amine base to provide tosylate G2. Alcohols of the formula A5 are then reacted with tosylate G2 in the presence of a base to provide the N-Boc-protected amines of the formula G3. The protecting group is removed by reacting compounds of the formula G3 with trifluoroacetic acid, and the resulting amines G4 may be reacted with alkyl halides, alkyl acid chlorides, and alkyl sulfonyl chlorides in the presence of an amine base to provide compounds of the invention.

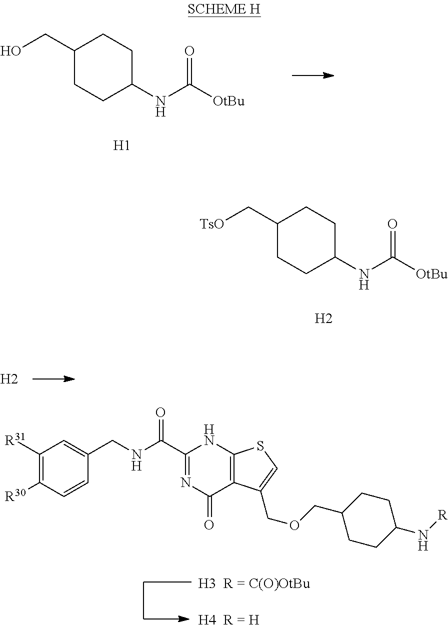

According to Scheme H, tert-butyl trans-(4-hydroxymethyl)cyclohexylcarbamate or tert-butyl cis-(4-hydroxymethyl)cyclohexylcarbamate is reacted with toluenesulfonyl chloride in the presence of an amine base to provide tosylates of the formula H2. Alcohols of the formula A5 are then reacted with the corresponding tosylates H2 in the presence of a base to provide the N-Boc-protected amines H3. The protecting group is removed by reacting compounds of the formula H3 with trifluoroacetic acid, and the resulting amines H4 may be reacted with alkyl halides, alkyl acid chlorides, and alkyl sulfonyl chlorides in the presence of an amine base to provide compounds of the invention.

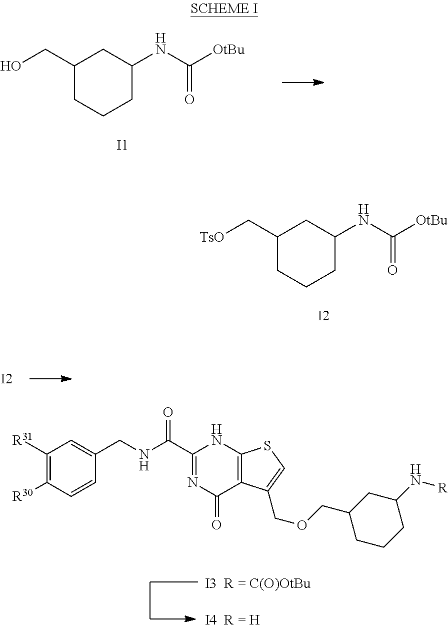

According to Scheme I, tert-butyl trans-(3-hydroxymethyl)cyclohexylcarbamate or tert-butyl cis-(3-hydroxymethyl)cyclohexylcarbamate (commercially available from AMRI, Albany, N.Y.) is reacted with toluenesulfonyl chloride in the presence of an amine base to provide tosylates of the formula I2. Alcohols of the formula A5 are then reacted with the corresponding tosylates I2 in the presence of a base to provide the N-Boc-protected amines I3. The protecting group is removed by reacting compounds of the formula I3 with trifluoroacetic acid, and the resulting amines I4 may be reacted with alkyl halides, alkyl acid chlorides, and alkyl sulfonyl chlorides in the presence of an amine base to provide compounds of the invention.

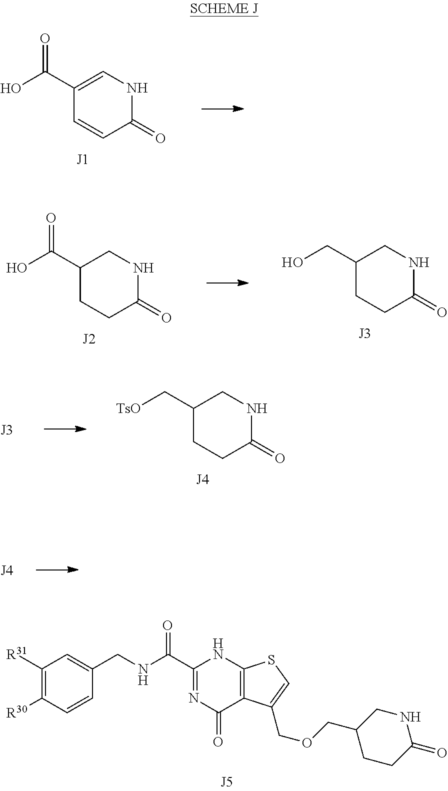

According to Scheme J, 6-hydroxynicotinic acid is reacted with hydrogen in the presence of a palladium catalyst to afford carboxylic acid J2. The resulting acid is treated with 1,1′-carbonyldiimidazole and sodium borohydride to provide alcohol J3 which is then reacted with toluenesulfonyl chloride in the presence of an amine base to provide tosylate J4. Alcohols of the formula A5 are then reacted with tosylate J4 in the presence of a base to provide the compounds of the formula J5.

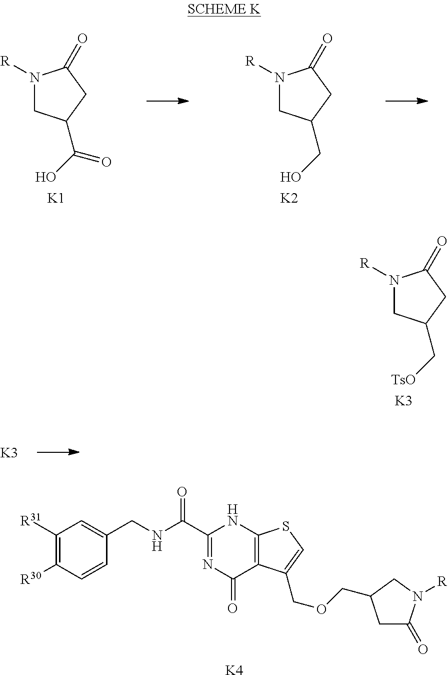

According to Scheme K, lactam carboxylic acids of the formula K1 (where R may include but is not limited to hydrogen, methyl, ethyl, and iso-propyl) prepared as described by E. Valentin et. al. (Tetrahedron Asymmetry, 2001, 12, 3241-3249) are reacted with 1,1′-carbonyldiimidazole and sodium borohydride to provide alcohols of the formula K2. The resulting alcohols are reacted with toluenesulfonyl chloride in the presence of an amine base to provide tosylates of the formula K3. Alcohols of the formula A5 are then reacted with the corresponding tosylates K3 in the presence of a base to provide the compounds of the formula K4.

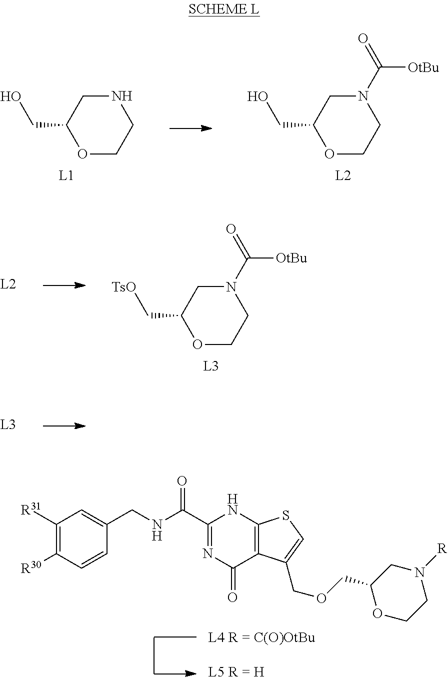

Morpholinyl-substituted examples of the invention may be prepared as described in Scheme L. (S)-2-Hydroxymethylmorpholine may be prepared according to procedures described in J. Med. Chem. 1998, 41, 1934-1942 and converted to N-Boc-(S)-2-hydroxymethylmorpholine (L2) under common conditions (NaOH, di-tert-butyl dicarbonate, water/THF). The resulting alcohol is reacted with toluenesulfonyl chloride in the presence of an amine base to provide tosylate L3. Alcohols of the formula A5 are then reacted with the corresponding tosylate L3 in the presence of a base to provide the N-Boc-protected amines of the formula L4. The protecting group is removed by treating L4 with trifluoroacetic acid, and the resulting amines L5 may be reacted with alkyl halides, alkyl acid chlorides, and alkyl sulfonyl chlorides in the presence of an amine base to provide compounds of the invention. In a similar manner, products bearing an (R)-configuration may be prepared starting from (R)-2-hydroxymethylmorpholine.

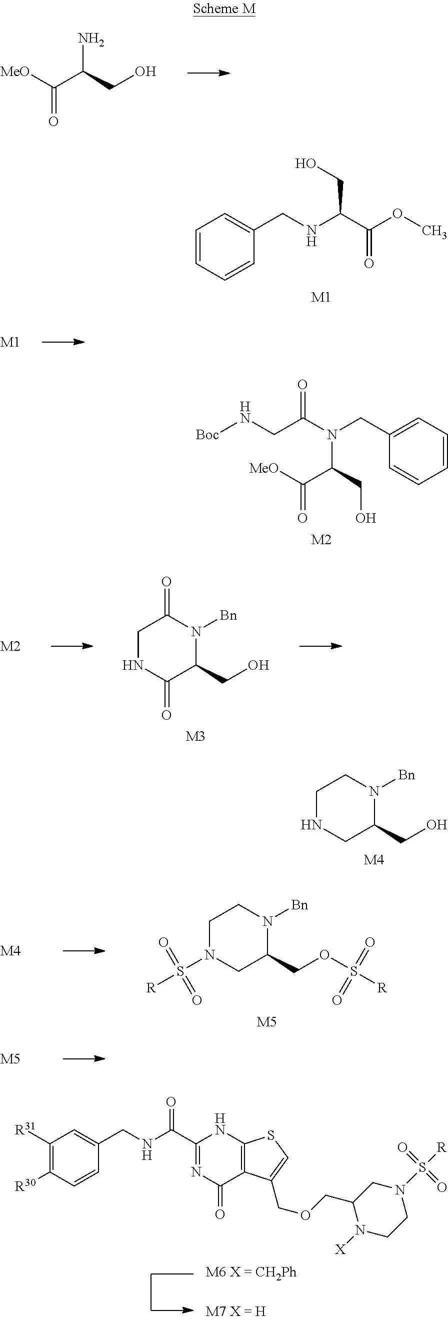

Piperazinyl-substituted examples of the invention may be prepared as described in Scheme M. L-Serine methyl ester is treated with benzaldehyde and NaBH(OAc)₃ to afford benzylamine M1 which is subsequently coupled (BDP, 1-HOBT, diisopropylethylamine) with N-(tert-butoxycarbonyl)glycine to provide M2. The tert-butylcarbamate is removed under acidic conditions (HCl/chloroform) and the resulting product is cyclized under basic conditions (5% aq. NaHCO3) to provide piperizine-2,5-dione M3. Piperizine M4 is then prepared by reduction of M3 with a metal hydride (e.g. lithium aluminium hydride) and the product is subsequently reacted with alkyl sulfonyl chlorides in the presence of an amine base to provide compounds of the formula M5 where R may be but not limited to methyl, ethyl, propyl, and iso-propyl. Alcohols of the formula A5 are then reacted with the resulting sulfonate esters of formula M5 in the presence of a base to provide compounds of the formula M6. Hydrogenation of M6 in the presence of a catalyst such as palladium on carbon in an appropriate solvent (e.g. methanol, ethanol, or tetrahydrofuran) affords compounds of the formula M7. Furthermore, compounds of the formula M7 where X is an alkyl substituent (e.g. methyl, ethyl, and propyl) may be prepared by subsequent reaction with an appropriate alkanal and a reducing agent (e.g. NaBH(OAc)₃).

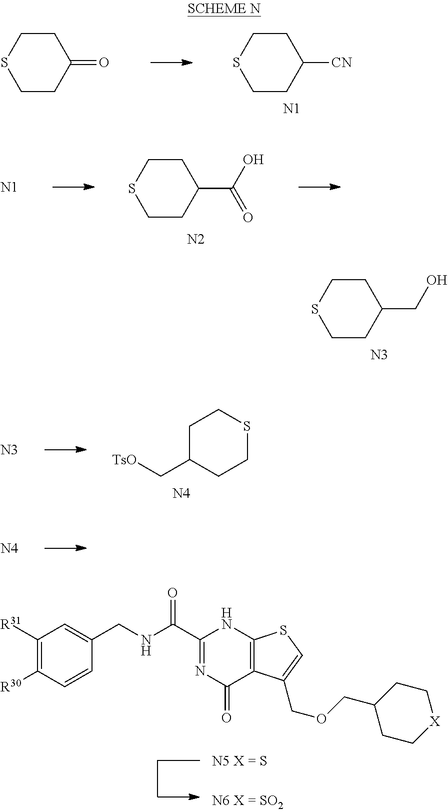

Tetrahydrothiopyran-1,1-dione-substituted examples of the invention may be prepared as described in Scheme N. Tetrahydrothiopyran-4-one is reacted with lithium chloride and samarium diiodide to afford tetrahydro-2H-thiopyran-4-carbonitrile (N1) which is then hydrolyzed under basic conditions (e.g. NaOH) to provide the corresponding carboxylic acid N2. The carboxylic acid is reacted with 1,1′-carbonyldiimidazole and sodium borohydride to provide alcohol N3. Tosylate N4 is then prepared by treating N3 with toluenesulfonyl chloride in the presence of an amine base. Alcohols of the formula A5 are then reacted with the resulting tosylate N4 in the presence of a base to provide compounds of the formula N5. Oxidation of N5 in the presence of oxone provides tetrahydrothiopyran-1,1-dione derivatives of the formula N6.

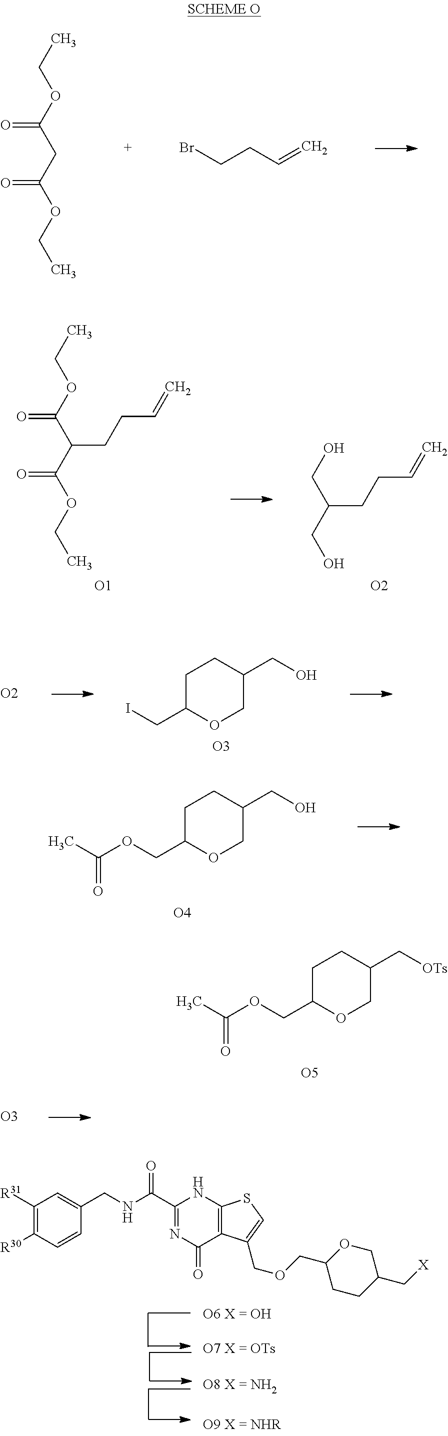

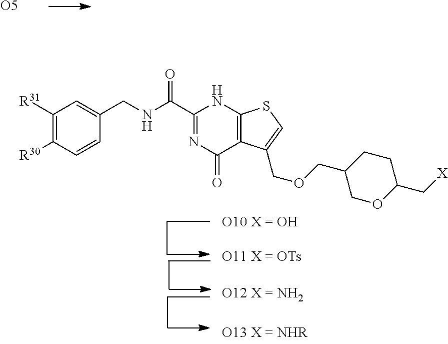

Tetrahydropyran-substituted examples of the invention may be prepared as described in Scheme O. Diethyl malonate is reacted with 4-bromo-1-butene in the presence of a base to give diester O1. Diester O1 is then reduced to diol O2 by treatment with lithium aluminium hydride. Diol O2 is converted to [2-(iodomethyl)tetrahydro-2H-pyran-5-yl]methanol O3 upon treatment with iodide in the presence of base. Iodide O3 is reacted with an acetate salt to displace the halogen and provide alcohol O4. Alcohol O4 is then treated with toluenesulfonyl chloride in an amine base to provide tosylate O5. Alcohols of the formula A5 are then reacted with pyran O3 in the presence of base to provide compounds O6. Alcohols O6 are then treated with toluenesulfonyl chloride in the presence of base to provide tosylates O7 which may then be reacted with a various nucleophiles to provide compounds of the invention. Additionally, tosylates O7 may be reacted with ammonia in a solvent such as methanol to provide amines O8 which may be further reacted with alkyl halides, alkyl acid chlorides, and alkyl sulfonyl chlorides in the presence of a base to provide compounds O9 of the invention. Alternately, alcohols of the formula A5 are reacted with the tosylate O5 in the presence of base followed by hydrolysis of the acetate to provide alcohols O10. The alcohols of O10 are then treated with toluenesulfonyl chloride in the presence of base to provide tosylates O11 which may then be further reacted with various nucleophiles to provide compounds of the invention. Additionally, tosylates of the formula O11 may be reacted with ammonia in a protic solvent to provide amines O12 which may be further reacted with alkyl halides, alkyl acid chlorides, and alkyl sulfonyl chlorides in the presence of amine base to provide the compounds of formula O13 of the invention.

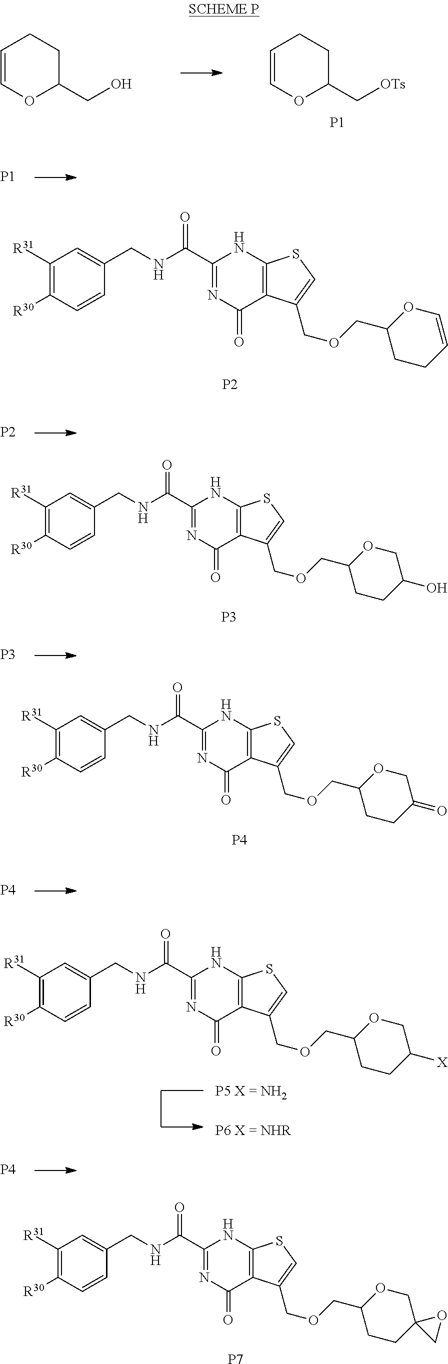

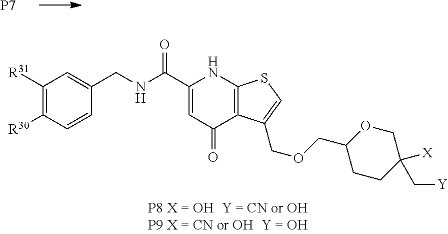

Additional tetrahydropyran-substituted examples of the invention may be prepared as described in Scheme P. Tosylate P1 is prepared by treating 2-hydroxymethyl-3,4-dihydro-2H-pyran with toluenesulfonyl chloride in the presence of an amine base. Alcohols of the formula A5 are then reacted with tosylate P1 in the presence of base to provide the dihydropyran ethers P2. Compounds P2 are oxidized to alcohols P3 by treatment with borane followed by sodium hydroxide and hydrogen peroxide. Alcohols P3 are further oxidized to ketones P4 using Swern conditions. Ketones of the formula P4 may be converted to the primary amines P5 by reductive alkylation using ammonium acetate and a borohydride reducing agent. Amines of the formula P5 may be further reacted with alkyl halides, alkyl acid chlorides, and alkyl sulfonyl chlorides in the presence of amine base to provide compounds P6 of the invention. Alternatively, ketones of the formula P4 may be converted to epoxides of the formula P7 by treatment with trimethylsulfoxonium iodide and an alkoxide base. The epoxides may be reacted with various nucleophiles (where the nucleophile may be but is not limited to cyanide or hydroxide) to provide compounds P8 and P9 of the invention.

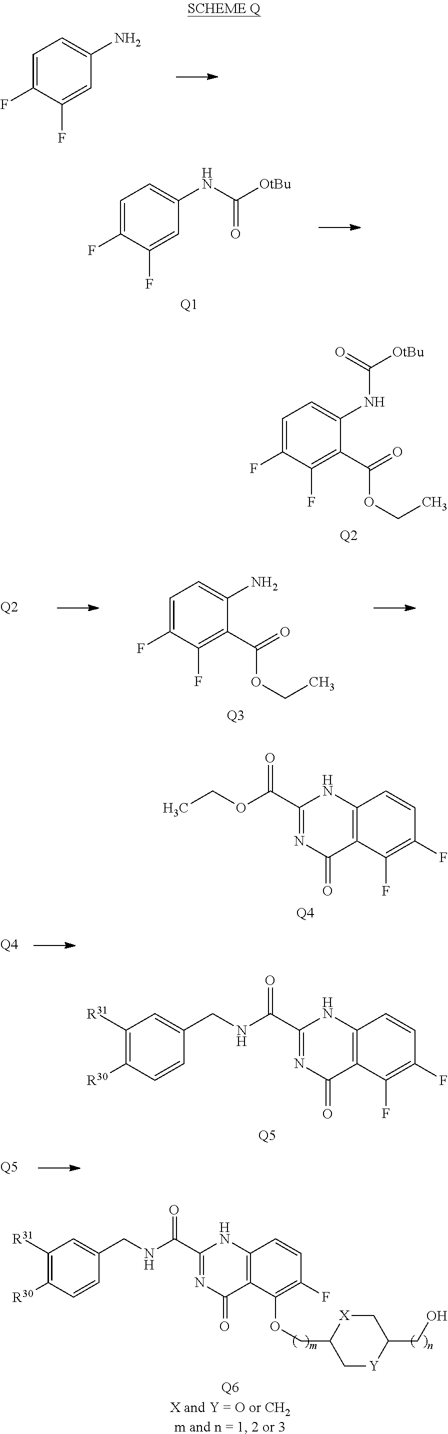

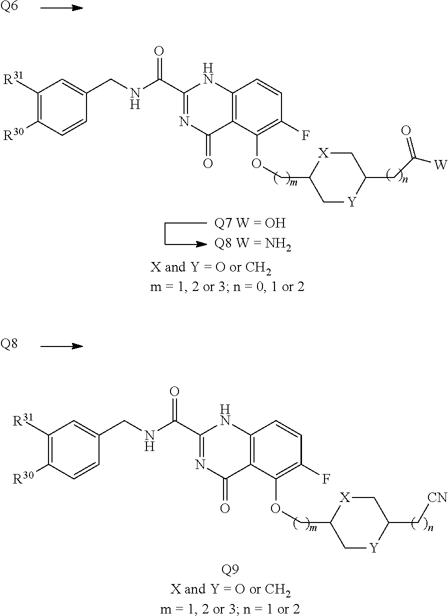

According to Scheme Q, 3,4-difluoroaniline is protected to give the tert-butyl carbamate Q1. The carbamate Q1 is treated with n-butyllithium at low temperature and followed by reaction with ethyl chloroformate to give the ethyl ester Q2. Deprotection of the amine functionality of carbamate Q2 with HCl in dioxane gives amine of formula Q3. The amine of formula Q3 is then reacted with ethyl cyanoformate at elevated temperatures to provide cyclized compound of formula Q4. Reaction of the ester Q4 with substituted benzylamines provides the amides of formula Q5. The C-5 fluorine of the formula of Q5 can be displaced with various alcohols in the presence of base to provide compounds of the invention. The fluoride of formula Q5 can be reacted with various alcohols (where the alcohol may be exemplified by but is not limited to 1,4-dioxane-2,5-diylmethanol or 1,4-cyclohexanedimethanol) in the presence of base to provide ethers Q6. Alcohols of the formula Q6 can be oxidized to the corresponding acids of the formula Q7 by treatment with pyridinium dichromate. Treatment of acids of the formula Q7 with oxalyl chloride followed by reaction with ammonia affords the primary amides of the formula Q8. Conversion of the amides of formula Q8 by treatment the phosphorous oxychloride provides the nitriles of the formula Q9 of the present invention.

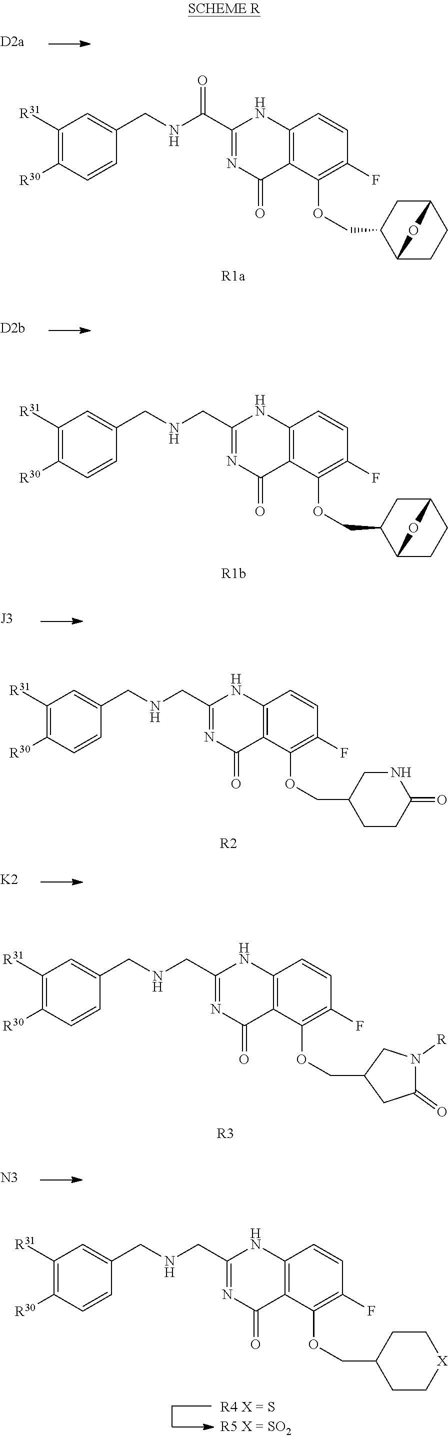

According to Scheme R, the C-5 fluorine of Q5 can be displaced with various alcohols in the presence of base to provide ether compounds of the invention. Reaction of compound of formula Q5 with alcohols D2a or D2b provides ethers R1a and R1b. Similarly, reaction of compound of formula Q5 with alcohol J3 provides ethers R2. Alternatively, compound of formula Q5 can be reacted with alcohols K2 to provide ethers R3 (where R may include but is not limited to hydrogen, methyl, ethyl, and iso-propyl). Likewise, reaction of alcohol N3 with compound of formula Q5 provides thioethers R4. Oxidation of R4 in the presence of oxone provides tetrahydrothiopyran-1,1-dione derivatives of the formula R5.

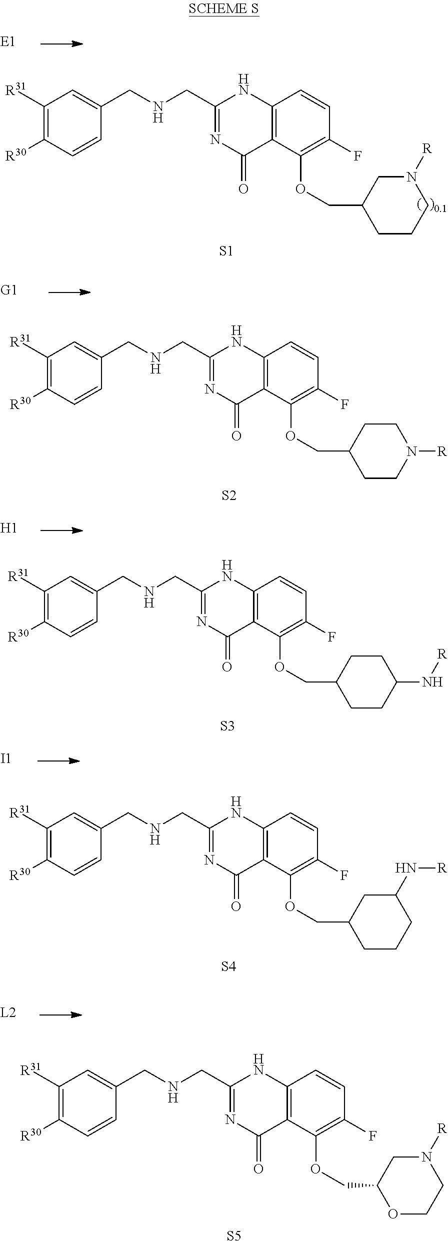

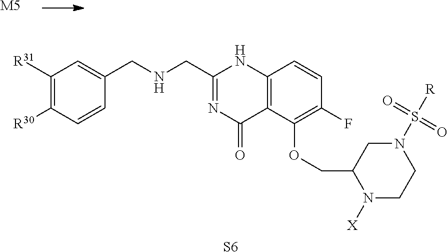

As shown in Scheme S, the C-5 fluorine of Q5 can be displaced with various alcohols containing protected amine functionality in the presence of base to provide ether compounds of the invention. After removal of the protecting group, the resulting amines may be reacted with alkyl halides, alkyl acid chlorides, and alkyl sulfonyl chlorides in the presence of an amine base to provide compounds of the present invention. In the case of the reaction of compounds of formula M5, the sulfonate esters may be converted to the alcohols prior to reaction with the compound of formula Q5 to provide the protected ethers. Hydrogenation in the presence of a catalyst such as palladium on carbon in an appropriate solvent (e.g. methanol, ethanol, or tetrahydrofuran) affords compounds of the formula S6 where X is hydrogen. Furthermore, compounds of the formula S6 where X is an alkyl substituent (e.g. methyl, ethyl, and propyl) may be prepared by subsequent reaction with an appropriate alkanal and a reducing agent (e.g. NaBH(OAc)₃).

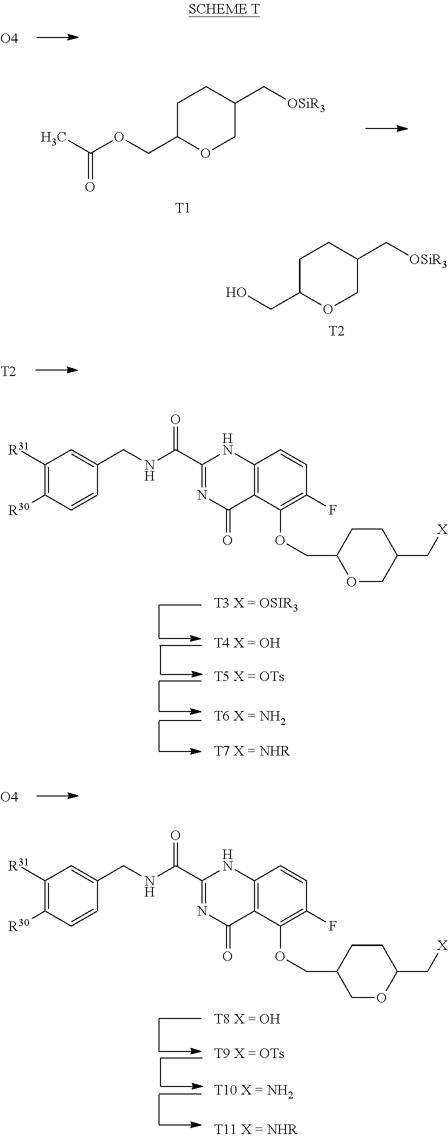

Following procedures analogous to those described in Scheme O, the tetrahydropyran-substituted examples of the invention may be prepared as described in Scheme T. The alcohol of O4 was protected as the corresponding silyl ether T1 by treatment with the requisite silyl chloride in the presence of an amine base. The acetate protecting group of T1 is then cleaved to provide alcohol of the formula T2. Alcohols T2 and O4 are reacted with the compound of formula Q5 in the presence of base to provide the ethers of formula T3 and T8 respectively. The silyl protecting group of compound of formula T3 is then removed to provide alcohols of formula T4. The alcohols of formulas T4 and T8 are then treated with toluenesulfonyl chloride in an amine base to provide tosylates of formulas T5 and T9 which may then be reacted with a various nucleophiles to provide compounds of the invention. Additionally, tosylates of formulas T5 and T9 may be reacted with ammonia in a solvent such as methanol to provide amines T6 and T10 which may be further reacted with alkyl halides, alkyl acid chlorides, and alkyl sulfonyl chlorides in the presence of a base to provide compounds of formulas T7 and T11 of the invention.

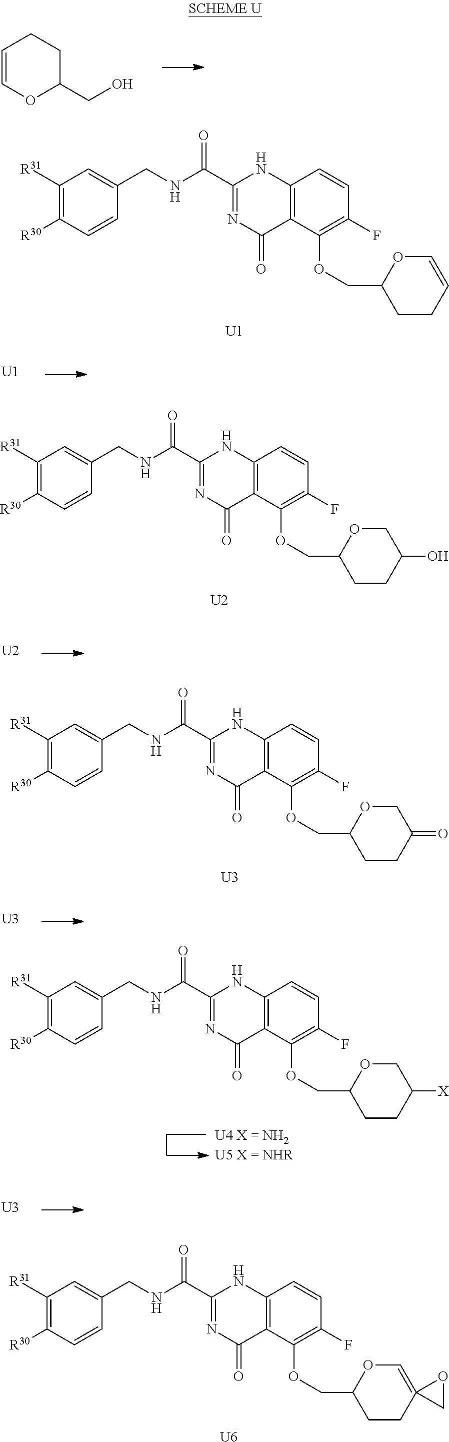

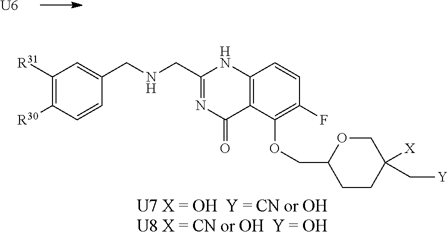

Following procedures analogous to those described in Scheme P, additional tetrahydropyran-substituted examples of the invention may be prepared as depicted in Scheme U. Reaction of 2-hydroxymethyl-3,4-dihydro-2H-pyran in the presence of base with the compounds of formula Q5 provides the alkenes of formula U1. Compounds of formulas U1 are oxidized to alcohols U2 by treatment with borane followed by sodium hydroxide and hydrogen peroxide. Alcohols of formula U2 are further oxidized to ketones of formula U3 using Swern conditions. Ketones of formula U3 may be converted to the primary amines of formula U4 by reductive alkylation using ammonium acetate and a borohydride reducing agent. Amines of the formula U4 may be further reacted with alkyl halides, alkyl acid chlorides, and alkyl sulfonyl chlorides in the presence of amine base to provide compounds of formula U5 of the invention. Alternatively, ketones of formula U3 may be converted to epoxides of formula U6 by treatment with trimethylsulfoxonium iodide and an alkoxide base. The epoxides of formula U6 may be reacted with various nucleophiles (where the nucleophile may be but is not limited to cyanide or hydroxide) to provide compounds of formulas U7 and U8 of the invention.

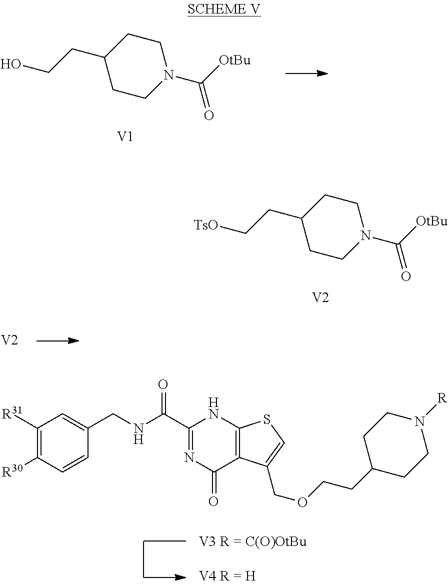

According to Scheme V, N-Boc 4-(2-hydroxyethyl)piperidine is reacted with toluenesulfonyl chloride in the presence of an amine base to provide tosylate V2. Alcohols of the formula A5 are then reacted with tosylate V2 in the presence of a base to provide the N-Boc-protected amines of the formula V3. The protecting group of the formula V3 is removed under acidic conditions and the resulting amines of formula V4 may be reacted with alkyl halides, alkyl acid chlorides, and alkyl sulfonyl chlorides in the presence of an amine base to provide compounds of the invention.

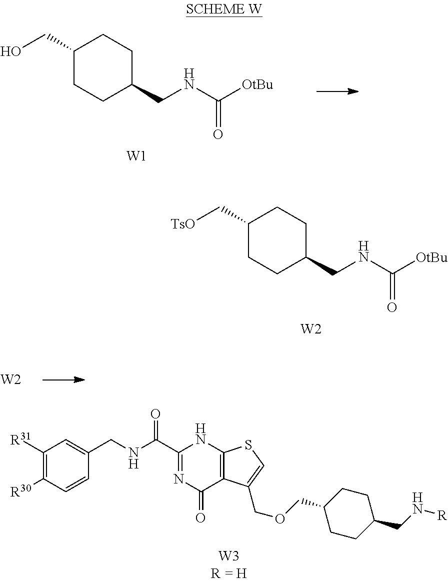

According to Scheme W, tert-butyl(trans 4-hydroxymethylcyclohexylmethyl)carbamate is reacted with toluenesulfonyl chloride in the presence of an amine base to provide tosylate W2. Alcohols of the formula A5 are then reacted with tosylate W2 in the presence of a base and deprotected to provide amines of the formula W3. The amines of formula W3 may be reacted with alkyl halides, alkyl acid chlorides, and alkyl sulfonyl chlorides in the presence of an amine base to provide compounds of the invention.

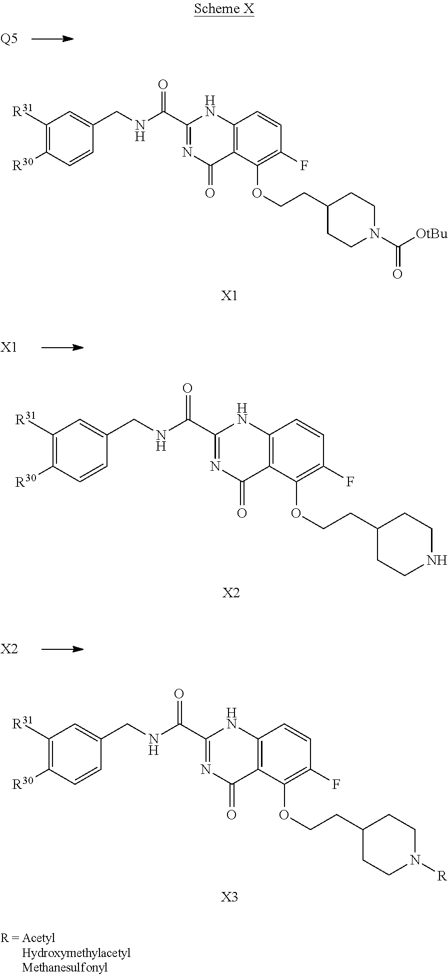

According to Scheme X, the fluoride of formula Q5 can be reacted with various alcohols (where the alcohol may be exemplified by but is not limited to N-boc-4-piperidineethanol) in the presence of base to provide ethers of formula XI. Protected amines of the formula XI can be deprotected to the corresponding amine of the formula X2 by treatment with trifluoroacetic acid. Treatment of the amines of the formula X2 with reactive chlorides (where the reactive chloride may be exemplified by but is not limited to acetyl chloride, acetoxyacetyl chloride, and methanesulfonyl chloride) in the presence of base and removal of protecting groups (where appropriate) by treatment with aqueous base (where the base may be exemplified by lithium hydroxide) provides the amides/sulfonamides of the formula X3 of the present invention.

### Detailed Preparative Methods

The detailed examples below illustrate preparation of compounds of this invention. Other compounds of this invention may be prepared using the methods illustrated in these examples, either alone or in combination with techniques generally known in the art. The following examples are merely illustrative, and not limiting to the remainder of this disclosure in any way.

### Example 1

### 5-({[trans-4-Aminocyclohexyl]methoxy}methyl)-N-(3-methoxybenzyl)-4-oxo-3,4-dihydrothieno[2,3-d]pyrimidine-2-carboxamide

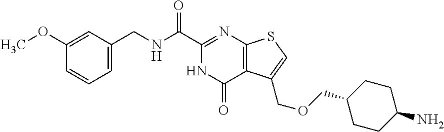

### Step 1: Preparation of ethyl 2-(3-carbamoyl-4-methylthiophen-2-ylamino)-2-oxoacetate

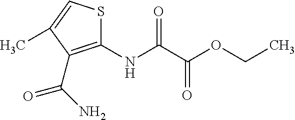

To an oven-dried 3N, 1-L round-bottom flask under an atmosphere of argon and equipped with a stir bar, addition funnel and thermometer was added 2-amino-4-methylthiophene-3-carboxamide (25 g). The flask was back-filled with argon and treated with anhydrous pyridine (160 mL). The resulting tan suspension was placed in an ice-water bath and treated dropwise via addition funnel with ethyl chlorooxoacetate (21.3 mL) as a solution in dry acetonitrile (53 mL) over 30 min. The reaction temperature was maintained at ˜10° C. during the course of the addition. Upon completion of addition, the addition funnel was rinsed with 25 mL of dry acetonitrile. The resulting thick mass was allowed to slowly warm to room temperature overnight. Water (250 mL) was then slowly added to the reaction mixture with vigorous stirring. The resulting free flowing solid was filtered and washed with water (4×100 mL). The solid was dried in a vacuum desiccator in vacuo to afford the title compound as a solid (29.8 g). ¹H NMR (400 MHz, DMSO-d₆) δ ppm 12.82 (1H, s), 6.81 (1H, s), 4.31 (2H, q, J=7.0 Hz), 2.38 (3H, s), 1.31 (3H, t, J=7.1 Hz).

### Step 2: Preparation of ethyl 5-methyl-4-oxo-3,4-dihydrothieno[2,3-d]jpyrimidine-2-carboxylate

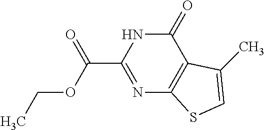

To a flask containing ethyl 2-(3-carbamoyl-4-methylthiophen-2-ylamino)-2-oxoacetate (29.8 g, 116.3 mmol) was added glacial acetic acid (300 mL) under argon. The reaction was heated to vigorous reflux for 20 hr. The reaction was cooled to room temperature and then placed in an ice bath for 15 min. The resulting solid was filtered, washed with hexanes (5×120 mL) followed by water (5×120 mL). The solid was dried in a vacuum oven at 60° C. for 18 h to afford the title compound as an off-white solid (16.6 g). ¹H NMR (400 MHz, DMSO-d₆) δ ppm 12.68 (1H, br. s.), 7.35 (1H, d, J=1.3 Hz), 4.33 (2H, q, J=7.2 Hz), 2.47 (3H, d, J=1.1 Hz), 1.31 (3H, t, J=7.1 Hz).

### Step 3: Preparation of ethyl 5-(bromomethyl)-4-oxo-3,4-dihydrothieno[2,3-d]pyrimidine-2-carboxylate

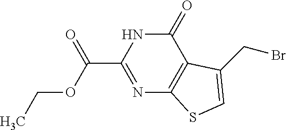

To a 1-L round-bottom flask equipped with a nitrogen inlet and a reflux condenser was charged ethyl 5-methyl-4-oxo-3,4-dihydrothieno[2,3-d]jpyrimidine-2-carboxylate (12.9 g, 54.4 mmol), N-bromosuccinimide (10.2 g, 57.1 mmol, freshly re-crystallized from water) and 2,2′-azobis(isobutyronitrile) (0.9 g, 5.4 mmol). The flask was back-filled with nitrogen and charged with carbon tetrachloride (410 mL). The reaction was placed in a pre-heated oil bath (80° C.) and vigorously stirred. After ˜2 hr at reflux, the reaction mixture was cooled to room temperature and concentrated under reduced pressure. The residue was partitioned between EtOAc (300 mL) and water (200 mL). Organic layer was washed with two additional portions of water (200 mL each). The combined aqueous phases were back-extracted with EtOAc (100 mL). The combined organic layers were washed with brine (100 mL), dried over magnesium sulfate, filtered and concentrated under reduced pressure to give a solid. The solid was dissolved in a hot EtOAc (200 mL) and allowed to slowly cool to room temperature. The resulting solid was filtered and dried to afford the crude title compound as a tan solid (9.8 g) which was used without further purification in the next step. ¹H NMR (400 MHz, DMSO-d₆) δ ppm 12.95 (1H, br. s.), 7.90 (1H, s), 4.97 (2H, s), 4.26-4.48 (2H, m), 1.24-1.46 (3H, m).

### Step 4: Preparation of ethyl 5-(hydroxymethyl)-4-oxo-3,4-dihydrothieno[2,3-d]jpyrimidine-2-carboxylate

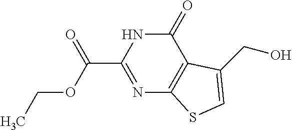

To a stirred solution of crude ethyl 5-(bromomethyl)-4-oxo-3,4-dihydrothieno[2,3-d]pyrimidine-2-carboxylate (9.8 g) in acetonitrile (500 mL) was added 10% aqueous potassium carbonate (100 mL). The reaction mixture was stirred for 18 hr at room temperature. The reaction was filtered and the precipitant was washed with acetonitrile (200 mL). The filtrates were combined, acidified with trifluoroacetic acid to pH ˜3 and concentrated to remove the volatile organic solvent. The aqueous mixture was extracted with EtOAc (3×50 mL). The combined EtOAc layers were dried over magnesium sulfate, filtered, and evaporated to afford a crude solid. The crude solid was purified using normal phase with 0-50% EtOAc in 1:1 hexane:CH₂Cl₂ to afford the title compound as a solid (1.6 g). ¹H NMR (400 MHz, DMSO-d₆) δ ppm 12.87 (1H, br. s.), 7.55 (1H, s), 5.12-5.56 (1H, m), 4.79 (2H, s), 4.36 (2H, q, J=7.0 Hz), 1.34 (3H, t, J=7.1 Hz).

### Step 5: Preparation of 5-(hydroxymethyl)-N-(3-methoxybenzyl)-4-oxo-3,4-dihydrothieno[2,3-d]pyrimidine-2-carboxamide

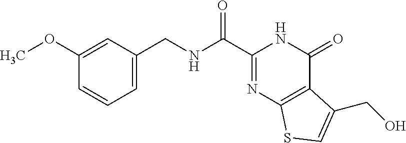

To a flask containing ethyl 5-(hydroxymethyl)-4-oxo-3,4-dihydrothieno[2,3-d]pyrimidine-2-carboxylate (1.6 g) was charged absolute ethanol (25 mL) and 3-methoxybenzylamine (2.4 mL). The reaction mixture was heated to reflux under nitrogen for 2 hours. The resulting orange mixture was cooled to room temperature and concentrated under reduced pressure. The residue was dissolved in EtOAc (50 mL) and washed sequentially with 1 N aqueous HCl (2×25 mL), water (25 mL) and brine (25 mL). The organic phase was dried over magnesium sulfate, filtered and concentrated under reduced pressure to give a tan solid. The solid was triturated with a small volume of CH₂Cl₂, filtered, and dried to provide the title compound as an off-white solid (1.41 g). ¹H NMR (400 MHz, DMSO-d₆) δ ppm 12.45 (1H, br. s.), 9.64 (1H, t, J=6.3 Hz), 7.48 (1H, s), 7.24 (1H, t, J=8.1 Hz), 6.86-6.94 (2H, m), 6.78-6.85 (1H, m), 5.31 (1H, t, J=5.6 Hz), 4.79 (2H, d, J=4.6 Hz), 4.42 (2H, d, J=6.4 Hz), 3.73 (3H, s).

### Step 6: Preparation of trans-4-[(tert-butoxycarbonyl)amino]cyclohexylmethyl 4-methylbenzenesulfonate

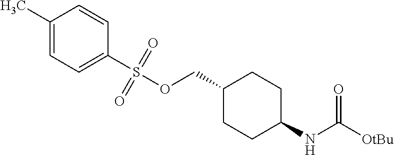

To a solution of tert-butyl trans-(4-hydroxymethyl)cyclohexylcarbamate (0.92 g) in 5 mL of pyridine at 0° C. was slowly added tosyl chloride (0.84 g). The reaction mixture was slowly warmed to room temperature and a cloudy suspension resulted. After 5 hours, the reaction mixture was cooled to 0° C. and treated slowly with 20 mL of water. The resulting white precipitant was filtered, washed with cold water (2×5 mL), air-dried, and finally dried in vacuo to provide the title compound as a white solid (1.43 g). LC/MS (5-100% CH₃CN:H₂O gradient over 4 min) 3.7 min, m/z 406 (M+Na). ¹H NMR (400 MHz, DMSO-d₆) δ ppm 7.77 (2H, d, J=8.4 Hz), 7.48 (2H, d, J=8.5 Hz), 6.69 (1H, d, J=7.7 Hz), 3.82 (2H, d, J=6.3 Hz), 2.95-3.21 (1H, m), 2.42 (3H, s), 1.62-1.84 (2H, m), 1.54-1.67 (2H, m), 1.42-1.56 (1H, m), 1.35 (9H, s), 0.96-1.14 (2H, m), 0.77-0.97 (2H, m).

### Step 7: Preparation of 5-({[trans-4-aminocyclohexyl]methoxy}methyl)-N-(3-methoxybenzyl)-4-oxo-3,4-dihydrothieno[2,3-d]pyrimidine-2-carboxamide

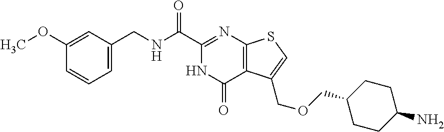

To an oven-dried 3-neck, round-bottom flask equipped with a stir bar, nitrogen inlet on a reflux condenser, glass and rubber stoppers was charged 0.20 g of 60% by wt dispersion of sodium hydride in oil. The flask was back-filled with nitrogen and treated with dry THF (5 mL). The resulting grey suspension was cooled to 0° C. and was slowly treated with 5-(hydroxymethyl)-N-(3-methoxybenzyl)-4-oxo-3,4-dihydrothieno[2,3-d]pyrimidine-2-carboxamide (0.45 g) as a tan suspension in 8 mL of dry THF in a dropwise fashion. After 10 min, the reaction was warmed to room temperature; after 15 min, the resulting suspension was cooled to 0° C. and treated dropwise with trans-4-[(tert-butoxycarbonyl)amino]cyclohexylmethyl 4-methylbenzenesulfonate (0.65 g) as a solution in 5 mL of dry THF. After 5 min, the reaction mixture was warmed to room temperature. After ˜4 hours, the reaction mixture was treated with 5 mL of dry N,N-dimethylformamide. After ˜2 hours, the reaction mixture was heated to 60° C. overnight. The resulting orange suspension was cooled to room temperature and quenched with 5 mL of a 1 N aqueous hydrogen chloride solution diluted with 5 mL of brine. The separated aqueous phase (pH ˜7) was extracted with THF (2×25 mL) and once with 1:1 THF-EtOAc mixture (25 mL). The organic phases were combined and washed with brine (2×20 mL), dried over sodium sulfate, filtered and concentrated under reduced pressure to give an orange residue. The residue was adsorbed onto silica gel and purified by silica gel flash column chromatography eluting with 5-25% MeOH (saturated with ammonia) in CH₂Cl₂. The product containing fractions were combined to give the title compound as a tan solid (0.19 g). LC/MS (5-100% CH₃CN:H₂O gradient over 4 min) 2.7 min, m/z 457 (M+H). ¹H NMR (400 MHz, DMSO-d₆) δ ppm 9.07 (1H, t, J=6.3 Hz), 7.22 (1H, t, J=8.0 Hz), 7.00 (1H, s), 6.84-6.91 (2H, m), 6.75-6.84 (1H, m), 4.78 (2H, s), 4.39 (2H, d, J=6.3 Hz), 3.72 (3H, s), 3.34 (2H, d, J=6.3 Hz), 2.73-2.84 (1H, m), 1.73-1.92 (4H, m), 1.43-1.60 (1H, m), 0.90-1.29 (4H, m).

### Example 2

### 5-({[trans-4-Aminocyclohexyl]methoxy}methyl)-N-(4-fluoro-3-methoxybenzyl)-4-oxo-3,4-dihydrothieno[2,3-d]pyrimidine-2-carboxamide

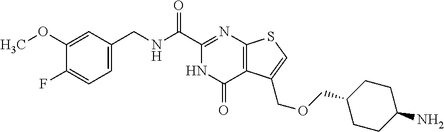

### Step 1: Preparation of N-(4-fluoro-3-methoxybenzyl)-5-(hydroxymethyl)-4-oxo-3,4-dihydrothieno[2,3-d]pyrimidine-2-carboxamide

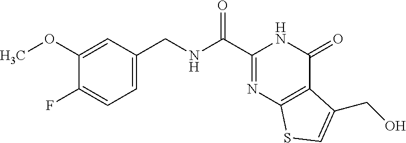

To a flask containing ethyl 5-(hydroxymethyl)-4-oxo-3,4-dihydrothieno[2,3-d]pyrimidine-2-carboxylate (prepared as in step 4 of the synthesis of 5-({[trans-4-aminocyclohexyl]methoxy}methyl)-N-(3-methoxybenzyl)-4-oxo-3,4-dihydrothieno[2,3-d]pyrimidine-2-carboxamide, Example 1) (1.5 g) was charged abs. ethanol (25 mL), N,N-diisopropylethylamine (2.8 mL) and 4-fluoro-3-methoxyamine HCl salt (1.95 g). The reaction mixture was heated to reflux under nitrogen overnight. The resulting orange suspension was cooled to room temperature and concentrated under reduced pressure. The residue was partitioned between THF-EtOAc (1:1, ˜100 mL) and 1 N aqueous HCl. The layers were separated and the organic phase washed with brine (2×), dried over sodium sulfate, filtered and concentrated under reduced pressure to give a tan solid. The solid was dissolved in hot EtOH/EtOAc (˜200 mL), hot filtered and left to cool to room temperature. The mixture was concentrated and the residue triturated with ˜20 mL hot EtOAc, filtered, washed with hexanes, and dried in vacuo to provide a tan solid. The solid was combined with the trituration filtrate and purified by silica gel flash column chromatography eluting with 4-8% MeOH in DCM to give the title compound as an off-white solid (0.95 g). LC/MS (5-100% CH₃CN:H₂O gradient over 4 min) 2.8 min, m/z 364 (M+H). ¹H NMR (400 MHz, DMSO-d₆) δ ppm 12.41 (1H, br. s.), 9.65 (1H, t, J=6.3 Hz), 7.48 (1H, s), 7.03-7.25 (2H, m), 6.82-6.94 (1H, m), 5.32 (1H, t, J=5.7 Hz), 4.78 (2H, d, J=4.8 Hz), 4.41 (2H, d, J=6.3 Hz), 3.81 (3H, s).

### Step 2: Preparation of 5-({[trans-4-aminocyclohexyl]methoxy}methyl)-N-(4-fluoro-3-methoxybenzyl)-4-oxo-3,4-dihydrothieno[2,3-d]pyrimidine-2-carboxamide

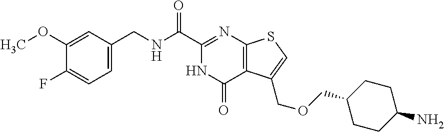

To a dry 3-neck, round-bottom flask equipped with a stir bar, nitrogen inlet on a reflux condenser, glass and rubber stoppers was charged 0.20 g of 60% by wt dispersion of sodium hydride in oil. The flask was back-filled with nitrogen and treated with dry THF (3 mL). The resulting grey suspension was cooled to 0° C. and was slowly treated with N-(4-fluoro-3-methoxybenzyl)-5-(hydroxymethyl)-4-oxo-3,4-dihydrothieno[2,3-d]pyrimidine-2-carboxamide (0.48 g) as a tan solution in 6 mL of dry N,N-dimethylformaide in a dropwise fashion. After 10 min, the reaction was warmed to room temperature; after 45 min, the resulting suspension was re-cooled to 0° C. and treated dropwise with trans-4-[(tert-butoxycarbonyl)amino]cyclohexylmethyl 4-methylbenzenesulfonate (prepared as described in Step 6 of the synthesis of 5-({[trans-4-aminocyclohexyl]methoxy}methyl)-N-(3-methoxybenzyl)-4-oxo-3,4-dihydrothieno[2,3-d]pyrimidine-2-carboxamide, Example 1) (0.65 g) as a solution in 6 mL of dry THF. After 10 min, the reaction mixture was warmed to room temperature. After ˜0.5 hours, the reaction mixture was heated to 60° C. for 3 hours. The resulting orange suspension was cooled to room temperature and quenched with 6 mL of a 1 N aqueous hydrogen chloride solution diluted with 10 mL of brine. The separated aqueous phase (pH ˜7) was extracted with THF (2×25 mL) and with 1:1 THF-EtOAc mixture (2×20 mL). The organic phases were combined and washed with brine (2×20 mL), dried over sodium sulfate, filtered and concentrated under reduced pressure to give an orange residue. The residue was adsorbed onto silica gel and purified by silica gel flash column chromatography eluting with 20-30% MeOH (2 M ammonia) in CH₂Cl₂. The product containing fractions were combined to give the title compound as a tan solid (0.10 g). LC/MS (5-100% CH₃CN:H₂O gradient over 4 min) 2.8 min, m/z 475 (M+H). ¹H NMR (400 MHz, DMSO-d₆) δ ppm 9.12-9.19 (1H, m), 7.02-7.19 (3H, m), 6.83-6.90 (1H, m), 4.78 (2H, s), 4.39 (2H, d, J=6.0 Hz), 3.81 (3H, s), 3.28-3.38 (2H, m), 2.77-2.90 (1H, m), 1.71-1.95 (4H, m), 1.46-1.60 (1H, m), 0.83-1.29 (4H, m).

### Example 3

### 6-Fluoro-5-{[(trans-2,5)-5-(hydroxymethyl)-1,4-dioxan-2-yl]methoxy}-N-(3-methoxybenzyl)-4-oxo-3,4-dihydroquinazoline-2-carboxamide

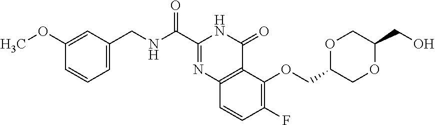

### Step 1: Preparation of N-(tert-butoxycarbonyl)-3,4-difluoroaniline

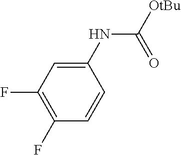

To a flask equipped with a reflux condenser was charged 3,4-difluoroaniline (13.2 g) and dry THF (100 mL). The resulting solution was treated with di-tert-butyl dicarbonate (24.5 g) and heated to 60° C. for 16 hours. The solution was cooled to room temperature and concentrated under reduced pressure. The resulting residue was dissolved in EtOAc (150 mL) and washed successively with 1% aqueous HCl solution (2×100 mL) and 5% aqueous sodium bicarbonate solution (2×100 mL). The organic layer was dried over magnesium sulfate, filtered and concentrated under reduced pressure to afford a tan residue. The residue was crystallized from hexanes to provide the title compound as a white solid (19.6 g). ¹H NMR (300 MHz, CDCl₃) δ ppm 7.38-7.48 (1H, m), 6.99-7.10 (1H, m), 6.87-6.94 (1H, m), 6.58 (1H, br. s.), 1.51 (9H, s).

### Step 2: Preparation of ethyl 6-N-(tert-butoxycarbonylamino)-2,3-difluorobenzoate

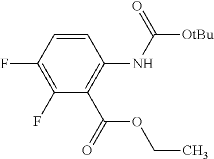

To a dry flask equipped with an addition funnel and under a nitrogen atmosphere was charged N-(tert-butoxy carbonyl)-3,4-difluoroaniline (19.6 g) and dry THF (200 mL). The resulting solution was cooled in dry ice/acetone bath to −78° C. and treated dropwise with a n-butyllithium solution (139 mL, 1.6 M in hexanes) over 1 hr with constant stirring. The reaction mixture was then treated dropwise with ethyl chloroformate (9.0 mL) as solution in dry THF (60 mL) via addition funnel over 45 minutes. After stirring for an additional hour, the reaction mixture was warmed to 0° C. and treated dropwise with saturated aqueous ammonium chloride (200 mL) over 15 minutes. The reaction mixture was warmed to room temperature and diluted with EtOAc (500 mL) and water (500 mL). The layers were separated and the aqueous phase was extracted with EtOAc (250 mL). The organic layers were combined and washed with brine (2×250 mL), dried over sodium sulfate, filtered and concentrated under reduced pressure to afford a gold oil. The oil was placed in a kugelrohr for 1 hour at 40° C. (7 mm Hg vacuum) then cooled to room temperature to provide the title compound as a gold solid (28.5 g) which was used directly in the next step without further purification.

### Step 3: Preparation of ethyl 5,6-difluoroanthranilate hydrochloride

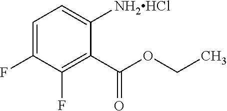

To a flask equipped with an addition funnel was charged with ethyl 6-N-(tert-butoxycarbonylamino)-2,3-difluorobenzoate (28.5 g) and EtOAc (50 mL). The resulting solution was cooled to 0° C. and treated with 4 N HCl in dioxane (125 mL) via addition funnel over 10 minutes. The reaction mixture was warmed to room temperature. After 1 hour at room temperature, the solution was treated with ethyl ether (80 mL) and the resulting suspension filtered. The collected solid was triturated with 1:1 ethyl ether:ethanol (100 mL), ethyl ether (2×100 mL) and finally dried in a vacuum oven (30° C.) to give the title compound as a light yellow solid (12.6 g). ¹H NMR (300 MHz, DMSO-d₆) δ ppm 8.7 (3H, br. s.), 7.36-7.47 (1H, m), 6.76-6.83 (1H, m), 4.33 (2H, q, J=7.1 Hz), 1.31 (3H, t, J=7.3 Hz).

### Step 4: Preparation of ethyl 5,6-difluoro-4-oxo-3,4-dihydroquinazoline-2-carboxylate

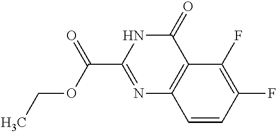

To a flask equipped with a reflux condenser under a nitrogen atmosphere was charged ethyl 5,6-difluoroanthranilate hydrochloride (12.6 g) and 1 N HCl in acetic acid (250 mL). The resulting mixture was treated with ethyl cyanoformate (5.8 mL) and heated to 80° C. After 3 hours, the reaction mixture was cooled to room temperature and concentrated under reduced pressure to give a yellow pasty solid. The solid was treated with absolute ethanol (50 mL) and the resulting suspension filtered. The collected solid was washed with a small volume of ethanol and finally dried in vacuo to give the title compound as an off-white solid (10.1 g). ¹H NMR (300 MHz, DMSO-d₆) δ ppm 12.8 (1H, br. s.), 7.91-8.03 (1H, m), 7.66-7.73 (1H, m), 4.39 (2H, q, J=7.2 Hz), 1.36 (3H, t, J=7.1 Hz).

### Step 5: Preparation of N-(3-methoxybenzyl)-5,6-difluoro-4-oxo-3,4-dihydroquinazoline-2-carboxamide

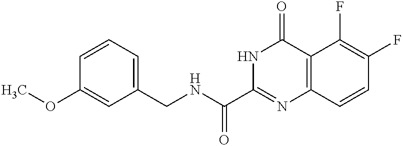

To a flask under a nitrogen atmosphere was charged ethyl 5,6-difluoro-4-oxo-3,4-dihydroquinazoline-2-carboxylate (10.1 g) and THF (180 mL). The resulting mixture was treated with triethylamine (6.7 mL) and 3-methoxybenzylamine (7.7 g). After stirring 2 days at room temperature, the mixture was concentrated under reduced pressure to give a pasty solid. The solid was treated with EtOAc (750 mL) and 10% aqueous potassium carbonate (150 mL), and the resulting suspension filtered. The collected solid was triturated with ethyl ether (200 mL), 1:1 ethyl ether:ethanol (200 mL) and finally dried in a vac oven to give an off-white solid (12.5 g). A sample of this solid (11 g) was treated with THF (155 mL) and 10% aqueous potassium carbonate (220 mL). The mixture was diluted with EtOAc (1650 mL), THF (1100 mL) and 10% aqueous potassium carbonate (550 mL). The resulting mixture was separated and the organic phase was washed with water (2×550 mL), brine (550 mL). The organic layer was then dried over sodium sulfate, filtered, concentrated under reduced pressure and dried in a vac oven (50° C.) to give a pale yellow solid. This solid was triturated with ethyl ether (50 mL), filtered and finally dried in a vac oven (50° C.) to give the title compound as a pale yellow solid (4.71 g). ¹H NMR (300 MHz, DMSO-d₆) δ ppm 12.4 (1H, br. s.), 9.51 (1H, t, J=6.4 Hz), 7.85-7.98 (1H, m), 7.55-7.63 (1H, m), 7.17-7.26 (1H, m), 6.86-6.94 (2H, m), 6.76-6.83 (1H, m), 4.44 (2H, d, J=6.3 Hz), 3.72 (3H, s).

### Step 6: Preparation of 6-fluoro-5-{[(trans-2,5)-5-(hydroxymethyl)-1,4-dioxan-2-yl]methoxy}-N-(3-methoxybenzyl)-4-oxo-3,4-dihydroquinazoline-2-carboxamide

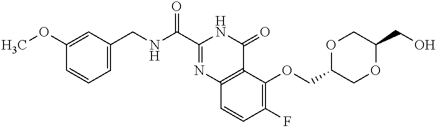

To a dry flask under an atmosphere of argon was added sodium hydride (120 mg, 60% dispersion in oil) and the flask was flushed with argon. The flask was charged with 2 mL of N,N-dimethylacetamide and flushed with argon. After stirring for 5 min at room temperature, a solution of the trans-1,4-dioxane-2,5-diyldimethanol (J. Am. Chem. Soc. 1954, 76 (24), 6401) (222 mg, 1.5 mmol) in 1 mL of N,N-dimethylacetamide was added via syringe. The mixture was stirred for 5 min at room temperature and a solution of N-(3-methoxybenzyl)-5,6-difluoro-4-oxo-3,4-dihydroquinazoline-2-carboxamide (173 mg, 0.5 mmol) in 1 mL of N,N-dimethylacetamide was added via syringe and the reaction was heated to 80° C. After 30 min, the reaction was cooled to room temperature, the pH was lowered to ˜4 with 1M hydrochloric acid, and then diluted with EtOAc (50 mL). The organic layer was washed with water (20 mL), brine (20 mL), dried over magnesium sulfate, filtered, and evaporated to afford an off-white solid. The solid was purified using reverse phase chromatography to afford the title compound as a white solid (122 mg). LC/MS 5-100% acetonitrile/TFA-water/TFA (4 min gradient) 2.68 min, m/z 474 (M+H). ¹H NMR (400 MHz, DMSO-d₆) δ ppm 12.14 (1H, br. s.), 9.49 (1H, t, J=6.4 Hz), 7.81 (1H, dd, J=10.5, 9.1 Hz), 7.54 (1H, dd, J=9.1, 4.6 Hz), 7.24 (1H, t, J=8.2 Hz), 6.86-6.95 (2H, m), 6.77-6.86 (1H, m), 4.44 (2H, d, J=6.4 Hz), 3.93-4.13 (3H, m), 3.76-3.88 (2H, m), 3.73 (3H, s), 3.36-3.57 (3H, m), 3.27-3.36 (2H, m).

### Example 4

### N-(3-methoxybenzyl)-5-(((trans-4-(hydroxymethyl)cyclohexyl)methoxy)methyl)-4-oxo-3,4-dihydrothieno[2,3-d]pyrimidine-2-carboxamide

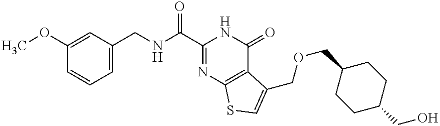

n-Butyl lithium in hexanes (2.5 N, 1.80 mL) was added to dry trans-1,4-cyclohexanedimethanol (770 mg) in anhydrous THF (25 mL) under nitrogen. After a few minutes a solution of ethyl 5-(bromomethyl)-4-oxo-3,4-dihydrothieno[2,3-d]pyrimidine-2-carboxylate (prepared as described in Step 3 of the synthesis of 5-({[trans-4-aminocyclohexyl]methoxy}methyl)-N-(3-methoxybenzyl)-4-oxo-3,4-dihydrothieno[2,3-d]pyrimidine-2-carboxamide) (492 mg, vacuum dried) in anhydrous THF (15 mL) was added. After stirring at room temperature for 4 hours, 3-methoxybenzylamine (950 mg) was added. After stirring under nitrogen overnight the mixture was concentrated, acidified with trifluoroacetic acid, and purified by reverse phase (C-18) chromatography to give the title compound as a white solid (120 mg, 16%). LC/MS: 5%-95% CH₃CN:H₂O gradient over 6 min: 4.29 min, m/z 472 (M+H). ¹H NMR (400 MHz, DMSO-d₆) δ ppm 12.31 (1H, br. s.), 9.57 (1H, br. s.), 7.50 (1H, s), 7.23 (1H, t, J=8.1 Hz), 6.87-6.93 (2H, m), 6.82 (1H, br. s.), 4.76 (2H, s), 4.42 (2H, d, J=5.9 Hz), 4.28 (1H, br. s.), 3.73 (3H, s), 3.36 (2H, d, J=6.2 Hz), 3.20 (2H, d, J=5.9 Hz), 1.69-1.84 (4H, m), 1.54 (1H, br. s.), 1.29 (1H, br. s.), 0.80-1.01 (4H, m).

### Example 5

### Trans-4-(((2-((3-Methoxybenzyl)carbamoyl)-4-oxo-3,4-dihydrothieno[2,3-d]pyrimidin-5-yl)methoxy)methyl)cyclohexanecarboxylic acid

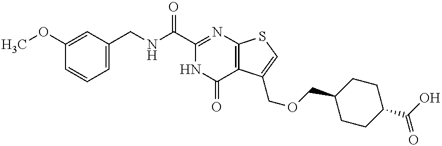

N-(3-Methoxybenzyl)-5-(((trans-4-(hydroxymethyl)cyclohexyl)methoxy)methyl)-4-oxo-3,4-dihydrothieno[2,3-d]pyrimidine-2-carboxamide (prepared as described in Example 4) (55 mg) was dissolved in anhydrous dimethylformamide (2.0 mL) and pyridinium dichromate (526 mg) was added. After five hours the mixture was diluted with water (10 mL) and extracted with methylene chloride (2×15 mL). The extract was washed with water (10 mL), dried over magnesium sulfate, filtered and concentrated to give a dark residue. Purification by reverse phase (C-18) chromatography gave the title compound as a tan solid (49 mg, 87%). LC-MS 5%-95% CH₃CN:H₂O gradient over 6 min: 4.42 min, m/z 486 (M+H). ¹H NMR (400 MHz, DMSO-d₆) δ ppm 12.29 (1H, s), 11.91 (1H, s), 9.57 (1H, t, J=5.9 Hz), 7.51 (1H, s), 7.24 (1H, t, J=7.9 Hz), 6.89-6.93 (2H, m), 6.82 (1H, d, J=7.3 Hz), 4.77 (2H, s), 4.42 (2H, d, J=6.2 Hz), 3.74 (3H, s), 3.36 (2H, d, J=6.2 Hz), 2.13 (1H, t, J=12.4 Hz), 1.90 (2H, d, J=14.3 Hz), 1.81 (2H, d, J=13.5 Hz), 1.57 (1H, br. s.), 1.23-1.37 (2H, m), 1.00 (2H, q, J=12.2 Hz).

### Example 6

### N-(3-Methoxybenzyl)-5-(((trans-4-cyanocyclohexyl)methoxy)methyl)-4-oxo-3,4-dihydrothieno[2,3-d]pyrimidine-2-carboxamide

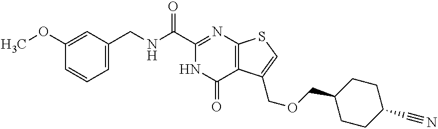

trans-4-(((2-((3-Methoxybenzyl)carbamoyl)-4-oxo-3,4-dihydrothieno[2,3-d]pyrimidin-5-yl)methoxy)methyl)cyclohexanecarboxylic acid (prepared as described in Example 5) (30 mg) was dissolved in anhydrous THF (2.0 mL) under nitrogen and oxalyl chloride (0.10 mL) was added, followed by dimethylformamide (one drop). After one half hour the mixture was concentrated to dryness. The residue was stirred in anhydrous THF (1.0 mL) and 7M ammonia in MeOH (0.3 mL) was added. After ½ hour the mixture was diluted with water (5 mL), concentrated to remove THF, and the liquid was decanted from the solid. The solid was washed once with water and dried. The residue was dissolved in anhydrous dimethylformamide (1.0 mL) and phosphorus oxychloride (0.050 mL) was added under nitrogen. After one minute the mixture was poured into sodium bicarbonate (0.25 g) in ice water (25 mL). The mixture was diluted with water (10 mL) and extracted with methylene chloride (2×15 mL). The extract was washed with water (10 mL), dried over magnesium sulfate, filtered and concentrated. Purification by reverse phase (C-18) chromatography gave the title compound as a white solid (7.4 mg, 26%). LC-MS 5%-95% CH₃CN:H₂O gradient over 6 min: 4.80 min, m/z 467 (M+H). ¹H NMR (400 MHz, DMSO-d₆) δ ppm 12.29 (1H, br. s.), 9.56 (1H, t, J=6.2 Hz), 7.50 (1H, s), 7.24 (1H, t, J=8.1 Hz), 6.87-6.95 (2H, m), 6.82 (1H, d, J=7.7 Hz), 4.76 (2H, s), 4.42 (2H, d, J=6.2 Hz), 3.74 (3H, s), 3.35 (2H, d, J=6.2 Hz), 2.61 (1H, t, J=11.9 Hz), 2.01 (2H, dd, J=13.0, 2.4 Hz), 1.78 (2H, dd, J=13.2, 2.2 Hz), 1.63 (1H, br. s.), 1.42-1.55 (2H, m), 0.94-1.09 (2H, m).

### Example 7

### N-(3-Methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide

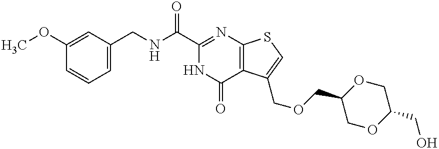

2.5 N n-Butyl lithium in hexanes (1.90 mL) was added to dry trans-1,4-dioxane-2,5-dimethanol (704 mg) in anhydrous THF (50 mL). A solution of ethyl 5-(bromomethyl)-4-oxo-3,4-dihydrothieno[2,3-d]pyrimidine-2-carboxylate (prepared as described in Step 3 of the synthesis of 5-({[trans-4-aminocyclohexyl]methoxy}methyl)-N-(3-methoxybenzyl)-4-oxo-3,4-dihydrothieno[2,3-d]pyrimidine-2-carboxamide) (488 mg, vacuum dried) in anhydrous THF (15 mL) was added. After stirring three days at room temperature 3-methoxybenzylamine (1.20 g) was added. After stirring overnight the mixture was diluted with water and acetonitrile then acidified with trifluoroacetic acid. Purification by reverse phase (C-18) followed by silica gel flash chromatography (3 g silica, eluted with acetonitrile, then THF) gave the title compound as a white solid (55 mg, 7%). LC-MS 5%-95% CH₃CN:H₂O gradient over 6 min: 3.64 min, m/z 476 (M+H). ¹H NMR (400 MHz, DMSO-d₆) δ ppm 3.30-3.68 (m, 8H) 3.74 (s, 3H) 3.76-3.85 (m, 2H) 4.42 (d, J=6.22 Hz, 2H) 4.64 (br. s., 1H) 4.80 (s, 2H) 6.82 (d, J=7.69 Hz, 1H) 6.88-6.97 (m, 2H) 7.24 (t, J=8.05 Hz, 1H).

### Example 8

### Trans-5-(((2-((3-Methoxybenzyl)carbamoyl)-4-oxo-3,4-dihydrothieno[2,3-d]pyrimidin-5-yl)methoxy)methyl)-1,4-dioxane-2-carboxylic acid

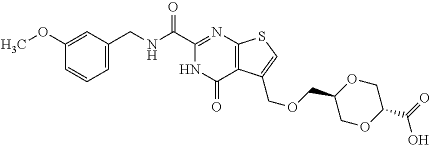

N-(3-Methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide (prepared as described in Example 7) (29 mg) was dissolved in anhydrous dimethylformamide (1.0 mL) and pyridinium dichromate (297 mg) was added. The mixture was stirred under nitrogen for 3 hours at 37 degrees. The mixture was diluted with water (10 mL) and extracted with methylene chloride (2×15 mL). The extract was washed with water (10 mL), dried over magnesium sulfate, filtered and concentrated to give a dark residue. Purification by reverse phase (C-18) chromatography gave the title compound as a white solid (12.5 mg, 41%). LC-MS 5%-95% CH₃CN:H₂O gradient over 6 min: 3.72 min, m/z 490 (M+H). ¹H NMR (400 MHz, DMSO-d₆) δ ppm 9.57 (1H, br. s.), 7.55 (1H, s), 7.23 (1H, t, J=8.1 Hz), 6.91 (2H, br. s.), 6.79-6.85 (1H, m), 4.81 (2H, s), 4.42 (2H, d, J=5.9 Hz), 4.09 (1H, d, J=8.1

Hz), 3.97 (1H, d, J=11.3 Hz), 3.89 (1H, d, J=11.3 Hz), 3.73 (3H, s), 3.69 (1H, br. s.), 3.69 (1H, s), 3.38-3.65 (4H, m).

### Example 9

### N-(4-Fluoro-3-methoxybenzyl)-5-(((trans-4-(hydroxymethyl)cyclohexyl)methoxy)methyl)-4-oxo-3,4-dihydrothieno[2,3-d]pyrimidine-2-carboxamide

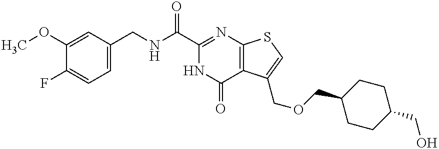

2.5 N n-Butyl lithium in hexanes (10.0 mL) was added to dry trans-1,4-cyclohexanedimethanol (3.94 g) in anhydrous THF (100 mL) under nitrogen. After 10 minutes a solution of ethyl 5-(bromomethyl)-4-oxo-3,4-dihydrothieno[2,3-d]pyrimidine-2-carboxylate (prepared as described in Step 3 of the synthesis of ethyl 5-(hydroxymethyl)-4-oxo-3,4-dihydrothieno[2,3-d]jpyrimidine-2-carboxylate) (2.53 g, vacuum dried) in anhydrous THF (100 mL) was added. After stirring four hours at room temperature 3-methoxy-4-fluorobenzylamine (5.58 g) was added. After stirring overnight water (2 mL) was added to give a clear yellow solution. The mixture was concentrated and the residue was washed with hexane, then ether. The residue was acidified (TFA) and washed twice with water. The thick oily residue was stirred with dimethylsulfoxide (2 mL), acetonitrile (10 mL), and water (5 mL). On vigorous stirring the mixture crystallized to give a slurry. The mixture was further diluted with water (40 mL) and filtered to give a tan solid. Purification by reverse phase (C-18) chromatography gave the title compound as a white solid (450 mg, 11%). LC-MS m/z 490 (M+H). ¹H NMR (400 MHz, DMSO-d₆) δ ppm 12.29 (1H, br. s.), 9.58 (1H, t, J=6.2 Hz), 7.50 (1H, s), 7.09-7.18 (2H, m), 6.89 (1H, br. s.), 4.76 (2H, s), 4.42 (2H, d, J=6.2 Hz), 3.82 (3H, s), 3.36 (2H, d, J=6.6 Hz), 3.21 (2H, d, J=6.2 Hz), 1.68-1.85 (4H, m), 1.53 (1H, br. s.), 1.30 (1H, br. s.), 0.80-1.02 (4H, m).

### Example 10

### Trans-4-(((2-((4-Fluoro-3-methoxybenzyl)carbamoyl)-4-oxo-3,4-dihydrothieno[2,3-d]pyrimidin-5-yl)methoxy)methyl)cyclohexanecarboxylic acid

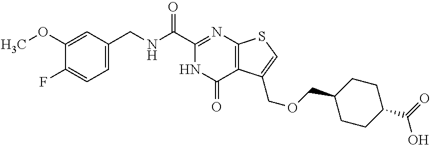

N-(4-Fluoro-3-methoxybenzyl)-5-(((trans-4-(hydroxymethyl)cyclohexyl)methoxy)methyl)-4-oxo-3,4-dihydrothieno[2,3-d]pyrimidine-2-carboxamide (prepared as described in Example 9) (244 mg) was dissolved in anhydrous dimethylformamide (5.0 mL) and pyridinium dichromate (2.70 g) was added. After stirring at room temperature for two hours the mixture was diluted with water (50 mL) and extracted with methylene chloride (3×50 mL). The extract was washed with water (2×50 mL), dried over magnesium sulfate, filtered and concentrated to give a dark residue. The residue was purified by reverse phase (C-18) chromatography to give the title compound as a white solid (158 mg, 63%). LC-MS 5%-95% CH₃CN:H₂O gradient over 6 min: 4.40 min, m/z 504 (M+H). ¹H NMR (400 MHz, DMSO-d₆) δ ppm 0.91-1.03 (m, 2H) 1.22-1.34 (m, 2H) 1.53 (s, 1H) 1.79 (d, J=11.71 Hz, 2H) 1.88 (d, J=11.71 Hz, 2H) 2.10 (t, J=11.71 Hz, 1H) 3.34 (d, J=6.59 Hz, 2H) 3.80 (s, 3H) 4.40 (d, J=5.86 Hz, 2H) 4.75 (s, 2H) 6.87 (br. s., 1H) 7.07-7.18 (m, 2H) 7.49 (s, 1H) 9.56 (t, J=5.86 Hz, 1H).

### Example 11

### N-(4-Fluoro-3-methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide

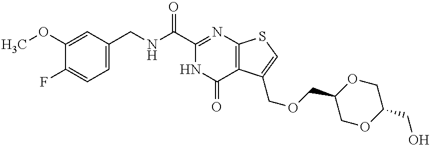

2.5 N n-Butyl lithium in hexanes (7.80 mL) was added to dry trans-1,4-dioxane-2,5-dimethanol (2.89 g) in anhydrous THF (100 mL). Ethyl 5-(bromomethyl)-4-oxo-3,4-dihydrothieno[2,3-d]pyrimidine-2-carboxylate (prepared as described in Step 3 of the synthesis of 5-({[trans-4-aminocyclohexyl]methoxy}methyl)-N-(3-methoxybenzyl)-4-oxo-3,4-dihydrothieno[2,3-d]pyrimidine-2-carboxamide) (2.00 g, vacuum dried) was added. After stirring two days at room temperature 4-fluoro-3-methoxybenzylamine (3.18 g) was added. After stirring at room temperature for four days the mixture was concentrated to remove tetrahydrofuran and acidified with trifluoroacetic acid. Purification by reverse phase (C-18) chromatography gave the title compound as a light yellow solid (727 mg, 23%). LC-MS 5%-95% CH₃CN:H₂O gradient over 6 min: 3.70 min, m/z 494 (M+H). ¹H NMR (400 MHz, DMSO-d₆) δ ppm 9.58 (1H, t, J=6.0 Hz), 7.54 (1H, s), 7.09-7.20 (2H, m), 6.90 (1H, br. s.), 4.80 (2H, s), 4.42 (2H, d, J=6.2 Hz), 3.78-3.88 (5H, m), 3.62 (1H, br. s.), 3.30-3.59 (8H, m).

### Example 12

### Trans-5-(((2-((4-Fluoro-3-methoxybenzyl)carbamoyl)-4-oxo-3,4-dihydrothieno[2,3-d]pyrimidin-5-yl)methoxy)methyl)-1,4-dioxane-2-carboxylic acid

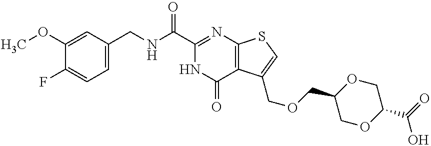

N-(4-Fluoro-3-methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide (prepared as described in Example 11) (476 mg) was dissolved in anhydrous dimethylformamide (10 mL) and pyridinium dichromate (4.81 g) was added. The mixture was stirred under nitrogen at 45 degrees for 2 hours. The mixture was diluted with water (100 mL) and extracted with methylene chloride (3×50 mL). The extract was washed with water (50 mL) and concentrated to give a dark oil. The oil was purified by reverse phase (C-18) chromatography to give the title compound as give a white solid (367 mg, 75%). LC-MS 5%-95% CH₃CN:H₂O gradient over 6 min: 3.81 min. m/z 508 (M+H). ¹H NMR (400 MHz, DMSO-d₆) δ ppm 12.34 (1H, br. s.), 9.59 (1H, t, J=5.7 Hz), 7.55 (1H, s), 7.09-7.18 (2H, m), 6.84-6.92 (1H, m), 4.81 (2H, s), 4.42 (2H, d, J=6.2 Hz), 4.09 (1H, d, J=8.1 Hz), 3.97 (1H, d, J=11.0 Hz), 3.89 (1H, d, J=11.3 Hz), 3.82 (3H, s), 3.69 (1H, br. s.), 3.38-3.61 (4H, m).

### Example 13

### N-(4-Fluoro-3-methoxybenzyl)-5-(((trans-4-carbamoylcyclohexyl)methoxy)methyl)-4-oxo-3,4-dihydrothieno[2,3-d]pyrimidine-2-carboxamide

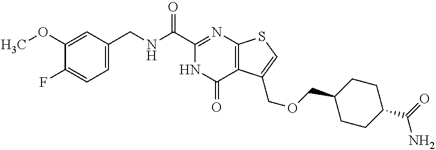

trans-5-(((2-((4-Fluoro-3-methoxybenzyl)carbamoyl)-4-oxo-3,4-dihydrothieno[2,3-d]pyrimidin-5-yl)methoxy)methyl)-1,4-dioxane-2-carboxylic acid (prepared as described in Example 12) (138 mg) was dissolved in anhydrous THF (10 mL) under nitrogen. Oxalyl chloride (0.30 mL) was added, followed by dimethylformamide (one drop). After stirring at room temperature for 10 minutes the mixture was concentrated under vacuum, diluted with anhydrous THF (15 mL) under nitrogen and cooled (acetone/dry ice bath). 7M ammonia in MeOH (0.50 mL) was added in one portion. The mixture was stirred and allowed to warm to room temperature. Water (15 mL) was added and the solution was concentrated to remove THF. The resulting slurry was filtered and the solid was washed with water and dried to give a yellow solid. Purification by reverse phase (C-18) chromatography gave the title compound as a white solid (70 mg, 51%). LC-MS 5%-95% CH₃CN:H₂O gradient over 6 min: 3.98 min, m/z 503 (M+H). ¹H NMR (400 MHz, DMSO-d₆) δ ppm 12.28 (1H, br. s.), 9.58 (1H, t, J=6.2 Hz), 7.51 (1H, s), 7.07-7.18 (2H, m), 6.90 (1H, d, J=4.4 Hz), 6.57 (1H, br. s.), 4.77 (2H, s), 4.42 (2H, d, J=5.9 Hz), 3.82 (3H, s), 3.36 (2H, d, J=6.2 Hz), 1.97-2.08 (1H, m), 1.78 (5H, t, J=14.3 Hz), 1.56 (1H, br. s.), 1.27-1.38 (2H, m), 0.89-1.03 (2H, m).

### Example 14

### N-(4-Fluoro-3-methoxybenzyl)-5-(((trans-4-cyanocyclohexyl)methoxy)methyl)-4-oxo-3,4-dihydrothieno[2,3-d]pyrimidine-2-carboxamide

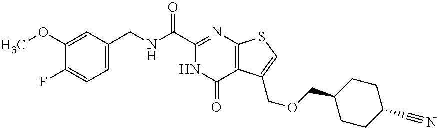

N-(4-Fluoro-3-methoxybenzyl)-5-(((trans-4-carbamoylcyclohexyl)methoxy)methyl)-4-oxo-3,4-dihydrothieno[2,3-d]pyrimidine-2-carboxamide (prepared as described in Example 13) (48 mg) was stirred in anhydrous dimethylformamide (5 mL). The slurry was warmed to dissolve the solid, and then cooled quickly to room temperature (ice bath). Phosphorus oxychloride (0.100 mL) was added under nitrogen and the clear solution was stirred at room temperature for 1½ minutes. The mixture was quenched by adding sodium bicarbonate (0.50 g) in ice water (15 mL). The resulting slurry was filtered and the solid was washed with water and dried to give the title compound as a white solid (45 mg, 97%). LC-MS 5%-95% CH₃CN:H₂O gradient over 6 min: 4.79 min, m/z 485 (M+H). ¹H NMR (400 MHz, DMSO-d₆) δ ppm 12.29 (1H, br. s.), 9.58 (1H, t, J=6.0 Hz), 7.50 (1H, s), 7.15 (2H, dt, J=8.0, 3.9 Hz), 6.90 (1H, s), 4.76 (2H, s), 4.42 (2H, d, J=6.2 Hz), 3.82 (3H, s), 3.35 (2H, d, J=6.2 Hz), 2.55-2.66 (1H, m), 1.97-2.05 (2H, m), 1.74-1.83 (2H, m), 1.63 (1H, s), 1.43-1.55 (2H, m), 0.97-1.08 (2H, m).

### Example 15

### N-(4-Fluoro-3-methoxybenzyl)-5-(((trans-5-carbamoyl-1,4-dioxan-2-yl)methoxy)methyl)-4-oxo-3,4-dihydrothieno[2,3-d]pyrimidine-2-carboxamide

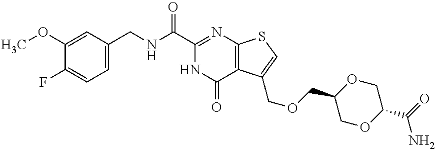

trans-5-(((2-((4-Fluoro-3-methoxybenzyl)carbamoyl)-4-oxo-3,4-dihydrothieno[2,3-d]pyrimidin-5-yl)methoxy)methyl)-1,4-dioxane-2-carboxylic acid (prepared as described in Example 12) (333 mg) was dissolved in anhydrous tetrahydrofuran (5 mL) under nitrogen. Oxalyl chloride (0.30 mL) was added, followed by dimethylformamide (0.010 mL). After two minutes the mixture was concentrated to dryness. The residue was dissolved in anhydrous tetrahydrofuran (5 mL). The solution was cooled (dry ice/acetone bath) and 7M ammonia in methanol (1.0 mL) was added and the mixture was allowed to warm to room temperature. After stirring a few minutes the mixture was concentrated to remove tetrahydrofuran and the water layer was decanted from an oil. The aqueous layer was extracted once with methylene chloride (5 mL). The extract was added to the oil in acetonitrile. The mixture was acidified (TFA, 0.3 mL) and concentrated to dryness to give a yellow solid. The solid was purified by reverse phase (C-18) chromatography to give the title compound as a white solid (27 mg, 8%). LC-MS 5%-95% CH₃CN:H₂O gradient over 6 min: 3.70 min, m/z 507 (M+H). ¹H NMR (400 MHz, DMSO-d₆) δ ppm 12.34 (1H, br. s.), 9.60 (1H, t, J=6.4 Hz), 7.55 (1H, s), 7.29 (1H, br. s.), 7.10-7.21 (3H, m), 6.90 (1H, br. s.), 4.81 (2H, s), 4.42 (2H, d, J=5.9 Hz), 3.96 (1H, d, J=13.5 Hz), 3.91 (2H, d, J=10.6 Hz), 3.82 (3H, s), 3.69 (1H, br. s.), 3.52-3.62 (2H, m), 3.35-3.50 (2H, m).

### Example 16

### N-(3-Methoxybenzyl)-5-[({trans-4-[(methylsulfonyl)amino]cyclohexyl}methoxy)methyl]-4-oxo-3,4-dihydrothieno[2,3-d]pyrimidine-2-carboxamide

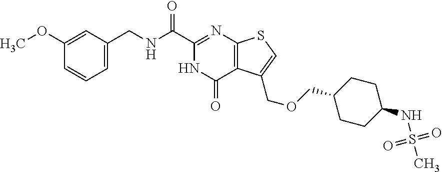

To a stirring mixture of 5-{[(trans-4-aminocyclohexyl)methoxy]methyl}-N-(3-methoxybenzyl)-4-oxo-3,4-dihydrothieno[2,3-d]pyrimidine-2-carboxamide (prepared as described in Example 1) (0.09 g) in 1 mL of CH₂Cl₂ and 1 mL of N,N-dimethylacetamide at room temperature was added 0.048 mL of N,N-diisopropylethylamine followed by 0.018 mL of methanesulfonyl chloride. After 1 hour, the reaction mixture was treated with 0.020 mL of N,N-diisopropylethylamine and 0.005 mL of methanesulfonyl chloride. After 0.5 hour, the reaction mixture was diluted with EtOAc (˜50 mL) and washed sequentially with pH 4 phosphate buffer (2×10 mL) and brine. The separated organic phase was dried over sodium sulfate, filtered and concentrated under reduced pressure to give a tan residue. Purification by flash column silica chromatography eluting with 2-4% MeOH in CH₂Cl₂ provided an off-white solid which was crystallized from EtOAc:hexanes to afford the title compound as an off-white solid (0.027 g). LC/MS (5-100% CH₃CN:H₂O gradient over 4 min) 3.2 min, m/z 535 (M+H). ¹H NMR (400 MHz, DMSO-d₆) δ ppm 12.41 (1H, s), 9.64 (1H, t, J=6.4 Hz), 7.52 (1H, s), 7.24 (1H, t, J=8.1 Hz), 6.98 (1H, d, J=7.3 Hz), 6.89-6.94 (2H, m), 6.77-6.86 (1H, m), 4.77 (2H, s), 4.42 (2H, d, J=6.3 Hz), 3.74 (3H, s), 3.30-3.38 (2H, m), 2.98-3.12 (1H, m), 2.90 (3H, s), 1.86-1.96 (2H, m), 1.71-1.84 (2H, m), 1.43-1.61 (1H, m), 1.13-1.30 (2H, m), 0.99-1.11 (2H, m).

### Example 17

### 5-({[trans-4-(Acetylamino)cyclohexyl]methoxy}methyl)-N-(3-methoxybenzyl)-4-oxo-3,4-dihydrothieno[2,3-d]pyrimidine-2-carboxamide

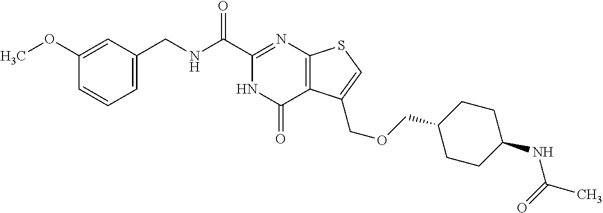

To a stirring mixture of 5-{[(trans-4-aminocyclohexyl)methoxy]methyl}-N-(3-methoxybenzyl)-4-oxo-3,4-dihydrothieno[2,3-d]pyrimidine-2-carboxamide (prepared as described in Example 1) (0.093 g) in 1 mL of CH₂Cl₂ and 1 mL of N,N-dimethylacetamide at room temperature was added 0.055 mL of N,N-diisopropylethylamine followed by 0.022 mL of acetyl chloride. After 2 hours, the reaction mixture was diluted with EtOAc (˜50 mL) and washed sequentially with pH 4 phosphate buffer (2×10 mL) and brine. The separated organic phase was dried over sodium sulfate, filtered and concentrated under reduced pressure to give a tan residue. Purification by reverse phase chromatography provided the title compound as an off-white solid (0.016 g). LC/MS (5-95% CH₃CN:H₂O gradient over 4 min, then 2 min 95%) 3.3 min, m/z 499 (M+H). ¹H NMR (400 MHz, DMSO-d₆) δ ppm 12.40 (1H, s), 9.64 (1H, t, J=6.5 Hz), 7.69 (1H, d, J=7.7 Hz), 7.52 (1H, s), 7.24 (1H, t, J=7.9 Hz), 6.85-6.96 (2H, m), 6.76-6.85 (1H, m), 4.76 (2H, s), 4.41 (2H, d, J=6.5 Hz), 3.73 (3H, s), 3.39-3.56 (1H, m), 3.36 (2H, d, J=6.5 Hz), 1.65-1.85 (4H, m), 1.76 (3H, s) 0.89-1.20 (5H, m).

### Example 18

### N-(4-Fluoro-3-methoxybenzyl)-5-[({trans-4-[(methylsulfonyl)amino]cyclohexyl}methoxy)methyl]-4-oxo-3,4-dihydrothieno[2,3-d]pyrimidine-2-carboxamide

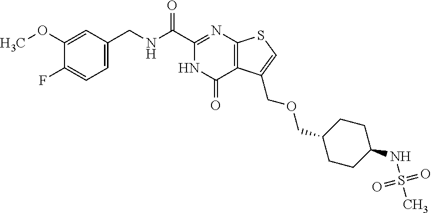

To a stirring mixture of 5-{[(trans-4-aminocyclohexyl)methoxy]methyl}-N-(4-fluoro-3-methoxybenzyl)-4-oxo-3,4-dihydrothieno[2,3-d]pyrimidine-2-carboxamide (prepared as described in Example 2) (0.12 g) in 1 mL of CH₂Cl₂ and 1 mL of N,N-dimethylacetamide at room temperature was added 0.060 mL of N,N-diisopropylethylamine followed by 0.024 mL of methanesulfonyl chloride. After 1 hour, the reaction mixture was treated with 0.004 mL of methanesulfonyl chloride. After 2 hours, the reaction mixture was diluted with EtOAc containing a small volume of MeOH and washed sequentially with pH 4 phosphate buffer (2×20 mL) and brine (2×10 mL). The separated organic phase was dried over sodium sulfate, filtered and concentrated under reduced pressure to give a tan residue. Purification by reverse phase chromatography provided the title compound as an off-white solid (0.024 g). LC/MS (5-100% CH₃CN:H₂O gradient over 4 min) 3.2 min, m/z 553 (M+H). ¹H NMR (400 MHz, DMSO-d₆) δ ppm 12.41 (1H, s), 9.65 (1H, t, J=6.5 Hz), 7.52 (1H, s), 7.10-7.20 (2H, m), 6.98 (1H, d, J=7.3 Hz), 6.83-6.92 (1H, m), 4.76 (2H, s), 4.41 (2H, d, J=6.3 Hz), 3.82 (3H, s), 3.30-3.40 (2H, m), 2.97-3.10 (1H, m), 2.89 (3H, s), 1.83-1.96 (2H, m), 1.77 (2H, d, J=12.3 Hz), 1.43-1.58 (1H, m), 1.10-1.28 (2H, m), 0.95-1.12 (2H, m).

### Example 19

### 5-({[trans-4-(Acetylamino)cyclohexyl]methoxy}methyl)-N-(4-fluoro-3-methoxybenzyl)-4-oxo-3,4-dihydrothieno[2,3-d]pyrimidine-2-carboxamide

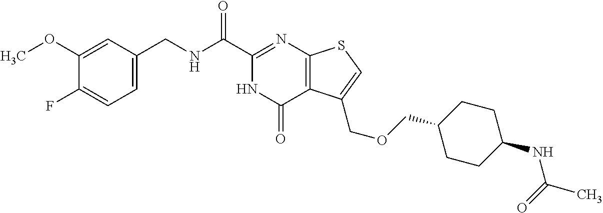

To a stirring mixture of 5-{[(trans-4-aminocyclohexyl)methoxy]methyl}-N-(4-fluoro-3-methoxybenzyl)-4-oxo-3,4-dihydrothieno[2,3-d]pyrimidine-2-carboxamide (prepared as described in Example 2) (0.10 g) in 1 mL of CH₂Cl₂ and 1 mL of N,N-dimethylacetamide at room temperature was added 0.055 mL of N,N-diisopropylethylamine followed by 0.020 mL of acetyl chloride. After 1.5 hours, the reaction mixture was diluted with EtOAc (˜60 mL) and washed sequentially with pH 4 phosphate buffer (2×15 mL) and brine (2×10 mL). The separated organic phase was dried over sodium sulfate, filtered and concentrated under reduced pressure to give a tan residue. Purification by reverse phase chromatography provided the title compound as an off-white solid (0.048 g). LC/MS (5-100% CH₃CN:H₂O gradient over 4 min) 3.2 min, m/z 517 (M+H). ¹H NMR (400 MHz, DMSO-d₆) δ ppm 12.41 (1H, br. s.), 9.65 (1H, t, J=6.3 Hz), 7.70 (1H, d, J=7.7 Hz), 7.52 (1H, s), 7.04-7.23 (2H, m), 6.80-6.96 (1H, m), 4.76 (2H, s), 4.41 (2H, d, J=6.3 Hz), 3.82 (3H, s), 3.39-3.54 (1H, m), 3.36 (2H, d, J=6.3 Hz), 1.70-1.86 (4H, m), 1.76 (3H, s), 1.45-1.61 (1H, m), 0.74-1.35 (4H, m).

### Example 20

### 2-((trans-1,4)-4-(((2-((3-methoxybenzyl)carbamoyl)-4-oxo-3,4-dihydrothieno[2,3-d]pyrimidin-5-yl)methoxy)methyl)cyclohexylamino)-2-oxoethyl acetate

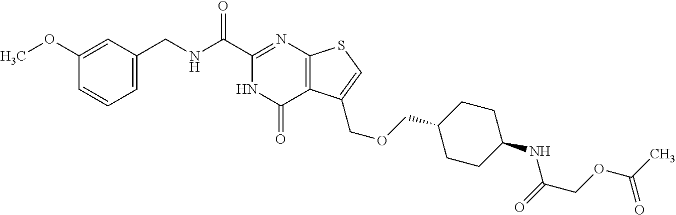

To a yellow suspension of N-(3-methoxybenzyl)-5-((((trans-1,4)-4-aminocyclohexyl)methoxy)methyl)-4-oxo-3,4-dihydrothieno[2,3-d]pyrimidine-2-carboxamide (prepared as described in Example 1) (0.11 g, 0.17 mmol) in CH₂Cl₂ (1.5 mL) containing triethylamine (0.048 mL, 0.35 mmol) and N,N-dimethyl-4-aminopyridine (4.24 mg, 0.035 mmol) was added N,N-dimethylacetamide (1.5 mL). The yellow suspension was treated with acetoxyacetyl chloride (0.017 mL, 0.16 mmol). The reaction was stirred for 2 hr at room temperature and then diluted with EtOAc (50 mL). The organic layer was washed with saturated sodium bicarbonate (2×10 mL), brine (10 mL), dried over magnesium sulfate, filtered and concentrated under reduced pressure to afford a solid. The material was triturated under EtOAc (10 mL), filtered, washed with EtOAc (10 mL), and air-dried to provide the title compound as a white solid (61 mg). LC/MS 15-95% acetonitrile/TFA-water/TFA (5 min gradient) 3.2 min, m/z 557 (M+H). ¹H NMR (400 MHz, DMSO-d₆) δ ppm 12.40 (1H, br. s.), 9.63 (1H, t, J=6.4 Hz), 7.83 (1H, d, J=7.8 Hz), 7.51 (1H, s), 7.24 (1H, t, J=8.2 Hz), 6.86-6.96 (2H, m), 6.75-6.86 (1H, m), 4.76 (2H, s), 4.41 (2H, d, J=6.4 Hz), 4.39 (2H, s), 3.73 (3H, s), 3.41-3.61 (1H, m), 3.36 (2H, d, J=6.4 Hz), 2.07 (3H, s), 1.78 (4H, dd, J=9.5, 5.0 Hz), 1.46-1.62 (1H, m), 1.12-1.29 (2H, m), 0.94-1.10 (2H, m).

### Example 21

### N-(3-Methoxybenzyl)-5-((((trans-1,4)-4-(2-hydroxyacetamido)cyclohexyl)methoxy)methyl)-4-oxo-3,4-dihydrothieno[2,3-d]pyrimidine-2-carboxamide

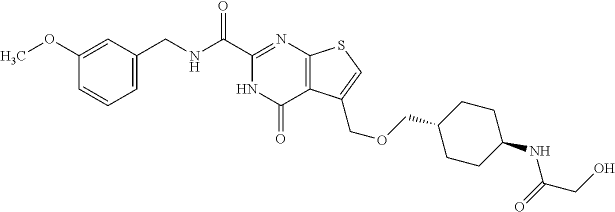

To a suspension of 2-((trans-1,4)-4-(((2-((3-methoxybenzyl)carbamoyl)-4-oxo-3,4-dihydrothieno[2,3-d]pyrimidin-5-yl)methoxy)methyl)cyclohexylamino)-2-oxoethyl acetate (prepared as described in Example 20) (58 mg, 0.10 mmol) in THF (3 mL) was added lithium hydroxide (0.313 mL, 1M solution). After stirring 2 hr at room temperature, the pH was adjusted to ˜7 with 1M potassium hydrogen sulfate and the volatile organic solvent was removed in vacuo. The mixture was extracted with 3×15 mL of EtOAc. The combined organic layers were dried over magnesium sulfate, filtered, and evaporated to afford the title compound as white solid (14 mg). LC/MS 15-95% acetonitrile/TFA-water/TFA (5 min gradient) 3.18 min, m/z 515 (M+H). ¹H NMR (400 MHz, DMSO-d₆) δ ppm 12.40 (1H, br. s.), 9.63 (1H, t, J=6.4 Hz), 7.52 (1H, s), 7.41 (1H, d, J=8.3 Hz), 7.24 (1H, t, J=8.2 Hz), 6.86-6.94 (2H, m), 6.78-6.85 (1H, m), 5.39 (1H, t, J=5.9 Hz), 4.77 (2H, s), 4.41 (2H, d, J=6.2 Hz), 3.76 (2H, d, J=5.6 Hz), 3.73 (3H, s), 3.48-3.63 (1H, m), 3.37 (2H, d, J=6.4 Hz), 1.70-1.86 (4H, m), 1.54 (1H, dd, J=6.3, 2.8 Hz), 1.21-1.36 (2H, m), 0.96-1.12 (2H, m).

### Example 22

### 2-((trans-1,4)-4-(((2-((4-fluoro-3-methoxybenzyl)carbamoyl)-4-oxo-3,4-dihydrothieno[2,3-d]pyrimidin-5-yl)methoxy)methyl)cyclohexylamino)-2-oxoethyl acetate

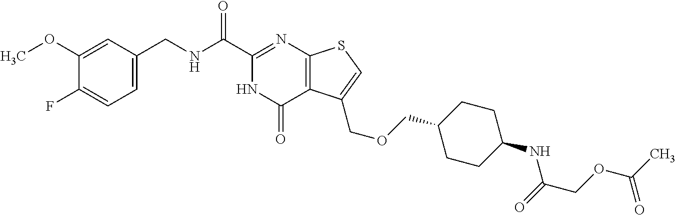

To a suspension of N-(4-fluoro-3-methoxybenzyl)-5-((((trans-1,4)-4-aminocyclohexyl)methoxy)methyl)-4-oxo-3,4-dihydrothieno[2,3-d]pyrimidine-2-carboxamide (prepared as described in Example 2) (0.119 g, 0.21 mmol) in CH₂Cl₂ (1.5 mL) was added triethylamine (0.058 mL, 0.42 mmol), N,N-dimethyl-4-aminopyridine (5.1 mg, 0.042 mmol) and N,N-dimethylacetamide (1.5 mL). The reaction mixture was treated with acetoxyacetyl chloride (0.020 mL, 0.19 mmol), stirred for 2 hr at room temperature then diluted with EtOAc (50 mL). The organic layer was washed with saturated sodium bicarbonate (2×10 mL), brine (10 mL), dried over magnesium sulfate, filtered and concentrated under reduced pressure to afford the title compound as a white solid (0.11 g). LC/MS 5-100% acetonitrile/TFA-water/TFA (5 min gradient) 3.22 min, m/z 575 (M+H).

### Example 23

### N-(4-Fluoro-3-methoxybenzyl)-5-((((trans-1,4)-4-(2-hydroxyacetamido)cyclohexyl)methoxy)methyl)-4-oxo-3,4-dihydrothieno[2,3-d]pyrimidine-2-carboxamide

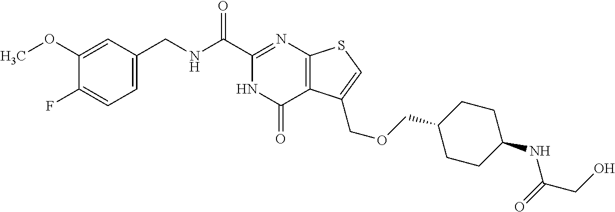

To a suspension of 2-((trans-1,4)-4-(((2-((4-fluoro-3-methoxybenzyl)carbamoyl)-4-oxo-3,4-dihydrothieno[2,3-d]pyrimidin-5-yl)methoxy)methyl)cyclohexylamino)-2-oxoethyl acetate (prepared as described in Example 22) (108 mg, 0.188 mmol) in THF (3 mL) was added lithium hydroxide (0.56 mL, 1M solution). After stirring 2 hr at room temperature, the pH was adjusted to ˜7 with 1M potassium hydrogen sulfate and the volatile organic solvent was removed in vacuo. The mixture was extracted with 3×15 mL EtOAc. The combined organic layers were dried over magnesium sulfate, filtered, and evaporated to afford a white solid. The white solid was purified using reverse phase chromatography to afford the title compound as a white solid (37 mg). LC/MS 5-100% acetonitrile/TFA-water/TFA (4 min gradient) 3.02 min, m/z 533 (M+H). ¹H NMR (400 MHz, DMSO-d₆) δ ppm 12.40 (1H, s), 9.64 (1H, t, J=6.3 Hz), 7.52 (1H, s), 7.41 (1H, d, J=8.3 Hz), 7.09-7.21 (2H, m), 6.83-6.96 (1H, m), 4.76 (2H, s), 4.41 (2H, d, J=6.2 Hz), 3.81 (3H, s), 3.76 (2H, s), 3.55-3.64 (1H, m), 3.37 (2H, d, J=6.4 Hz), 1.69-1.85 (4H, m), 1.47-1.62 (1H, m), 1.17-1.36 (2H, m), 0.95-1.13 (2H, m).

### Example 24

### 5-({[trans-4-aminomethylcyclohexyl]methoxy}methyl)-N-(3-methoxybenzyl)-4-oxo-3,4-dihydrothieno[2,3-d]pyrimidine-2-carboxamide

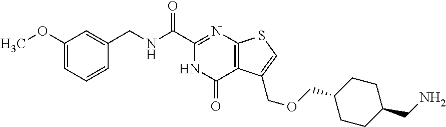

### Step 1: Preparation of trans-4-[(tert-butoxycarbonyl)aminomethyl]cyclohexylmethyl 4-methylbenzenesulfonate

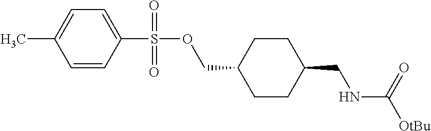

To a solution of tert-butyl(trans-4-hydroxymethylcyclohexylmethyl)carbamate (0.50 g) in 3 mL of pyridine at 0° C. was slowly added tosyl chloride (0.44 g). The reaction mixture was slowly warmed to room temperature and a cloudy suspension resulted. After 4 hours, the reaction mixture was cooled to 0° C. and treated slowly with 30 mL of water. The resulting white precipitant was filtered, washed with cold water (2×3 mL), air-dried, and finally dried in vacuo to provide the title compound as a white solid (0.7 g). LC/MS (5-100% CH₃CN/TFA:H₂O/TFA gradient over 4 min) 3.8 min, m/z 420 (M+Na). ¹H NMR (400 MHz, DMSO-d₆) δ ppm 7.78 (2H, d), 7.48 (2H, d), 6.78 (1H, t), 3.82 (2H, d), 2.73 (1H, t), 2.43 (3H, s), 1.4-1.7 (5H, m), 1.36 (9H, s), 1.15-1.3 (1H, m), 0.7-0.9 (4H, m).

### Step 2: Preparation of 5-({[trans-4-aminomethylcyclohexyl]methoxy}methyl)-N-(3-methoxybenzyl)-4-oxo-3,4-dihydrothieno[2,3-d]pyrimidine-2-carboxamide

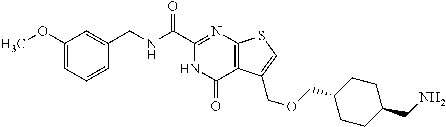

To an oven-dried 3-neck, round-bottom flask equipped with a stir bar, nitrogen inlet on a reflux condenser, glass and rubber stoppers was charged 0.24 g (60% by wt dispersion in oil) of sodium hydride. The flask was back-filled with nitrogen and treated with dry DMF (2 mL). The resulting grey suspension was cooled to 0° C. and was slowly treated with 5-(hydroxymethyl)-N-(3-methoxybenzyl)-4-oxo-3,4-dihydrothieno[2,3-d]pyrimidine-2-carboxamide (prepared as described in step 5 of the synthesis of 5-({[trans-4-Aminocyclohexyl]methoxy}methyl)-N-(3-methoxybenzyl)-4-oxo-3,4-dihydrothieno[2,3-d]pyrimidine-2-carboxamide, Example 1) (0.31 g) as a tan solution in 5 mL of dry DMF in a dropwise fashion. After 10 min, the reaction was warmed to room temperature. After 1 hr, the resulting suspension was cooled to 0° C. and treated dropwise with trans-4-[(tert-butoxycarbonyl)aminomethyl]cyclohexylmethyl 4-methylbenzenesulfonate (0.64 g) as a solution in 3 mL of dry DMF. After 10 min, the reaction mixture was warmed to room temperature. After 15 minutes, the reaction mixture was heated to 45° C. After 45 minutes, the resulting reaction suspension was cooled to room temperature, quenched with 7 mL of a 1 N aqueous hydrogen chloride solution, diluted with 17 mL of brine, and partitioned with 30 mL THF. The layers were separated and the aqueous phase (pH ˜7) was extracted with THF (30 mL) and 1:1 THF-ethyl acetate mixture (2×30 mL). The organic phases were combined and washed with brine (2×20 mL), dried over sodium sulfate, filtered and concentrated under reduced pressure to give a yellow residue. The residue was adsorbed onto silica gel and purified by silica gel flash column chromatography eluting with 10-30% methanol (7M ammonia) in CH₂Cl₂. The product containing fractions were combined to give the title compound as a tan solid (54 mg). LC/MS (5-100% CH₃CN/TFA:H₂O/TFA gradient over 4 min) 2.8 min, m/z 471 (M+H).

### Example 25

### N-(3-methoxybenzyl)-5-[({trans-4-[(methylsulfonyl)aminomethyl]cyclohexyl}methoxy)methyl]-4-oxo-3,4-dihydrothieno[2,3-d]pyrimidine-2-carboxamide

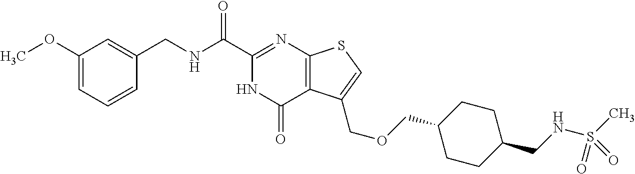

To a stirring mixture of 5-{[(trans-4-aminomethylcyclohexyl)methoxy]methyl}-N-(3-methoxybenzyl)-4-oxo-3,4-dihydrothieno[2,3-d]pyrimidine-2-carboxamide (prepared as described in Example 24) (54 mg) in 1 mL of CH₂Cl₂ and 1 mL of N,N-dimethylacetamide at room temperature was added 0.029 mL of N,N-diisopropylethylamine followed by 0.011 mL of methanesulfonyl chloride. After 2 hours, the reaction mixture was diluted with ethyl acetate (50 mL) containing a small volume of methanol and washed sequentially with pH 4 phosphate buffer (2×20 mL) and brine. The separated organic phase was dried over sodium sulfate, filtered and concentrated under reduced pressure to give a tan residue. The residue was adsorbed onto silica and purified by flash column silica chromatography eluting with 2-4% methanol in CH₂Cl₂. The product containing fractions were combined and purified by reverse phase column chromatography to provide the title compound. LC/MS (5-100% CH₃CN:H₂O gradient over 4 min) 3.3 min, m/z 549 (M+H). ¹H NMR (400 MHz, DMSO-d₆) δ ppm 9.43 (1H, m), 7.37 (1H, s), 7.20 (1H, t, J=8.1 Hz), 6.82-6.90 (3H, m), 6.74-6.81 (1H, m), 4.73 (2H, s), 4.38 (2H, d, J=6.4 Hz), 3.70 (3H, s), 3.30-3.36 (2H, m), 2.81 (3H, s), 2.73 (2H, t, J=6.4 Hz), 1.67-1.80 (4H, m), 1.45-1.57 (1H, m), 1.25-1.39 (1H, m), 0.76-1.00 (4H, m).

### Example 26

### N-(3-methoxybenzyl)-4-oxo-5-[(2-piperidin-4-ylethoxy)methyl]-3,4-dihydrothieno[2,3-d]pyrimidine-2-carboxamide hydrochloride

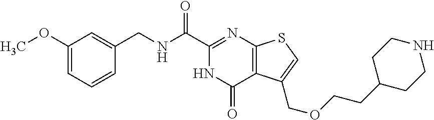

### Step 1: Preparation of tert-butyl 4-(2-{[(4-methylphenyl)sulfonyl]oxy}ethyl)piperidine-1-carboxylate

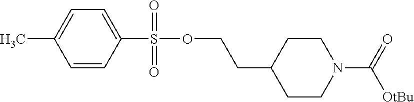

To a solution of tert-butyl 4-(2-hydroxyethyl)piperidine-1-carboxylate (2.29 g) in 12 mL of pyridine at 0° C. was slowly added tosyl chloride (2.2 g). The reaction mixture was slowly warmed to room temperature and a cloudy suspension resulted. After stirring overnight, the reaction mixture was cooled to 0° C. and treated slowly with 120 mL of water. The resulting mixture was partitioned with diethyl ether (50 mL) and the layers separated. The aqueous phase was extracted with diethyl ether (2×50 mL) and the combined organic layer was washed with 1 M aqueous potassium hydrogen sulfate (2×), saturated aqueous sodium bicarbonate, brine, dried over sodium sulfate, filtered, and concentrated under reduced pressure to give a viscous oil. The oil was purified by flash column silica chromatography eluting with 30 to 40% ethyl acetate in heptanes. The product containing fractions were combined and concentrated under reduced pressure to provide the title compound as a clear, colorless, viscous oil (0.95 g). LC/MS (5-100% CH₃CN/TFA:H₂O/TFA gradient over 4 min, then 2 min hold) 4.5 min, m/z 406 (M+Na). ¹H NMR (400 MHz, CDCl₃) δ ppm 7.81 (2H, d), 7.37 (2H, d), 4.0-4.2 (4H, m), 2.63 (2H, m), 2.48 (3H, s), 1.5-1.7 (5H, m), 1.46 (9H, s), 0.9-1.2 (2H, m).

### Step 2: Preparation of tert-butyl 4-{2-[(2-{[(3-methoxybenzyl)amino]carbonyl}-4-oxo-3,4-dihydrothieno[2,3-d]pyrimidin-5-yl)methoxy]ethyl}piperidine-1-carboxylate

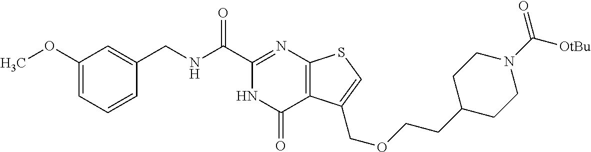

To an oven-dried 3-neck, round-bottom flask equipped with a stir bar, nitrogen inlet on a reflux condenser, glass and rubber stoppers was charged 0.15 g (60% by wt dispersion in oil) of sodium hydride. The flask was back-filled with nitrogen and treated with dry DMF (3 mL). The resulting grey suspension was cooled to 0° C. and was slowly treated with 5-(hydroxymethyl)-N-(3-methoxybenzyl)-4-oxo-3,4-dihydrothieno[2,3-d]pyrimidine-2-carboxamide (prepared as described in step 5 of the synthesis of 5-({[trans-4-aminocyclohexyl]methoxy}methyl)-N-(3-methoxybenzyl)-4-oxo-3,4-dihydrothieno[2,3-d]pyrimidine-2-carboxamide, Example 1) (0.31 g) as a tan solution in 6 mL of dry DMF in a dropwise fashion. After 10 min, the reaction was warmed to room temperature. After 30 min, the resulting suspension was cooled to 0° C. and treated dropwise with tert-butyl 4-(2-{[(4-methylphenyl)sulfonyl]oxy}ethyl)piperidine-1-carboxylate (0.62 g) as a solution in 3 mL of dry DMF. After 10 min, the reaction mixture was warmed to room temperature. After 4 hours, the resulting reaction suspension was quenched with 4 mL of a 1 N aqueous hydrogen chloride solution diluted with 16 mL of brine, and partitioned with 50 mL of 1:1 THF-ethyl acetate. The layers were separated and the aqueous phase was neutralized and extracted with 1:1 THF-ethyl acetate mixture (2×25 mL). The organic phases were combined and washed with brine (25 mL), dried over sodium sulfate, filtered and concentrated under reduced pressure to give an orange residue. The residue was adsorbed onto silica gel and purified by silica gel flash column chromatography eluting with 2-6% methanol in ethyl acetate. The product containing fractions were combined and concentrated under reduced pressure to a white solid. The solid was crystallized from ethyl acetate/hexanes to give the title compound as a white solid (0.25 g). LC/MS (5-100% CH₃CN/TFA:H₂O/TFA gradient over 4 min, then 2 min hold) 4.5 min, m/z 557 (M+H). ¹H NMR (400 MHz, DMSO-d₆) δ ppm 12.42 (1H, br. s.), 9.65 (1H, t, J=6.4 Hz), 7.51 (1H, s), 7.18-7.30 (1H, m), 6.85-6.93 (2H, m), 6.78-6.86 (1H, m), 4.74-4.80 (2H, m), 4.41 (2H, d, J=6.3 Hz), 3.82-3.98 (2H, m), 3.73 (3 H, s), 3.53-3.62 (2H, m), 2.56-2.78 (2H, m), 1.44-1.72 (5H, m), 1.37 (9H, s), 0.91-1.08 (2H, m).

### Step 3: Preparation of N-(3-methoxybenzyl)-4-oxo-5-[(2-piperidin-4-ylethoxy)methyl]-3,4-dihydrothieno[2,3-d]pyrimidine-2-carboxamide hydrochloride

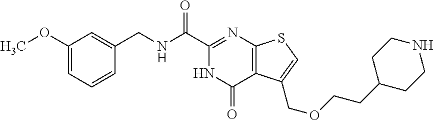

To a flask containing tert-butyl 4-{2-[(2-{[(3-methoxybenzyl)amino]carbonyl}-4-oxo-3,4-dihydrothieno[2,3-d]pyrimidin-5-yl)methoxy]ethyl}piperidine-1-carboxylate (208 mg) was charged a solution of HCl in dioxane (4 M, 1 mL). The resulting clear, yellow solution was allowed to stir at room temperature. After ˜1 hr, the reaction was a thick mass and was concentrated under reduced pressure. The resulting residue was triturated with diethyl ether (3×5 mL), air-dried and finally dried in vacuo to give the title compound as an off-white solid (0.18 g). LC/MS (5-100% CH₃CN/TFA:H₂O/TFA gradient over 4 min) 2.8 min, m/z 457 (M+H). ¹H NMR (400 MHz, DMSO-d₆) δ ppm 12.43 (1H, s), 9.64 (1H, t, J=6.4 Hz), 8.64 (1H, br. s.), 8.34 (1H, br. s.), 7.52 (1H, s), 7.15-7.30 (1H, m), 6.87-6.96 (2H, m), 6.78-6.86 (1H, m), 4.77 (2H, s), 4.42 (2H, d, J=6.3 Hz), 3.73 (3H, s), 3.54-3.64 (2H, m), 3.18-3.26 (2H, m), 2.75-2.91 (2H, m), 1.77-1.88 (2H, m), 1.63-1.77 (1H, m), 1.47-1.57 (2H, m), 1.23-1.38 (2H, m).

### Example 27

### N-(3-methoxybenzyl)-5-({2-[1-(methylsulfonyl)piperidin-4-yl]ethoxy}methyl)-4-oxo-3,4-dihydrothieno[2,3-d]pyrimidine-2-carboxamide

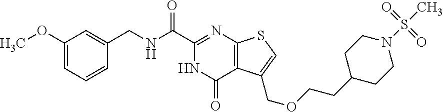

To a stirring mixture of N-(3-methoxybenzyl)-4-oxo-5-[(2-piperidin-4-ylethoxy)methyl]-3,4-dihydrothieno[2,3-d]pyrimidine-2-carboxamide hydrochloride (prepared as described in example 26) (70 mg) in 1.5 mL of CH₂Cl₂ and 1.5 mL of N,N-dimethylacetamide at room temperature was added 0.045 mL of N,N-diisopropylethylamine followed by 0.015 mL of methanesulfonyl chloride. After 2 hours, the reaction mixture was treated with 0.005 mL of methanesulfonyl chloride. After 1 hour, the reaction mixture was treated with 0.030 mL of N,N-diisopropylethylamine. After 30 min, the reaction mixture was diluted with ethyl acetate (50 mL) and partitioned against pH 4 phosphate buffer (25 mL). The separatory funnel was treated with additional water and ethyl acetate. The separated aqueous phase containing a precipitant was extracted with ethyl acetate and dichloromethane (3×50 mL). The organic phases were combined and washed with pH 4 phosphate buffer followed by brine. The separated organic phase was dried over sodium sulfate, filtered and concentrated under reduced pressure to give a white solid. This solid was crystallized from methanol and ethyl acetate to provide the title compound as a white solid (66 mg). LC/MS (5-100% CH₃CN:H₂O gradient over 4 min) 3.7 min, m/z 535 (M+H). ¹H NMR (400 MHz, DMSO-d₆) δ ppm 12.41 (1H, s), 9.63 (1H, t, J=6.3 Hz), 7.51 (1H, s), 7.24 (1H, t, J=8.2 Hz), 6.85-6.94 (2H, m), 6.77-6.85 (1H, m), 4.77 (2H, s), 4.41 (2H, d, J=6.4 Hz), 3.73 (3H, s), 3.55-3.63 (2H, m), 3.45-3.55 (2H, m), 2.82 (3H, s), 2.58-2.73 (2H, m), 1.76 (2H, d, J=11.5 Hz), 1.47-1.61 (3H, m), 1.09-1.27 (2H, m).

### Example 28

### 5-([2-(1-Acetylpiperidin-4-yl)ethoxy]methyl)-N-(3-methoxybenzyl)-4-oxo-3,4-dihydrothieno[2,3-d]pyrimidine-2-carboxamide

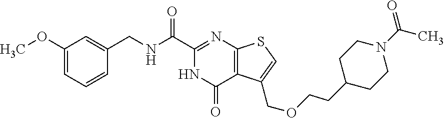

To a stirring mixture of N-(3-methoxybenzyl)-4-oxo-5-[(2-piperidin-4-ylethoxy)methyl]-3,4-dihydrothieno[2,3-d]pyrimidine-2-carboxamide hydrochloride (prepared as described in example 26) (70 mg) in 1.5 mL of CH₂Cl₂ and 1.5 mL of N,N-dimethylacetamide at room temperature was added 0.045 mL of N,N-diisopropylethylamine followed by 0.013 mL of acetyl chloride. After 2 hours, the reaction mixture was treated with 0.002 mL of acetyl chloride. After 1 hour, the reaction mixture was treated with 0.030 mL of N,N-diisopropylethylamine. After 30 min, the reaction mixture was diluted with ethyl acetate (50 mL) and partitioned against pH 4 phosphate buffer (25 mL). The organic phase was washed with pH 4 phosphate buffer, brine, dried over sodium sulfate, filtered and concentrated under reduced pressure to give a viscous, oily solid. This solid was crystallized from ethyl acetate and heptanes to give a residue. This residue was crystallized from toluene to give an off-white solid. This solid was purified by reverse phase column chromatography to provide the title compound as a white solid (38 mg). LC/MS (5-100% CH₃CN:H₂O gradient over 4 min) 3.3 min, m/z 499 (M+H). ¹H NMR (400 MHz, DMSO-d₆) δ ppm 12.42 (1H, s), 9.65 (1H, t, J=6.3 Hz), 7.52 (1H, s), 7.24 (1H, t, J=8.1 Hz), 6.87-6.93 (2H, m), 6.82 (1H, dd, J=8.8, 2.0 Hz), 4.77 (2H, s), 4.41 (2H, d, J=6.5 Hz), 4.27-4.37 (1H, m), 3.73-3.80 (1H, m), 3.73 (3H, s), 3.59 (2H, t, J=6.5 Hz), 2.91-3.02 (1H, m), 2.42-2.48 (1H, m), 1.96 (3H, s), 1.58-1.73 (3H, m), 1.46-1.55 (2H, m), 0.86-1.14 (2H, m).

### Example 29

### 6-fluoro-5-{[trans-4-(hydroxymethyl)cyclohexyl]methoxy}-N-(3-methoxybenzyl)-4-oxo-3,4-dihydroquinazoline-2-carboxamide

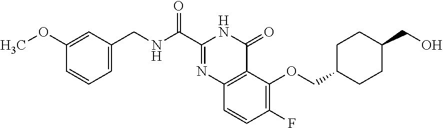

To a dry flask under an atmosphere of argon was added sodium hydride (120 mg, 60% dispersion in oil) and the flask was flushed with argon. The flask was charged with 2 mL of N,N-dimethylacetamide and flushed with argon. After stirring for 5 min at room temperature, a solution of the trans-cyclohexane-1,4-diyldimethanol (222 mg, 1.5 mmol) in 1 mL of N,N-dimethylacetamide was added via syringe. The mixture was stirred for 5 min at room temperature and a solution of N-(3-methoxybenzyl)-5,6-difluoro-4-oxo-3,4-dihydroquinazoline-2-carboxamide (prepared as described in step 5 of the synthesis of 6-fluoro-5-{[(trans-2,5)-5-(hydroxymethyl)-1,4-dioxan-2-yl]methoxy}-N-(3-methoxybenzyl)-4-oxo-3,4-dihydroquinazoline-2-carboxamide, Example 3) (173 mg, 0.5 mmol) in 1 mL of N,N-dimethylacetamide was added via syringe and the reaction was heated to 60° C. After 90 min, the reaction was cooled to room temperature, the pH was lowered to ˜4 with 1M hydrochloric acid, and then diluted with ethyl acetate (50 mL). The organic layer was washed with water (20 mL), brine (20 mL), dried over magnesium sulfate, filtered, and evaporated to afford an off-white solid. The solid was purified using reverse phase chromatography to afford the title compound as a white solid (104 mg). ¹H NMR (400 MHz, DMSO-d₆) δ ppm 12.07 (1H, br. s.), 9.49 (1H, t, J=6.4 Hz), 7.79 (1H, dd, J=10.5, 9.1 Hz), 7.46-7.56 (1H, m), 7.24 (1H, t, J=8.1 Hz), 6.88-6.96 (2H, m), 6.77-6.87 (1H, m), 4.44 (2H, d, J=6.4 Hz), 4.36 (1H, t, J=5.2 Hz), 3.88 (2H, d, J=6.2 Hz), 3.74 (3H, s), 3.22 (2H, t, J=5.8 Hz), 1.85-1.96 (2H, m), 1.65-1.82 (4H, m), 0.98-1.14 (2H, m), 0.83-0.98 (2H, m).

### Example 30

### 6-fluoro-N-(3-methoxybenzyl)-4-oxo-5-(2-piperidin-4-ylethoxy)-3,4-dihydroquinazoline-2-carboxamide

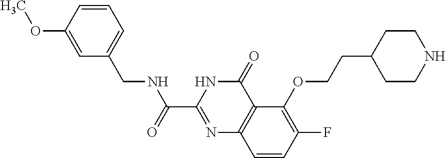

### Step 1: Preparation of tert-butyl 4-{2-[(6-fluoro-2-{[(3-methoxybenzyl)amino]carbonyl}-4-oxo-3,4-dihydroquinazolin-5-yl)oxy]ethyl}piperidine-1-carboxylate

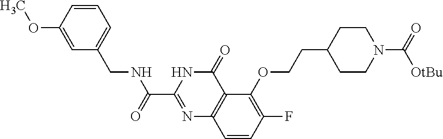

To a dry flask under an atmosphere of argon was added sodium hydride (360 mg, 60% dispersion in oil) and the flask was flushed with argon. The flask was charged with 6 mL of N,N-dimethylacetamide and flushed with argon. After stirring for 5 min at room temperature, a solution of N-boc-4-piperidineethanol (1.03 g, 4.5 mmol) in 3 mL of N,N-dimethylacetamide was added via syringe. The mixture was stirred for 5 min at room temperature and a solution of N-(3-methoxybenzyl)-5,6-difluoro-4-oxo-3,4-dihydroquinazoline-2-carboxamide (prepared as described in step 5 of the synthesis of 6-fluoro-5-{[(trans-2,5)-5-(hydroxymethyl)-1,4-dioxan-2-yl]methoxy}-N-(3-methoxybenzyl)-4-oxo-3,4-dihydroquinazoline-2-carboxamide, Example 3) (518 mg, 1.5 mmol) in 3 mL of N,N-dimethylacetamide was added via syringe and the reaction was heated to 60° C. After 60 min, the reaction was cooled to room temperature, the pH was lowered to ˜4 with 1M hydrochloric acid, and then diluted with ethyl acetate (50 mL). The organic layer was washed with water (20 mL), brine (20 mL), dried over magnesium sulfate, filtered, and evaporated to afford a solid. The solid was purified using normal phase chromatography (1:1 hexane:dichloromethane/ethyl acetate mixtures) to afford the title compound as a clear glass (0.9 g). LC/MS 15-95% acetonitrile/tfa-water/tfa (5 min gradient) 4.2 min, m/z 555 (M+H). ¹H NMR (400 MHz, DMSO-d₆) δ ppm 11.99 (1H, br. s.), 9.43 (1H, t), 7.79 (1H, t), 7.49-7.57 (1H, m), 7.20-7.28 (1H, m), 6.87-6.97 (2H, m), 6.78-6.87 (1H, m), 4.45 (2H, d, J=6.2 Hz), 4.11 (2H, t), 3.83-4.00 (2H, m), 3.74 (3H, s), 2.71 (2H, br. s.), 1.65-1.84 (5H, m), 1.39 (9H, s), 0.90-1.13 (2H, m).

### Step 2: Preparation of 6-fluoro-N-(3-methoxybenzyl)-4-oxo-5-(2-piperidin-4-ylethoxy)-3,4-dihydroquinazoline-2-carboxamide

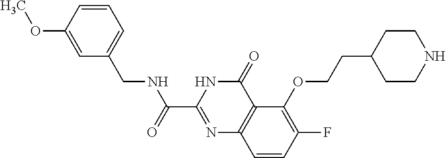

To a solution of tert-butyl 4-{2-[(6-fluoro-2-{[(3-methoxybenzyl)amino]carbonyl}-4-oxo-3,4-dihydroquinazolin-5-yl)oxy]ethyl}piperidine-1-carboxylate (0.9 g, 1.62 mmol) in 6 mL of dichloromethane was added 2 mL of trifluoroacetic acid. The reaction was stirred at room temperature for 30 min and evaporated to dryness. The residue was dissolved in 1 mL of dichloromethane and precipitated in 5 mL of ethyl ether. The supernatant was decanted and the residual solvents were removed in vacuo to afford the trifluoroacetate salt of title compound as a white solid (740 mg). LC/MS 15-95% acetonitrile/tfa-water/tfa (5 min gradient) 2.4 min, m/z 455 (M+H). ¹H NMR (400 MHz, DMSO-d₆) δ ppm 9.50 (1H, t, J=6.4 Hz), 7.82 (1H, dd, J=10.2, 9.1 Hz), 7.49-7.60 (1H, m), 7.24 (1H, t, J=8.1 Hz), 6.86-6.96 (2H, m), 6.79-6.86 (1H, m), 4.44 (2H, d, J=6.2 Hz), 4.10 (2H, t, J=6.3 Hz), 3.73 (3H, s), 3.18-3.37 (2H, m), 2.77-2.99 (2H, m), 1.82-2.03 (3H, m), 1.62-1.80 (2H, m), 1.18-1.44 (2H, m).

### Example 31

### 6-fluoro-N-(3-methoxybenzyl)-5-{2-[1-(methylsulfonyl)piperidin-4-yl]ethoxy}-4-oxo-3,4-dihydroquinazoline-2-carboxamide

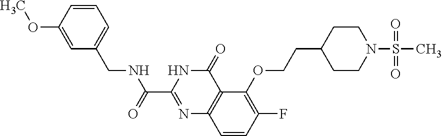

To a suspension of the trifluoroacetate salt of 6-fluoro-N-(3-methoxybenzyl)-4-oxo-5-(2-piperidin-4-ylethoxy)-3,4-dihydroquinazoline-2-carboxamide (prepared as described in Example 30) (150 mg, 0.33 mol) in 4 mL of dichloromethane were added triethylamine (0.146 mL, 1.05 mmol) and methanesulfonyl chloride (0.031 mL, 0.4 mmol). The reaction was stirred at room temperature for 0.5 h and then diluted with dichloromethane (50 mL). The mixture was washed with saturated sodium bicarbonate (20 mL). The organic layer was dried over magnesium sulfate and concentrated to afford an off-white solid (161 mg). The solid was purified using reverse phase chromatography to afford the title compound. LC/MS 15-95% acetonitrile/tfa-water/tfa (5 min gradient) 3.5 min, m/z 533 (M+H). ¹H NMR (400 MHz, DMSO-d₆) δ ppm 9.40 (1H, t, J=5.9 Hz), 7.76 (1H, t, J=9.8 Hz), 7.44-7.55 (1H, m), 7.20 (1H, t, J=8.1 Hz), 6.83-6.92 (2H, m), 6.79 (1H, d, J=7.5 Hz), 4.41 (2H, d, J=6.2 Hz), 4.08 (2H, t, J=5.0 Hz), 3.70 (3H, s), 3.51 (2H, d, J=11.9 Hz), 2.80 (3H, s), 2.58-2.74 (2H, m), 1.82 (2H, d, J=12.4 Hz), 1.70 (3H, br. s.), 1.11-1.29 (2H, m).

### Example 32

### 5-[2-(1-acetylpiperidin-4-yl)ethoxy]-6-fluoro-N-(3-methoxybenzyl)-4-oxo-3,4-dihydroquinazoline-2-carboxamide

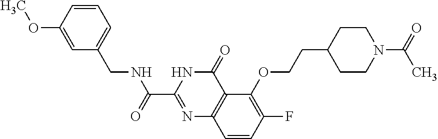

To a suspension of the trifluoroacetate salt of 6-fluoro-N-(3-methoxybenzyl)-4-oxo-5-(2-piperidin-4-ylethoxy)-3,4-dihydroquinazoline-2-carboxamide (prepared as described in Example 30) (188 mg, 0.33 mol) in 4 mL of dichloromethane were added triethylamine (0.146 mL, 1.05 mmol), 4-(dimethylamino)pyridine (8 mg, 0.2 mmol), and acetyl chloride (0.023 mL, 0.32 mmol). The reaction was stirred at room temperature for 0.5 h and then diluted with dichloromethane (50 mL). The mixture was washed with saturated sodium bicarbonate (20 mL). The organic layer was dried over magnesium sulfate and concentrated to afford an off-white solid (141 mg). The solid was purified using reverse phase chromatography to afford the title compound. LC/MS 15-95% acetonitrile/tfa-water/tfa (5 min gradient) 3.2 min, m/z 497 (M+H). ¹H NMR (400 MHz, DMSO-d₆) δ ppm 9.40 (1H, t, J=6.4 Hz), 7.75 (1H, t, J=9.9 Hz), 7.45-7.55 (1H, m), 7.20 (1H, t, J=8.1 Hz), 6.83-6.92 (2H, m), 6.74-6.83 (1H, m), 4.41 (2H, d, J=6.2 Hz), 4.26-4.36 (1H, m), 4.08 (2H, t, J=6.3 Hz), 3.72-3.81 (1H, m), 3.70 (3H, s), 2.90-3.02 (1H, m), 1.94 (3H, s), 1.60-1.87 (5H, m), 0.83-1.13 (2H, m).

### Example 33

### 2-(4-{2-[(6-fluoro-2-{[(3-methoxybenzyl)amino]carbonyl}-4-oxo-3,4-dihydroquinazolin-5-yl)oxy]ethyl}piperidin-1-yl)-2-oxoethyl acetate

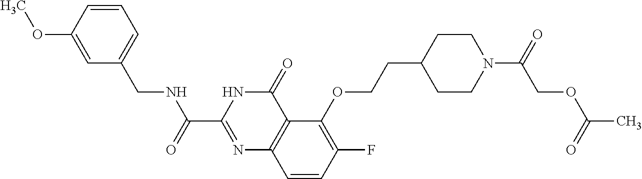

To a suspension of the trifluoroacetate salt of 6-fluoro-N-(3-methoxybenzyl)-4-oxo-5-(2-piperidin-4-ylethoxy)-3,4-dihydroquinazoline-2-carboxamide (prepared as described in Example 30) (148 mg, 0.26 mmol) in 4 mL of dichloromethane were added triethylamine (0.146 mL, 1.05 mmol), 4-(dimethylamino)pyridine (6.4 mg, 0.2 mmol), and acetoxyacetyl chloride (0.035 mL, 0.33 mmol). The reaction was stirred at room temperature for 0.5 h and then diluted with dichloromethane (50 mL). The mixture was washed with saturated sodium bicarbonate (20 mL). The organic layer was dried over magnesium sulfate and concentrated to afford the title compound as an off-white solid (145 mg). LC/MS 15-95% acetonitrile/tfa-water/tfa (5 min gradient) 3.3 min, m/z 555 (M+H). ¹H NMR (400 MHz, DMSO-d₆) δ ppm 9.46 (1H, t, J=6.4 Hz), 7.77 (1H, dd, J=10.3, 9.0 Hz), 7.45-7.57 (1H, m), 7.21 (1H, t, J=8.2 Hz), 6.84-6.93 (2H, m), 6.75-6.84 (1H, m), 4.72 (2H, d, J=1.3 Hz), 4.41 (2H, d, J=6.2 Hz), 4.25 (1H, d, J=13.2 Hz), 4.07 (2H, t, J=6.3 Hz), 3.70 (3H, s), 3.66 (1H, d, J=13.7 Hz), 2.95 (1H, t, J=12.9 Hz), 2.55 (1H, t, J=11.7 Hz), 2.04 (3H, s), 1.57-1.90 (5H, m), 0.86-1.22 (2H, m).

### Example 34

### 6-fluoro-5-[2-(1-glycoloylpiperidin-4-yl)ethoxy]-N-(3-methoxybenzyl)-4-oxo-3,4-dihydroquinazoline-2-carboxamide

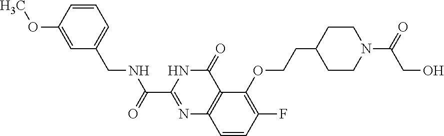

To a solution of 2-(4-{2-[(6-fluoro-2-{[(3-methoxybenzyl)amino]carbonyl}-4-oxo-3,4-dihydroquinazolin-5-yl)oxy]ethyl}piperidin-1-yl)-2-oxoethyl acetate (prepared as described in Example 33) (131 mg, 0.24 mmol) in 4 mL tetrahydrofuran was added 1M lithium hydroxide (0.71 mL, 0.71 mmol). The reaction was stirred for 1 h at room temperature. The pH was adjusted to ˜7 with 1M potassium hydrogen sulfate and the solvent was removed in vacuo. The mixture was extracted with ethyl acetate (3×15 mL). The combined organic layers were dried over magnesium sulfate, filtered, and evaporated to afford a white solid. The solid was purified using reverse phase chromatography to afford the title compound (106 mg). LC/MS 15-95% acetonitrile/tfa-water/tfa (5 min gradient) 3.1 min, m/z 513 (M+H). ¹H NMR (400 MHz, DMSO-d₆) δ ppm 9.39 (1H, t, J=6.3 Hz), 7.75 (1H, t, J=9.8 Hz), 7.43-7.55 (1H, m), 7.20 (1H, t, J=8.1 Hz), 6.83-6.94 (2H, m), 6.79 (1H, d, J=8.2 Hz), 4.41 (2H, d, J=6.2 Hz), 4.23-4.37 (2H, m), 3.94-4.13 (4H, m), 3.70 (3H, s), 3.61 (1H, d, J=12.8 Hz), 2.83-2.98 (1H, m), 2.58 (1H, t, J=11.6 Hz), 1.59-1.90 (5H, m), 0.89-1.20 (2H, m).

### Example 35

### 6-fluoro-5-{2-[1-(2-hydroxyethyl)piperidin-4-yl]ethoxy}-N-(3-methoxybenzyl)-4-oxo-3,4-dihydroquinazoline-2-carboxamide

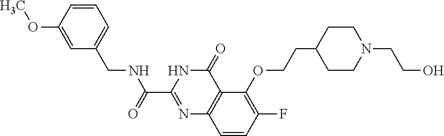

To a suspension of the trifluoroacetate salt of 6-fluoro-N-(3-methoxybenzyl)-4-oxo-5-(2-piperidin-4-ylethoxy)-3,4-dihydroquinazoline-2-carboxamide (prepared as described in Example 30) (400 mg, 0.70 mmol) in 8 mL ethanol was added triethylamine (0.39 mL, 2.81 mmol) followed by bromoethanol (0.2 mL, 2.81 mmol) and the reaction was refluxed for 16 h. The reaction was diluted with 20 mL tetrahydrofuran and PS-thiophenol resin (4.2 g@1.47 mmol/g) was added. The mixture was stirred at room temperature for 16 h. The resin was filtered, washed with tetrahydrofuran (50 mL) and ethanol (25 mL). The filtrate was evaporated and the residue was partitioned between ethyl acetate (50 mL) and water (10 mL). The aqueous layer was extracted with ethyl acetate (2×50 mL). The combined organic layer was washed with 1M sodium hydroxide (50 mL), brine (50 mL), dried over magnesium sulfate, filtered, and evaporated to afford a solid. The solid was purified using reverse phase chromatography. Combined fractions were passed through carbonate cartridges (StrataSpheres™ SPE PL-HCO₃ MP) and evaporated to afford the title compound as an off-white foam (102 mg). LC/MS 5-100% acetonitrile/tfa-water/tfa (4 min gradient) 2.9 min, m/z 499 (M+H). ¹H NMR (400 MHz, DMSO-d₆) δ ppm 9.40 (1H, t, J=6.3 Hz), 7.75 (1H, dd, J=10.4, 9.1 Hz), 7.44-7.54 (1H, m), 7.22 (1H, t, J=8.1 Hz), 6.85-6.93 (2H, m), 6.75-6.85 (1H, m), 4.42 (2H, d, J=6.2 Hz), 4.07 (2H, t, J=6.5 Hz), 3.71 (3H, s), 3.46 (2H, t, J=6.3 Hz), 2.82 (2H, d, J=11.5 Hz), 2.35 (2H, t, J=6.3 Hz), 1.86-2.01 (2H, m), 1.60-1.76 (4H, m), 1.43-1.61 (1H, m), 1.07-1.25 (2H, m).

### Example 36

### 5-{[(trans 2,5)-5-(aminomethyl)-1,4-dioxan-2-yl]methoxy}-6-fluoro-N-(3-methoxybenzyl)-4-oxo-3,4-dihydroquinazoline-2-carboxamide

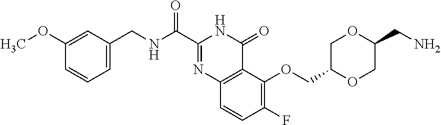

### Step 1: Preparation of [(trans 2,5)-5-{[(6-fluoro-2-{[(3-methoxybenzyl)amino]carbonyl}-4-oxo-3,4-dihydroquinazolin-5-yl)oxy]methyl}-1,4-dioxan-2-yl]methyl 4-methylbenzenesulfonate

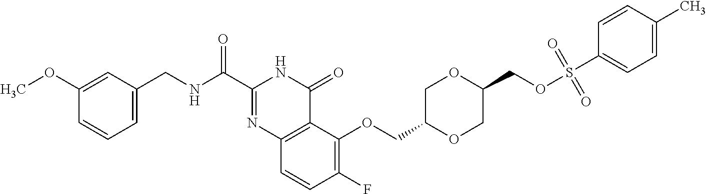

To a solution of 6-fluoro-5-{[(trans-2,5)-5-(hydroxymethyl)-1,4-dioxan-2-yl]methoxy}-N-(3-methoxybenzyl)-4-oxo-3,4-dihydroquinazoline-2-carboxamide (prepared as described in Example 3) (820 mg, 1.73 mmol) in 8 mL pyridine was added 4-(dimethylamino)pyridine (21 mg, 0.17 mmol). A solution of tosyl chloride (495 mg, 2.6 mmol) in 2 mL pyridine was added and the reaction was stirred at room temperature for 16 h. Water (20 mL) was added followed by ethyl acetate (20 mL). The organic layer was washed with brine (10 mL), dried over magnesium sulfate, and evaporated to afford light yellow solid (1.06 g). The solid was crystallized from methanol to afford a light yellow solid (200 mg). LC/MS 15-95% acetonitrile/tfa-water/tfa (5 min gradient) 4.0 min, m/z 628 (M+H). ¹H NMR (400 MHz, DMSO-d₆) δ ppm 12.14 (1H, br. s.), 9.49 (1H, t, J=6.3 Hz), 7.72-7.89 (3H, m), 7.42-7.62 (3H, m), 7.24 (1H, t, J=8.1 Hz), 6.86-6.98 (2H, m), 6.82 (1H, dd, J=9.1, 1.6 Hz), 4.44 (2H, d, J=6.4 Hz), 3.86-4.11 (4H, m), 3.56-3.87 (7H, m), 3.46-3.55 (1H, m), 3.25-3.33 (1H, m), 2.43 (3H, s).

### Step 2: Preparation of 5-{[(trans 2,5)-5-(aminomethyl)-1,4-dioxan-2-yl]methoxy}-6-fluoro-N-(3-methoxybenzyl)-4-oxo-3,4-dihydroquinazoline-2-carboxamide

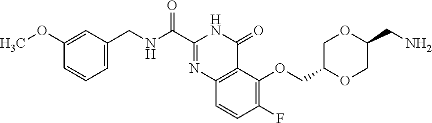

To a solution of [(trans-2,5)-5-{[(6-fluoro-2-{[(3-methoxybenzyl)amino]carbonyl}-4-oxo-3,4-dihydroquinazolin-5-yl)oxy]methyl}-1,4-dioxan-2-yl]methyl 4-methylbenzenesulfonate (210 mg, 0.34 mmol) in dimethylsulfoxide was added concentrated ammonium hydroxide (0.15 mL). The reaction was stirred at 80° C. for 2 days. Water (10 mL) was added and then the reaction mix was extracted with ethyl acetate (3×10 mL). The combined organic layer was dried over magnesium sulfate, filtered, and evaporated to afford a light yellow solid (169 mg). The solid was purified using reverse phase chromatography to afford the title compound. LC/MS 15-95% acetonitrile/tfa-water/tfa (5 min gradient) 2.3 min, m/z 473 (M+H). ¹H NMR (400 MHz, DMSO-d₆) δ ppm 9.36 (1H, t, J=5.7 Hz), 7.73 (1H, dd, J=10.2, 9.1 Hz), 7.44-7.58 (1H, m), 7.24 (1H, t, J=8.1 Hz), 6.87-6.99 (2H, m), 6.77-6.87 (1H, m), 4.44 (2H, d, J=5.9 Hz), 4.05-4.16 (1H, m), 3.95-4.05 (2H, m), 3.77-3.88 (2H, m), 3.74 (3H, s), 3.51 (1H, t, J=10.9 Hz), 3.36-3.47 (1H, m), 3.28 (1H, t, J=10.9 Hz), 2.58 (2H, d, J=5.7 Hz).

### In Vitro MMP Inhibition Analysis

Matrix Metalloproteinase inhibitor compounds were analyzed in in vitro MMP Inhibition assays to determine their ability to inhibit the MMP cleavage of peptide substrates. Inhibition constants (Kᵢ) were calculated from the assayed compound-MMP interactions.

Human recombinant MMP-1, MMP-2, MMP-3, MMP-7, MMP-9, MMP-12, MMP-13, MMP-14 and MMP-15 were used in these assays. The enzymes are prepared following known laboratory procedures. Protocols for the preparation and use of these enzymes are available in the scientific literature. See, e.g., Enzyme Nomenclature (Academic Press, San Diego, Calif., 1992) and the citations therein. See also Frije et al., J. Biol. Chem., 26(24), 16766-73 (1994).

In addition, many MMPs may be purchased from suppliers. For example, MMP-1, MMP-2, MMP-3, MMP-7, MMP-8, MMP-9, MMP-10, MMP-11, MMP-12, MMP-13, MMP-14, MMP-15, MMP-16, MMP-24, MMP-25, and MMP-26 are commercially available from R&D Systems in their 2006 catalog. Available in the 2006 Millipore Chemicon catalog are MMP-1, MMP-2, MMP-3, MMP-7, MMP-8, MMP-9, MMP-13, MMP-14, MMP-15, MMP-16, MMP-17 and MMP-24.

The MMP-1 proenzyme may be purified from spent media of MMP-1-transfected HT-1080 cells and the protein purified on a zinc chelating column. The MMP-2 proenzyme may be purified by gelatin Sepharose chromatography from MMP-2-transfected p2AHT2 cells. The MMP-9 proenzyme may be purified by gelatin Sepharose chromatography from spent media of MMP-9-transfected HT1080 cells.

The catalytic domain MMP-3 cDNA may be used to express the catalytic domain enzyme in E. coli inclusion bodies. Then the enzyme is solubilized in urea, purified on a preparative C-14 reverse phase HPLC column, and refolded in the presence of zinc acetate and purified for use.

The MMP-7 cDNA may be used to express the enzyme in E. coli inclusion bodies. Then the enzyme is solubilized in urea, purified on a preparative C-14 reverse phase HPLC column, and refolded in the presence of zinc acetate and purified for use.

The MMP-13 may be obtained as a proenzyme from a full-length cDNA clone using baculovirus expression, as described by V. A. Luckow, “Insect Cell Expression Technology,” Protein Engineering: Principles and Practice, pp. 183-218 (edited by J. L. Cleland et al., Wiley-Liss, Inc., 1996). The expressed proenzyme was first purified over a heparin agarose column, and then over a chelating zinc chloride column. Further details on baculovirus expression systems may be found in, for example, Luckow et al., J. Virol., 67, 4566-79 (1993). See also O'Reilly et al, Baculovirus Expression Vectors: A Laboratory Manual (W. H. Freeman and Co., New York, N.Y., 1992). See also King et al., The Baculovirus Expression System: A Laboratory Guide (Chapman & Hall, London, England, 1992).

The MMP-14 cDNA may be used to express the catalytic domain enzyme in E. coli inclusion bodies. Then the enzyme is solubilized in urea, purified on a preparative C-14 reverse phase HPLC column, and refolded in the presence of zinc acetate and purified for use.

The catalytic domain of MMP-12 and MMP-15 enzymes were purchased commercially for these assays.

All full length MMPs were activated using 4-aminophenylmercuric acetate (“APMA”, Sigma Chemical, St. Louis, Mo.) or trypsin. MMP-9 also was activated using human recombinant MMP-3 following standard cloning and purification techniques.

The fluorogenic, methoxycoumarin-containing polypeptide substrate MCA-ArgProLeuGlyLeuDpaAlaArgGluArgNH₂ was used as the MMP substrate in the MMP inhibition assays for human recombinant MMP-1, MMP-2, MMP-7, MMP-9, MMP-12, MMP-13, MMP-14 and MMP-15. Here, “MCA” is 7-methoxycoumarin-4-yl acetyl and “Dpa” is 3-(2,4-dinitrophenyl)-L-2,3-di-aminopropionyl group. In the absence of MMP inhibitory activity, the substrate is cleaved at the Gly-Leu peptide bond. The cleavage separates the highly fluorogenic peptide from the 2,4-dinitrophenyl quencher, resulting in an increase of fluorescent intensity.

For the human MMP-1, MMP-2, MMP-7, MMP-9, MMP-12, MMP-13, MMP-14 and MMP-15 assays, dilutions of the inhibitors (or salts thereof) were prepared in 100% dimethyl sulfoxide (DMSO). The stock solutions were diluted in Buffer A (100 mM Tris-HCl, 100 mM NaCl, 10 mM CaCl₂, 0.005% polyoxyethylene 23 lauryl ether, pH 7.5) to obtain solutions with different compound concentrations, i.e., assay solutions with different concentrations of the assayed MMP inhibitory compound in 1% DMSO. The experiment controls contained the same amount of Buffer A/DMSO as the assayed sample, but contained no inhibitor.

The fluorogenic, methoxycoumarin-containing polypeptide substrate Mca-Arg-Pro-Lys-Pro-Val-Glu-Nva-Trp-Arg-Lys(Dnp)-NH₂ (Bachem catalog number M-2110) was used as the MMP substrate in the MMP inhibition assays for catalytic domain of human recombinant MMP-3 (which refers to the catalytic domain of stromelysin). Here, “MCA” is 7-methoxycoumarin-4-yl acetyl and “Dpa” is 3-(2,4-dinitrophenyl)-L-2,3-di-aminopropionyl group. In the absence of MMP inhibitory activity, the substrate is cleaved at the Gly-Leu peptide bond. The cleavage separates the highly fluorogenic peptide from the 2,4-dinitrophenyl quencher, resulting in an increase of fluorescent intensity.

For the human MMP-3 assay, dilutions of the inhibitors (or salts thereof) were prepared in 100% DMSO. The stock solutions were diluted in Buffer B (50 mM N-morpholinoethane sulfonate (“MES”), 100 mM NaCl, 10 mM CaCl₂, 0.005% polyoxyethylene 23 lauryl ether, pH 6.0)) to obtain solutions with different compound concentrations, i.e., assay solutions with different concentrations of the assayed MMP inhibitory compound in 1% DMSO. The experiment controls contained the same amount of Buffer B/DMSO as the assayed sample, but contained no inhibitor.

To determine Kᵢ, the inhibitor samples are incubated at room temperature for 1 hr in the presence of enzyme and then 4 μM of appropriate MMP substrate was added, and samples were analyzed on a Tecan SpectraFlour Plus plate reader. The excitation wavelength is 330 nm, and the emission (fluorescence) wavelength is 420 nm. In the absence of MMP inhibitory activity, the substrate is cleaved at the Gly-Leu bond resulting in an increase of relative fluorescence. Inhibition is observed as a reduced rate of increase in relative fluorescence.

The inhibitors are analyzed using a single low enzyme concentration with a single substrate concentration fixed at or below the Kₘ. This protocol is a modification of method by Knight et al., FEBS Lett., 296(3), 263-266 (1992). Apparent inhibitory constants are determined by non-linear regression of reaction velocity as a function of inhibitor and enzyme concentration using Morrison's equation, as described by Kuzmic, Anal. Biochem. 286, 45-50 (2000). Modifications were made in the non-linear regression method to allow a common control reaction rate and effective enzyme concentration to be shared between all dose-response relationships on a given assay plate. Since the substrate concentration was chosen to be at or below the Kₘ, the apparent Kᵢ's from this analysis were reported as Kᵢ's without correction for the influence of substrate.

| | hMMP Kᵢ (nM) | | | | | | | | |
|---|---|---|---|---|---|---|---|---|---|
| Example No. | 01/FL | 02/FL | 03/CD | 07/CD | 09/FL | 12/CD | 13/FL | 14/CD | 15/CD |
| 1 | | | | | | | 13.0 | | |
| 3 | | | 8580 | | | | 16.4 | | |
| 4 | | | 337 | | | | 0.471 | | |
| 5 | | | 389 | | | | 0.221 | | |
| 6 | | | 468 | | | | 0.747 | | |
| 7 | | | 1640 | | | >10000 | 11.4 | >10000 | >10000 |
| 8 | | | 1310 | | | | 4.16 | | |
| 9 | | | 1350 | | | >10000 | 0.88 | >10000 | >10000 |
| 10 | | | 163 | | | | 0.143 | | |
| 11 | | | 663 | | | | 11.0 | | |
| 12 | | | 668 | | | | 1.62 | | |
| 13 | | | 258 | | | | 0.141 | | |
| 14 | | | 350 | | | | 0.479 | | |
| 15 | | | 960 | | | | 4.04 | | |
| 16 | | | 533 | | | | 0.41 | | |
| 17 | | | 656 | | | | 1.62 | | |
| 18 | | | 1000 | | | >10000 | 0.732 | >10000 | >10000 |
| 19 | | | 1220 | | | >10000 | 2.48 | >10000 | >10000 |
| 21 | | | 672 | | | | 1.25 | | |
| 23 | | | 453 | | | | 0.582 | | |
| 25 | | | 512 | | | | 0.566 | | |
| 26 | | | | | | | 9.28 | | |
| 27 | | | 779 | | | | 1.78 | | |
| 28 | | | 787 | | | | 3.13 | | |
| 29 | | | 1600 | | | | 0.374 | | |
| 30 | | | 9420 | | | | 1.91 | | |
| 31 | | | 1280 | | | | 0.085 | | |
| 32 | | | 1950 | | | | 0.184 | | |
| 34 | >10000 | 5650 | 1570 | >10000 | >10000 | 6530 | 0.118 | >10000 | >10000 |
| 35 | | | 5200 | | | | 4.15 | | |
| 36 | | | | | | | 19.9 | | |

### Cartilage Damage In Vitro Assay

Human articular cartilage (HAC) was dissected from knees obtained from Asterand from patients undergoing knee replacement. Apparently normal (not fibrillated) cartilage was cut in small pieces (˜2 mm) and cultured (basically as in Billinghurst et al 1997 or Dahlberg 2000, with some modifications) in 96 well plates with 200 μl of DMEM media (Gibco BRL high glucose, 25 mM Hepes, containing 2 mM L-glutamine and 1 mM Sodium Pyruvate) freshly supplemented with 1×HL-1 (Bio Whitaker) and 5 μg/ml ascorbic acid (Sigma), and with or without 0.1 ng/ml IL-1β (R &D Systems, Minneapolis, Minn.)+50 μg/ml Oncostatin M (R & D Systems). Some cartilage wells were incubated in the presence of serially diluted MMP inhibitors, tested in quadruplicate at 6 concentrations. Media were replaced every 3 to 5 days and cartilage was cultured for a total of 18-22 days. Conditioned media were frozen and analyzed later for hydroxyproline content (or type II collagen degradation biomarker TIINE).

### TIINE Sandwich Immunoassay for Culture Media.

A chemiluminescent sandwich immunoassay was developed with the neoepitope 9A4 antibody (recognizing the conserved sequence at the C-terminus of the ¾ type II collagen fragment GPPGPQG following collagenase cleavage) and the capture 5109 antibody (recognizing the type II collagen specific epitope GEPGDDGPS) as detailed elsewhere (Nemirovskiy et al., Anal. Biochem. 2007; 361(1):93-101) utilizing the proprietary, chemiluminescent Bioveris technology (Bioveris Corporation, Gaithersburg, Md.). This immunoassay utilizes a sandwich format on a paramagnetic bead support phase in which the sandwich complex is bound to the bead in suspension, passed through a flow cell, and captured by a magnet. The process effectively separates the analyte from the sample, decreasing background interference and eliminating wash steps required in other formats. Briefly, serially diluted culture supernatant samples were assayed in a 96-well plate. In this one step assay, 25 μL of sample, 25 μL of streptavidin beads (0.4 mg/mL), 25 μL of each antibody at 1 μg/mL (Biotin conjugated mouse 5109 anti-capture and BVTAG labeled 9A4 anti-neoepitope) and 100 μL of assay buffer (DPBS, 0.1% BSA, 0.05% Tween 20, pH 7.4) were incubated for 2 hours at room temperature before reading on the Bioveris M384 Analyzer. Values were calculated from a standard curve prepared from either human 45-mer or bovine 30-mer (0.14-100 ng/ml) neoepitope peptide.

Cartilage Damage and/or Joint Degeneration In Vivo Assay: Surgical-Induce Medial Meniscus Tear Rat OA Model

Adult male Sprague Dawley rats (Charles River, Wilmington, Mass.) weighing 275-300 g were allowed to acclimate for 1 week prior to surgery. OA was surgically induced as described (Bendele, “Animal models of osteoarthritis,” J. Musculoskelet. Neuronal. Interact. 1 (2001) (4), pp. 363-376). Briefly, animals were anesthetized with isoflurane and the right knee joint prepped for surgery. The medial collateral ligament (MCL) was exposed by blunt dissection and transected to reflect the meniscus toward the femur. The joint space was visualized and the meniscus was cut through the full thickness at its narrowest point to simulate a complete tear. The skin was closed with stainless steel staples. Compound or vehicle treatment started the day of surgery and continued for 3 to 4 weeks. The animals are then sacrificed, the femoro-tibial joint space lavaged with 100 ul sterile saline to collect synovial fluid, and the total joint removed and fixed in 10% formalin for histology. Scoring was performed by hispopathology quantification.

1. A compound of Formula I: 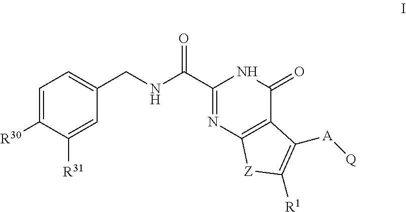
wherein:
Z is —CH═CH— or —S—;
A is —(CH₂)ₘO(CH₂)ₙ, wherein m is 0, 1, 2, or 3 and n is 1, 2, or 3;
Q is 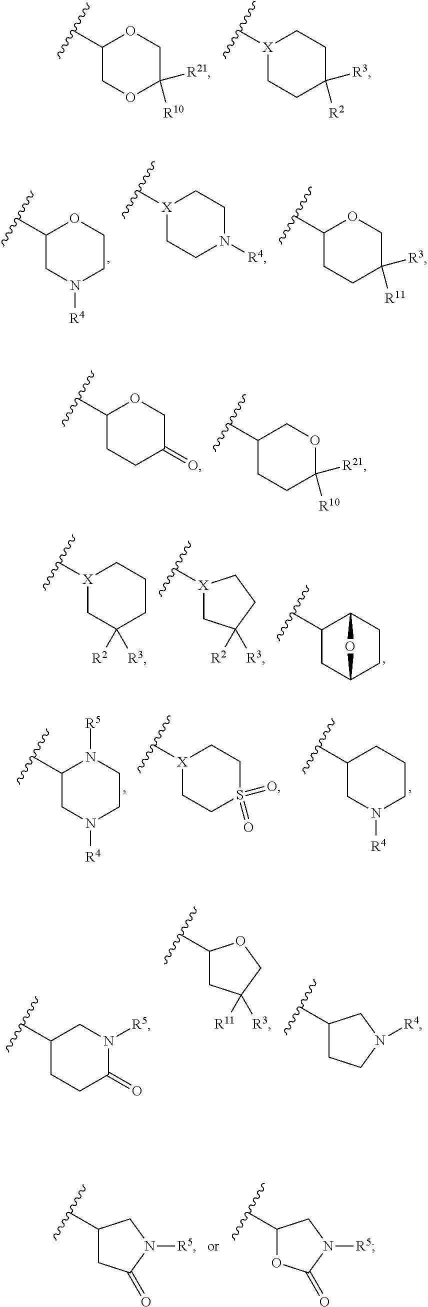
X is N or CH, provided that when X is N, n is 2 or 3;
R¹ is H or F;
R² is H, CN, —OR⁵, R¹², C(═O)R⁷, —NR⁸R³³, —NR⁸C(═O)R⁹, —NR⁸SO₂R³⁴, or —SO₂R¹²;
R³ is H, CN, or —OR²²;
R⁴ is H, —(C₁₋₆ alkylene)R⁶, —C(═O)R⁹, or —SO₂R¹²;
R⁵ is H or —(C₁₋₆ alkyl), wherein said C₁₋₆ alkyl may be substituted by one or more R²⁶ substituents;
R⁶ is H, CN, —OR²³, —SO₂R³⁶, —NR²⁴C(═O)R²³, —NR²⁴SO₂R³⁵, or 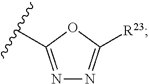
R⁷ is —(C₁₋₆ alkyl), —(C₁₋₆ alkylene)OH, —NHR²⁴, or —OR²⁵;
R⁹ is —(C₁₋₆ alkylene)R²⁸, —NHR²⁴, or —OR²⁵;
R¹⁰ is H, CN, R¹², or —C(═O)R⁷;
R¹¹ is H, CN, —OR⁵, R¹², —C(═O)R⁷, —NR⁸R³³, —NR⁸C(═O)R⁹, —NR⁸SO₂R³⁴, or —SO₂R¹²;
R¹² is —(C₁₋₆ alkyl), wherein said C₁₋₆ alkyl may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of said C₁₋₆ alkyl is not substituted by more than one CN or more than one —OR²³;
R⁸, R²¹, R²², R²⁴, R²⁵, and R³³ are independently H or —(C₁₋₆ alkyl);
R²³ is H, —(C₁₋₆ alkyl), or —(C₁₋₆ alkylene)OH;
R²⁶ is H, OH, halo, NH₂, or SH;
R²⁸ is H or —OR²⁹;
R²⁹ is H or —C(═O)(C₁₋₆ alkyl);
R³⁰ is H or F;
R³¹ is Cl, Br, —OR³², (C₁₋₆ alkyl), —OCH₂CH₂OR²⁵, —(C₃₋₆ cycloalkyl), or CN;
R³² is —(C₁₋₆ alkyl) optionally substituted with one, two, or three F;
R³⁴ is —(C₁₋₆ alkyl); and
R³⁵ is —(C₁₋₆ alkyl) or —(C₁₋₆ hydroxyalkyl);
or a pharmaceutically acceptable salt thereof;
provided that if Q is 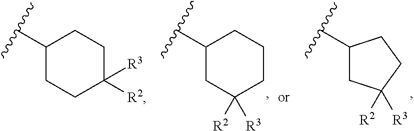
R² and R³ are not both H.

2. A compound according to claim 1 wherein Q is

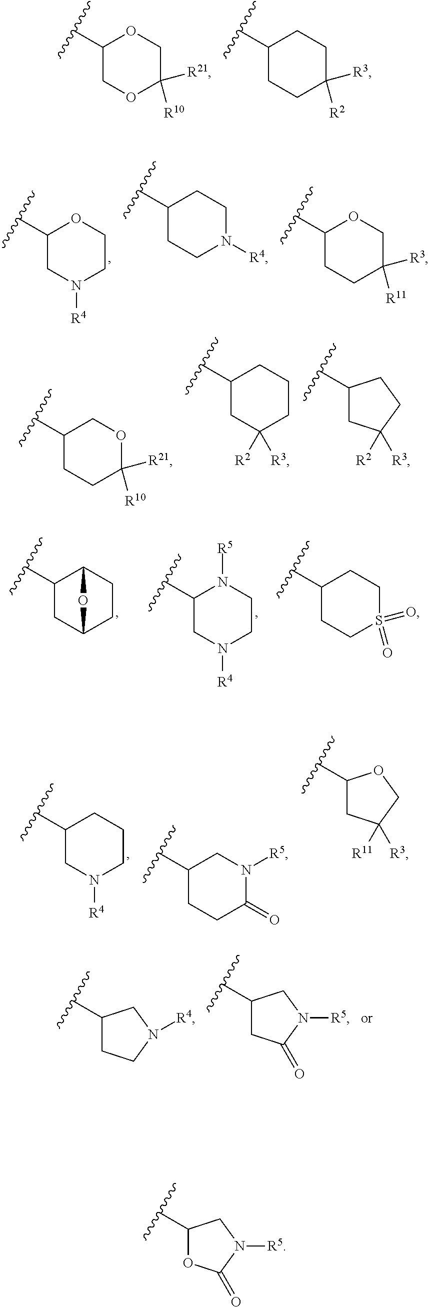

3. A compound according to claim 1 or 2 wherein:
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸, R²¹, R²², R²⁴, R²⁵, and R³³ are independently H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁶ is H, OH, F, Cl, Br, NH₂, or SH;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

4. A compound according to claim 1 of Formula II:

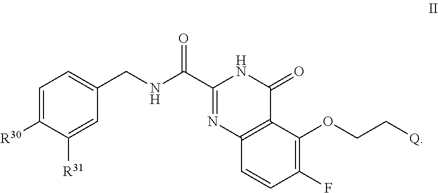

5. A compound according to claim 4 wherein Q is

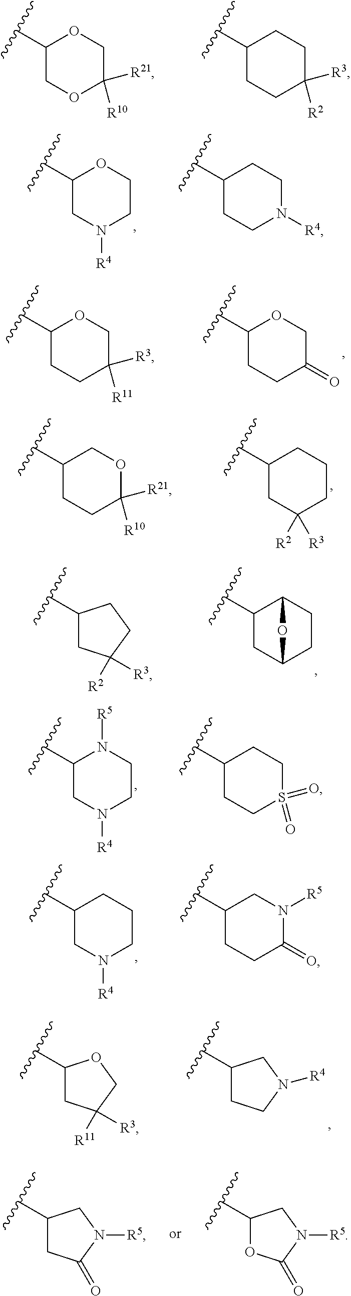

6. A compound according to claim 5 wherein Q is

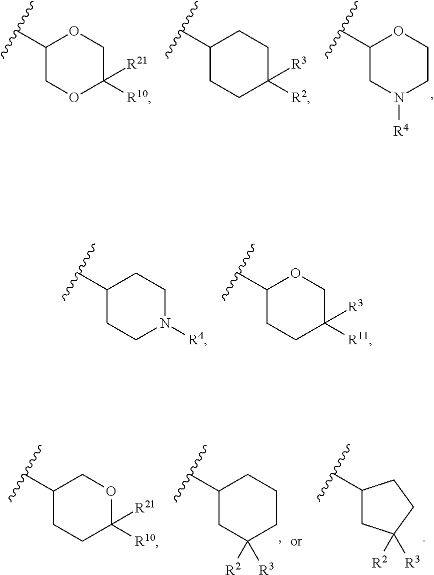

7. A compound according to claim 6 wherein Q is

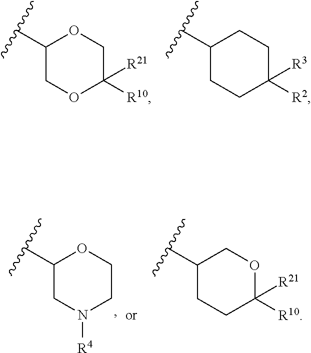

8. A compound according to claim 7 wherein Q is

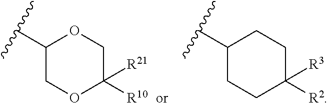

9. A compound according to claim 4, 5, 6, 7, or 8 wherein
R¹⁰ is CN, R¹², or —C(═O)R⁷; and
R¹¹ is CN, —OR¹², R¹², —C(═O)R⁷, —NR⁸C(═O)R⁹, —NR⁸SO₂R³⁴, or —SO₂R¹².

10. A compound according to claim 5 wherein
Q is 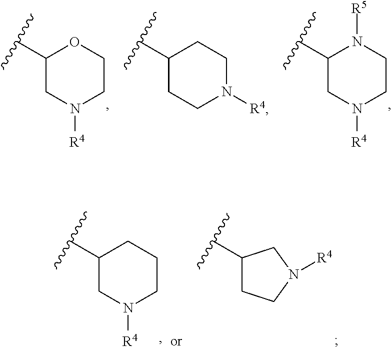
and
R⁴ is —C(═O)R⁹ or —SO₂R¹².

11. A compound according to claim 5 wherein
Q is

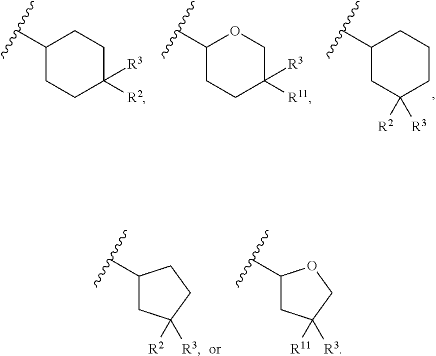

12. A compound according to claim 5 wherein
Q is 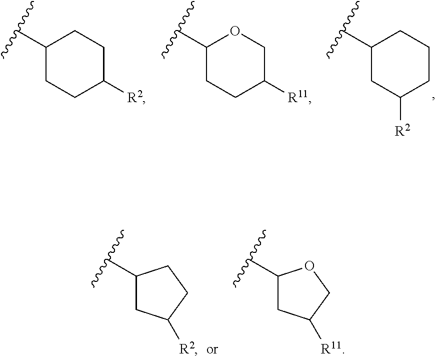
R² is CN, OH, R¹², —C(═O)R⁷, —NR⁸R³³, —NR⁸C(═O)R⁹, or —NR⁸SO₂R³⁴; and
R¹¹ is CN, OH, R¹², —C(═O)R⁷, —NR⁸C(═O)R⁹, or —NR⁸SO₂R³⁴.

13. A compound according to claim 5 wherein
Q is 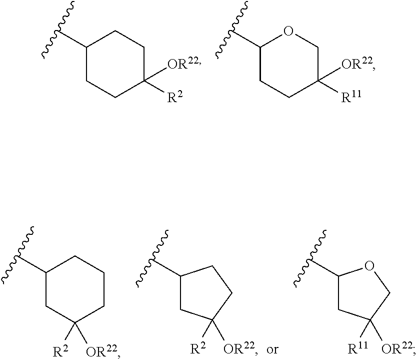
and
R² and R¹¹ are R¹² or —C(═O)R⁷.

14. A compound according to claim 5 wherein
Q is 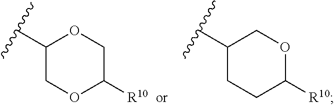
and
R¹⁰ is R¹² or —C(═O)R⁷.

15. A compound according to claim 4 of Formula IIA:

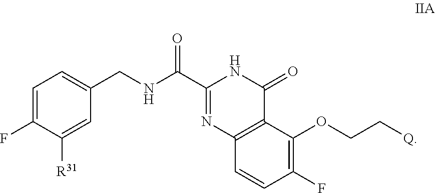

16. A compound according to claim 15 of Formula IIB:

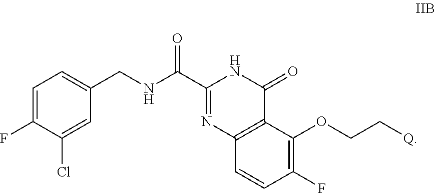

17. A compound according to claim 15 of Formula IIC:

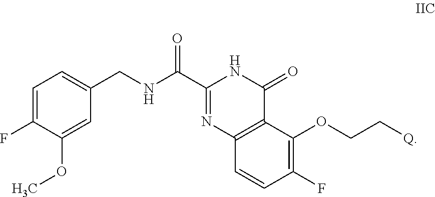

18. A compound according to claim 4 selected from:
N-(4-fluoro-3-methoxybenzyl)-5-[2-(4-acetylmorpholin-2-yl)ethoxy]-6-fluoro-4-oxo-3,4-dihydroquinazoline-2-carboxamide;
N-(4-fluoro-3-chlorobenzyl)-5-[2-(4-acetylmorpholin-2-yl)ethoxy]-6-fluoro-4-oxo-3,4-dihydroquinazoline-2-carboxamide;
N-(3-methoxybenzyl)-5-[2-(4-acetylmorpholin-2-yl)ethoxy]-6-fluoro-4-oxo-3,4-dihydroquinazoline-2-carboxamide;
N-(3-chlorobenzyl)-5-[2-(4-acetylmorpholin-2-yl)ethoxy]-6-fluoro-4-oxo-3,4-dihydroquinazoline-2-carboxamide;
N-(4-fluoro-3-methoxybenzyl)-6-fluoro-5-[2-(morpholin-2-yl)ethoxy]-4-oxo-3,4-dihydroquinazoline-2-carboxamide;
N-(4-fluoro-3-chlorobenzyl)-6-fluoro-5-[2-(morpholin-2-yl)ethoxy]-4-oxo-3,4-dihydroquinazoline-2-carboxamide;
N-(3-methoxybenzyl)-6-fluoro-5-[2-(morpholin-2-yl)ethoxy]-4-oxo-3,4-dihydroquinazoline-2-carboxamide;
N-(3-chlorobenzyl)-6-fluoro-5-[2-(morpholin-2-yl)ethoxy]-4-oxo-3,4-dihydroquinazoline-2-carboxamide;
N-(4-fluoro-3-methoxybenzyl)-6-fluoro-5-{2-[4-(methylsulfonyl)morpholin-2-yl]ethoxy}-4-oxo-3,4-dihydroquinazoline-2-carboxamide;
N-(4-fluoro-3-chlorobenzyl)-6-fluoro-5-{2-[4-(methylsulfonyl)morpholin-2-yl]ethoxy}-4-oxo-3,4-dihydroquinazoline-2-carboxamide;
N-(3-methoxybenzyl)-6-fluoro-5-{2-[4-(methylsulfonyl)morpholin-2-yl]ethoxy}-4-oxo-3,4-dihydroquinazoline-2-carboxamide;
N-(3-chlorobenzyl)-6-fluoro-5-{2-[4-(methylsulfonyl)morpholin-2-yl]ethoxy}-4-oxo-3,4-dihydroquinazoline-2-carboxamide;
N-(4-fluoro-3-methoxybenzyl)-5-[2-(4-acetylpiperazin-2-yl)ethoxy]-6-fluoro-4-oxo-3,4-dihydroquinazoline-2-carboxamide;
N-(4-fluoro-3-chlorobenzyl)-5-[2-(4-acetylpiperazin-2-yl)ethoxy]-6-fluoro-4-oxo-3,4-dihydroquinazoline-2-carboxamide;
N-(3-methoxybenzyl)-5-[2-(4-acetylpiperazin-2-yl)ethoxy]-6-fluoro-4-oxo-3,4-dihydroquinazoline-2-carboxamide;
N-(3-chlorobenzyl)-5-[2-(4-acetylpiperazin-2-yl)ethoxy]-6-fluoro-4-oxo-3,4-dihydroquinazoline-2-carboxamide;
N-(4-fluoro-3-methoxybenzyl)-6-fluoro-5-{2-[4-(methylsulfonyl)piperazin-2-yl]ethoxy}-4-oxo-3,4-dihydroquinazoline-2-carboxamide;
N-(4-fluoro-3-chlorobenzyl)-6-fluoro-5-{2-[4-(methylsulfonyl)piperazin-2-yl]ethoxy}-4-oxo-3,4-dihydroquinazoline-2-carboxamide;
N-(3-methoxybenzyl)-6-fluoro-5-{2-[4-(methylsulfonyl)piperazin-2-yl]ethoxy}-4-oxo-3,4-dihydroquinazoline-2-carboxamide;
N-(3-chlorobenzyl)-6-fluoro-5-{2-[4-(methylsulfonyl)piperazin-2-yl]ethoxy}-4-oxo-3,4-dihydroquinazoline-2-carboxamide;
N-(4-fluoro-3-methoxybenzyl)-5-{2-[5-cyano-5-(hydroxymethyl)-tetrahydro-2H-pyran-2-yl]ethoxy}-6-fluoro-4-oxo-3,4-dihydroquinazoline-2-carboxamide;
N-(4-fluoro-3-chlorobenzyl)-5-{2-[5-cyano-5-(hydroxymethyl)-tetrahydro-2H-pyran-2-yl]ethoxy}-6-fluoro-4-oxo-3,4-dihydroquinazoline-2-carboxamide;
N-(3-methoxybenzyl)-5-{2-[5-cyano-5-(hydroxymethyl)-tetrahydro-2H-pyran-2-yl]ethoxy}-6-fluoro-4-oxo-3,4-dihydroquinazoline-2-carboxamide;
N-(3-chlorobenzyl)-5-{2-[5-cyano-5-(hydroxymethyl)-tetrahydro-2H-pyran-2-yl]ethoxy}-6-fluoro-4-oxo-3,4-dihydroquinazoline-2-carboxamide;
N-(4-fluoro-3-methoxybenzyl)-6-fluoro-5-{2-[5-(hydroxymethyl)-tetrahydro-2H-pyran-2-yl]ethoxy}-4-oxo-3,4-dihydroquinazoline-2-carboxamide;
N-(4-fluoro-3-chlorobenzyl)-6-fluoro-5-{2-[5-(hydroxymethyl)-tetrahydro-2H-pyran-2-yl]ethoxy}-4-oxo-3,4-dihydroquinazoline-2-carboxamide;
N-(3-methoxybenzyl)-6-fluoro-5-{2-[5-(hydroxymethyl)-tetrahydro-2H-pyran-2-yl]ethoxy}-4-oxo-3,4-dihydroquinazoline-2-carboxamide;
N-(3-chlorobenzyl)-6-fluoro-5-{2-[5-(hydroxymethyl)-tetrahydro-2H-pyran-2-yl]ethoxy}-4-oxo-3,4-dihydroquinazoline-2-carboxamide;
N-(4-fluoro-3-methoxybenzyl)-6-fluoro-4-oxo-5-[2-(tetrahydro-2H-pyran-2-yl)ethoxy]-3,4-dihydroquinazoline-2-carboxamide;
N-(4-fluoro-3-chlorobenzyl)-6-fluoro-4-oxo-5-[2-(tetrahydro-2H-pyran-2-yl)ethoxy]-3,4-dihydroquinazoline-2-carboxamide;
N-(3-methoxybenzyl)-6-fluoro-4-oxo-5-[2-(tetrahydro-2H-pyran-2-yl)ethoxy]-3,4-dihydroquinazoline-2-carboxamide;
N-(3-chlorobenzyl)-6-fluoro-4-oxo-5-[2-(tetrahydro-2H-pyran-2-yl)ethoxy]-3,4-dihydroquinazoline-2-carboxamide;
N-(4-fluoro-3-methoxybenzyl)-6-fluoro-5-{2-[5-(2-hydroxyacetamido)-tetrahydro-2H-pyran-2-yl]ethoxy}-4-oxo-3,4-dihydroquinazoline-2-carboxamide;
N-(4-fluoro-3-chlorobenzyl)-6-fluoro-5-{2-[5-(2-hydroxyacetamido)-tetrahydro-2H-pyran-2-yl]ethoxy}-4-oxo-3,4-dihydroquinazoline-2-carboxamide;
N-(3-methoxybenzyl)-6-fluoro-5-{2-[5-(2-hydroxyacetamido)-tetrahydro-2H-pyran-2-yl]ethoxy}-4-oxo-3,4-dihydroquinazoline-2-carboxamide;
N-(3-chlorobenzyl)-6-fluoro-5-{2-[5-(2-hydroxyacetamido)-tetrahydro-2H-pyran-2-yl]ethoxy}-4-oxo-3,4-dihydroquinazoline-2-carboxamide;
N-(4-fluoro-3-methoxybenzyl)-5-[2-(5-acetamido-tetrahydro-2H-pyran-2-yl)ethoxy]-6-fluoro-4-oxo-3,4-dihydroquinazoline-2-carboxamide;
N-(4-fluoro-3-chlorobenzyl)-5-[2-(5-acetamido-tetrahydro-2H-pyran-2-yl)ethoxy]-6-fluoro-4-oxo-3,4-dihydroquinazoline-2-carboxamide;
N-(3-methoxybenzyl)-5-[2-(5-acetamido-tetrahydro-2H-pyran-2-yl)ethoxy]-6-fluoro-4-oxo-3,4-dihydroquinazoline-2-carboxamide;
N-(3-chlorobenzyl)-5-[2-(5-acetamido-tetrahydro-2H-pyran-2-yl)ethoxy]-6-fluoro-4-oxo-3,4-dihydroquinazoline-2-carboxamide;
N-(4-fluoro-3-methoxybenzyl)-6-fluoro-5-{2-[5-(methylsulfonamido)-tetrahydro-2H-pyran-2-yl]ethoxy}-4-oxo-3,4-dihydroquinazoline-2-carboxamide;
N-(4-fluoro-3-chlorobenzyl)-6-fluoro-5-{2-[5-(methylsulfonamido)-tetrahydro-2H-pyran-2-yl]ethoxy}-4-oxo-3,4-dihydroquinazoline-2-carboxamide;
N-(3-methoxybenzyl)-6-fluoro-5-{2-[5-(methylsulfonamido)-tetrahydro-2H-pyran-2-yl]ethoxy}-4-oxo-3,4-dihydroquinazoline-2-carboxamide;
N-(3-chlorobenzyl)-6-fluoro-5-{2-[5-(methylsulfonamido)-tetrahydro-2H-pyran-2-yl]ethoxy}-4-oxo-3,4-dihydroquinazoline-2-carboxamide;
N-(4-fluoro-3-methoxybenzyl)-6-fluoro-5-{2-[5-(hydroxymethyl)-5-methoxy-tetrahydro-2H-pyran-2-yl]ethoxy}-4-oxo-3,4-dihydroquinazoline-2-carboxamide;
N-(4-fluoro-3-chlorobenzyl)-6-fluoro-5-{2-[5-(hydroxymethyl)-5-methoxy-tetrahydro-2H-pyran-2-yl]ethoxy}-4-oxo-3,4-dihydroquinazoline-2-carboxamide;
N-(3-methoxybenzyl)-6-fluoro-5-{2-[5-(hydroxymethyl)-5-methoxy-tetrahydro-2H-pyran-2-yl]ethoxy}-4-oxo-3,4-dihydroquinazoline-2-carboxamide;
N-(3-chlorobenzyl)-6-fluoro-5-{2-[5-(hydroxymethyl)-5-methoxy-tetrahydro-2H-pyran-2-yl]ethoxy}-4-oxo-3,4-dihydroquinazoline-2-carboxamide;
N-(4-fluoro-3-methoxybenzyl)-6-fluoro-5-{2-[5-hydroxy-5-(hydroxymethyl)-tetrahydro-2H-pyran-2-yl]ethoxy}-4-oxo-3,4-dihydroquinazoline-2-carboxamide;
N-(4-fluoro-3-chlorobenzyl)-6-fluoro-5-{2-[5-hydroxy-5-(hydroxymethyl)-tetrahydro-2H-pyran-2-yl]ethoxy}-4-oxo-3,4-dihydroquinazoline-2-carboxamide;
N-(3-methoxybenzyl)-6-fluoro-5-{2-[5-hydroxy-5-(hydroxymethyl)-tetrahydro-2H-pyran-2-yl]ethoxy}-4-oxo-3,4-dihydroquinazoline-2-carboxamide;
N-(3-chlorobenzyl)-6-fluoro-5-{2-[5-hydroxy-5-(hydroxymethyl)-tetrahydro-2H-pyran-2-yl]ethoxy}-4-oxo-3,4-dihydroquinazoline-2-carboxamide;
6-fluoro-N-(3-methoxy-4-fluorobenzyl)-4-oxo-5-(2-piperidin-4-ylethoxy)-3,4-dihydroquinazoline-2-carboxamide;
6-fluoro-N-(3-chloro-4-fluorobenzyl)-4-oxo-5-(2-piperidin-4-ylethoxy)-3,4-dihydroquinazoline-2-carboxamide;
6-fluoro-N-(3-methoxybenzyl)-4-oxo-5-(2-piperidin-4-ylethoxy)-3,4-dihydroquinazoline-2-carboxamide;
6-fluoro-N-(3-chlorobenzyl)-4-oxo-5-(2-piperidin-4-ylethoxy)-3,4-dihydroquinazoline-2-carboxamide;
5-[2-(1-acetyl piperidin-4-yl)ethoxy]-6-fluoro-N-(3-methoxy-4-fluorobenzyl)-4-oxo-3,4-dihydroquinazoline-2-carboxamide;
5-[2-(1-acetylpiperidin-4-yl)ethoxy]-6-fluoro-N-(3-chloro-4-fluorobenzyl)-4-oxo-3,4-dihydroquinazoline-2-carboxamide;
5-[2-(1-acetylpiperidin-4-yl)ethoxy]-6-fluoro-N-(3-methoxybenzyl)-4-oxo-3,4-dihydroquinazoline-2-carboxamide PF-04544352-00;
5-[2-(1-acetylpiperidin-4-yl)ethoxy]-6-fluoro-N-(3-chlorobenzyl)-4-oxo-3,4-dihydroquinazoline-2-carboxamide;
2-(4-{2-[(6-fluoro-2-{[(3-methoxy-4-fluorobenzyl)amino]carbonyl}-4-oxo-3,4-dihydroquinazolin-5-yl)oxy]ethyl}piperidin-1-yl)-2-oxoethyl acetate;
2-(4-{2-[(6-fluoro-2-{[(3-chloro-4-fluorobenzyl)amino]carbonyl}-4-oxo-3,4-dihydroquinazolin-5-yl)oxy]ethyl}piperidin-1-yl)-2-oxoethyl acetate;
2-(4-{2-[(6-fluoro-2-{[(3-methoxybenzyl)amino]carbonyl}-4-oxo-3,4-dihydroquinazolin-5-yl)oxy]ethyl}piperidin-1-yl)-2-oxoethyl acetate;
2-(4-{2-[(6-fluoro-2-{[(3-chlorobenzyl)amino]carbonyl}-4-oxo-3,4-dihydroquinazolin-5-yl)oxy]ethyl}piperidin-1-yl)-2-oxoethyl acetate;
6-fluoro-5-{2-[1-(2-hydroxyethyl)piperidin-4-yl]ethoxy}-N-(3-methoxy-4-fluorobenzyl)-4-oxo-3,4-dihydroquinazoline-2-carboxamide;
6-fluoro-5-{2-[1-(2-hydroxyethyl)piperidin-4-yl]ethoxy}-N-(3-chloro4-fluorobenzyl)-4-oxo-3,4-dihydroquinazoline-2-carboxamide;
6-fluoro-5-{2-[1-(2-hydroxyethyl)piperidin-4-yl]ethoxy}-N-(3-methoxybenzyl)-4-oxo-3,4-dihydroquinazoline-2-carboxamide;
6-fluoro-5-{2-[1-(2-hydroxyethyl)piperidin-4-yl]ethoxy}-N-(3-chlorobenzyl)-4-oxo-3,4-dihydroquinazoline-2-carboxamide;
N-(4-fluoro-3-methoxybenzyl)-6-fluoro-5-(2-{1-[(5-methyl-1,3,4-oxadiazol-2-yl)methyl]piperidin-4-yl}ethoxy)-4-oxo-3,4-dihydroquinazoline-2-carboxamide;
N-(4-fluoro-3-chlorobenzyl)-6-fluoro-5-(2-{1-[(5-methyl-1,3,4-oxadiazol-2-yl)methyl]piperidin-4-yl}ethoxy)-4-oxo-3,4-dihydroquinazoline-2-carboxamide;
N-(3-methoxybenzyl)-6-fluoro-5-(2-{1-[(5-methyl-1,3,4-oxadiazol-2-yl)methyl]piperidin-4-yl}ethoxy)-4-oxo-3,4-dihydroquinazoline-2-carboxamide;
N-(3-chlorobenzyl)-6-fluoro-5-(2-{1-[(5-methyl-1,3,4-oxadiazol-2-yl)methyl]piperidin-4-yl}ethoxy)-4-oxo-3,4-dihydroquinazoline-2-carboxamide;
N-(4-fluoro-3-methoxybenzyl)-6-fluoro-5-{2-[1-(2-hydroxyacetyl)piperidin-4-yl]ethoxy}-4-oxo-3,4-dihydroquinazoline-2-carboxamide;
N-(4-fluoro-3-chlorobenzyl)-6-fluoro-5-{2-[1-(2-hydroxyacetyl)piperidin-4-yl]ethoxy}-4-oxo-3,4-dihydroquinazoline-2-carboxamide;
N-(3-methoxybenzyl)-6-fluoro-5-{2-[1-(2-hydroxyacetyl)piperidin-4-yl]ethoxy}-4-oxo-3,4-dihydroquinazoline-2-carboxamideN-(3-chlorobenzyl)-6-fluoro-5-{2-[1-(2-hydroxyacetyl)piperidin-4-yl]ethoxy}-4-oxo-3,4-dihydroquinazoline-2-carboxamide;
N-(4-fluoro-3-methoxybenzyl)-6-fluoro-5-{2-[1-(methylsulfonyl)piperidin-4-yl]ethoxy}-4-oxo-3,4-dihydroquinazoline-2-carboxamide;
N-(4-fluoro-3-chlorobenzyl)-6-fluoro-5-{2-[1-(methylsulfonyl)piperidin-4-yl]ethoxy}-4-oxo-3,4-dihydroquinazoline-2-carboxamide;
N-(3-methoxybenzyl)-6-fluoro-5-{2-[1-(methylsulfonyl)piperidin-4-yl]ethoxy}-4-oxo-3,4-dihydroquinazoline-2-carboxamide;
N-(3-chlorobenzyl)-6-fluoro-5-{2-[1-(methylsulfonyl)piperidin-4-yl]ethoxy}-4-oxo-3,4-dihydroquinazoline-2-carboxamide;
N-(4-fluoro-3-methoxybenzyl)-6-fluoro-5-{2-[3-(2-hydroxyacetamido)cyclohexyl]ethoxy}-4-oxo-3,4-dihydroquinazoline-2-carboxamide;
N-(4-fluoro-3-chlorobenzyl)-6-fluoro-5-{2-[3-(2-hydroxyacetamido)cyclohexyl]ethoxy}-4-oxo-3,4-dihydroquinazoline-2-carboxamide;
N-(3-methoxybenzyl)-6-fluoro-5-{2-[3-(2-hydroxyacetamido)cyclohexyl]ethoxy}-4-oxo-3,4-dihydroquinazoline-2-carboxamide;
N-(3-chlorobenzyl)-6-fluoro-5-{2-[3-(2-hydroxyacetamido)cyclohexyl]ethoxy}-4-oxo-3,4-dihydroquinazoline-2-carboxamide;
N-(4-fluoro-3-methoxybenzyl)-6-fluoro-5-{2-[4-(2-hydroxyacetamido)-tetrahydrofuran-2-yl]ethoxy}-4-oxo-3,4-dihydroquinazoline-2-carboxamide;
N-(4-fluoro-3-chlorobenzyl)-6-fluoro-5-{2-[4-(2-hydroxyacetamido)-tetrahydrofuran-2-yl]ethoxy}-4-oxo-3,4-dihydroquinazoline-2-carboxamide;
N-(3-methoxybenzyl)-6-fluoro-5-{2-[4-(2-hydroxyacetamido)-tetrahydrofuran-2-yl]ethoxy}-4-oxo-3,4-dihydroquinazoline-2-carboxamide;
N-(3-chlorobenzyl)-6-fluoro-5-{2-[4-(2-hydroxyacetamido)-tetrahydrofuran-2-yl]ethoxy}-4-oxo-3,4-dihydroquinazoline-2-carboxamide;
N-(4-fluoro-3-methoxybenzyl)-5-[2-(4-acetamido-tetrahydrofuran-2-yl)ethoxy]-6-fluoro-4-oxo-3,4-dihydroquinazoline-2-carboxamide;
N-(4-fluoro-3-chlorobenzyl)-5-[2-(4-acetamido-tetrahydrofuran-2-yl)ethoxy]-6-fluoro-4-oxo-3,4-dihydroquinazoline-2-carboxamide;
N-(3-methoxybenzyl)-5-[2-(4-acetamido-tetrahydrofuran-2-yl)ethoxy]-6-fluoro-4-oxo-3,4-dihydroquinazoline-2-carboxamide;
N-(3-chlorobenzyl)-5-[2-(4-acetamido-tetrahydrofuran-2-yl)ethoxy]-6-fluoro-4-oxo-3,4-dihydroquinazoline-2-carboxamide;
N-(4-fluoro-3-methoxybenzyl)-6-fluoro-5-{2-[4-(methylsulfonamido)-tetrahydrofuran-2-yl]ethoxy}-4-oxo-3,4-dihydroquinazoline-2-carboxamide;
N-(4-fluoro-3-chlorobenzyl)-6-fluoro-5-{2-[4-(methylsulfonamido)-tetrahydrofuran-2-yl]ethoxy}-4-oxo-3,4-dihydroquinazoline-2-carboxamide;
N-(3-methoxybenzyl)-6-fluoro-5-{2-[4-(methylsulfonamido)-tetrahydrofuran-2-yl]ethoxy}-4-oxo-3,4-dihydroquinazoline-2-carboxamide;
N-(3-chlorobenzyl)-6-fluoro-5-{2-[4-(methylsulfonamido)-tetrahydrofuran-2-yl]ethoxy}-4-oxo-3,4-dihydroquinazoline-2-carboxamide;
N-(4-fluoro-3-methoxybenzyl)-6-fluoro-5-{2-[4-hydroxy-4-(hydroxymethyl)-tetrahydrofuran-2-yl]ethoxy}-4-oxo-3,4-dihydroquinazoline-2-carboxamide;
N-(4-fluoro-3-chlorobenzyl)-6-fluoro-5-{2-[4-hydroxy-4-(hydroxymethyl)-tetrahydrofuran-2-yl]ethoxy}-4-oxo-3,4-dihydroquinazoline-2-carboxamide;
N-(3-methoxybenzyl)-6-fluoro-5-{2-[4-hydroxy-4-(hydroxymethyl)-tetrahydrofuran-2-yl]ethoxy}-4-oxo-3,4-dihydroquinazoline-2-carboxamide;
N-(3-chlorobenzyl)-6-fluoro-5-{2-[4-hydroxy-4-(hydroxymethyl)-tetrahydrofuran-2-yl]ethoxy}-4-oxo-3,4-dihydroquinazoline-2-carboxamide;
N-(4-fluoro-3-methoxybenzyl)-6-fluoro-5-(2-{6-[(2-hydroxyacetamido)methyl]-tetrahydro-2H-pyran-3-yl}ethoxy)-4-oxo-3,4-dihydroquinazoline-2-carboxamide;
N-(4-fluoro-3-chlorobenzyl)-6-fluoro-5-(2-{6-[(2-hydroxyacetamido)methyl]-tetrahydro-2H-pyran-3-yl}ethoxy)-4-oxo-3,4-dihydroquinazoline-2-carboxamide;
N-(3-methoxybenzyl)-6-fluoro-5-(2-{6-[(2-hydroxyacetamido)methyl]-tetrahydro-2H-pyran-3-yl}ethoxy)-4-oxo-3,4-dihydroquinazoline-2-carboxamide;
N-(3-chlorobenzyl)-6-fluoro-5-(2-{6-[(2-hydroxyacetamido)methyl]-tetrahydro-2H-pyran-3-yl}ethoxy)-4-oxo-3,4-dihydroquinazoline-2-carboxamide;
N-(4-fluoro-3-methoxybenzyl)-5-{2-[6-(acetamidomethyl)-tetrahydro-2H-pyran-3-yl]ethoxy}-6-fluoro-4-oxo-3,4-dihydroquinazoline-2-carboxamide;
N-(4-fluoro-3-chlorobenzyl)-5-{2-[6-(acetamidomethyl)-tetrahydro-2H-pyran-3-yl]ethoxy}-6-fluoro-4-oxo-3,4-dihydroquinazoline-2-carboxamide;
N-(3-methoxybenzyl)-5-{2-[6-(acetamidomethyl)-tetrahydro-2H-pyran-3-yl]ethoxy}-6-fluoro-4-oxo-3,4-dihydroquinazoline-2-carboxamide;
N-(3-chlorobenzyl)-5-{2-[6-(acetamidomethyl)-tetrahydro-2H-pyran-3-yl]ethoxy}-6-fluoro-4-oxo-3,4-dihydroquinazoline-2-carboxamide;
N-(4-fluoro-3-methoxybenzyl)-6-fluoro-5-{2-[6-(methylsulfonamidomethyl)-tetrahydro-2H-pyran-3-yl]ethoxy}-4-oxo-3,4-dihydroquinazoline-2-carboxamide;
N-(4-fluoro-3-chlorobenzyl)-6-fluoro-5-{2-[6-(methylsulfonamidomethyl)-tetrahydro-2H-pyran-3-yl]ethoxy}-4-oxo-3,4-dihydroquinazoline-2-carboxamide;
N-(3-methoxybenzyl)-6-fluoro-5-{2-[6-(methylsulfonamidomethyl)-tetrahydro-2H-pyran-3-yl]ethoxy}-4-oxo-3,4-dihydroquinazoline-2-carboxamide;
N-(3-chlorobenzyl)-6-fluoro-5-{2-[6-(methylsulfonamidomethyl)-tetrahydro-2H-pyran-3-yl]ethoxy}-4-oxo-3,4-dihydroquinazoline-2-carboxamide;
N-(4-fluoro-3-methoxybenzyl)-6-fluoro-5-{2-[6-(hydroxymethyl)-tetrahydro-2H-pyran-3-yl]ethoxy}-4-oxo-3,4-dihydroquinazoline-2-carboxamide;
N-(4-fluoro-3-chlorobenzyl)-6-fluoro-5-{2-[6-(hydroxymethyl)-tetrahydro-2H-pyran-3-yl]ethoxy}-4-oxo-3,4-dihydroquinazoline-2-carboxamide;
N-(3-methoxybenzyl)-6-fluoro-5-{2-[6-(hydroxymethyl)-tetrahydro-2H-pyran-3-yl]ethoxy}-4-oxo-3,4-dihydroquinazoline-2-carboxamide;
N-(3-chlorobenzyl)-6-fluoro-5-{2-[6-(hydroxymethyl)-tetrahydro-2H-pyran-3-yl]ethoxy}-4-oxo-3,4-dihydroquinazoline-2-carboxamide;
N-(4-fluoro-3-methoxybenzyl)-6-fluoro-4-oxo-5-[2-(tetrahydro-2H-1,1′-dioxothiopyran-4-yl)ethoxy]-3,4-dihydroquinazoline-2-carboxamide;
N-(4-fluoro-3-chlorobenzyl)-6-fluoro-4-oxo-5-[2-(tetrahydro-2H-1,1′-dioxothiopyran-4-yl)ethoxy]-3,4-dihydroquinazoline-2-carboxamide;
N-(3-methoxybenzyl)-6-fluoro-4-oxo-5-[2-(tetrahydro-2H-1,1′-dioxothiopyran-4-yl)ethoxy]-3,4-dihydroquinazoline-2-carboxamide;
N-(3-chlorobenzyl)-6-fluoro-4-oxo-5-[2-(tetrahydro-2H-1,1′-dioxothiopyran-4-yl)ethoxy]-3,4-dihydroquinazoline-2-carboxamide;
6-fluoro-5-{[4-(hydroxymethyl)cyclohexyl]methoxy}-N-(3-methoxy-4-fluorobenzyl)-4-oxo-3,4-dihydroquinazoline-2-carboxamide;
6-fluoro-5-{[4-(hydroxymethyl)cyclohexyl]methoxy}-N-(3-chloro-4-fluorobenzyl)-4-oxo-3,4-dihydroquinazoline-2-carboxamide;
6-fluoro-5-{[4-(hydroxymethyl)cyclohexyl]methoxy}-N-(3-methoxybenzyl)-4-oxo-3,4-dihydroquinazoline-2-carboxamide;
6-fluoro-5-{[4-(hydroxymethyl)cyclohexyl]methoxy}-N-(3-chlorobenzyl)-4-oxo-3,4-dihydroquinazoline-2-carboxamide;
N-(4-fluoro-3-methoxybenzyl)-6-fluoro-5-(2-{4-[(5-methyl-1,3,4-oxadiazol-2-yl)methyl]cyclohexyl}ethoxy)-4-oxo-3,4-dihydroquinazoline-2-carboxamide;
N-(4-fluoro-3-chlorobenzyl)-6-fluoro-5-(2-{4-[(5-methyl-1,3,4-oxadiazol-2-yl)methyl]cyclohexyl}ethoxy)-4-oxo-3,4-dihydroquinazoline-2-carboxamide;
N-(3-methoxybenzyl)-6-fluoro-5-(2-{4-[(5-methyl-1,3,4-oxadiazol-2-yl)methyl]cyclohexyl}ethoxy)-4-oxo-3,4-dihydroquinazoline-2-carboxamide;
N-(3-chlorobenzyl)-6-fluoro-5-(2-{4-[(5-methyl-1,3,4-oxadiazol-2-yl)methyl]cyclohexyl}ethoxy)-4-oxo-3,4-dihydroquinazoline-2-carboxamide;
N-(4-fluoro-3-methoxybenzyl)-6-fluoro-5-{2-[4-(methylsulfonamidomethyl)cyclohexyl]ethoxy}-4-oxo-3,4-dihydroquinazoline-2-carboxamide;
N-(4-fluoro-3-chlorobenzyl)-6-fluoro-5-{2-[4-(methylsulfonamidomethyl)cyclohexyl]ethoxy}-4-oxo-3,4-dihydroquinazoline-2-carboxamide;
N-(3-methoxybenzyl)-6-fluoro-5-{2-[4-(methylsulfonamidomethyl)cyclohexyl]ethoxy}-4-oxo-3,4-dihydroquinazoline-2-carboxamide;
N-(3-chlorobenzyl)-6-fluoro-5-{2-[4-(methylsulfonamidomethyl)cyclohexyl]ethoxy}-4-oxo-3,4-dihydroquinazoline-2-carboxamide;
N-(4-fluoro-3-methoxybenzyl)-6-fluoro-5-(2-(4-hydroxy-4-(hydroxymethyl)cyclohexyl]ethoxy}-4-oxo-3,4-dihydroquinazoline-2-carboxamide;
N-(4-fluoro-3-chlorobenzyl)-6-fluoro-5-(2-(4-hydroxy-4-(hydroxymethyl)cyclohexyl]ethoxy}-4-oxo-3,4-dihydroquinazoline-2-carboxamide;
N-(3-methoxybenzyl)-6-fluoro-5-(2-(4-hydroxy-4-(hydroxymethyl)cyclohexyl]ethoxy}-4-oxo-3,4-dihydroquinazoline-2-carboxamide;
N-(3-chlorobenzyl)-6-fluoro-5-{2-[4-hydroxy-4-(hydroxymethyl)cyclohexyl]ethoxy}-4-oxo-3,4-dihydroquinazoline-2-carboxamide;
N-(4-fluoro-3-methoxybenzyl)-5-[2-(4-cyanocyclohexyl)ethoxy]-6-fluoro-4-oxo-3,4-dihydroquinazoline-2-carboxamide;
N-(4-fluoro-3-chlorobenzyl)-5-[2-(4-cyanocyclohexyl)ethoxy]-6-fluoro-4-oxo-3,4-dihydroquinazoline-2-carboxamide;
N-(3-methoxybenzyl)-5-[2-(4-cyanocyclohexyl)ethoxy]-6-fluoro-4-oxo-3,4-dihydroquinazoline-2-carboxamide;
N-(3-chlorobenzyl)-5-[2-(4-cyanocyclohexyl)ethoxy]-6-fluoro-4-oxo-3,4-dihydroquinazoline-2-carboxamide;
2-[4-(2-{2-[(4-fluoro-3-methoxybenzyl)carbamoyl]-6-fluoro-4-oxo-3,4-dihydroquinazolin-5-yloxy}ethyl)cyclohexylamino]-2-oxoethyl acetate;
2-[4-(2-{2-[(4-fluoro-3-chlorobenzyl)carbamoyl]-6-fluoro-4-oxo-3,4-dihydroquinazolin-5-yloxy}ethyl)cyclohexylamino]-2-oxoethyl acetate;
2-[4-(2-{2-[(3-methoxybenzyl)carbamoyl]-6-fluoro-4-oxo-3,4-dihydroquinazolin-5-yloxy}ethyl)cyclohexylamino]-2-oxoethyl acetate;
2-[4-(2-{2-[(3-chlorobenzyl)carbamoyl]-6-fluoro-4-oxo-3,4-dihydroquinazolin-5-yloxy}ethyl)cyclohexylamino]-2-oxoethyl acetate;
N-(4-fluoro-3-methoxybenzyl)-6-fluoro-5-{2-[4-(2-hydroxyacetamido)cyclohexyl]ethoxy}-4-oxo-3,4-dihydroquinazoline-2-carboxamide;
N-(4-fluoro-3-chlorobenzyl)-6-fluoro-5-{2-[4-(2-hydroxyacetamido)cyclohexyl]ethoxy}-4-oxo-3,4-dihydroquinazoline-2-carboxamide;
N-(3-methoxybenzyl)-6-fluoro-5-{2-[4-(2-hydroxyacetamido)cyclohexyl]ethoxy}-4-oxo-3,4-dihydroquinazoline-2-carboxamide;
N-(3-chlorobenzyl)-6-fluoro-5-{2-[4-(2-hydroxyacetamido)cyclohexyl]ethoxy}-4-oxo-3,4-dihydroquinazoline-2-carboxamide;
N-(4-fluoro-3-methoxybenzyl)-5-[2-(4-acetamidocyclohexyl)ethoxy]-6-fluoro-4-oxo-3,4-dihydroquinazoline-2-carboxamide;
N-(4-fluoro-3-chlorobenzyl)-5-[2-(4-acetamidocyclohexyl)ethoxy]-6-fluoro-4-oxo-3,4-dihydroquinazoline-2-carboxamide;
N-(3-methoxybenzyl)-5-[2-(4-acetamidocyclohexyl)ethoxy]-6-fluoro-4-oxo-3,4-dihydroquinazoline-2-carboxamide;
N-(3-chlorobenzyl)-5-[2-(4-acetamidocyclohexyl)ethoxy]-6-fluoro-4-oxo-3,4-dihydroquinazoline-2-carboxamide;
N-(4-fluoro-3-methoxybenzyl)-6-fluoro-5-{2-[4-(methylsulfonamido)cyclohexyl]ethoxy}-4-oxo-3,4-dihydroquinazoline-2-carboxamide;
N-(4-fluoro-3-chlorobenzyl)-6-fluoro-5-{2-[4-(methylsulfonamido)cyclohexyl]ethoxy}-4-oxo-3,4-dihydroquinazoline-2-carboxamide;
N-(3-methoxybenzyl)-6-fluoro-5-{2-[4-(methylsulfonamido)cyclohexyl]ethoxy}-4-oxo-3,4-dihydroquinazoline-2-carboxamide;
N-(3-chlorobenzyl)-6-fluoro-5-{2-[4-(methylsulfonamido)cyclohexyl]ethoxy}-4-oxo-3,4-dihydroquinazoline-2-carboxamide;
N-(4-fluoro-3-methoxybenzyl)-6-fluoro-5-{2-[5-(2-hydroxypropan-2-yl)-1,4-dioxan-2-yl]ethoxy}-4-oxo-3,4-dihydroquinazoline-2-carboxamide;
N-(4-fluoro-3-chlorobenzyl)-6-fluoro-5-{2-[5-(2-hydroxypropan-2-yl)-1,4-dioxan-2-yl]ethoxy}-4-oxo-3,4-dihydroquinazoline-2-carboxamide;
N-(3-methoxybenzyl)-6-fluoro-5-{2-[5-(2-hydroxypropan-2-yl)-1,4-dioxan-2-yl]ethoxy}-4-oxo-3,4-dihydroquinazoline-2-carboxamide;
N-(3-chlorobenzyl)-6-fluoro-5-{2-[5-(2-hydroxypropan-2-yl)-1,4-dioxan-2-yl]ethoxy}-4-oxo-3,4-dihydroquinazoline-2-carboxamide;
N-(4-fluoro-3-methoxybenzyl)-5-[2-(5-carbamoyl-1,4-dioxan-2-yl)ethoxy]-6-fluoro-4-oxo-3,4-dihydroquinazoline-2-carboxamide;
N-(4-fluoro-3-chlorobenzyl)-5-[2-(5-carbamoyl-1,4-dioxan-2-yl)ethoxy]-6-fluoro-4-oxo-3,4-dihydroquinazoline-2-carboxamide;
N-(3-methoxybenzyl)-5-[2-(5-carbamoyl-1,4-dioxan-2-yl)ethoxy]-6-fluoro-4-oxo-3,4-dihydroquinazoline-2-carboxamide;
N-(3-chlorobenzyl)-5-[2-(5-carbamoyl-1,4-dioxan-2-yl)ethoxy]-6-fluoro-4-oxo-3,4-dihydroquinazoline-2-carboxamide;
5-(2-{5-[(hydroxymethyl)carbamoyl]-1,4-dioxan-2-yl}ethoxy)-N-(4-fluoro-3-methoxybenzyl)-6-fluoro-4-oxo-3,4-dihydroquinazoline-2-carboxamide;
5-(2-{5-[(hydroxymethyl)carbamoyl]-1,4-dioxan-2-yl}ethoxy)-N-(4-fluoro-3-chlorobenzyl)-6-fluoro-4-oxo-3,4-dihydroquinazoline-2-carboxamide;
5-(2-{5-[(hydroxymethyl)carbamoyl]-1,4-dioxan-2-yl}ethoxy)-N-(3-methoxybenzyl)-6-fluoro-4-oxo-3,4-dihydroquinazoline-2-carboxamide;
5-(2-{5-[(hydroxymethyl)carbamoyl]-1,4-dioxan-2-yl}ethoxy)-N-(3-chlorobenzyl)-6-fluoro-4-oxo-3,4-dihydroquinazoline-2-carboxamide;
N-(4-fluoro-3-methoxybenzyl)-6-fluoro-5-{2-[5-(methylcarbamoyl)-1,4-dioxan-2-yl]ethoxy}-4-oxo-3,4-dihydroquinazoline-2-carboxamide;
N-(4-fluoro-3-chlorobenzyl)-6-fluoro-5-{2-[5-(methylcarbamoyl)-1,4-dioxan-2-yl]ethoxy}-4-oxo-3,4-dihydroquinazoline-2-carboxamide;
N-(3-methoxybenzyl)-6-fluoro-5-{2-[5-(methylcarbamoyl)-1,4-dioxan-2-yl]ethoxy}-4-oxo-3,4-dihydroquinazoline-2-carboxamide;
N-(3-chlorobenzyl)-6-fluoro-5-{2-[5-(methylcarbamoyl)-1,4-dioxan-2-yl]ethoxy}-4-oxo-3,4-dihydroquinazoline-2-carboxamide;
5-(2-{2-[(4-fluoro-3-methoxybenzyl)carbamoyl]-6-fluoro-4-oxo-3,4-dihydroquinazolin-5-yloxy}ethyl)-1,4-dioxane-2-carboxylic acid;
5-(2-{2-[(4-fluoro-3-chlorobenzyl)carbamoyl]-6-fluoro-4-oxo-3,4-dihydroquinazolin-5-yloxy}ethyl)-1,4-dioxane-2-carboxylic acid;
5-(2-{2-[(3-methoxybenzyl)carbamoyl]-6-fluoro-4-oxo-3,4-dihydroquinazolin-5-yloxy}ethyl)-1,4-dioxane-2-carboxylic acid;
5-(2-{2-[(3-chlorobenzyl)carbamoyl]-6-fluoro-4-oxo-3,4-dihydroquinazolin-5-yloxy}ethyl)-1,4-dioxane-2-carboxylic acid;
N-(4-fluoro-3-methoxybenzyl)-5-{2-[5-(cyanomethyl)-1,4-dioxan-2-yl]ethoxy}-6-fluoro-4-oxo-3,4-dihydroquinazoline-2-carboxamide;
N-(4-fluoro-3-chlorobenzyl)-5-{2-[5-(cyanomethyl)-1,4-dioxan-2-yl]ethoxy}-6-fluoro-4-oxo-3,4-dihydroquinazoline-2-carboxamide;
N-(3-methoxybenzyl)-5-{2-[5-(cyanomethyl)-1,4-dioxan-2-yl]ethoxy}-6-fluoro-4-oxo-3,4-dihydroquinazoline-2-carboxamide;
N-(3-chlorobenzyl)-5-{2-[5-(cyanomethyl)-1,4-dioxan-2-yl]ethoxy}-6-fluoro-4-oxo-3,4-dihydroquinazoline-2-carboxamide;
N-(4-fluoro-3-methoxybenzyl)-6-fluoro-5-{2-[5-(methylsulfonamidomethyl)-1,4-dioxan-2-yl]ethoxy}-4-oxo-3,4-dihydroquinazoline-2-carboxamide;
N-(4-fluoro-3-chlorobenzyl)-6-fluoro-5-{2-[5-(methylsulfonamidomethyl)-1,4-dioxan-2-yl]ethoxy}-4-oxo-3,4-dihydroquinazoline-2-carboxamide;
N-(3-methoxybenzyl)-6-fluoro-5-{2-[5-(methylsulfonamidomethyl)-1,4-dioxan-2-yl]ethoxy}-4-oxo-3,4-dihydroquinazoline-2-carboxamide;
N-(3-chlorobenzyl)-6-fluoro-5-{2-[5-(methylsulfonamidomethyl)-1,4-dioxan-2-yl]ethoxy}-4-oxo-3,4-dihydroquinazoline-2-carboxamide;
N-(4-fluoro-3-methoxybenzyl)-6-fluoro-5-{2-[5-(hydroxymethyl)-1,4-dioxan-2-yl]ethoxy}-4-oxo-3,4-dihydroquinazoline-2-carboxamide;
N-(4-fluoro-3-chlorobenzyl)-6-fluoro-5-{2-[5-(hydroxymethyl)-1,4-dioxan-2-yl]ethoxy}-4-oxo-3,4-dihydroquinazoline-2-carboxamide;
N-(3-methoxybenzyl)-6-fluoro-5-{2-[5-(hydroxymethyl)-1,4-dioxan-2-yl]ethoxy}-4-oxo-3,4-dihydroquinazoline-2-carboxamide;
N-(3-chlorobenzyl)-6-fluoro-5-{2-[5-(hydroxymethyl)-1,4-dioxan-2-yl]ethoxy}-4-oxo-3,4-dihydroquinazoline-2-carboxamide;
N-(4-fluoro-3-methoxybenzyl)-6-fluoro-5-{2-[5-(methylsulfonylmethyl)-1,4-dioxan-2-yl]ethoxy}-4-oxo-3,4-dihydroquinazoline-2-carboxamide;
N-(4-fluoro-3-chlorobenzyl)-6-fluoro-5-{2-[5-(methylsulfonylmethyl)-1,4-dioxan-2-yl]ethoxy}-4-oxo-3,4-dihydroquinazoline-2-carboxamide;
N-(3-methoxybenzyl)-6-fluoro-5-{2-[5-(methylsulfonylmethyl)-1,4-dioxan-2-yl]ethoxy}-4-oxo-3,4-dihydroquinazoline-2-carboxamide;
N-(3-chlorobenzyl)-6-fluoro-5-{2-[5-(methylsulfonylmethyl)-1,4-dioxan-2-yl]ethoxy}-4-oxo-3,4-dihydroquinazoline-2-carboxamide;
N-(4-fluoro-3-methoxybenzyl)-5-[2-(5-cyano-1,4-dioxan-2-yl)ethoxy]-6-fluoro-4-oxo-3,4-dihydroquinazoline-2-carboxamide;
N-(4-fluoro-3-chlorobenzyl)-5-[2-(5-cyano-1,4-dioxan-2-yl)ethoxy]-6-fluoro-4-oxo-3,4-dihydroquinazoline-2-carboxamide;
N-(3-methoxybenzyl)-5-[2-(5-cyano-1,4-dioxan-2-yl)ethoxy]-6-fluoro-4-oxo-3,4-dihydroquinazoline-2-carboxamide;
N-(3-chlorobenzyl)-5-[2-(5-cyano-1,4-dioxan-2-yl)ethoxy]-6-fluoro-4-oxo-3,4-dihydroquinazoline-2-carboxamide;
N-(4-fluoro-3-methoxybenzyl)-6-fluoro-5-{2-[5-(2-hydroxypropan-2-yl)-1,4-dioxan-2-yl]methoxy}-4-oxo-3,4-dihydroquinazoline-2-carboxamide;
N-(4-fluoro-3-chlorobenzyl)-6-fluoro-5-{2-[5-(2-hydroxypropan-2-yl)-1,4-dioxan-2-yl]methoxy}-4-oxo-3,4-dihydroquinazoline-2-carboxamide;
N-(3-methoxybenzyl)-6-fluoro-5-{2-[5-(2-hydroxypropan-2-yl)-1,4-dioxan-2-yl]methoxy}-4-oxo-3,4-dihydroquinazoline-2-carboxamide;
N-(3-chlorobenzyl)-6-fluoro-5-{2-[5-(2-hydroxypropan-2-yl)-1,4-dioxan-2-yl]methoxy}-4-oxo-3,4-dihydroquinazoline-2-carboxamide;
N-(4-fluoro-3-methoxybenzyl)-5-[2-(5-carbamoyl-1,4-dioxan-2-yl)methoxy]-6-fluoro-4-oxo-3,4-dihydroquinazoline-2-carboxamide;
N-(4-fluoro-3-chlorobenzyl)-5-[2-(5-carbamoyl-1,4-dioxan-2-yl)methoxy]-6-fluoro-4-oxo-3,4-dihydroquinazoline-2-carboxamide;
N-(3-methoxybenzyl)-5-[2-(5-carbamoyl-1,4-dioxan-2-yl)methoxy]-6-fluoro-4-oxo-3,4-dihydroquinazoline-2-carboxamide;
N-(3-chlorobenzyl)-5-[2-(5-carbamoyl-1,4-dioxan-2-yl)methoxy]-6-fluoro-4-oxo-3,4-dihydroquinazoline-2-carboxamide;
5-(2-{5-[(hydroxymethyl)carbamoyl]-1,4-dioxan-2-yl}methoxy)-N-(4-fluoro-3-methoxybenzyl)-6-fluoro-4-oxo-3,4-dihydroquinazoline-2-carboxamide;
5-(2-{5-[(hydroxymethyl)carbamoyl]-1,4-dioxan-2-yl}methoxy)-N-(4-fluoro-3-chlorobenzyl)-6-fluoro-4-oxo-3,4-dihydroquinazoline-2-carboxamide;
5-(2-{5-[(hydroxymethyl)carbamoyl]-1,4-dioxan-2-yl}methoxy)-N-(3-methoxybenzyl)-6-fluoro-4-oxo-3,4-dihydroquinazoline-2-carboxamide;
5-(2-{5-[(hydroxymethyl)carbamoyl]-1,4-dioxan-2-yl}methoxy)-N-(3-chlorobenzyl)-6-fluoro-4-oxo-3,4-dihydroquinazoline-2-carboxamide;
N-(4-fluoro-3-methoxybenzyl)-6-fluoro-5-{2-[5-(methylcarbamoyl)-1,4-dioxan-2-yl]methoxy}-4-oxo-3,4-dihydroquinazoline-2-carboxamide;
N-(4-fluoro-3-chlorobenzyl)-6-fluoro-5-{2-[5-(methylcarbamoyl)-1,4-dioxan-2-yl]methoxy}-4-oxo-3,4-dihydroquinazoline-2-carboxamide;
N-(3-methoxybenzyl)-6-fluoro-5-{2-[5-(methylcarbamoyl)-1,4-dioxan-2-yl]methoxy}-4-oxo-3,4-dihydroquinazoline-2-carboxamide;
N-(3-chlorobenzyl)-6-fluoro-5-{2-[5-(methylcarbamoyl)-1,4-dioxan-2-yl]methoxy}-4-oxo-3,4-dihydroquinazoline-2-carboxamide;
5-(2-{2-[(4-fluoro-3-methoxybenzyl)carbamoyl]-6-fluoro-4-oxo-3,4-dihydroquinazolin-5-yloxy}methyl)-1,4-dioxane-2-carboxylic acid;
5-(2-{2-[(4-fluoro-3-chlorobenzyl)carbamoyl]-6-fluoro-4-oxo-3,4-dihydroquinazolin-5-yloxy}methyl)-1,4-dioxane-2-carboxylic acid;
5-(2-{2-[(3-methoxybenzyl)carbamoyl]-6-fluoro-4-oxo-3,4-dihydroquinazolin-5-yloxy}methyl)-1,4-dioxane-2-carboxylic acid;
5-(2-{2-[(3-chlorobenzyl)carbamoyl]-6-fluoro-4-oxo-3,4-dihydroquinazolin-5-yloxy}methyl)-1,4-dioxane-2-carboxylic acid;
N-(4-fluoro-3-methoxybenzyl)-5-{2-[5-(cyanomethyl)-1,4-dioxan-2-yl]methoxy}-6-fluoro-4-oxo-3,4-dihydroquinazoline-2-carboxamide;
N-(4-fluoro-3-chlorobenzyl)-5-{2-[5-(cyanomethyl)-1,4-dioxan-2-yl]methoxy}-6-fluoro-4-oxo-3,4-dihydroquinazoline-2-carboxamide;
N-(3-methoxybenzyl)-5-{2-[5-(cyanomethyl)-1,4-dioxan-2-yl]methoxy}-6-fluoro-4-oxo-3,4-dihydroquinazoline-2-carboxamide;
N-(3-chlorobenzyl)-5-{2-[5-(cyanomethyl)-1,4-dioxan-2-yl]methoxy}-6-fluoro-4-oxo-3,4-dihydroquinazoline-2-carboxamide;
N-(4-fluoro-3-methoxybenzyl)-6-fluoro-5-{2-[5-(methylsulfonamidomethyl)-1,4-dioxan-2-yl]methoxy}-4-oxo-3,4-dihydroquinazoline-2-carboxamide;
N-(4-fluoro-3-chlorobenzyl)-6-fluoro-5-{2-[5-(methylsulfonamidomethyl)-1,4-dioxan-2-yl]methoxy}-4-oxo-3,4-dihydroquinazoline-2-carboxamide;
N-(3-methoxybenzyl)-6-fluoro-5-{2-[5-(methylsulfonamidomethyl)-1,4-dioxan-2-yl]methoxy}-4-oxo-3,4-dihydroquinazoline-2-carboxamide;
N-(3-chlorobenzyl)-6-fluoro-5-{2-[5-(methylsulfonamidomethyl)-1,4-dioxan-2-yl]methoxy}-4-oxo-3,4-dihydroquinazoline-2-carboxamide;
N-(4-fluoro-3-methoxybenzyl)-6-fluoro-5-{2-[5-(hydroxymethyl)-1,4-dioxan-2-yl]methoxy}-4-oxo-3,4-dihydroquinazoline-2-carboxamide;
N-(4-fluoro-3-chlorobenzyl)-6-fluoro-5-{2-[5-(hydroxymethyl)-1,4-dioxan-2-yl]methoxy}-4-oxo-3,4-dihydroquinazoline-2-carboxamide;
N-(3-methoxybenzyl)-6-fluoro-5-{2-[5-(hydroxymethyl)-1,4-dioxan-2-yl]methoxy}-4-oxo-3,4-dihydroquinazoline-2-carboxamide N-(3-chlorobenzyl)-6-fluoro-5-{2-[5-(hydroxymethyl)-1,4-dioxan-2-yl]methoxy}-4-oxo-3,4-dihydroquinazoline-2-carboxamide;
N-(4-fluoro-3-methoxybenzyl)-6-fluoro-5-{2-[5-(methylsulfonylmethyl)-1,4-dioxan-2-yl]methoxy}-4-oxo-3,4-dihydroquinazoline-2-carboxamide;
N-(4-fluoro-3-chlorobenzyl)-6-fluoro-5-{2-[5-(methylsulfonylmethyl)-1,4-dioxan-2-yl]methoxy}-4-oxo-3,4-dihydroquinazoline-2-carboxamide;
N-(3-methoxybenzyl)-6-fluoro-5-{2-[5-(methylsulfonylmethyl)-1,4-dioxan-2-yl]methoxy}-4-oxo-3,4-dihydroquinazoline-2-carboxamide;
N-(3-chlorobenzyl)-6-fluoro-5-{2-[5-(methylsulfonylmethyl)-1,4-dioxan-2-yl]methoxy}-4-oxo-3,4-dihydroquinazoline-2-carboxamide;
N-(4-fluoro-3-methoxybenzyl)-5-[2-(5-cyano-1,4-dioxan-2-yl)methoxy]-6-fluoro-4-oxo-3,4-dihydroquinazoline-2-carboxamide;
N-(4-fluoro-3-chlorobenzyl)-5-[2-(5-cyano-1,4-dioxan-2-yl)methoxy]-6-fluoro-4-oxo-3,4-dihydroquinazoline-2-carboxamide;
N-(3-methoxybenzyl)-5-[2-(5-cyano-1,4-dioxan-2-yl)methoxy]-6-fluoro-4-oxo-3,4-dihydroquinazoline-2-carboxamide;
N-(3-chlorobenzyl)-5-[2-(5-cyano-1,4-dioxan-2-yl)methoxy]-6-fluoro-4-oxo-3,4-dihydroquinazoline-2-carboxamide;
5-[(5-(aminomethyl)-1,4-dioxan-2-yl)methoxy]-6-fluoro-N-(3-methoxy-4-fluorobenzyl)-4-oxo-3,4-dihydroquinazoline-2-carboxamide;
5-[(5-(aminomethyl)-1,4-dioxan-2-yl)methoxy]-6-fluoro-N-(3-chloro-4-fluorobenzyl)-4-oxo-3,4-dihydroquinazoline-2-carboxamide;
5-[(5-(aminomethyl)-1,4-dioxan-2-yl)methoxy]-6-fluoro-N-(3-methoxybenzyl)-4-oxo-3,4-dihydroquinazoline-2-carboxamide;
5-[(5-(aminomethyl)-1,4-dioxan-2-yl)methoxy]-6-fluoro-N-(3-chlorobenzyl)-4-oxo-3,4-dihydroquinazoline-2-carboxamide;
N-(4-fluoro-3-methoxybenzyl)-5-{2-[(1S,4R)-7-oxa-bicyclo[2.2.1]heptan-2-yl]ethoxy}-6-fluoro-4-oxo-3,4-dihydroquinazoline-2-carboxamide;
N-(4-fluoro-3-chlorobenzyl)-5-{2-[(1S,4R)-7-oxa-bicyclo[2.2.1]heptan-2-yl]ethoxy}-6-fluoro-4-oxo-3,4-dihydroquinazoline-2-carboxamide;
N-(3-methoxybenzyl)-5-{2-[(1S,4R)-7-oxa-bicyclo[2.2.1]heptan-2-yl]ethoxy}-6-fluoro-4-oxo-3,4-dihydroquinazoline-2-carboxamide;
N-(3-chlorobenzyl)-5-{2-[(1S,4R)-7-oxa-bicyclo[2.2.1]heptan-2-yl]ethoxy}-6-fluoro-4-oxo-3,4-dihydroquinazoline-2-carboxamide;
N-(4-fluoro-3-methoxybenzyl)-6-fluoro-5-{2-[3-hydroxy-3-(hydroxymethyl)cyclopentyl]ethoxy}-4-oxo-3,4-dihydroquinazoline-2-carboxamide;
N-(4-fluoro-3-chlorobenzyl)-6-fluoro-5-{2-[3-hydroxy-3-(hydroxymethyl)cyclopentyl]ethoxy}-4-oxo-3,4-dihydroquinazoline-2-carboxamide;
N-(3-methoxybenzyl)-6-fluoro-5-{2-[3-hydroxy-3-(hydroxymethyl)cyclopentyl]ethoxy}-4-oxo-3,4-dihydroquinazoline-2-carboxamide;
N-(3-chlorobenzyl)-6-fluoro-5-{2-[3-hydroxy-3-(hydroxymethyl)cyclopentyl]ethoxy}-4-oxo-3,4-dihydroquinazoline-2-carboxamide;
N-(4-fluoro-3-methoxybenzyl)-5-[2-(3-cyanocyclopentyl)ethoxy]-6-fluoro-4-oxo-3,4-dihydroquinazoline-2-carboxamide;
N-(4-fluoro-3-chlorobenzyl)-5-[2-(3-cyanocyclopentyl)ethoxy]-6-fluoro-4-oxo-3,4-dihydroquinazoline-2-carboxamide;
N-(3-methoxybenzyl)-5-[2-(3-cyanocyclopentyl)ethoxy]-6-fluoro-4-oxo-3,4-dihydroquinazoline-2-carboxamide;
N-(3-chlorobenzyl)-5-[2-(3-cyanocyclopentyl)ethoxy]-6-fluoro-4-oxo-3,4-dihydroquinazoline-2-carboxamide;
N-(4-fluoro-3-methoxybenzyl)-6-fluoro-5-{2-[3-(2-hydroxyacetamido)cyclopentyl]ethoxy}-4-oxo-3,4-dihydroquinazoline-2-carboxamide;
N-(4-fluoro-3-chlorobenzyl)-6-fluoro-5-{2-[3-(2-hydroxyacetamido)cyclopentyl]ethoxy}-4-oxo-3,4-dihydroquinazoline-2-carboxamide;
N-(3-methoxybenzyl)-6-fluoro-5-{2-[3-(2-hydroxyacetamido)cyclopentyl]ethoxy}-4-oxo-3,4-dihydroquinazoline-2-carboxamide;
N-(3-chlorobenzyl)-6-fluoro-5-{2-[3-(2-hydroxyacetamido)cyclopentyl]ethoxy}-4-oxo-3,4-dihydroquinazoline-2-carboxamide;
N-(4-fluoro-3-methoxybenzyl)-5-[2-(3-acetamidocyclopentyl)ethoxy]-6-fluoro-4-oxo-3,4-dihydroquinazoline-2-carboxamide;
N-(4-fluoro-3-chlorobenzyl)-5-[2-(3-acetamidocyclopentyl)ethoxy]-6-fluoro-4-oxo-3,4-dihydroquinazoline-2-carboxamide;
N-(3-methoxybenzyl)-5-[2-(3-acetamidocyclopentyl)ethoxy]-6-fluoro-4-oxo-3,4-dihydroquinazoline-2-carboxamide;
N-(3-chlorobenzyl)-5-[2-(3-acetamidocyclopentyl)ethoxy]-6-fluoro-4-oxo-3,4-dihydroquinazoline-2-carboxamide;
N-(4-fluoro-3-methoxybenzyl)-6-fluoro-5-{2-[3-(methylsulfonamido)cyclopentyl]ethoxy}-4-oxo-3,4-dihydroquinazoline-2-carboxamide;
N-(4-fluoro-3-chlorobenzyl)-6-fluoro-5-{2-[3-(methylsulfonamido)cyclopentyl]ethoxy}-4-oxo-3,4-dihydroquinazoline-2-carboxamide;
N-(3-methoxybenzyl)-6-fluoro-5-{2-[3-(methylsulfonamido)cyclopentyl]ethoxy}-4-oxo-3,4-dihydroquinazoline-2-carboxamide; or
N-(3-chlorobenzyl)-6-fluoro-5-{2-[3-(methylsulfonamido)cyclopentyl]ethoxy}-4-oxo-3,4-dihydroquinazoline-2-carboxamide.

19. A compound according to claim 4 selected from:
6-fluoro-N-(3-methoxybenzyl)-4-oxo-5-(2-piperidin-4-ylethoxy)-3,4-dihydroquinazoline-2-carboxamide;
2-(4-{2-[(6-fluoro-2-{[(3-methoxybenzyl)amino]carbonyl}-4-oxo-3,4-dihydroquinazolin-5-yl)oxy]ethyl}piperidin-1-yl)-2-oxoethyl acetate;
6-fluoro-5-{2-[1-(2-hydroxyethyl)piperidin-4-yl]ethoxy}-N-(3-methoxybenzyl)-4-oxo-3,4-dihydroquinazoline-2-carboxamide;
N-(3-methoxybenzyl)-6-fluoro-5-{2-[1-(2-hydroxyacetyl)piperidin-4-yl]ethoxy}-4-oxo-3,4-dihydroquinazoline-2-carboxamide;
N-(3-methoxybenzyl)-6-fluoro-5-{2-[1-(methylsulfonyl)piperidin-4-yl]ethoxy}-4-oxo-3,4-dihydroquinazoline-2-carboxamide;
6-fluoro-5-{[4-(hydroxymethyl)cyclohexyl]methoxy}-N43-methoxybenzyl)-4-oxo-3,4-dihydroquinazoline-2-carboxamide;
N-(3-methoxybenzyl)-6-fluoro-5-{2-[5-(hydroxymethyl)-1,4-dioxan-2-yl]methoxy}-4-oxo-3,4-dihydroquinazoline-2-carboxamide; or
5-[(5-(aminomethyl)-1,4-dioxan-2-yl)methoxy]-6-fluoro-N43-methoxybenzyl)-4-oxo-3,4-dihydroquinazoline-2-carboxamide;
or a pharmaceutically acceptable salt thereof.

20. A compound according to claim 4 selected from:
6-fluoro-N-(3-methoxybenzyl)-4-oxo-5-(2-piperidin-4-ylethoxy)-3,4-dihydroquinazoline-2-carboxamide;
2-(4-{2-[(6-fluoro-2-{[(3-methoxybenzyl)amino]carbonyl}-4-oxo-3,4-dihydroquinazolin-5-yl)oxy]ethyl}piperidin-1-yl)-2-oxoethyl acetate;
6-fluoro-5-{2-[1-(2-hydroxyethyl)piperidin-4-yl]ethoxy}-N43-methoxybenzyl)-4-oxo-3,4-dihydroquinazoline-2-carboxamide;
6-fluoro-5-[2-(1-glycoloylpiperidin-4-yl)ethoxy]-N-(3-methoxybenzyl)-4-oxo-3,4-dihydroquinazoline-2-carboxamide;
6-fluoro-N-(3-methoxybenzyl)-5-{2-[1-(methylsulfonyl)piperidin-4-yl]ethoxy}-4-oxo-3,4-dihydroquinazoline-2-carboxamide;
6-fluoro-5-{[trans-4-(hydroxymethyl)cyclohexyl]methoxy}-N43-methoxybenzyl)-4-oxo-3,4-dihydroquinazoline-2-carboxamide;
6-Fluoro-5-{[(trans-2,5)-5-(hydroxymethyl)-1,4-dioxan-2-yl]methoxy}-N43-methoxybenzyl)-4-oxo-3,4-dihydroquinazoline-2-carboxamide; and
5-{[(trans 2,5)-5-(aminomethyl)-1,4-dioxan-2-yl]methoxy}-6-fluoro-N43-methoxybenzyl)-4-oxo-3,4-dihydroquinazoline-2-carboxamide;
or a pharmaceutically acceptable salt thereof.

21. A compound according to claim 4, 5, 6, 7, 9, or 15 wherein:
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁶ is H, OH, F, Cl, Br, NH₂, or SH;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

22. A compound according to claim 8 wherein:
R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁶ is H, OH, F, Cl, Br, NH₂, or SH;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

23. A compound according to claim 10 wherein:
R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁶ is H, OH, F, Cl, Br, NH₂, or SH;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

24. A compound according to claim 11 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

25. A compound according to claim 12 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

26. A compound according to claim 13 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

27. A compound according to claim 14 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

28. A compound according to claim 16 or 17 wherein:
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁶ is H, OH, F, Cl, Br, NH₂, or SH;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

29. A compound according to claim 1 of Formula III:

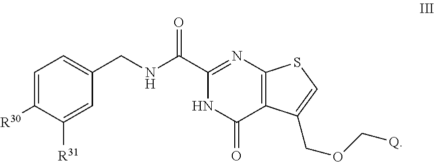

30. A compound according to claim 29 wherein Q is

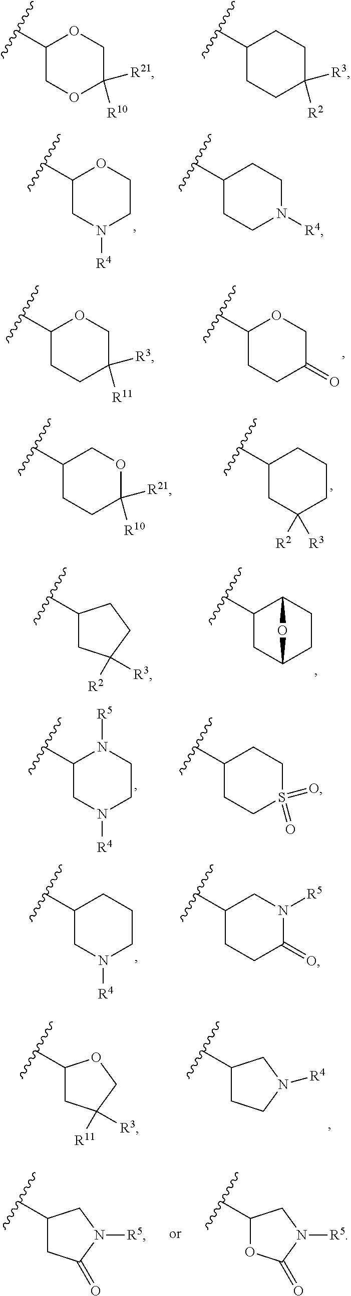

31. A compound according to claim 30 wherein Q is

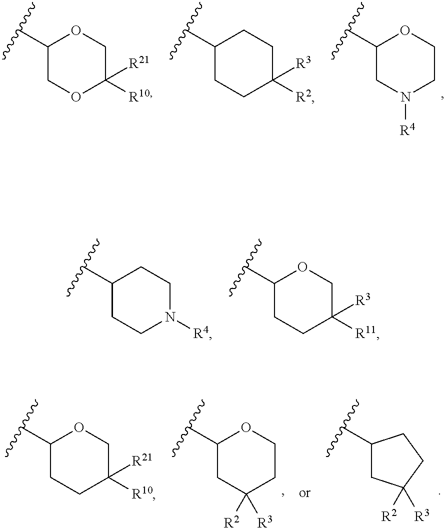

32. A compound according to claim 30 wherein Q is

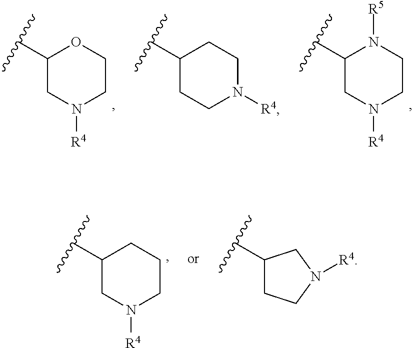

33. A compound according to claim 30 wherein Q is

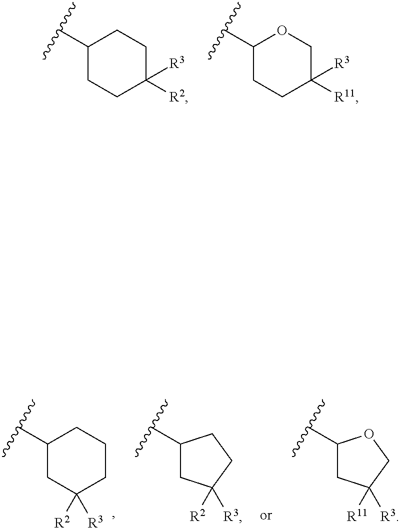

34. A compound according to claim 29 wherein Q is

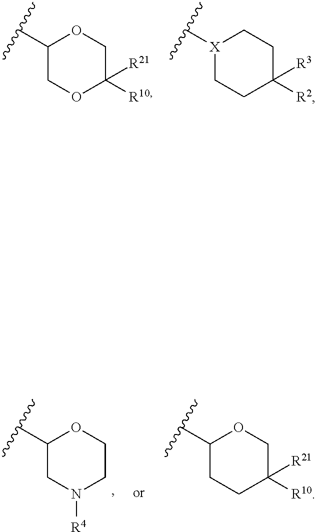

35. A compound according to claim 34 wherein Q is

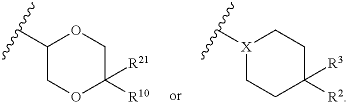

36. A compound according to claim 33 wherein Q is 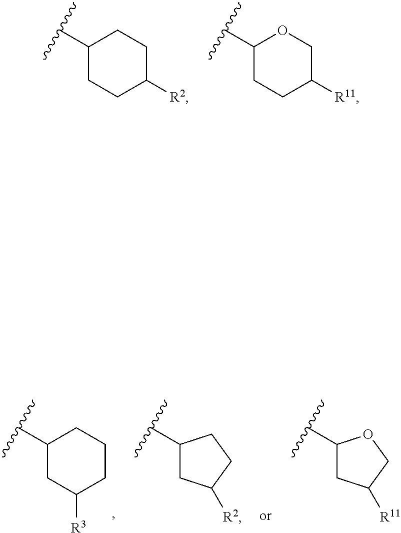
R² is CN, OH, R¹², —C(═O)R⁷, —NR⁸C(═O)R⁹, or —NR⁸SO₂R³⁴; and
R¹¹ is H, CN, OH, R¹², —C(═O)R⁷, —NR⁸C(═O)R⁹, or —NR⁸SO₂R³⁴.

37. A compound according to claim 33 wherein Q is 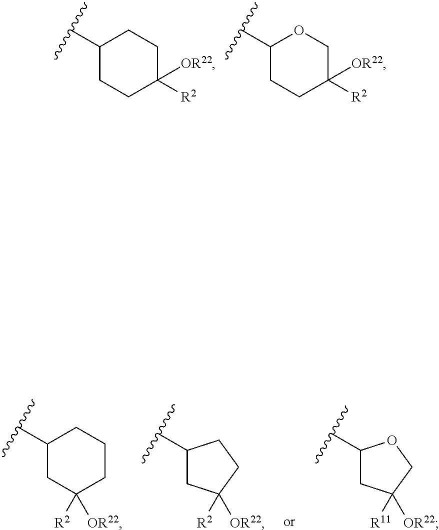
and
R² and R¹¹ are R¹² or —C(═O)R⁷.

38. A compound according to claim 30 wherein Q is 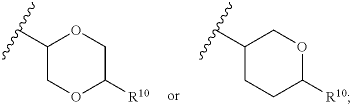
and
R¹⁰ is H, R¹², or —C(═O)R⁷.

39. A compound according to claim 29 of Formula IIIA:

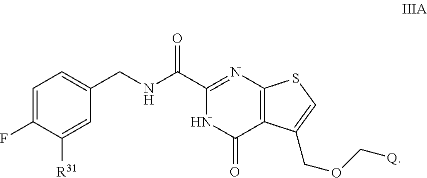

40. A compound according to claim 39 wherein R³¹ is Cl or —OCH₃.

41. A compound according to claim 29 selected from the group consisting of:
N-(4-fluoro-3-methoxybenzyl)-4-oxo-5-{[(tetrahydro-2H-1,1′-dioxothiopyran-4-yl)methoxy]methyl}-3,4-dihydrothieno[2,3-d]pyrimidine-2-carboxamide;
N-(4-fluoro-3-chlorobenzyl)-4-oxo-5-{[(tetrahydro-2H-1,1′-dioxothiopyran-4-yl)methoxy]methyl}-3,4-dihydrothieno[2,3-d]pyrimidine-2-carboxamide;
N-(3-methoxybenzyl)-4-oxo-5-{[(tetrahydro-2H-1,1′-dioxothiopyran-4-yl)methoxy]methyl}-3,4-dihydrothieno[2,3-d]pyrimidine-2-carboxamide;
N-(3-chlorobenzyl)-4-oxo-5-{[(tetrahydro-2H-1,1′-dioxothiopyran-4-yl)methoxy]methyl}-3,4-dihydrothieno[2,3-d]pyrimidine-2-carboxamide;
N-(4-fluoro-3-methoxybenzyl)-5-({[5-(2-hydroxypropan-2-yl)-1,4-dioxan-2-yl]methoxy}methyl)-4-oxo-3,4-dihydrothieno[2,3-d]pyrimidine-2-carboxamide;
N-(4-fluoro-3-chlorobenzyl)-5-({[5-(2-hydroxypropan-2-yl)-1,4-dioxan-2-yl]methoxy}methyl)-4-oxo-3,4-dihydrothieno[2,3-d]pyrimidine-2-carboxamide;
N-(3-methoxybenzyl)-5-({[5-(2-hydroxypropan-2-yl)-1,4-dioxan-2-yl]methoxy}methyl)-4-oxo-3,4-dihydrothieno[2,3-d]pyrimidine-2-carboxamide;
N-(3-chlorobenzyl)-5-({[5-(2-hydroxypropan-2-yl)-1,4-dioxan-2-yl]methoxy}methyl)-4-oxo-3,4-dihydrothieno[2,3-d]pyrimidine-2-carboxamide;
5-[({2-[(4-fluoro-3-methoxybenzyl)carbamoyl]-4-oxo-3,4-dihydrothieno[2,3-d]pyrimidin-5-yl}methoxy)methyl]-1,4-dioxane-2-carboxylic acid5-[({2-[(4-fluoro-3-chlorobenzyl)carbamoyl]-4-oxo-3,4-dihydrothieno[2,3-d]pyrimidin-5-yl}methoxy)methyl]-1,4-dioxane-2-carboxylic acid;
5-[({2-[(3-methoxybenzyl)carbamoyl]-4-oxo-3,4-dihydrothieno[2,3-d]pyrimidin-5-yl}methoxy)methyl]-1,4-dioxane-2-carboxylic acid;
5-[({2-[(3-chlorobenzyl)carbamoyl]-4-oxo-3,4-dihydrothieno[2,3-d]pyrimidin-5-yl}methoxy)methyl]-1,4-dioxane-2-carboxylic acid;
N-(4-fluoro-3-methoxybenzyl)-5-{[(5-carbamoyl-1,4-dioxan-2-yl)methoxy]methyl}-4-oxo-3,4-dihydrothieno[2,3-d]pyrimidine-2-carboxamide;
N-(4-fluoro-3-chlorobenzyl)-5-{[(5-carbamoyl-1,4-dioxan-2-yl)methoxy]methyl}-4-oxo-3,4-dihydrothieno[2,3-d]pyrimidine-2-carboxamide;
N-(3-methoxybenzyl)-5-{[(5-carbamoyl-1,4-dioxan-2-yl)methoxy]methyl}-4-oxo-3,4-dihydrothieno[2,3-d]pyrimidine-2-carboxamide;
N-(3-chlorobenzyl)-5-{[(5-carbamoyl-1,4-dioxan-2-yl)methoxy]methyl}-4-oxo-3,4-dihydrothieno[2,3-d]pyrimidine-2-carboxamide;
5-[({5-[(hydroxymethyl)carbamoyl)-1,4-dioxan-2-yl)methoxy)methyl)-N-(3-methoxybenzyl)-4-oxo-3,4-dihydrothieno[2,3-d]pyrimidine-2-carboxamide;
5-[({5-[(hydroxymethyl)carbamoyl)-1,4-dioxan-2-yl)methoxy)methyl)-N-(3-chlorobenzyl)-4-oxo-3,4-dihydrothieno[2,3-d]pyrimidine-2-carboxamide;
5-[({5-[(hydroxymethyl)carbamoyl)-1,4-dioxan-2-yl)methoxy)methyl)-N-(3-methoxybenzyl)-4-oxo-3,4-dihydrothieno[2,3-d]pyrimidine-2-carboxamide;
5-[({5-[(hydroxymethyl)carbamoyl)-1,4-dioxan-2-yl)methoxy)methyl)-N-(3-chlorobenzyl)-4-oxo-3,4-dihydrothieno[2,3-d]pyrimidine-2-carboxamide;
N-(3-methoxybenzyl)-5-({[5-(methylcarbamoyl)-1,4-dioxan-2-yl]methoxy}methyl)-4-oxo-3,4-dihydrothieno[2,3-d]pyrimidine-2-carboxamide;
N-(3-chlorobenzyl)-5-({[5-(methylcarbamoyl)-1,4-dioxan-2-yl]methoxy}methyl)-4-oxo-3,4-dihydrothieno[2,3-d]pyrimidine-2-carboxamide;
N-(3-methoxybenzyl)-5-({[5-(methylcarbamoyl)-1,4-dioxan-2-yl]methoxy}methyl)-4-oxo-3,4-dihydrothieno[2,3-d]pyrimidine-2-carboxamide;
N-(3-chlorobenzyl)-5-({[5-(methylcarbamoyl)-1,4-dioxan-2-yl]methoxy}methyl)-4-oxo-3,4-dihydrothieno[2,3-d]pyrimidine-2-carboxamide;
N-(4-fluoro-3-methoxybenzyl)-5-({[5-(cyanomethyl)-1,4-dioxan-2-yl]methoxy}methyl)-4-oxo-3,4-dihydrothieno[2,3-d]pyrimidine-2-carboxamide;
N-(4-fluoro-3-chlorobenzyl)-5-({[5-(cyanomethyl)-1,4-dioxan-2-yl]methoxy}methyl)-4-oxo-3,4-dihydrothieno[2,3-d]pyrimidine-2-carboxamide;
N-(3-methoxybenzyl)-5-({[5-(cyanomethyl)-1,4-dioxan-2-yl]methoxy}methyl)-4-oxo-3,4-dihydrothieno[2,3-d]pyrimidine-2-carboxamide;
N-(3-chlorobenzyl)-5-({[5-(cyanomethyl)-1,4-dioxan-2-yl]methoxy}methyl)-4-oxo-3,4-dihydrothieno[2,3-d]pyrimidine-2-carboxamide;
N-(4-fluoro-3-methoxybenzyl)-5-({[5-(methylsulfonamidomethyl)-1,4-dioxan-2-yl]methoxy}methyl)-4-oxo-3,4-dihydrothieno[2,3-d]pyrimidine-2-carboxamide;
N-(4-fluoro-3-chlorobenzyl)-5-({[5-(methylsulfonamidomethyl)-1,4-dioxan-2-yl]methoxy}methyl)-4-oxo-3,4-dihydrothieno[2,3-d]pyrimidine-2-carboxamide;
N-(3-methoxybenzyl)-5-({[5-(methylsulfonamidomethyl)-1,4-dioxan-2-yl]methoxy}methyl)-4-oxo-3,4-dihydrothieno[2,3-d]pyrimidine-2-carboxamide;
N-(3-chlorobenzyl)-5-({[5-(methylsulfonamidomethyl)-1,4-dioxan-2-yl]methoxy}methyl)-4-oxo-3,4-dihydrothieno[2,3-d]pyrimidine-2-carboxamide;
N-(4-fluoro-3-methoxybenzyl)-5-({[5-(hydroxymethyl)-1,4-dioxan-2-yl]methoxy}methyl)-4-oxo-3,4-dihydrothieno[2,3-d]pyrimidine-2-carboxamide;
N-(4-fluoro-3-chlorobenzyl)-5-({[5-(hydroxymethyl)-1,4-dioxan-2-yl]methoxy}methyl)-4-oxo-3,4-dihydrothieno[2,3-d]pyrimidine-2-carboxamide;
N-(3-methoxybenzyl)-5-({[5-(hydroxymethyl)-1,4-dioxan-2-yl]methoxy}methyl)-4-oxo-3,4-dihydrothieno[2,3-d]pyrimidine-2-carboxamide;
N-(3-chlorobenzyl)-5-({[5-(hydroxymethyl)-1,4-dioxan-2-yl]methoxy}methyl)-4-oxo-3,4-dihydrothieno[2,3-d]pyrimidine-2-carboxamide;
N-(4-fluoro-3-methoxybenzyl)-5-({[5-(methylsulfonylmethyl)-1,4-dioxan-2-yl]methoxy}methyl)-4-oxo-3,4-dihydrothieno[2,3-d]pyrimidine-2-carboxamide;
N-(4-fluoro-3-chlorobenzyl)-5-({[5-(methylsulfonylmethyl)-1,4-dioxan-2-yl]methoxy}methyl)-4-oxo-3,4-dihydrothieno[2,3-d]pyrimidine-2-carboxamide;
N-(3-methoxybenzyl)-5-({[5-(methylsulfonylmethyl)-1,4-dioxan-2-yl]methoxy}methyl)-4-oxo-3,4-dihydrothieno[2,3-d]pyrimidine-2-carboxamide;
N-(3-chlorobenzyl)-5-({[5-(methylsulfonylmethyl)-1,4-dioxan-2-yl]methoxy}methyl)-4-oxo-3,4-dihydrothieno[2,3-d]pyrimidine-2-carboxamide;
N-(4-fluoro-3-methoxybenzyl)-5-{[(5-cyano-1,4-dioxan-2-yl)methoxy]methyl}-4-oxo-3,4-dihydrothieno[2,3-d]pyrimidine-2-carboxamide;
N-(4-fluoro-3-chlorobenzyl)-5-{[(5-cyano-1,4-dioxan-2-yl)methoxy]methyl}-4-oxo-3,4-dihydrothieno[2,3-d]pyrimidine-2-carboxamide;
N-(3-methoxybenzyl)-5-{[(5-cyano-1,4-dioxan-2-yl)methoxy]methyl}-4-oxo-3,4-dihydrothieno[2,3-d]pyrimidine-2-carboxamide;
N-(3-chlorobenzyl)-5-{[(5-cyano-1,4-dioxan-2-yl)methoxy]methyl}-4-oxo-3,4-dihydrothieno[2,3-d]pyrimidine-2-carboxamide;
5-({[4-aminomethylcyclohexyl]methoxy}methyl)-N-(3-methoxy-4-fluorobenzyl)-4-oxo-3,4-dihydrothieno[2,3-d]pyrimidine-2-carboxamide;
5-({[4-aminomethylcyclohexyl]methoxy}methyl)-N-(3-chloro-4-fluorobenzyl)-4-oxo-3,4-dihydrothieno[2,3-d]pyrimidine-2-carboxamide;
5-({[4-aminomethylcyclohexyl]methoxy}methyl)-N-(3-methoxybenzyl)-4-oxo-3,4-dihydrothieno[2,3-d]pyrimidine-2-carboxamide;
5-({[4-aminomethylcyclohexyl]methoxy}methyl)-N-(3-chlorobenzyl)-4-oxo-3,4-dihydrothieno[2,3-d]pyrimidine-2-carboxamide;
N-(4-fluoro-3-methoxybenzyl)-5-({[4-(methylcarbamoyl)cyclohexyl]methoxy}methyl)-4-oxo-3,4-dihydrothieno[2,3-d]pyrimidine-2-carboxamide;
N-(4-fluoro-3-chlorobenzyl)-5-({[4-(methylcarbamoyl)cyclohexyl]methoxy}methyl)-4-oxo-3,4-dihydrothieno[2,3-d]pyrimidine-2-carboxamide;
N-(3-methoxybenzyl)-5-({[4-(methylcarbamoyl)cyclohexyl]methoxy}methyl)-4-oxo-3,4-dihydrothieno[2,3-d]pyrimidine-2-carboxamide;
N-(3-chlorobenzyl)-5-({[4-(methylcarbamoyl)cyclohexyl]methoxy}methyl)-4-oxo-3,4-dihydrothieno[2,3-d]pyrimidine-2-carboxamide;
5-[({4-[(hydroxymethyl)carbamoyl]cyclohexyl}methoxy)methyl]-N-(4-fluoro-3-methoxybenzyl)-4-oxo-3,4-dihydrothieno[2,3-d]pyrimidine-2-carboxamide;
5-[({4-[(hydroxymethyl)carbamoyl]cyclohexyl}methoxy)methyl]-N-(4-fluoro-3-chlorobenzyl)-4-oxo-3,4-dihydrothieno[2,3-d]pyrimidine-2-carboxamide;
5-[({4-[(hydroxymethyl)carbamoyl]cyclohexyl}methoxy)methyl]-N-(3-methoxybenzyl)-4-oxo-3,4-dihydrothieno[2,3-d]pyrimidine-2-carboxamide;
5-[({4-[(hydroxymethyl)carbamoyl]cyclohexyl}methoxy)methyl]-N-(3-chlorobenzyl)-4-oxo-3,4-dihydrothieno[2,3-d]pyrimidine-2-carboxamide;
2-{4-[({2-[(4-fluoro-3-methoxybenzyl)carbamoyl]-4-oxo-3,4-dihydrothieno[2,3-d]pyrimidin-5-yl}methoxy)methyl]cyclohexylamino}-2-oxoethyl acetate;
2-{4-[({2-[(4-fluoro-3-chlorobenzyl)carbamoyl]-4-oxo-3,4-dihydrothieno[2,3-d]pyrimidin-5-yl}methoxy)methyl]cyclohexylamino}-2-oxoethyl acetate;
2-{4-[({2-[(3-methoxybenzyl)carbamoyl]-4-oxo-3,4-dihydrothieno[2,3-d]pyrimidin-5-yl}methoxy)methyl]cyclohexylamino}-2-oxoethyl acetate;
2-{4-[({2-[(3-chlorobenzyl)carbamoyl]-4-oxo-3,4-dihydrothieno[2,3-d]pyrimidin-5-yl}methoxy)methyl]cyclohexylamino}-2-oxoethyl acetate;
2-[4-({2-[(4-fluoro-3-methoxybenzyl)carbamoyl]-4-oxo-3,4-dihydrothieno[2,3-d]pyrimidin-5-yl}methoxy)methyl]cyclohexanecarboxylic acid;
2-[4-({2-[(4-fluoro-3-chlorobenzyl)carbamoyl]-4-oxo-3,4-dihydrothieno[2,3-d]pyrimidin-5-yl}methoxy)methyl]cyclohexanecarboxylic acid;
2-[4-({2-[(3-methoxybenzyl)carbamoyl]-4-oxo-3,4-dihydrothieno[2,3-d]pyrimidin-5-yl}methoxy)methyl]cyclohexanecarboxylic acid;
2-[4-({2-[(3-chlorobenzyl)carbamoyl]-4-oxo-3,4-dihydrothieno[2,3-d]pyrimidin-5-yl}methoxy)methyl]cyclohexanecarboxylic acid;
N-(4-fluoro-3-methoxybenzyl)-5-({[4-(2-hydroxyacetamido)cyclohexyl]methoxy}methyl)-4-oxo-3,4-dihydrothieno[2,3-d]pyrimidine-2-carboxamide;
N-(4-fluoro-3-chlorobenzyl)-5-({[4-(2-hydroxyacetamido)cyclohexyl]methoxy}methyl)-4-oxo-3,4-dihydrothieno[2,3-d]pyrimidine-2-carboxamide;
N-(3-methoxybenzyl)-5-({[4-(2-hydroxyacetamido)cyclohexyl]methoxy}methyl)-4-oxo-3,4-dihydrothieno[2,3-d]pyrimidine-2-carboxamide;
N-(3-chlorobenzyl)-5-({[4-(2-hydroxyacetamido)cyclohexyl]methoxy}methyl)-4-oxo-3,4-dihydrothieno[2,3-d]pyrimidine-2-carboxamide;
N-(4-fluoro-3-methoxybenzyl)-5-({[4-(2-hydroxypropan-2-yl)cyclohexyl]methoxy}methyl)-4-oxo-3,4-dihydrothieno[2,3-d]pyrimidine-2-carboxamide;
N-(4-fluoro-3-chlorobenzyl)-5-({[4-(2-hydroxypropan-2-yl)cyclohexyl]methoxy}methyl)-4-oxo-3,4-dihydrothieno[2,3-d]pyrimidine-2-carboxamide;
N-(3-methoxybenzyl)-5-({[4-(2-hydroxypropan-2-yl)cyclohexyl]methoxy}methyl)-4-oxo-3,4-dihydrothieno[2,3-d]pyrimidine-2-carboxamide;
N-(3-chlorobenzyl)-5-({[4-(2-hydroxypropan-2-yl)cyclohexyl]methoxy}methyl)-4-oxo-3,4-dihydrothieno[2,3-d]pyrimidine-2-carboxamide;
N-(4-fluoro-3-methoxybenzyl)-5-({[4-(hydroxymethyl)cyclohexyl]methoxy}methyl)-4-oxo-3,4-dihydrothieno[2,3-d]pyrimidine-2-carboxamide;
N-(4-fluoro-3-chlorobenzyl)-5-({[4-(hydroxymethyl)cyclohexyl]methoxy}methyl)-4-oxo-3,4-dihydrothieno[2,3-d]pyrimidine-2-carboxamide;
N-(3-methoxybenzyl)-5-({[4-(hydroxymethyl)cyclohexyl]methoxy}methyl)-4-oxo-3,4-dihydrothieno[2,3-d]pyrimidine-2-carboxamide;
N-(3-chlorobenzyl)-5-({[4-(hydroxymethyl)cyclohexyl]methoxy}methyl)-4-oxo-3,4-dihydrothieno[2,3-d]pyrimidine-2-carboxamide;
N-(4-fluoro-3-methoxybenzyl)-5-({[4-(methylsulfonamido)cyclohexyl]methoxy}methyl)-4-oxo-3,4-dihydrothieno[2,3-d]pyrimidine-2-carboxamide;
N-(4-fluoro-3-chlorobenzyl)-5-({[4-(methylsulfonamido)cyclohexyl]methoxy}methyl)-4-oxo-3,4-dihydrothieno[2,3-d]pyrimidine-2-carboxamide;
N-(3-methoxybenzyl)-5-({[4-(methylsulfonamido)cyclohexyl]methoxy}methyl)-4-oxo-3,4-dihydrothieno[2,3-d]pyrimidine-2-carboxamide;
N-(3-chlorobenzyl)-5-({[4-(methylsulfonamido)cyclohexyl]methoxy}methyl)-4-oxo-3,4-dihydrothieno[2,3-d]pyrimidine-2-carboxamide;
N-(4-fluoro-3-methoxybenzyl)-5-({[4-(methylsulfonamidomethyl)cyclohexyl]methoxy}methyl)-4-oxo-3,4-dihydrothieno[2,3-d]pyrimidine-2-carboxamide;
N-(4-fluoro-3-chlorobenzyl)-5-({[4-(methylsulfonamidomethyl)cyclohexyl]methoxy}methyl)-4-oxo-3,4-dihydrothieno[2,3-d]pyrimidine-2-carboxamide;
N-(3-methoxybenzyl)-5-({[4-(methylsulfonamidomethyl)cyclohexyl]methoxy}methyl)-4-oxo-3,4-dihydrothieno[2,3-d]pyrimidine-2-carboxamide;
N-(3-chlorobenzyl)-5-({[4-(methylsulfonamidomethyl)cyclohexyl]methoxy}methyl)-4-oxo-3,4-dihydrothieno[2,3-d]pyrimidine-2-carboxamide;
N-(4-fluoro-3-methoxybenzyl)-5-({[4-(methylsulfonyl)cyclohexyl]methoxy}methyl)-4-oxo-3,4-dihydrothieno[2,3-d]pyrimidine-2-carboxamide;
N-(4-fluoro-3-chlorobenzyl)-5-({[4-(methylsulfonyl)cyclohexyl]methoxy}methyl)-4-oxo-3,4-dihydrothieno[2,3-d]pyrimidine-2-carboxamide;
N-(3-methoxybenzyl)-5-({[4-(methylsulfonyl)cyclohexyl]methoxy}methyl)-4-oxo-3,4-dihydrothieno[2,3-d]pyrimidine-2-carboxamide;
N-(3-chlorobenzyl)-5-({[4-(methylsulfonyl)cyclohexyl]methoxy}methyl)-4-oxo-3,4-dihydrothieno[2,3-d]pyrimidine-2-carboxamide;
N-(4-fluoro-3-methoxybenzyl)-5-({[4-(methylsulfonylmethyl)cyclohexyl]methoxy}methyl)-4-oxo-3,4-dihydrothieno[2,3-d]pyrimidine-2-carboxamide;
N-(4-fluoro-3-chlorobenzyl)-5-({[4-(methylsulfonylmethyl)cyclohexyl]methoxy}methyl)-4-oxo-3,4-dihydrothieno[2,3-d]pyrimidine-2-carboxamide;
N-(3-methoxybenzyl)-5-({[4-(methylsulfonylmethyl)cyclohexyl]methoxy}methyl)-4-oxo-3,4-dihydrothieno[2,3-d]pyrimidine-2-carboxamide;
N-(3-chlorobenzyl)-5-({[4-(methylsulfonylmethyl)cyclohexyl]methoxy}methyl)-4-oxo-3,4-dihydrothieno[2,3-d]pyrimidine-2-carboxamide;
N-(4-fluoro-3-methoxybenzyl)-5-({[4-acetamidocyclohexyl)methoxy]methyl}-4-oxo-3,4-dihydrothieno[2,3-d]pyrimidine-2-carboxamide;
N-(4-fluoro-3-chlorobenzyl)-5-{[(4-acetamidocyclohexyl)methoxy]methyl}-4-oxo-3,4-dihydrothieno[2,3-d]pyrimidine-2-carboxamide;
N-(3-methoxybenzyl)-5-{[(4-acetamidocyclohexyl)methoxy]methyl}-4-oxo-3,4-dihydrothieno[2,3-d]pyrimidine-2-carboxamide;
N-(3-chlorobenzyl)-5-{[(4-acetamidocyclohexyl)methoxy]methyl}-4-oxo-3,4-dihydrothieno[2,3-d]pyrimidine-2-carboxamide;
N-(4-fluoro-3-methoxybenzyl)-5-{[(4-carbamoylcyclohexyl)methoxy]methyl}-4-oxo-3,4-dihydrothieno[2,3-d]pyrimidine-2-carboxamide;
N-(4-fluoro-3-chlorobenzyl)-5-{[(4-carbamoylcyclohexyl)methoxy]methyl}-4-oxo-3,4-dihydrothieno[2,3-d]pyrimidine-2-carboxamide;
N-(3-methoxybenzyl)-5-{[(4-carbamoylcyclohexyl)methoxy]methyl}-4-oxo-3,4-dihydrothieno[2,3-d]pyrimidine-2-carboxamide;
N-(3-chlorobenzyl)-5-{[(4-carbamoylcyclohexyl)methoxy]methyl}-4-oxo-3,4-dihydrothieno[2,3-d]pyrimidine-2-carboxamide;
N-(4-fluoro-3-methoxybenzyl)-5-{[(4-cyanocyclohexyl)methoxy]methyl}-4-oxo-3,4-dihydrothieno[2,3-d]pyrimidine-2-carboxamide;
N-(4-fluoro-3-chlorobenzyl)-5-{[(4-cyanocyclohexyl)methoxy]methyl}-4-oxo-3,4-dihydrothieno[2,3-d]pyrimidine-2-carboxamide;
N-(3-methoxybenzyl)-5-{[(4-cyanocyclohexyl)methoxy]methyl}-4-oxo-3,4-dihydrothieno[2,3-d]pyrimidine-2-carboxamide;
N-(3-chlorobenzyl)-5-{[(4-cyanocyclohexyl)methoxy]methyl}-4-oxo-3,4-dihydrothieno[2,3-d]pyrimidine-2-carboxamide;
N-(4-fluoro-3-methoxybenzyl)-5-{[(4-hydroxycyclohexyl)methoxy]methyl}-4-oxo-3,4-dihydrothieno[2,3-d]pyrimidine-2-carboxamide;
N-(4-fluoro-3-chlorobenzyl)-5-{[(4-hydroxycyclohexyl)methoxy]methyl}-4-oxo-3,4-dihydrothieno[2,3-d]pyrimidine-2-carboxamide;
N-(3-methoxybenzyl)-5-{[(4-hydroxycyclohexyl)methoxy]methyl}-4-oxo-3,4-dihydrothieno[2,3-d]pyrimidine-2-carboxamide;
N-(3-chlorobenzyl)-5-{[(4-hydroxycyclohexyl)methoxy]methyl}-4-oxo-3,4-dihydrothieno[2,3-d]pyrimidine-2-carboxamide;
N-(4-fluoro-3-methoxybenzyl)-5-{[(4-aminocyclohexyl)methoxy]methyl}-4-oxo-3,4-dihydrothieno[2,3-d]pyrimidine-2-carboxamide;
N-(4-fluoro-3-chlorobenzyl)-5-{[(4-aminocyclohexyl)methoxy]methyl}-4-oxo-3,4-dihydrothieno[2,3-d]pyrimidine-2-carboxamide;
N-(3-methoxybenzyl)-5-{[(4-aminocyclohexyl)methoxy]methyl}-4-oxo-3,4-dihydrothieno[2,3-d]pyrimidine-2-carboxamide;
N-(3-chlorobenzyl)-5-{[(4-aminocyclohexyl)methoxy]methyl}-4-oxo-3,4-dihydrothieno[2,3-d]pyrimidine-2-carboxamide;
N-(4-fluoro-3-methoxybenzyl)-5-({[3-(methylcarbamoyl)cyclopentyl]methoxy}methyl)-4-oxo-3,4-dihydrothieno[2,3-d]pyrimidine-2-carboxamide;
N-(4-fluoro-3-chlorobenzyl)-5-({[3-(methylcarbamoyl)cyclopentyl]methoxy}methyl)-4-oxo-3,4-dihydrothieno[2,3-d]pyrimidine-2-carboxamide;
N-(3-methoxybenzyl)-5-({[3-(methylcarbamoyl)cyclopentyl]methoxy}methyl)-4-oxo-3,4-dihydrothieno[2,3-d]pyrimidine-2-carboxamide;
N-(3-chlorobenzyl)-5-({[3-(methylcarbamoyl)cyclopentyl]methoxy}methyl)-4-oxo-3,4-dihydrothieno[2,3-d]pyrimidine-2-carboxamide;
5-[({3-[(hydroxymethyl)carbamoyl]cyclopentyl}methoxy)methyl]-N-(4-fluoro-3-methoxybenzyl)-4-oxo-3,4-dihydrothieno[2,3-d]pyrimidine-2-carboxamide;
5-[({3-[(hydroxymethyl)carbamoyl]cyclopentyl}methoxy)methyl]-N-(4-fluoro-3-chlorobenzyl)-4-oxo-3,4-dihydrothieno[2,3-d]pyrimidine-2-carboxamide;
5-[({3-[(hydroxymethyl)carbamoyl]cyclopentyl}methoxy)methyl]-N-(3-methoxybenzyl)-4-oxo-3,4-dihydrothieno[2,3-d]pyrimidine-2-carboxamide;
5-[({3-[(hydroxymethyl)carbamoyl]cyclopentyl}methoxy)methyl]-N-(3-chlorobenzyl)-4-oxo-3,4-dihydrothieno[2,3-d]pyrimidine-2-carboxamide;
3-[({2-[(4-fluoro-3-methoxybenzyl)carbamoyl]-4-oxo-3,4-dihydrothieno[2,3-d]pyrimidin-5-yl}methoxy)methyl]cyclopentanecarboxylic acid;
3-[({2-[(4-fluoro-3-chlorobenzyl)carbamoyl]-4-oxo-3,4-dihydrothieno[2,3-d]pyrimidin-5-yl}methoxy)methyl]cyclopentanecarboxylic acid;
3-[({2-[(3-methoxybenzyl)carbamoyl]-4-oxo-3,4-dihydrothieno[2,3-d]pyrimidin-5-yl}methoxy)methyl]cyclopentanecarboxylic acid;
3-[({2-[(3-chlorobenzyl)carbamoyl]-4-oxo-3,4-dihydrothieno[2,3-d]pyrimidin-5-yl}methoxy)methyl]cyclopentanecarboxylic acid;
N-(4-fluoro-3-methoxybenzyl)-5-({[3-(2-hydroxyacetamido)cyclopentyl]methoxy}methyl)-4-oxo-3,4-dihydrothieno[2,3-d]pyrimidine-2-carboxamide;
N-(4-fluoro-3-chlorobenzyl)-5-({[3-(2-hydroxyacetamido)cyclopentyl]methoxy}methyl)-4-oxo-3,4-dihydrothieno[2,3-d]pyrimidine-2-carboxamide;
N-(3-methoxybenzyl)-5-({[3-(2-hydroxyacetamido)cyclopentyl]methoxy}methyl)-4-oxo-3,4-dihydrothieno[2,3-d]pyrimidine-2-carboxamide;
N-(3-chlorobenzyl)-5-({[3-(2-hydroxyacetamido)cyclopentyl]methoxy}methyl)-4-oxo-3,4-dihydrothieno[2,3-d]pyrimidine-2-carboxamide;
N-(4-fluoro-3-methoxybenzyl)-5-({[3-(2-hydroxypropan-2-yl)cyclopentyl]methoxy}methyl)-4-oxo-3,4-dihydrothieno[2,3-d]pyrimidine-2-carboxamide;
N-(4-fluoro-3-chlorobenzyl)-5-({[3-(2-hydroxypropan-2-yl)cyclopentyl]methoxy}methyl)-4-oxo-3,4-dihydrothieno[2,3-d]pyrimidine-2-carboxamide;
N-(3-methoxybenzyl)-5-({[3-(2-hydroxypropan-2-yl)cyclopentyl]methoxy}methyl)-4-oxo-3,4-dihydrothieno[2,3-d]pyrimidine-2-carboxamide;
N-(3-chlorobenzyl)-5-({[3-(2-hydroxypropan-2-yl)cyclopentyl]methoxy}methyl)-4-oxo-3,4-dihydrothieno[2,3-d]pyrimidine-2-carboxamide;
N-(4-fluoro-3-methoxybenzyl)-5-({[3-(hydroxymethyl)cyclopentyl]methoxy}methyl)-4-oxo-3,4-dihydrothieno[2,3-d]pyrimidine-2-carboxamide;
N-(4-fluoro-3-chlorobenzyl)-5-({[3-(hydroxymethyl)cyclopentyl]methoxy}methyl)-4-oxo-3,4-dihydrothieno[2,3-d]pyrimidine-2-carboxamide;
N-(3-methoxybenzyl)-5-({[3-(hydroxymethyl)cyclopentyl]methoxy}methyl)-4-oxo-3,4-dihydrothieno[2,3-d]pyrimidine-2-carboxamide;
N-(3-chlorobenzyl)-5-({[3-(hydroxymethyl)cyclopentyl]methoxy}methyl)-4-oxo-3,4-dihydrothieno[2,3-d]pyrimidine-2-carboxamide;
N-(4-fluoro-3-methoxybenzyl)-5-({[3-(methylsulfonamido)cyclopentyl]methoxy}methyl)-4-oxo-3,4-dihydrothieno[2,3-d]pyrimidine-2-carboxamide;
N-(4-fluoro-3-chlorobenzyl)-5-({[3-(methylsulfonamido)cyclopentyl]methoxy}methyl)-4-oxo-3,4-dihydrothieno[2,3-d]pyrimidine-2-carboxamide;
N-(3-methoxybenzyl)-5-({[3-(methylsulfonamido)cyclopentyl]methoxy}methyl)-4-oxo-3,4-dihydrothieno[2,3-d]pyrimidine-2-carboxamide;
N-(3-chlorobenzyl)-5-({[3-(methylsulfonamido)cyclopentyl]methoxy}methyl)-4-oxo-3,4-dihydrothieno[2,3-d]pyrimidine-2-carboxamide;
N-(4-fluoro-3-methoxybenzyl)-5-({[3-(methylsulfonamidomethyl)cyclopentyl]methoxy}methyl)-4-oxo-3,4-dihydrothieno[2,3-d]pyrimidine-2-carboxamide;
N-(4-fluoro-3-chlorobenzyl)-5-({[3-(methylsulfonamidomethyl)cyclopentyl]methoxy}methyl)-4-oxo-3,4-dihydrothieno[2,3-d]pyrimidine-2-carboxamide;
N-(3-methoxybenzyl)-5-({[3-(methylsulfonamidomethyl)cyclopentyl]methoxy}methyl)-4-oxo-3,4-dihydrothieno[2,3-d]pyrimidine-2-carboxamide;
N-(3-chlorobenzyl)-5-({[3-(methylsulfonamidomethyl)cyclopentyl]methoxy}methyl)-4-oxo-3,4-dihydrothieno[2,3-d]pyrimidine-2-carboxamide;
N-(4-fluoro-3-methoxybenzyl)-5-({[3-(methylsulfonyl)cyclopentyl]methoxy}methyl)-4-oxo-3,4-dihydrothieno[2,3-d]pyrimidine-2-carboxamide;
N-(4-fluoro-3-chlorobenzyl)-5-({[3-(methylsulfonyl)cyclopentyl]methoxy}methyl)-4-oxo-3,4-dihydrothieno[2,3-d]pyrimidine-2-carboxamide;
N-(3-methoxybenzyl)-5-({[3-(methylsulfonyl)cyclopentyl]methoxy}methyl)-4-oxo-3,4-dihydrothieno[2,3-d]pyrimidine-2-carboxamide;
N-(3-chlorobenzyl)-5-({[3-(methylsulfonyl)cyclopentyl]methoxy}methyl)-4-oxo-3,4-dihydrothieno[2,3-d]pyrimidine-2-carboxamide;
N-(4-fluoro-3-methoxybenzyl)-5-({[3-(methylsulfonylmethyl)cyclopentyl]methoxy}methyl)-4-oxo-3,4-dihydrothieno[2,3-d]pyrimidine-2-carboxamide;
N-(4-fluoro-3-chlorobenzyl)-5-({[3-(methylsulfonylmethyl)cyclopentyl]methoxy}methyl)-4-oxo-3,4-dihydrothieno[2,3-d]pyrimidine-2-carboxamide;
N-(3-methoxybenzyl)-5-({[3-(methylsulfonylmethyl)cyclopentyl]methoxy}methyl)-4-oxo-3,4-dihydrothieno[2,3-d]pyrimidine-2-carboxamide;
N-(3-chlorobenzyl)-5-({[3-(methylsulfonylmethyl)cyclopentyl]methoxy}methyl)-4-oxo-3,4-dihydrothieno[2,3-d]pyrimidine-2-carboxamide;
N-(4-fluoro-3-methoxybenzyl)-5-{[(3-acetamidocyclopentyl)methoxy]methyl}-4-oxo-3,4-dihydrothieno[2,3-d]pyrimidine-2-carboxamide;
N-(4-fluoro-3-chlorobenzyl)-5-{[(3-acetamidocyclopentyl)methoxy]methyl}-4-oxo-3,4-dihydrothieno[2,3-d]pyrimidine-2-carboxamide;
N-(3-methoxybenzyl)-5-{[(3-acetamidocyclopentyl)methoxy]methyl}-4-oxo-3,4-dihydrothieno[2,3-d]pyrimidine-2-carboxamide;
N-(3-chlorobenzyl)-5-{[(3-acetamidocyclopentyl)methoxy]methyl}-4-oxo-3,4-dihydrothieno[2,3-d]pyrimidine-2-carboxamide;
N-(4-fluoro-3-methoxybenzyl)-5-{[(3-carbamoylcyclopentyl)methoxy]methyl}-4-oxo-3,4-dihydrothieno[2,3-d]pyrimidine-2-carboxamide;
N-(4-fluoro-3-chlorobenzyl)-5-{[(3-carbamoylcyclopentyl)methoxy]methyl}-4-oxo-3,4-dihydrothieno[2,3-d]pyrimidine-2-carboxamide;
N-(3-methoxybenzyl)-5-{[(3-carbamoylcyclopentyl)methoxy]methyl}-4-oxo-3,4-dihydrothieno[2,3-d]pyrimidine-2-carboxamide;
N-(3-chlorobenzyl)-5-{[(3-carbamoylcyclopentyl)methoxy]methyl}-4-oxo-3,4-dihydrothieno[2,3-d]pyrimidine-2-carboxamide;
N-(4-fluoro-3-methoxybenzyl)-5-{[(3-cyanocyclopentyl)methoxy]methyl}-4-oxo-3,4-dihydrothieno[2,3-d]pyrimidine-2-carboxamide;
N-(4-fluoro-3-chlorobenzyl)-5-{[(3-cyanocyclopentyl)methoxy]methyl}-4-oxo-3,4-dihydrothieno[2,3-d]pyrimidine-2-carboxamide;
N-(3-methoxybenzyl)-5-{[(3-cyanocyclopentyl)methoxy]methyl}-4-oxo-3,4-dihydrothieno[2,3-d]pyrimidine-2-carboxamide;
N-(3-chlorobenzyl)-5-{[(3-cyanocyclopentyl)methoxy]methyl}-4-oxo-3,4-dihydrothieno[2,3-d]pyrimidine-2-carboxamide;
N-(4-fluoro-3-methoxybenzyl)-5-{[(3-hydroxycyclopentyl)methoxy]methyl}-4-oxo-3,4-dihydrothieno[2,3-d]pyrimidine-2-carboxamide;
N-(4-fluoro-3-chlorobenzyl)-5-{[(3-hydroxycyclopentyl)methoxy]methyl}-4-oxo-3,4-dihydrothieno[2,3-d]pyrimidine-2-carboxamide;
N-(3-methoxybenzyl)-5-{[(3-hydroxycyclopentyl)methoxy]methyl}-4-oxo-3,4-dihydrothieno[2,3-d]pyrimidine-2-carboxamide;
N-(3-chlorobenzyl)-5-{[(3-hydroxycyclopentyl)methoxy]methyl}-4-oxo-3,4-dihydrothieno[2,3-d]pyrimidine-2-carboxamide;
N-(4-fluoro-3-methoxybenzyl)-5-({[1-(2-hydroxyacetyl)piperidin-4-yl]methoxy}methyl)-4-oxo-3,4-dihydrothieno[2,3-d]pyrimidine-2-carboxamide;
N-(4-fluoro-3-chlorobenzyl)-5-({[1-(2-hydroxyacetyl)piperidin-4-yl]methoxy}methyl)-4-oxo-3,4-dihydrothieno[2,3-d]pyrimidine-2-carboxamide;
N-(3-methoxybenzyl)-5-({[1-(2-hydroxyacetyl)piperidin-4-yl]methoxy}methyl)-4-oxo-3,4-dihydrothieno[2,3-d]pyrimidine-2-carboxamide;
N-(3-chlorobenzyl)-5-({[1-(2-hydroxyacetyl)piperidin-4-yl]methoxy}methyl)-4-oxo-3,4-dihydrothieno[2,3-d]pyrimidine-2-carboxamide;
N-(4-fluoro-3-methoxybenzyl)-5-{[(1-acetylpiperidin-4-yl)methoxy]methyl}-4-oxo-3,4-dihydrothieno[2,3-d]pyrimidine-2-carboxamide;
N-(4-fluoro-3-chlorobenzyl)-5-{[(1-acetylpiperidin-4-yl)methoxy]methyl}-4-oxo-3,4-dihydrothieno[2,3-d]pyrimidine-2-carboxamide;
N-(3-methoxybenzyl)-5-{[(1-acetylpiperidin-4-yl)methoxy]methyl}-4-oxo-3,4-dihydrothieno[2,3-d]pyrimidine-2-carboxamide;
N-(3-chlorobenzyl)-5-{[(1-acetylpiperidin-4-yl)methoxy]methyl}-4-oxo-3,4-dihydrothieno[2,3-d]pyrimidine-2-carboxamide;
N-(4-fluoro-3-methoxybenzyl)-5-[({1-[(5-methyl-1,3,4-oxadiazol-2-yl)methyl]piperidin-4-yl}methoxy)methyl]-4-oxo-3,4-dihydrothieno[2,3-d]pyrimidine-2-carboxamide;
N-(4-fluoro-3-chlorobenzyl)-5-[({1-[(5-methyl-1,3,4-oxadiazol-2-yl)methyl]piperidin-4-yl}methoxy)methyl]-4-oxo-3,4-dihydrothieno[2,3-d]pyrimidine-2-carboxamide;
N-(3-methoxybenzyl)-5-[({1-[(5-methyl-1,3,4-oxadiazol-2-yl)methyl]piperidin-4-yl}methoxy)methyl]-4-oxo-3,4-dihydrothieno[2,3-d]pyrimidine-2-carboxamide;
N-(3-chlorobenzyl)-5-[({1-[(5-methyl-1,3,4-oxadiazol-2-yl)methyl]piperidin-4-yl}methoxy)methyl]-4-oxo-3,4-dihydrothieno[2,3-d]pyrimidine-2-carboxamide;
N-(3-methoxy-4-fluorobenzyl)-4-oxo-5-[(2-piperidin-4-ylethoxy)methyl]-3,4-dihydrothieno[2,3-d]pyrimidine-2-carboxamide hydrochloride;
N-(3-chloro-4-fluorobenzyl)-4-oxo-5-[(2-piperidin-4-ylethoxy)methyl]-3,4-dihydrothieno[2,3-d]pyrimidine-2-carboxamide hydrochloride;
N-(3-methoxybenzyl)-4-oxo-5-[(2-piperidin-4-ylethoxy)methyl]-3,4-dihydrothieno[2,3-d]pyrimidine-2-carboxamide hydrochloride;
N-(3-chlorobenzyl)-4-oxo-5-[(2-piperidin-4-ylethoxy)methyl]-3,4-dihydrothieno[2,3-d]pyrimidine-2-carboxamide hydrochloride;
N-(4-fluoro-3-methoxybenzyl)-5-[({1-[2-(methylsulfonyl)ethyl]piperidin-4-yl}methoxy)methyl]-4-oxo-3,4-dihydrothieno[2,3-d]pyrimidine-2-carboxamide;
N-(4-fluoro-3-chlorobenzyl)-5-[({1-[2-(methylsulfonyl)ethyl]piperidin-4-yl}methoxy)methyl]-4-oxo-3,4-dihydrothieno[2,3-d]pyrimidine-2-carboxamide;
N-(3-methoxybenzyl)-5-[({1-[2-(methylsulfonyl)ethyl]piperidin-4-yl}methoxy)methyl]-4-oxo-3,4-dihydrothieno[2,3-d]pyrimidine-2-carboxamide;
N-(3-chlorobenzyl)-5-[({1-[2-(methylsulfonyl)ethyl]piperidin-4-yl}methoxy)methyl]-4-oxo-3,4-dihydrothieno[2,3-d]pyrimidine-2-carboxamide;
N-(4-fluoro-3-methoxybenzyl)-5-({[1-(2-hydroxyacetyl)piperidin-4-yl]methoxy}methyl)-4-oxo-3,4-dihydrothieno[2,3-d]pyrimidine-2-carboxamide;
N-(4-fluoro-3-chlorobenzyl)-5-({[1-(2-hydroxyacetyl)piperidin-4-yl]methoxy}methyl)-4-oxo-3,4-dihydrothieno[2,3-d]pyrimidine-2-carboxamide;
N-(3-methoxybenzyl)-5-({[1-(2-hydroxyacetyl)piperidin-4-yl]methoxy}methyl)-4-oxo-3,4-dihydrothieno[2,3-d]pyrimidine-2-carboxamide;
N-(3-chlorobenzyl)-5-({[1-(2-hydroxyacetyl)piperidin-4-yl]methoxy}methyl)-4-oxo-3,4-dihydrothieno[2,3-d]pyrimidine-2-carboxamide;
N-(4-fluoro-3-methoxybenzyl)-5-({[1-(2-hydroxyethyl)piperidin-4-yl]methoxy}methyl)-4-oxo-3,4-dihydrothieno[2,3-d]pyrimidine-2-carboxamide;
N-(4-fluoro-3-chlorobenzyl)-5-({[1-(2-hydroxyethyl)piperidin-4-yl]methoxy}methyl)-4-oxo-3,4-dihydrothieno[2,3-d]pyrimidine-2-carboxamide;
N-(3-methoxybenzyl)-5-({[1-(2-hydroxyethyl)piperidin-4-yl]methoxy}methyl)-4-oxo-3,4-dihydrothieno[2,3-d]pyrimidine-2-carboxamide;
N-(3-chlorobenzyl)-5-({[1-(2-hydroxyethyl)piperidin-4-yl]methoxy}methyl)-4-oxo-3,4-dihydrothieno[2,3-d]pyrimidine-2-carboxamide;
N-(4-fluoro-3-methoxybenzyl)-5-({[1-(methylsulfonyl)piperidin-4-yl]methoxy}methyl)-4-oxo-3,4-dihydrothieno[2,3-d]pyrimidine-2-carboxamide;
N-(4-fluoro-3-chlorobenzyl)-5-({[1-(methylsulfonyl)piperidin-4-yl]methoxy}methyl)-4-oxo-3,4-dihydrothieno[2,3-d]pyrimidine-2-carboxamide;
N-(3-methoxybenzyl)-5-({[1-(methylsulfonyl)piperidin-4-yl]methoxy}methyl)-4-oxo-3,4-dihydrothieno[2,3-d]pyrimidine-2-carboxamide;
N-(3-chlorobenzyl)-5-({[1-(methylsulfonyl)piperidin-4-yl]methoxy}methyl)-4-oxo-3,4-dihydrothieno[2,3-d]pyrimidine-2-carboxamide;
N-(4-fluoro-3-methoxybenzyl)-5-({[1-(methylsulfonylmethyl)piperidin-4-yl]methoxy}methyl)-4-oxo-3,4-dihydrothieno[2,3-d]pyrimidine-2-carboxamide;
N-(4-fluoro-3-chlorobenzyl)-5-({[1-(methylsulfonylmethyl)piperidin-4-yl]methoxy}methyl)-4-oxo-3,4-dihydrothieno[2,3-d]pyrimidine-2-carboxamide;
N-(3-methoxybenzyl)-5-({[1-(methylsulfonylmethyl)piperidin-4-yl]methoxy}methyl)-4-oxo-3,4-dihydrothieno[2,3-d]pyrimidine-2-carboxamide;
N-(3-chlorobenzyl)-5-({[1-(methylsulfonylmethyl)piperidin-4-yl]methoxy}methyl)-4-oxo-3,4-dihydrothieno[2,3-d]pyrimidine-2-carboxamide;
N-(4-fluoro-3-methoxybenzyl)-5-{[(1-acetylpiperidin-4-yl)methoxy]methyl}-4-oxo-3,4-dihydrothieno[2,3-d]pyrimidine-2-carboxamide;
N-(4-fluoro-3-chlorobenzyl)-5-{[(1-acetylpiperidin-4-yl)methoxy]methyl}-4-oxo-3,4-dihydrothieno[2,3-d]pyrimidine-2-carboxamide;
N-(3-methoxybenzyl)-5-{[(1-acetylpiperidin-4-yl)methoxy]methyl}-4-oxo-3,4-dihydrothieno[2,3-d]pyrimidine-2-carboxamide;
N-(3-chlorobenzyl)-5-{[(1-acetylpiperidin-4-yl)methoxy]methyl}-4-oxo-3,4-dihydrothieno[2,3-d]pyrimidine-2-carboxamide;
N-(4-fluoro-3-methoxybenzyl)-4-oxo-5-{[(tetrahydro-2H-pyran-2-yl)methoxy]methyl}-3,4-dihydrothieno[2,3-d]pyrimidine-2-carboxamide;
N-(4-fluoro-3-chlorobenzyl)-4-oxo-5-{[(tetrahydro-2H-pyran-2-yl)methoxy]methyl}-3,4-dihydrothieno[2,3-d]pyrimidine-2-carboxamide;
N-(3-methoxybenzyl)-4-oxo-5-{[(tetrahydro-2H-pyran-2-yl)methoxy]methyl}-3,4-dihydrothieno[2,3-d]pyrimidine-2-carboxamide;
N-(3-chlorobenzyl)-4-oxo-5-{[(tetrahydro-2H-pyran-2-yl)methoxy]methyl}-3,4-dihydrothieno[2,3-d]pyrimidine-2-carboxamide;
N-(4-fluoro-3-methoxybenzyl)-5-({[5-(hydroxymethyl)-tetrahydro-2H-pyran-2-yl]methoxy}methyl)-4-oxo-3,4-dihydrothieno[2,3-d]pyrimidine-2-carboxamide;
N-(4-fluoro-3-chlorobenzyl)-5-({[5-(hydroxymethyl)-tetrahydro-2H-pyran-2-yl]methoxy}methyl)-4-oxo-3,4-dihydrothieno[2,3-d]pyrimidine-2-carboxamide;
N-(3-methoxybenzyl)-5-({[5-(hydroxymethyl)-tetrahydro-2H-pyran-2-yl]methoxy}methyl)-4-oxo-3,4-dihydrothieno[2,3-d]pyrimidine-2-carboxamide;
N-(3-chlorobenzyl)-5-({[5-(hydroxymethyl)-tetrahydro-2H-pyran-2-yl]methoxy}methyl)-4-oxo-3,4-dihydrothieno[2,3-d]pyrimidine-2-carboxamide;
N-(4-fluoro-3-methoxybenzyl)-5-[(7-oxa-bicyclo[2.2.1]heptan-2-ylmethoxy)methyl]-4-oxo-3,4-dihydrothieno[2,3-d]pyrimidine-2-carboxamide;
N-(4-fluoro-3-chlorobenzyl)-5-[(7-oxa-bicyclo[2.2.1]heptan-2-ylmethoxy)methyl]-4-oxo-3,4-dihydrothieno[2,3-d]pyrimidine-2-carboxamide;
N-(3-methoxybenzyl)-5-[(7-oxa-bicyclo[2.2.1]heptan-2-ylmethoxy)methyl]-4-oxo-3,4-dihydrothieno[2,3-d]pyrimidine-2-carboxamide;
N-(3-chlorobenzyl)-5-[(7-oxa-bicyclo[2.2.1]heptan-2-ylmethoxy)methyl]-4-oxo-3,4-dihydrothieno[2,3-d]pyrimidine-2-carboxamide;
N-(4-fluoro-3-methoxybenzyl)-5-({[4-(methylsulfonyl)morpholin-2-yl]methoxy}methyl)-4-oxo-3,4-dihydrothieno[2,3-d]pyrimidine-2-carboxamide;
N-(4-fluoro-3-chlorobenzyl)-5-({[4-(methylsulfonyl)morpholin-2-yl]methoxy}methyl)-4-oxo-3,4-dihydrothieno[2,3-d]pyrimidine-2-carboxamide;
N-(3-methoxybenzyl)-5-({[4-(methylsulfonyl)morpholin-2-yl]methoxy}methyl)-4-oxo-3,4-dihydrothieno[2,3-d]pyrimidine-2-carboxamide;
N-(3-chlorobenzyl)-5-({[4-(methylsulfonyl)morpholin-2-yl]methoxy}methyl)-4-oxo-3,4-dihydrothieno[2,3-d]pyrimidine-2-carboxamide;
N-(4-fluoro-3-methoxybenzyl)-5-{[(4-acetylmorpholin-2-yl)methoxy]methyl}-4-oxo-3,4-dihydrothieno[2,3-d]pyrimidine-2-carboxamide;
N-(4-fluoro-3-chlorobenzyl)-5-{[(4-acetylmorpholin-2-yl)methoxy]methyl}-4-oxo-3,4-dihydrothieno[2,3-d]pyrimidine-2-carboxamide;
N-(3-methoxybenzyl)-5-{[(4-acetylmorpholin-2-yl)methoxy]methyl}-4-oxo-3,4-dihydrothieno[2,3-d]pyrimidine-2-carboxamide;
N-(3-chlorobenzyl)-5-{[(4-acetylmorpholin-2-yl)methoxy]methyl}-4-oxo-3,4-dihydrothieno[2,3-d]pyrimidine-2-carboxamide;
N-(4-fluoro-3-methoxybenzyl)-5-({[4-(methylsulfonyl)piperazin-2-yl]methoxy}methyl)-4-oxo-3,4-dihydrothieno[2,3-d]pyrimidine-2-carboxamide;
N-(4-fluoro-3-chlorobenzyl)-5-({[4-(methylsulfonyl)piperazin-2-yl]methoxy}methyl)-4-oxo-3,4-dihydrothieno[2,3-d]pyrimidine-2-carboxamide;
N-(3-methoxybenzyl)-5-({[4-(methylsulfonyl)piperazin-2-yl]methoxy}methyl)-4-oxo-3,4-dihydrothieno[2,3-d]pyrimidine-2-carboxamide;
N-(3-chlorobenzyl)-5-({[4-(methylsulfonyl)piperazin-2-yl]methoxy}methyl)-4-oxo-3,4-dihydrothieno[2,3-d]pyrimidine-2-carboxamide;
N-(4-fluoro-3-methoxybenzyl)-5-({[4-acetylpiperazin-2-yl)methoxy]methyl}-4-oxo-3,4-dihydrothieno[2,3-d]pyrimidine-2-carboxamide;
N-(4-fluoro-3-chlorobenzyl)-5-({[4-acetylpiperazin-2-yl)methoxy]methyl}-4-oxo-3,4-dihydrothieno[2,3-d]pyrimidine-2-carboxamide;
N-(3-methoxybenzyl)-5-({[4-acetylpiperazin-2-yl)methoxy]methyl}-4-oxo-3,4-dihydrothieno[2,3-d]pyrimidine-2-carboxamide;
N-(3-chlorobenzyl)-5-({[4-acetylpiperazin-2-yl)methoxy]methyl}-4-oxo-3,4-dihydrothieno[2,3-d]pyrimidine-2-carboxamide;
N-(4-fluoro-3-methoxybenzyl)-5-({[6-(hydroxymethyl)-tetrahydro-2H-pyran-3-yl]methoxy}methyl)-4-oxo-3,4-dihydrothieno[2,3-d]pyrimidine-2-carboxamide;
N-(4-fluoro-3-chlorobenzyl)-5-({[6-(hydroxymethyl)-tetrahydro-2H-pyran-3-yl]methoxy}methyl)-4-oxo-3,4-dihydrothieno[2,3-d]pyrimidine-2-carboxamide;
N-(3-methoxybenzyl)-5-({[6-(hydroxymethyl)-tetrahydro-2H-pyran-3-yl]methoxy}methyl)-4-oxo-3,4-dihydrothieno[2,3-d]pyrimidine-2-carboxamide; or
N-(3-chlorobenzyl)-5-({[6-(hydroxymethyl)-tetrahydro-2H-pyran-3-yl]methoxy}methyl)-4-oxo-3,4-dihydrothieno[2,3-d]pyrimidine-2-carboxamide;
or a pharmaceutically-acceptable salt thereof.

42. A compound according to claim 41 selected from the group consisting of:
5-[({2-[(4-fluoro-3-methoxybenzyl)carbamoyl]-4-oxo-3,4-dihydrothieno[2,3-d]pyrimidin-5-yl}methoxy)methyl]-1,4-dioxane-2-carboxylic acid;
5-[({2-[(3-methoxybenzyl)carbamoyl]-4-oxo-3,4-dihydrothieno[2,3-d]pyrimidin-5-yl}methoxy)methyl]-1,4-dioxane-2-carboxylic acid;
N-(4-fluoro-3-methoxybenzyl)-5-{[(5-carbamoyl-1,4-dioxan-2-yl)methoxy]methyl}-4-oxo-3,4-dihydrothieno[2,3-d]pyrimidine-2-carboxamide;
N-(4-fluoro-3-methoxybenzyl)-5-({[5-(hydroxymethyl)-1,4-dioxan-2-yl]methoxy}methyl)-4-oxo-3,4-dihydrothieno[2,3-d]pyrimidine-2-carboxamide;
N-(3-methoxybenzyl)-5-({[5-(hydroxymethyl)-1,4-dioxan-2-yl]methoxy}methyl)-4-oxo-3,4-dihydrothieno[2,3-d]pyrimidine-2-carboxamide;
5-({[4-aminomethylcyclohexyl]methoxy}methyl)-N-(3-methoxybenzyl)-4-oxo-3,4-dihydrothieno[2,3-d]pyrimidine-2-carboxamide;
2-{4-[({2-[(4-fluoro-3-methoxybenzyl)carbamoyl]-4-oxo-3,4-dihydrothieno[2,3-d]pyrimidin-5-yl}methoxy)methyl]cyclohexylamino}-2-oxoethyl acetate;
2-{4-[({2-[(3-methoxybenzyl)carbamoyl]-4-oxo-3,4-dihydrothieno[2,3-d]pyrimidin-5-yl}methoxy)methyl]cyclohexylamino}-2-oxoethyl acetate;
2-[4-({2-[(4-fluoro-3-methoxybenzyl)carbamoyl]-4-oxo-3,4-dihydrothieno[2,3-d]pyrimidin-5-yl}methoxy)methyl]cyclohexanecarboxylic acid;
2-[4-({2-[(3-methoxybenzyl)carbamoyl]-4-oxo-3,4-dihydrothieno[2,3-d]pyrimidin-5-yl}methoxy)methyl]cyclohexanecarboxylic acid;
N-(4-fluoro-3-methoxybenzyl)-5-({[4-(2-hydroxyacetamido)cyclohexyl]methoxy}methyl)-4-oxo-3,4-dihydrothieno[2,3-d]pyrimidine-2-carboxamide;
N-(3-methoxybenzyl)-5-({[4-(2-hydroxyacetamido)cyclohexyl]methoxy}methyl)-4-oxo-3,4-dihydrothieno[2,3-d]pyrimidine-2-carboxamide;
N-(4-fluoro-3-methoxybenzyl)-5-({[4-(hydroxymethyl)cyclohexyl]methoxy}methyl)-4-oxo-3,4-dihydrothieno[2,3-d]pyrimidine-2-carboxamide;
N-(3-methoxybenzyl)-5-({[4-(hydroxymethyl)cyclohexyl]methoxy}methyl)-4-oxo-3,4-dihydrothieno[2,3-d]pyrimidine-2-carboxamide;
N-(4-fluoro-3-methoxybenzyl)-5-({[4-(methylsulfonamido)cyclohexyl]methoxy}methyl)-4-oxo-3,4-dihydrothieno[2,3-d]pyrimidine-2-carboxamide;
N-(3-methoxybenzyl)-5-({[4-(methylsulfonamido)cyclohexyl]methoxy}methyl)-4-oxo-3,4-dihydrothieno[2,3-d]pyrimidine-2-carboxamide;
N-(3-methoxybenzyl)-5-({[4-(methylsulfonamidomethyl)cyclohexyl]methoxy}methyl)-4-oxo-3,4-dihydrothieno[2,3-d]pyrimidine-2-carboxamide;
N-(4-fluoro-3-methoxybenzyl)-5-({[4-acetamidocyclohexyl)methoxy]methyl}-4-oxo-3,4-dihydrothieno[2,3-d]pyrimidine-2-carboxamide;
N-(3-methoxybenzyl)-5-{[(4-acetamidocyclohexyl)methoxy]methyl}-4-oxo-3,4-dihydrothieno[2,3-d]pyrimidine-2-carboxamide;
N-(4-fluoro-3-methoxybenzyl)-5-{[(4-carbamoylcyclohexyl)methoxy]methyl}-4-oxo-3,4-dihydrothieno[2,3-d]pyrimidine-2-carboxamide;
N-(4-fluoro-3-methoxybenzyl)-5-{[(4-cyanocyclohexyl)methoxy]methyl}-4-oxo-3,4-dihydrothieno[2,3-d]pyrimidine-2-carboxamide;
N-(3-methoxybenzyl)-5-{[(4-cyanocyclohexyl)methoxy]methyl}-4-oxo-3,4-dihydrothieno[2,3-d]pyrimidine-2-carboxamide;
N-(3-methoxybenzyl)-5-{[(4-aminocyclohexyl)methoxy]methyl}-4-oxo-3,4-dihydrothieno[2,3-d]pyrimidine-2-carboxamide;
N-(3-methoxybenzyl)-5-{[(1-acetylpiperidin-4-yl)methoxy]methyl}-4-oxo-3,4-dihydrothieno[2,3-d]pyrimidine-2-carboxamide;
N-(3-methoxybenzyl)-4-oxo-5-[(2-piperidin-4-ylethoxy)methyl]-3,4-dihydrothieno[2,3-d]pyrimidine-2-carboxamide hydrochloride; and
N-(3-methoxybenzyl)-5-({[1-(methylsulfonyl)piperidin-4-yl]methoxy}methyl)-4-oxo-3,4-dihydrothieno[2,3-d]pyrimidine-2-carboxamide;
or a pharmaceutically acceptable salt thereof.

43. A compound according to claim 29 selected from the group consisting of:
Trans-5-(((2-((4-Fluoro-3-methoxybenzyl)carbamoyl)-4-oxo-3,4-dihydrothieno[2,3-d]pyrimidin-5-yl)methoxy)methyl)-1,4-dioxane-2-carboxylic acid;
Trans-5-(((2-((3-Methoxybenzyl)carbamoyl)-4-oxo-3,4-dihydrothieno[2,3-d]pyrimidin-5-yl)methoxy)methyl)-1,4-dioxane-2-carboxylic acid;
N-(4-Fluoro-3-methoxybenzyl)-5-(((trans-5-carbamoyl-1,4-dioxan-2-yl)methoxy)methyl)-4-oxo-3,4-dihydrothieno[2,3-d]pyrimidine-2-carboxamide;
N-(4-Fluoro-3-methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide;
N-(3-Methoxybenzyl)-5-(((trans-5-(hydroxymethyl)-1,4-dioxan-2-yl)methoxy)methyl)-4-oxo-3,4-dihydrothieno[2,3-d]pyrimidine-2-carboxamide;
5-({[trans-4-aminomethylcyclohexyl]methoxy}methyl)-N-(3-methoxybenzyl)-4-oxo-3,4-dihydrothieno[2,3-d]pyrimidine-2-carboxamide;
2-((trans-1,4)-4-(((2-((4-fluoro-3-methoxybenzyl)carbamoyl)-4-oxo-3,4-dihydrothieno[2,3-d]pyrimidin-5-yl)methoxy)methyl)cyclohexylamino)-2-oxoethyl acetate;
2-((trans-1,4)-4-(((2-((3-methoxybenzyl)carbamoyl)-4-oxo-3,4-dihydrothieno[2,3-d]pyrimidin-5-yl)methoxy)methyl)cyclohexylamino)-2-oxoethyl acetate;
Trans-4-(((2-((4-Fluoro-3-methoxybenzyl)carbamoyl)-4-oxo-3,4-dihydrothieno[2,3-d]pyrimidin-5-yl)methoxy)methyl)cyclohexanecarboxylic acid;
Trans-4-(((2-((3-Methoxybenzyl)carbamoyl)-4-oxo-3,4-dihydrothieno[2,3-d]pyrimidin-5-yl)methoxy)methyl)cyclohexanecarboxylic acid;
N-(4-Fluoro-3-methoxybenzyl)-5-((((trans-1,4)-4-(2-hydroxyacetamido)cyclohexyl)methoxy)methyl)-4-oxo-3,4-dihydrothieno[2,3-d]pyrimidine-2-carboxamide;
N-(3-Methoxybenzyl)-5-((((trans-1,4)-4-(2-hydroxyacetamido)cyclohexyl)methoxy)methyl)-4-oxo-3,4-dihydrothieno[2,3-d]pyrimidine-2-carboxamide;
N-(4-Fluoro-3-methoxybenzyl)-5-(((trans-4-(hydroxymethyl)cyclohexyl)methoxy)methyl)-4-oxo-3,4-dihydrothieno[2,3-d]pyrimidine-2-carboxamide;
N-(3-methoxybenzyl)-5-(((trans-4-(hydroxymethyl)cyclohexyl)methoxy)methyl)-4-oxo-3,4-dihydrothieno[2,3-d]pyrimidine-2-carboxamide;
N-(4-Fluoro-3-methoxybenzyl)-5-[({trans-4-[(methylsulfonyl)amino]cyclohexyl}methoxy)methyl]-4-oxo-3,4-dihydrothieno[2,3-d]pyrimidine-2-carboxamide;
N-(3-Methoxybenzyl)-5-[({trans-4-[(methylsulfonyl)amino]cyclohexyl}methoxy)methyl]-4-oxo-3,4-dihydrothieno[2,3-d]pyrimidine-2-carboxamide;
N-(3-methoxybenzyl)-5-[({trans-4-[(methylsulfonyl)aminomethyl]cyclohexyl}methoxy)methyl]-4-oxo-3,4-dihydrothieno[2,3-d]pyrimidine-2-carboxamide;
5-({[trans-4-(Acetylamino)cyclohexyl]methoxy}methyl)-N-(4-fluoro-3-methoxybenzyl)-4-oxo-3,4-dihydrothieno[2,3-d]pyrimidine-2-carboxamide;
5-({[trans-4-(Acetylamino)cyclohexyl]methoxy}methyl)-N-(3-methoxybenzyl)-4-oxo-3,4-dihydrothieno[2,3-d]pyrimidine-2-carboxamide;
N-(4-Fluoro-3-methoxybenzyl)-5-(((trans-4-carbamoylcyclohexyl)methoxy)methyl)-4-oxo-3,4-dihydrothieno[2,3-d]pyrimidine-2-carboxamide;
N-(4-Fluoro-3-methoxybenzyl)-5-(((trans-4-cyanocyclohexyl)methoxy)methyl)-4-oxo-3,4-dihydrothieno[2,3-d]pyrimidine-2-carboxamide;
N-(3-Methoxybenzyl)-5-(((trans-4-cyanocyclohexyl)methoxy)methyl)-4-oxo-3,4-dihydrothieno[2,3-d]pyrimidine-2-carboxamide;
5-({[trans-4-Aminocyclohexyl]methoxy}methyl)-N-(3-methoxybenzyl)-4-oxo-3,4-dihydrothieno[2,3-d]pyrimidine-2-carboxamide;
5-{[2-(1-Acetylpiperidin-4-yl)ethoxy]methyl}-N-(3-methoxybenzyl)-4-oxo-3,4-dihydrothieno[2,3-d]pyrimidine-2-carboxamide;
N-(3-methoxybenzyl)-4-oxo-5-[(2-piperidin-4-ylethoxy)methyl]-3,4-dihydrothieno[2,3-d]pyrimidine-2-carboxamide hydrochloride; and
N-(3-methoxybenzyl)-5-({2-[1-(methylsulfonyl)piperidin-4-yl]ethoxy}methyl)-4-oxo-3,4-dihydrothieno[2,3-d]pyrimidine-2-carboxamide;
or a pharmaceutically acceptable salt thereof.

44. A compound according to claim 29, 30, 31, or 39 wherein:
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸, R²¹, R²², R²⁴, R²⁵, and R³³ are independently H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁶ is H, OH, F, Cl, Br, NH₂, or SH;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

45. A compound according to claim 32 wherein:
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

46. A compound according to claim 33 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

47. A compound according to claim 34 wherein:
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁶, —NR⁸R³³, —NR²⁴C(═O)R²³, and —NR²⁴SO₂R³⁶, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

48. A compound according to claim 35 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

49. A compound according to claim 36 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

50. A compound according to claim 37 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

51. A compound according to claim 38 wherein:
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, —OCH₂CH₂OR²⁵, cyclopropyl, cyclobutyl, cyclopentyl, cyclohexyl, or CN;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

52. A compound according to claim 40 wherein:
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁵ is H or —(C₁₋₆ alkyl), wherein said C₁₋₆ alkyl may be substituted by one or more R²⁶ substituents, and wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —(C₁₋₆ alkylene)OH, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R⁸ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R⁹ is —(C₁₋₆ alkylene)R²⁸, —NHR²⁴, or —OR²⁵, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be optionally substituted by one or more substituents selected from CN, —OR²³, —SO₂R³⁵, —NR⁸R³³, —NR²⁴C(═O)R²³, and —NR²⁴SO₂R³⁵, provided that any one carbon atom of R¹² is not substituted by more than one CN or more than one —OR²³;
R²¹ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²² is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —(C₁₋₆ alkylene)OH, wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene;
R²⁴ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²⁶ is H, OH, F, Cl, Br, NH₂, or SH;
R²⁹ is H or —C(═O)(C₁₋₆ alkyl), wherein said C₁₋₆ alkyl is methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² is optionally substituted with one, two, or three F;
R³³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R³⁴ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁵ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH.

53. A pharmaceutical composition, comprising a compound according to any one of claims 1 through 52, or a pharmaceutically acceptable salt thereof, admixed with a pharmaceutically acceptable carrier, excipient, or diluent.

54. A method for inhibiting an MMP-13 enzyme in an animal, comprising administering to the animal an MMP-13 inhibiting amount of a compound according to any one of claims 1 through 52, or a pharmaceutically acceptable salt thereof.

55. A method for treating a disease mediated by an MMP-13 enzyme, comprising administering to a patient suffering from such a disease a nontoxic effective amount of a compound according to any one of claims 1 through 52, or a pharmaceutically acceptable salt thereof.

56. A method for treating arthritis, comprising administering to a patient suffering from an arthritis disease a nontoxic antiarthritic effective amount of a compound according to any one of claims 1 through 52, or a pharmaceutically acceptable salt thereof.

57. A method for treating osteoarthritis, comprising administering to a patient suffering from osteoarthritis a nontoxic effective amount of a compound according to any one of claims 1 through 52, or a pharmaceutically acceptable salt thereof.

58. A method for treating rheumatoid arthritis, comprising administering to a patient suffering from rheumatoid arthritis a nontoxic effective amount of a compound according to any one of claims 1 through 52, or a pharmaceutically acceptable salt thereof.

59. A method for treating psoriatic arthritis, comprising administering to a patient suffering from psoriatic arthritis a nontoxic effective amount of a compound according to any one of claims 1 through 52, or a pharmaceutically acceptable salt thereof.

60. A method for treating a cancer, comprising administering to a patient suffering from a cancer a nontoxic anti-cancer effective amount of a compound according to any one of claims 1 through 52, or a pharmaceutically acceptable salt thereof.

61. A method for treating inflammation, comprising administering to a patient suffering from inflammation a nontoxic effective amount of a compound according to any one of claims 1 through 52, or a pharmaceutically acceptable salt thereof.

62. A method for treating chronic obstructive pulmonary disease, comprising administering to a patient suffering from chronic obstructive pulmonary disease a nontoxic effective amount of a compound according to any one of claims 1 through 52, or a pharmaceutically acceptable salt thereof.

63. A method for treating psoriasis, comprising administering to a patient suffering from psoriasis a nontoxic effective amount of a compound according to any one of claims 1 through 52, or a pharmaceutically acceptable salt thereof.

64. A method for treating asthma, comprising administering to a patient suffering from asthma a nontoxic effective amount of a compound according to any one of claims 1 through 52, or a pharmaceutically acceptable salt thereof.

65. A method for treating inflammatory bowel disease, comprising administering to a patient suffering from inflammatory bowel disease a nontoxic effective amount of a compound according to any one of claims 1 through 52, or a pharmaceutically acceptable salt thereof.
